SEQUENCE LISTING (43 sequences)

SEQ ID NO: 1 - protein, 5 aa, synthetic construct
  REGION 1..5: note = VH CDR1
GYNMN

SEQ ID NO: 2 - protein, 7 aa, synthetic construct
  REGION 1..7: note = VH CDR2
HIDPYYG

SEQ ID NO: 3 - protein, 8 aa, synthetic construct
  REGION 1..8: note = VH CDR3
YCVKGGYY

SEQ ID NO: 4 - protein, 11 aa, synthetic construct
  REGION 1..11: note = VL CDR1
RASQDIGSSLN

SEQ ID NO: 5 - protein, 7 aa, synthetic construct
  REGION 1..7: note = VL CDR2
ATSSLDS

SEQ ID NO: 6 - protein, 9 aa, synthetic construct
  REGION 1..9: note = VL CDR3
LQYVSSPPT

SEQ ID NO: 7 - protein, 5 aa, synthetic construct
  REGION 1..5: note = VH CDR1
SYAIS

SEQ ID NO: 8 - protein, 17 aa, synthetic construct
  REGION 1..17: note = VH CDR2
GFDPEDGETIYAQKFQG

SEQ ID NO: 9 - protein, 17 aa, synthetic construct
  REGION 1..17: note = VH CDR3
GRSMVRGVIIPFNGMDV

SEQ ID NO: 10 - protein, 11 aa, synthetic construct
  REGION 1..11: note = VL CDR1
RASQSISSYLN

SEQ ID NO: 11 - protein, 7 aa, synthetic construct
  REGION 1..7: note = VL CDR2
AASSLQS

SEQ ID NO: 12 - protein, 9 aa, synthetic construct
  REGION 1..9: note = VL CDR3
QQSYSTPLT

SEQ ID NO: 13 - protein, 5 aa, synthetic construct
  REGION 1..5: note = VEGF VH CDR1
NYWMH

SEQ ID NO: 14 - protein, 17 aa, synthetic construct
  REGION 1..17: note = VEGF VH CDR2
DINPSNGRTSYKEKFKR

SEQ ID NO: 15 - protein, 12 aa, synthetic construct
  REGION 1..12: note = VEGF VH CDR3
HYDDKYYPLMDY

SEQ ID NO: 16 - protein, 6 aa, synthetic construct
  REGION 1..6: note = DLL4 VH CDR1
TAYYIH

SEQ ID NO: 17 - protein, 17 aa, synthetic construct
  REGION 1..17: note = DLL4 VH CDR2
YISNYNRATNYNQKFKG

SEQ ID NO: 18 - protein, 11 aa, synthetic construct
  REGION 1..11: note = DLL4 V4 CDR3
RDYDYDVGMDY

SEQ ID NO: 19 - protein, 15 aa, synthetic construct
  REGION 1..15: note = VL CDR1
RASESVDNYGISFMK

SEQ ID NO: 20 - protein, 7 aa, synthetic construct
  REGION 1..7: note = VL CDR2
AASNQGS

SEQ ID NO: 21 - protein, 12 aa, synthetic construct
  REGION 1..12: note = VL CDR3
QQSKEVPWTFGG

SEQ ID NO: 22 - protein, 120 aa, synthetic construct
  REGION 1..120: note = Heavy Chain
EVQLQQSGPELEKPGASVKLSCKASGYSFTGYNMNWVKQSHGKSLEWIGHIDPYYGDTSYNQKFRGKATLTVDKSSSTAYMQLKSLTSEDSAVYYCVKGGYYGHWYFDVWGAGTTVTVSS

SEQ ID NO: 23 - protein, 107 aa, synthetic construct
  REGION 1..107: note = Light Chain
DIQMTQSPSSLSASLGERVSLTCRASQDIGSSLNWLQQGPDGTIKRLIYATSSLDSGVPKRFSGSRSGSDYSLTISSLESEDFVDYYCLQYVSSPPTFGAGTKLELK

SEQ ID NO: 24 - protein, 119 aa, synthetic construct
  REGION 1..119: note = Heavy Chain
QVQLVQSGAEVKKPGASVKISCKASGYSFTAYYIHWVKQAPGQGLEWIGYISNYNRATNYNQKFKGRVTFTTDTSTSTAYMELRSLRSDDTAVYYCARDYDYDVGMDYWGQGTLVTVSS

SEQ ID NO: 25 - protein, 111 aa, synthetic construct
  REGION 1..111: note = Light Chain
DIVMTQSPDSLAVSLGERATISCRASESVDNYGISFMKWFQQKPGQPPKLLIYAASNQGSGVPDRFSGSGSGTDFTLTISSLQAEDVAVYYCQQSKEVPWTFGGGTKVEIK

SEQ ID NO: 26 - protein, 126 aa, synthetic construct
  REGION 1..126: note = Heavy Chain
QVQLVQSGAEVKKPGASVKVSCKASGGTFSSYAISWVRQAPGQGLEWMGGFDPEDGETIYAQKFQGRVTMTEDTSTDTAYMELSSLRSEDTAVYYCATGRSMVRGVIIPFNGMDVWGQGTTVTVSS

SEQ ID NO: 27 - protein, 107 aa, synthetic construct
  REGION 1..107: note = Light Chain
DIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 28 - protein, 8 aa, synthetic construct
  REGION 1..8: note = Heavy Chain CDR1
GFSLTSYG

SEQ ID NO: 29 - protein, 7 aa, synthetic construct
  REGION 1..7: note = Heavy Chain CDR2
IYADGST

SEQ ID NO: 30 - protein, 12 aa, synthetic construct
  REGION 1..12: note = Heavy Chain CDR3
ARAYGNYWYIDV

SEQ ID NO: 31 - protein, 6 aa, synthetic construct
  REGION 1..6: note = Light Chain CDR1
ESVSND

SEQ ID NO: 32 - skipped

SEQ ID NO: 33 - protein, 9 aa, synthetic construct
  REGION 1..9: note = Light Chain CDR3
HQAYSSPYT

SEQ ID NO: 34 - protein, 118 aa, synthetic construct
  REGION 1..118: note = Heavy Chain
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 35 - protein, 107 aa, synthetic construct
  REGION 1..107: note = Light Chain
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 36 - protein, 8 aa, synthetic construct
  REGION 1..8: note = Heavy Chain CDR1
GGTFSSYA

SEQ ID NO: 37 - protein, 8 aa, synthetic construct
  REGION 1..8: note = Heavy Chain CDR2
IIPMFDTA

SEQ ID NO: 38 - protein, 13 aa, synthetic construct
  REGION 1..13: note = Heavy Chain CDR3
ARAEHSSTGTFDY

SEQ ID NO: 39 - protein, 6 aa, synthetic construct
  REGION 1..6: note = Light Chain CDR1
QGISSW

SEQ ID NO: 40 - skipped

SEQ ID NO: 41 - protein, 9 aa, synthetic construct
  REGION 1..9: note = Light Chain CDR3
QQANHLPFT

SEQ ID NO: 42 - protein, 120 aa, synthetic construct
  REGION 1..120: note = Heavy Chain
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 43 - protein, 107 aa, synthetic construct
  REGION 1..107: note = Light Chain
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

(19) United States
(12) Patent Application Publication
(10) Pub. No.: US 2026/0038636 A1
(43) Pub. Date: Feb. 5, 2026

(54) METHODS OF TREATING CANCER

(71) Applicant: Feng Biosciences, Inc., Waltham, MA (US)

(72) Inventors: Laura E. Benjamin, Lunenburg, MA (US); Kristen Strand-Tibbitts, Westford, MA (US); Bronislaw Pytowsky, New York, NY (US); Rafael Rosengarten, San Ramon (CA); Luka Ausec, Kranj (SI); Miha Stajdohar, Ljubljana (SI); Matjaz Zganec, Ruse (SI)

(73) Assignee: Feng Biosciences, Inc., Waltham, MA (US)

(21) Appl. No.: 19/355,245

(22) Filed: Oct. 10, 2025

Related U.S. Application Data
(63) Continuation of application No. 17/089,234, filed on Nov. 4, 2020.
(60) Provisional application No. 62/932,307, filed on Nov. 7, 2019.
(60) Provisional application No. 63/008,367, filed on Apr. 10, 2020.
(60) Provisional application No. 63/060,471, filed on Aug. 3, 2020.
(60) Provisional application No. 63/070,131, filed on Aug. 25, 2020.

(51) Int. Cl. G16B 40/00; A61B 5/00; A61K 31/517; A61K 38/17; A61K 38/18; A61K 39/00; A61K 39/395; A61K 45/06; A61P 35/00; A61P 35/04; C07K 16/22; C07K 16/28; C12Q 1/6886; G01N 33/50; G16B 5/20

(52) U.S. Cl. CPC G16B 40/00; A61B 5/7264; A61K 31/517; A61K 38/177; A61K 38/1891; A61K 39/3955; A61K 45/06; A61P 35/00; A61P 35/04; C07K 16/22; C07K 16/2818; C07K 16/2863; C12Q 1/6886; G01N 33/5091; G16B 5/20; A61K 39/00; A61K 2039/505; C07K 2317/31; C07K 2317/76; C12Q 2539/00; C12Q 2545/10; C12Q 2565/00; C12Q 2600/112; C12Q 2600/158; G05B 2219/32335

(57) ABSTRACT

Provided herein are methods of treating a patient afflicted with a tumor according to the tumor's microenvironments (TME). Also provided are gene panels that can be used for identifying a human subject afflicted with a cancer suitable for treatment with a particular therapeutic agent based on the subject's TME.

### CROSS-REFERENCE TO RELATED APPLICATIONS

The present application claims the benefit of U.S. Nonprovisional patent application Ser. No. 17/089,234, filed Nov. 4, 2020; U.S. Provisional Patent Application No. 62/932,307, filed Nov. 7, 2019; U.S. Provisional Patent Application No. 63/008,367, filed Apr. 10, 2020; U.S. Provisional Patent Application No. 63/060,471, filed Aug. 3, 2020; and U.S. Provisional Patent Application No. 63/070,131, filed Aug. 25, 2020, all of which are herein incorporated by reference in their entireties.

### REFERENCE TO SEQUENCE LISTING SUBMITTED ELECTRONICALLY

This application contains a Sequence Listing, which has been submitted electronically in xml format and is hereby incorporated by reference in its entirety. Said xml copy, created on Oct. 10, 2025, is named SeqList2-365536-00114.xml and is 53,064 bytes in size.

### FIELD

The present disclosure relates to methods for classifying tumor microenvironments (TMEs) based on signature scores or predictive models derived from biomarker gene expression data, for identifying subpopulations of cancer patients with specific TMEs for treatment with particular therapies, and for treating patients having specific TMEs with targeted therapies.

### BACKGROUND

A critical problem in the clinical management of cancer is that cancers are highly heterogeneous. Biomarkers to select cancer patients who can receive the maximum benefit from a treatment have typically relied on immunohistochemistry or expression of a drug target (e.g., a receptor), genetic profiles for mutations (e.g., BRCA), or levels of circulating factors. Successful diagnostics have been developed for only a handful of drugs using this approach and have generally been used for targeted therapies to cancer cells, e.g., HERCEPTIN® (trastuzumab) as a treatment targeting cancers overexpressing the HER2/Neu receptor. Accurate prediction of an individual cancer responsiveness to a particular therapy is generally not achievable due to the multiple factors modulating such responsiveness, such as the presence or absence of particular receptors or other cell signaling switches. This tends to result in failed therapies or can lead to substantial overtreatment.

Prediction of clinical outcome in cancer is usually achieved by histopathological evaluation of tissue samples obtained during surgical resection of the primary tumor. Traditional tumor staging (AJCC/UICC-TNM classification) summarizes data on tumor burden (T), presence of cancer cells in draining and regional lymph nodes (N) and evidence for metastases (M). The current classification provides limited prognostic information, and does not predict response to therapy. Numerous patent applications have described methods for the prognosis of the survival time of a patient suffering from a solid cancer and/or methods for assessing the responsiveness of a patient suffering from a solid cancer to antitumoral treatment, e.g., by measuring immunological biomarkers. See, e.g., International Application Publications WO2015007625, WO2014023706, WO2014009535, WO2013186374, WO2013107907, WO2013107900, WO2012095448, WO2012072750 and WO2007045996, all of which are herein incorporated by reference in their entireties. Furthermore, anti-cancer agents can vary in their effectiveness based on the unique patient characteristics.

Accordingly, there is a need for targeted therapeutic strategies that identify patients who are more likely to respond to a particular anti-cancer agent and, thus, improve the clinical outcome for patients diagnosed with cancer.

### BRIEF SUMMARY

The present disclosure provides a method for determining the tumor microenvironment (TME), also known as stromal phenotype or stromal subtype, of a cancer in a subject in need thereof, comprising applying a machine-learning classifier to a plurality of RNA expression levels obtained from a gene panel from a tumor tissue sample from the subject, wherein the machine-learning classifier identifies the subject as exhibiting (i.e., being biomarker positive) or not exhibiting (i.e., being biomarker negative) a TME classification selected from the group consisting of IS (immune suppressed), A (angiogenic), IA (immune active), ID (immune desert), and combinations thereof.

Also provided is a method for treating a human subject afflicted with a cancer comprising administering a TME-class specific therapy to the subject, wherein, prior to the administration, the subject is identified as exhibiting (i.e., being biomarker-positive) or not exhibiting (i.e., being biomarker-negative) a TME determined by applying a machine-learning classifier to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof.

The present disclosure also provides a method for treating a human subject afflicted with a cancer comprising
(i) identifying, prior to the administration, a subject exhibiting (i.e., being biomarker-positive) or not exhibiting (i.e., being biomarker-negative) a TME by applying a machine-learning classifier to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof; and,
(ii) administering a TME-class specific therapy to the subject.

Also provided is a method for identifying a human subject afflicted with a cancer suitable for treatment with a TME-class specific therapy, the method comprising applying a machine-learning classifier to a plurality of RNA expression levels obtained from a gene panel from a tumor tissue sample obtained from the subject, wherein the presence (biomarker positivity, i.e., being biomarker-positive) or absence (biomarker negativity, i.e., being biomarker-negative) of a TME selected from the group consisting of IS (immune suppressed), A (angiogenic), IA (immune active), ID (immune desert), and combinations thereof, indicates that a TME-class specific therapy can be administered to treat the cancer.

In some aspects, the machine-learning classifier is a model obtained by Logistic Regression, Random Forest, Artificial Neural Network (ANN), Support Vector Machine (SVM), XGBoost (XGB), glmnet, cforest, Classification and Regression Trees for Machine-learning (CART), treebag, K-Nearest Neighbors (kNN), or a combination thereof. In some aspects, the machine-learning classifier is an ANN. In some aspects, the ANN is a feed-forward ANN. In some aspects, the ANN is a multi-layer perceptron.

In some aspects, the ANN comprises an input layer, a hidden layer, and an output layer. In some aspects, the input layer comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, or 100 nodes (neurons). In some aspects, each node (neuron) in the input layer corresponds to a gene in the gene panel. In some aspects, the gene panel is selected from the genes presented in TABLE 1 and TABLE 2 (or in any of the gene panels (Genesets) disclosed in FIGS. 28A-G), or from TABLE 5.

In some aspects, the gene panel comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 genes selected from TABLE 1 and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes selected from TABLE 2. In some aspects, the gene panel is a gene panel selected from TABLE 5 or from FIGS. 28A-G.

In some aspects, the sample comprises intratumoral tissue. In some aspects, the RNA expression levels are transcribed RNA expression levels. In some aspects, the RNA expression levels are determined using sequencing or any technology that measures RNA. In some aspects, the sequencing is Next Generation Sequencing (NGS). In some aspects, the NGS is selected from the group consisting of RNA-Seq, EdgeSeq, PCR, Nanostring, whole exome sequencing (WES) or combinations thereof. In some aspects, the RNA expression levels are determined using fluorescence. In some aspects, the RNA expression levels are determined using an Affymetrix microarray or an Agilent microarray. In some aspects, the RNA expression levels are subject to quantile normalization. In some aspects, the quantile normalization comprises binning input RNA level values into quantiles. In some aspects, the input RNA levels are binned into 100 quantiles, 150 quantiles, 200 quantiles, or more. In some aspects, the quantile normalization comprises quantile transforming the RNA expression levels to a normal output distribution function.

In some aspects, the ANN is trained with a training set comprising RNA expression levels for each gene in the gene panel in a plurality of samples obtained from a plurality of subjects, wherein each sample is assigned a TME classification. In some aspects, the TME classification assigned to each sample in the training set is determined by a population-based classifier. In some aspects, the population-based classifier comprises determining a Signature 1 score and a Signature 2 score by measuring the RNA expression levels for each gene in the gene panel in each sample in the training set; wherein the genes used to calculate Signature 1 are genes from TABLE 1 or FIGS. 28A-28G, or a combination thereof, and the genes used to calculate Signature 2 are genes from TABLE 2 or FIGS. 28A-28G, or a combination thereof; and wherein
(i) the TME classification assigned is IA if the Signature 1 score is negative and the Signature 2 score is positive (i.e., the subject would be considered IA biomarker-positive);
(ii) the TME classification assigned is IS if the Signature 1 score is positive and the Signature 2 score is positive (i.e., the subject would be considered IS biomarker-positive);
(iii) the TME classification assigned is ID if the Signature 1 score is negative and the Signature 2 score is negative (i.e., the subject would be considered ID biomarker-positive); and,
(iv) the TME classification assigned is A if the Signature 1 score is positive and the Signature 2 score is negative (i.e., the subject would be considered A biomarker-positive).

In some aspects, the calculation of a Signature 1 score comprises
(i) measuring the expression level for each gene from TABLE 1, or FIGS. 28A-28G, or a combination thereof, in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and, (iv) adding all the values obtained in step (iii) and dividing the resulting number by the square root of the number of genes in the gene panel;
wherein if the value obtained in (iv) is above zero, the signature score is a positive signature score, and wherein if the value obtained in (iv) is below zero, the signature score is a negative signature score.

In some aspects, the calculation of a Signature 2 score comprises
(i) measuring the expression level for each gene from TABLE 2, or FIGS. 28A-28G, or a combination thereof, in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and, (iv) adding all the values obtained in step (iii) and dividing the resulting number by the square root of the number of genes in the gene panel;
wherein if the value obtained in (iv) is above zero, the signature score is a positive signature score, and wherein if the value obtained in (iv) is below zero, the signature score is a negative signature score.

In some aspects, the ANN is trained by backpropagation. In some aspects, the hidden layer comprises 2 nodes (neurons). In some aspects, a sigmoid activation function is applied to the hidden layer. In some aspects, the sigmoid activation function is a hyperbolic tangent function. In some aspects, the output layer comprises 4 nodes (neurons). In some aspects, each one of the 4 output nodes (neurons) in the output layer corresponds to a TME output class, wherein the 4 TME output classes are IA (immune active), IS (immune suppressed), ID (immune desert), and A (angiogenic). In some aspects, the ANN methods disclosed herein further comprise applying a logistic regression classifier comprising a Softmax function to the output of the ANN, wherein the Softmax function assigns probabilities to each TME output class. In some aspects, the Softmax function is implemented through an additional neural network layer. In some aspects, the additional network layer is interposed between the hidden layer and the output layer. In some aspects, the additional network layer has the same number of nodes (neurons) as the output layer.

The present disclosure also provides an ANN for determining the tumor microenvironment (TME) of a cancer in a subject in need thereof, wherein the ANN identifies the subject as exhibiting (i.e., being biomarker-positive) or not exhibiting (i.e., being biomarker-negative) a TME selected from the group consisting of IS (immune suppressed), A (angiogenic), IA (immune active), ID (immune desert), and combinations thereof using as input RNA expression levels obtained from a gene panel from a tumor tissue sample from the subject, and wherein the presence or absence of a TME indicates that the subject can be effectively treated with TME-class specific therapy, which can be a drug, a combination of drugs, or a clinical therapy that has a mechanism of action that addresses the pathology.

In some aspects, the ANN is a feed-forward ANN. In some aspects, the ANN is a multi-layer perceptron. In some aspects, the ANN comprises an input layer, a hidden layer, and an output layer. In some aspects, the input layer comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, or 100 nodes (neurons). In some aspects, each node (neuron) in the input layer corresponds to a gene in the gene panel. In some aspects, the gene panel is selected from the genes presented in TABLE 1 and TABLE 2 (or in any of the gene panels (Genesets) disclosed in FIGS. 28A-G), or TABLE 5. In some aspects, the gene panel comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 genes selected from TABLE 1 and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes selected from TABLE 2. In some aspects, the gene panel is a gene panel selected from TABLE 5 or from FIGS. 28A-G. In some aspects, the sample comprises intratumoral tissue. In some aspects, the RNA expression levels are transcribed RNA expression levels. In some aspects, the RNA expression levels are determined using sequencing or any technology that measures RNA. In some aspects, the sequencing is Next Generation Sequencing (NGS). In some aspects, the NGS is selected from the group consisting of RNA-Seq, EdgeSeq, PCR, Nanostring, whole exome sequencing (WES) or combinations thereof.

In some aspects, the RNA expression levels are determined using fluorescence. In some aspects, the RNA expression levels are determined using an Affymetrix microarray or an Agilent microarray. In some aspects, RNA expression levels are subject to quantile normalization. In some aspects, the quantile normalization comprises binning input RNA level values into quantiles. In some aspects, the input RNA levels are binned into 100 quantiles, 150 quantiles, 200 quantiles, or more. In some aspects, the quantile normalization comprises quantile transforming the RNA expression levels to a normal output distribution function. In some aspects, the ANN is trained with a training set comprising RNA expression levels for each gene in the gene panel in a plurality of samples obtained from a plurality of subjects, wherein each sample is assigned a TME classification. In some aspects, the TME classification assigned to each sample in the training set is determined by a population-based classifier.

In some aspects, the population-based classifier comprises determining a Signature 1 score and a Signature 2 score by measuring the RNA expression levels for each gene in the gene panel in each sample in the training set; wherein the genes used to calculate Signature 1 are genes from TABLE 1, FIGS. 28A-28G, or a combination thereof, and the genes used to calculate Signature 2 are genes from TABLE 2, FIGS. 28A-28G, or a combination thereof; and wherein
(i) the TME classification assigned is IA if the Signature 1 score is negative and the Signature 2 score is positive (i.e., the subject would be considered IA biomarker-positive);
(ii) the TME classification assigned is IS if the Signature 1 score is positive and the Signature 2 score is positive (i.e., the subject would be considered IS biomarker-positive);
(iii) the TME classification assigned is ID if the Signature 1 score is negative and the Signature 2 score is negative (i.e., the subject would be considered ID biomarker-positive); and,
(iv) the TME classification assigned is A if the Signature 1 score is positive and the Signature 2 score is negative (i.e., the subject would be considered A biomarker-positive).

In some aspects, the calculation of a Signature 1 score comprises
(i) measuring the expression level for each gene from TABLE 1, FIGS. 28A-28G, or a combination thereof, in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and, (iv) adding all the values obtained in step (iii) and dividing the resulting number by the square root of the number of genes in the gene panel;
wherein if the value obtained in (iv) is above zero, the signature score is a positive signature score, and wherein if the value obtained in (iv) is below zero, the signature score is a negative signature score.

In some aspects, the calculation of a Signature 2 score comprises
(i) measuring the expression level for each gene from TABLE 2, FIGS. 28A-28G, or a combination thereof, in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and, (iv) adding all the values obtained in step (iii) and dividing the resulting number by the square root of the number of genes in the gene panel;
wherein if the value obtained in (iv) is above zero, the signature score is a positive signature score, and wherein if the value obtained in (iv) is below zero, the signature score is a negative signature score. In some aspects, the ANN is trained by backpropagation. In some aspects, the hidden layer comprises 2, 3, 4, or 5 nodes (neurons). In some aspects, a sigmoid activation function is applied to the hidden layer. In some aspects, the sigmoid activation function is a hyperbolic tangent function. In some aspects, the output layer comprises 4 nodes (neurons).

In some aspects, each one of the 4 output nodes in the output layer corresponds to a TME output class, wherein the 4 TME output classes are IA (immune active), IS (immune suppressed), ID (immune desert), and A (angiogenic). In some aspects, the ANN further comprising applying a logistic regression classifier comprising a Softmax function to the output of the ANN, wherein the Softmax function assigns probabilities to each TME output class. In some aspects, the Softmax function is implemented through an additional neural network layer. In some aspects, the additional network layer is interposed between the hidden layer and the output layer. In some aspects, the additional network layer has the same number of nodes as the output layer.

In some aspects of the methods and ANN of the present disclosure, the TME-class specific therapy is an IA-class TME therapy, an IS-class TME therapy, an ID-class TME therapy, an A-class TME therapy, or a combination thereof. In some aspects, assignment of a TME-class specific therapy is based on the presence of a specific stromal phenotype, e.g., if a subject presents an IA stromal phenotype (and therefore the subject is IA biomarker-positive), an IA-class TME therapy would be administered. In some aspects, assignment of a TME-class specific therapy is based on the absence of a specific stromal phenotype, e.g., if a subject does not present an IA stromal phenotype (and therefore the subject is IA biomarker-negative), an IA-class TME therapy would not be administered. In some aspects, assignment of a TME-class specific therapy is based on the presence and/or absence of two or more specific stromal phenotypes, e.g., if the subject presents A and IS stromal phenotypes (and therefore the subject is A and IS biomarker-positive) and does not present ID and IA stromal phenotypes (and therefore the subject is ID and IA biomarker-negative), then a particular TME therapy would be administered.

In some aspects, the IA-class TME therapy comprises a checkpoint modulator therapy. In some aspects, the checkpoint modulator therapy comprises administering an activator of a stimulatory immune checkpoint molecule. In some aspects, the activator of a stimulatory immune checkpoint molecule is an antibody molecule against GITR, OX-40, ICOS, 4-1BB, or a combination thereof. In some aspects, the checkpoint modulator therapy comprises the administration of a RORγ agonist. In some aspects, the checkpoint modulator therapy comprises the administration of an inhibitor of an inhibitory immune checkpoint molecule. In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, alone or a combination thereof, or in combination with an inhibitor of TIM-3, an inhibitor of LAG-3, an inhibitor of BTLA, an inhibitor of TIGIT, an inhibitor of VISTA, an inhibitor of TGF-β or its receptors, an inhibitor of LAIR1, an inhibitor of CD160, an inhibitor of 2B4, an inhibitor of GITR, an inhibitor of OX40, an inhibitor of 4-1BB (CD137), an inhibitor of CD2, an inhibitor of CD27, an inhibitor of CDS, an inhibitor of ICAM-1, an inhibitor of LFA-1 (CD11a/CD18), an inhibitor of ICOS (CD278), an inhibitor of CD30, an inhibitor of CD40, an inhibitor of BAFFR, an inhibitor of HVEM, an inhibitor of CD7, an inhibitor of LIGHT, an inhibitor of NKG2C, an inhibitor of SLAMF7, an inhibitor of NKp80, or a CD86 agonist. In some aspects, the anti-PD-1 antibody comprises nivolumab, pembrolizumab, cemiplimab, PDR001, CBT-501, CX-188, TSR-042, sintilimab, tislelizumab, or an antigen-binding portion thereof. In some aspects, the anti-PD-1 antibody cross-competes with nivolumab, pembrolizumab, cemiplimab, PDR001, CBT-501, CX-188, sintilimab, tislelizumab, or TSR-042 for binding to human PD-1. In some aspects, the anti-PD-1 antibody binds to the same epitope as nivolumab, pembrolizumab, cemiplimab, PDR001, CBT-501, CX-188, sintilimab, tislelizumab, or TSR-042. In some aspects, the anti-PD-L1 antibody comprises avelumab, atezolizumab, durvalumab, CX-072, LY3300054, or an antigen-binding portion thereof. In some aspects, the anti-PD-L1 antibody (e.g., sintilimab, tislelizumab, pembrolizumab, or an antigen binding portion thereof) cross-competes with avelumab, atezolizumab, or durvalumab for binding to human PD-L1. In some aspects, the anti-PD-L1 antibody binds to the same epitope as avelumab, atezolizumab, CX-072, LY3300054, or durvalumab. In some aspects, the check point modulator therapy comprises the administration of (i) an anti-PD-1 antibody selected from the group consisting of nivolumab, pembrolizumab, cemiplimab, PDR001, CBT-501, CX-188, sintilimab, tislelizumab, or TSR-042; (ii) an anti-PD-L1 antibody selected from the group consisting of avelumab, atezolizumab, CX-072, LY3300054, and durvalumab; or (iii) a combination thereof.

In some aspects, the IS-class TME therapy comprises the administration of (1) a checkpoint modulator therapy and an anti-immunosuppression therapy, and/or (2) an antiangiogenic therapy. In some aspects, the checkpoint modulator therapy comprises the administration of an inhibitor of an inhibitory immune checkpoint molecule. In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, or a combination thereof. In some aspects, the anti-PD-1 antibody comprises nivolumab, pembrolizumab, cemiplimab, PDR001, CBT-501, CX-188, TSR-042, sintilimab, tislelizumab, or an antigen-binding portion thereof. In some aspects, the anti-PD-1 antibody cross-competes with nivolumab, pembrolizumab, cemiplimab, PDR001, CBT-501, sintilimab, tislelizumab, CX-188, or TSR-042, for binding to human PD-1. In some aspects, the anti-PD-1 antibody binds to the same epitope as nivolumab, pembrolizumab, cemiplimab, PDR001, CBT-501, CX-188, sintilimab, tislelizumab, or TSR-042. In some aspects, the anti-PD-L1 antibody comprises avelumab, atezolizumab, CX-072, LY3300054, durvalumab, or an antigen-binding portion thereof. In some aspects, the anti-PD-L1 antibody cross-competes with avelumab, atezolizumab, CX-072, LY3300054, or durvalumab for binding to human PD-L1. In some aspects, the anti-PD-L1 antibody binds to the same epitope as avelumab, atezolizumab, CX-072, LY3300054, or durvalumab. In some aspects, the anti-CTLA-4 antibody comprises ipilimumab or the bispecific antibody XmAb20717 (anti PD-1/anti-CTLA-4), or an antigen-binding portion thereof. In some aspects, the anti-CTLA-4 antibody cross-competes with ipilimumab or the bispecific antibody XmAb20717 (anti PD-1/anti-CTLA-4) for binding to human CTLA-4. In some aspects, the anti-CTLA-4 antibody binds to the same CTLA-4 epitope as ipilimumab or the bispecific antibody XmAb20717 (anti PD-1/anti-CTLA-4). In some aspects, the checkpoint modulator therapy comprises the administration of (i) an anti-PD-1 antibody selected from the group consisting of nivolumab, pembrolizumab, cemiplimab, PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iii) an anti-CTLA-4 antibody, which is ipilimumab or the bispecific antibody XmAb20717 (anti PD-1/anti-CTLA-4), or (iv) a combination thereof. In some aspects, the antiangiogenic therapy comprises the administration of an anti-VEGF antibody selected from the group consisting of varisacumab, bevacizumab, navicixizumab (anti-DLL4/anti-VEGF bispecific), and a combination thereof.

In some aspects, the antiangiogenic therapy comprises the administration of an anti-VEGF antibody. In some aspects, the anti-VEGF antibody is an anti-VEGF bispecific antibody. In some aspects, the anti-VEGF bispecific antibody is an anti-DLL4/anti-VEGF bispecific antibody. In some aspects, the anti-DLL4/anti-VEGF bispecific antibody comprises navicixizumab. In some aspects, the antiangiogenic therapy comprises the administration of an anti-VEGFR antibody. In some aspects, the anti-VEGFR antibody is an anti-VEGFR2 antibody. In some aspects, the anti-VEGFR2 antibody comprises ramucirumab. In some aspects, the antiangiogenic therapy comprises the administration of navicixizumab, ABL101 (NOV1501), or ABT165.

In some aspects, the anti-immunosuppression therapy comprises the administration of an anti-PS antibody, anti-PS targeting antibody, antibody that binds β2-glycoprotein 1, inhibitor of PI3Kγ, adenosine pathway inhibitor, inhibitor of IDO, inhibitor of TIM, inhibitor of LAG3, inhibitor of TGF-β, CD47 inhibitor, or a combination thereof. In some aspects, the anti-PS targeting antibody is bavituximab, or an antibody that binds 02-glycoprotein 1. In some aspects, the PI3Kγ inhibitor is LY3023414 (samotolisib) or IPI-549. In some aspects, the adenosine pathway inhibitor is AB-928. In some aspects, the TGFβ inhibitor is LY2157299 (galunisertib) or the TGFβR1 inhibitor is LY3200882. In some aspects, the CD47 inhibitor is magrolimab (5F9). In some aspects, the CD47 inhibitor targets SIRPα.

In some aspects, the anti-immunosuppression therapy comprises the administration of an inhibitor of TIM-3, an inhibitor of LAG-3, an inhibitor of BTLA, an inhibitor of TIGIT, an inhibitor of VISTA, an inhibitor of TGF-β or its receptors, an inhibitor of LAIR1, an inhibitor of CD160, an inhibitor of 2B4, an inhibitor of GITR, an inhibitor of OX40, an inhibitor of 4-1BB (CD137), an inhibitor of CD2, an inhibitor of CD27, an inhibitor of CDS, an inhibitor of ICAM-1, an inhibitor of LFA-1 (CD11a/CD18), an inhibitor of ICOS (CD278), an inhibitor of CD30, an inhibitor of CD40, an inhibitor of BAFFR, an inhibitor of HVEM, an inhibitor of CD7, an inhibitor of LIGHT, an inhibitor of NKG2C, an inhibitor of SLAMF7, an inhibitor of NKp80, an agonist to CD86, or a combination thereof.

In some aspects, the ID-class TME therapy comprises the administration of a checkpoint modulator therapy concurrently or after the administration of a therapy that initiates an immune response. In some aspects, the therapy that initiates an immune response is a vaccine, a CAR-T, or a neo-epitope vaccine. In some aspects, the checkpoint modulator therapy comprises the administration of an inhibitor of an inhibitory immune checkpoint molecule. In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, or a combination thereof. In some aspects, the anti-PD-1 antibody comprises nivolumab, pembrolizumab, cemiplimab, PDR001, CBT-501, CX-188, sintilimab, tislelizumab, or TSR-042, or an antigen-binding portion thereof. In some aspects, the anti-PD-1 antibody cross-competes with nivolumab, pembrolizumab, cemiplimab, PDR001, CBT-501, CX-188, sintilimab, tislelizumab, or TSR-042, for binding to human PD-1. In some aspects, the anti-PD-1 antibody binds to the same epitope as nivolumab, pembrolizumab, cemiplimab, PDR001, CBT-501, CX-188, sintilimab, tislelizumab, or TSR-042. In some aspects, the anti-PD-L1 antibody comprises avelumab, atezolizumab, CX-072, LY3300054, durvalumab, or an antigen-binding portion thereof. In some aspects, the anti-PD-L1 antibody cross-competes with avelumab, atezolizumab, CX-072, LY3300054, or durvalumab for binding to human PD-L1. In some aspects, the anti-PD-L1 antibody binds to the same epitope as avelumab, atezolizumab, CX-072, LY3300054, or durvalumab. In some aspects, the anti-CTLA-4 antibody comprises ipilimumab or the bispecific antibody XmAb20717 (anti PD-1/anti-CTLA-4), or an antigen-binding portion thereof. In some aspects, the anti-CTLA-4 antibody cross-competes with ipilimumab or the bispecific antibody XmAb20717 (anti PD-1/anti-CTLA-4) for binding to human CTLA-4. In some aspects, the anti-CTLA-4 antibody binds to the same CTLA-4 epitope as ipilimumab or the bispecific antibody XmAb20717 (anti PD-1/anti-CTLA-4). In some aspects, the checkpoint modulator therapy comprises the administration of (i) an anti-PD-1 antibody selected from the group consisting of nivolumab, pembrolizumab, cemiplimab PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iv) an anti-CTLA-4 antibody, which is ipilimumab or the bispecific antibody XmAb20717 (anti PD-1/anti-CTLA-4), or (iii) a combination thereof.

In some aspects, the A-class TME therapy comprises a VEGF-targeted therapy and other anti-angiogenics, an inhibitor of angiopoietin 1 (Ang1), an inhibitor of angiopoietin 2 (Ang2), an inhibitor of DLL4, a bispecific of anti-VEGF and anti-DLL4, a TKI inhibitor, an anti-FGF antibody, an anti-FGFR1 antibody, an anti-FGFR2 antibody, a small molecule that inhibits FGFR1, a small molecule that inhibits FGFR2, an anti-PLGF antibody, a small molecule against a PLGF receptor, an antibody against a PLGF receptor, an anti-VEGFB antibody, an anti-VEGFC antibody, an anti-VEGFD antibody, an antibody to a VEGF/PLGF trap molecule such as aflibercept, or ziv-aflibercet, an anti-DLL4 antibody, or an anti-Notch therapy such as an inhibitor of gamma-secretase. In some aspects, the TKI inhibitor is selected from the group consisting of cabozantinib, vandetanib, tivozanib, axitinib, lenvatinib, sorafenib, regorafenib, sunitinib, fruquitinib, pazopanib, and any combination thereof. In some aspects, the TKI inhibitor is fruquintinib. In some aspects, the VEGF-targeted therapy comprises the administration of an anti-VEGF antibody or an antigen-binding portion thereof. In some aspects, the anti-VEGF antibody comprises varisacumab, bevacizumab, or an antigen-binding portion thereof. In some aspects, the anti-VEGF antibody cross-competes with varisacumab, or bevacizumab for binding to human VEGF A. In some aspects, the anti-VEGF antibody binds to the same epitope as varisacumab, or bevacizumab. In some aspects, the VEGF-targeted therapy comprises the administration of an anti-VEGFR antibody. In some aspects, the anti-VEGFR antibody is an anti-VEGFR2 antibody. In some aspects, the anti-VEGFR2 antibody comprises ramucirumab or an antigen-binding portion thereof.

In some aspects, the A-class TME therapy comprises the administration of an angiopoietin/TIE2-targeted therapy. In some aspects, the angiopoietin/TIE2-target therapy comprises the administration of endoglin and/or angiopoietin. In some aspects, the A-class TME therapy comprises the administration of a DLL4-targeted therapy. In some aspects, the DLL4-targeted therapy comprises the administration of navicixizumab, ABL101 (NOV1501), or ABT165.

In some aspects, the methods disclosed herein further comprise
(a) administering chemotherapy;
(b) performing surgery;
(c) administering radiation therapy; or,
(d) any combination thereof.

In some aspects, the cancer is a tumor. In some aspects, the tumor is a carcinoma. In some aspects, the tumor is selected from the group consisting of gastric cancer, colorectal cancer, liver cancer (hepatocellular carcinoma, HCC), ovarian cancer, breast cancer, NSCLC, bladder cancer, lung cancer, pancreatic cancer, head and neck cancer, lymphoma, uterine cancer, renal or kidney cancer, biliary cancer, anal cancer, prostate cancer, testicular cancer, urethral cancer, penile cancer, thoracic cancer, rectal cancer, brain cancer (glioma and glioblastoma), cervicalparotid cancer, esophageal cancer, gastroesophageal cancer, larynx cancer, thyroid cancer, adenocarcinomas, neuroblastomas, melanoma, and Merkel Cell carcinoma.

In some aspects, the cancer is relapsed. In some aspects, the cancer is refractory. In some aspects, the cancer is refractory following at least one prior therapy comprising administration of at least one anticancer agent. In some aspects, the cancer is metastatic. In some aspects, the administering effectively treats the cancer. In some aspects, the administering reduces the cancer burden. In some aspects, cancer burden is reduced by at least about 10%, at least about 20%, at least about 30%, at least about 40%, or about 50% compared to the cancer burden prior to the administration. In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration. In some aspects, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

In some aspects, the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration. In some aspects, the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

In some aspects, the administering improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject not exhibiting the TME. In some aspects, the administering improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not exhibiting the TME.

The present disclosure also provides a gene panel comprising at least an angiogenic biomarker gene from TABLE 1 and an immune biomarker gene from TABLE 2, for use in determining the tumor microenvironment of a tumor in a subject in need thereof using a machine-learning classifier comprising an ANN disclosed herein, wherein the tumor microenvironment is used for (i) identifying a subject suitable for an anticancer therapy; (ii) determining the prognosis of a subject undergoing anticancer therapy; (iii) initiating, suspending, or modifying the administration of an anticancer therapy; or, (iv) a combination thereof.

Also provided is a non-population based classifier comprising an ANN as disclosed herein for identifying a human subject afflicted with a cancer suitable for treatment with an anticancer therapy, wherein the machine-learning classifier identifies the subject as exhibiting a TME selected from IA, IS, ID, A-class TME, or a combination thereof, wherein (i) the therapy is an IA Class TME therapy if the TME is IA or predominantly IA; (ii) the therapy is an IS Class TME therapy if the TME is IS or predominantly IS; (iii) the therapy is an ID Class TME therapy if the TME is ID or predominantly ID; or (iv) the therapy is an A Class TME therapy if the TME is A or predominantly A. In some aspects, a subject can exhibit more than one TME, e.g., the subject can be biomarker-positive for IA and IS, or IA and ID, or IA and A, etc. A subject being biomarker-positive and/or biomarker-negative for more than one stromal phenotype can receive one or more TME-class specific therapies.

The present disclosure also provides an anticancer therapy for treating a cancer in a human subject in need thereof, wherein the subject is identified as exhibiting a TME selected from IA, IS, ID or A-class TME or a combination thereof, according to the machine-learning classifier comprising an ANN disclosed herein, wherein (i) the therapy is an IA-Class TME therapy if the TME is IA or predominantly IA; (ii) the therapy is an IS-Class TME therapy if the TME is IS or predominantly IS; (iii) the therapy is an ID-Class TME therapy if the TME is ID or predominantly ID; or (iv) the therapy is an A-Class TME therapy if the TME is A or predominantly A. In some aspects, a subject can exhibit more than one TME, e.g., the subject can be biomarker-positive for IA and IS, or IA and ID, or IA and A, etc. A subject being biomarker-positive and/or biomarker-negative for more than one stromal phenotype can receive one or more TME-class specific therapies.

Also provided is a method of assigning a TME class to a cancer in a subject in need thereof, the method comprising (i) generating a machine-learning model by training a machine-learning method with a training set comprising RNA expression levels for each gene in a gene panel in a plurality of samples obtained from a plurality of subjects, wherein each sample is assigned a TME classification; and, (ii) assigning, using the machine-learning model, the TME of the cancer in the subject, wherein the input to the machine-learning model comprises RNA expression levels for each gene in the gene panel in a test sample obtained from the subject.

Also provided is a method of assigning a TME class to a cancer in a subject in need thereof, the method comprising generating a machine-learning model by training a machine-learning method with a training set comprising RNA expression levels for each gene in a gene panel in a plurality of samples obtained from a plurality of subjects, wherein each sample is assigned a TME classification; wherein the machine-learning model assigns a TME class to the cancer in the subject using as input RNA expression levels for each gene in the gene panel in a test sample obtained from the subject.

The disclosure also provides a method of assigning a TME class to a cancer in a subject in need thereof, the method comprising using a machine-learning model to predict the TME of the cancer in the subject, wherein the machine-learning model is generated by training a machine-learning method with a training set comprising RNA expression levels for each gene in a gene panel in a plurality of samples obtained from a plurality of subjects, wherein each sample is assigned a TME classification.

In some aspects of the methods disclosed herein, the machine-learning model is generated by an ANN prepared as disclosed herein. In some aspects, the TME classification assigned to each sample in the training set is determined by a population-based classifier. In some aspects, the population-based classifier comprises determining a Signature 1 score and a Signature 2 score by measuring the RNA expression levels for each gene in the gene panel in each sample in the training set; wherein the genes used to calculate Signature 1 are genes from TABLE 1, FIGS. 28A-28G, or a combination thereof and the genes used to calculate Signature 2 are genes from TABLE 2, FIGS. 28A-28G, or a combination thereof; and wherein
(i) the TME classification assigned is IA if the Signature 1 score is negative and the Signature 2 score is positive (i.e., the subject would be considered IA biomarker-positive);
(ii) the TME classification assigned is IS if the Signature 1 score is positive and the Signature 2 score is positive (i.e., the subject would be considered IS biomarker-positive);
(iii) the TME classification assigned is ID if the Signature 1 score is negative and the Signature 2 score is negative (i.e., the subject would be considered ID biomarker-positive); and,
(iv) the TME classification assigned is A if the Signature 1 score is positive and the Signature 2 score is negative (i.e., the subject would be considered A biomarker-positive).

In some aspects, the calculation of a Signature 1 score comprises
(i) measuring the expression level for each gene from TABLE 1, or a subset thereof, or a subset of genes from FIGS. 28A-28G, in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and,
(iv) adding all the values obtained in step (iii) and dividing the resulting number by the square root of the number of genes in the gene panel;
wherein if the value obtained in (iv) is above zero, the signature score is a positive signature score, and wherein if the value obtained in (iv) is below zero, the signature score is a negative signature score.

In some aspects, the calculation of a Signature 2 score comprises
(i) measuring the expression level for each gene from TABLE 2, or a subset thereof, or a subset of genes from FIGS. 28A-28G, in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and, (iv) adding all the values obtained in step (iii) and dividing the resulting number by the square root of the number of genes in the gene panel;
wherein if the value obtained in (iv) is above zero, the signature score is a positive signature score, and wherein if the value obtained in (iv) is below zero, the signature score is a negative signature score.

In some aspects, the machine-learning model comprises a logistic regression classifier comprising a Softmax function applied to the output of the model, wherein the Softmax function assigns probabilities to each TME output class.

In some aspects, the method is implemented in a computer system comprising at least one processor and at least one memory, the at least one memory comprising instructions executed by the at least one processor to cause the at least one processor to implement the machine-learning model. In some aspects, the method further comprises (i) inputting, into the memory of the computer system, the machine-learning model; (ii) inputting, into the memory of the computer system, the gene panel input data corresponding to the subject, wherein the input data comprises RNA expression levels; (iii) executing the machine-learning model; or, (v) any combination thereof.

In some aspects, the probabilities of the logistic regression classifier are overlaid on a latent space plot of the activation scores of the nodes of the ANN model. In some aspects, the logistic regression classifier is trained on the latent space. In some aspects, the logistic regression classifier is optimized for PFS (Progression-Free Survival). In some aspects, the logistic regression classifier is optimized for BOR (Best Objective Response), ORR (Overall Response Rate), MSS/MSI-high (Microsatellite Stable/Microsatellite Instability-high) status, PD-1/PD-L1 status, PFS (Progression-Free Survival), NLR (Neutrophil Leukocyte Ratio), Tumor Mutation Burden (TMB) or any combination thereof.

### BRIEF DESCRIPTION OF THE DRAWINGS/FIGURES

FIG. 1 shows the normalization of three datasets prior to classification.

FIG. 2 is a risk curve comparison from Kaplan-Meier Plot of the ACRG dataset after classification of 298 patients into the four stromal subtypes (i.e., stromal phenotypes).

FIG. 3 is a risk curve comparison from Kaplan-Meier Plot of the TCGA dataset after classification of 388 patients into the four stromal subtypes (i.e., stromal phenotypes).

FIG. 4 is a risk curve comparison from Kaplan-Meier Plot of the Singapore dataset after classification of 192 patients into the four stromal subtypes (i.e., stromal phenotypes).

FIG. 5 is a risk curve comparison from Kaplan-Meier Plot of the three datasets (878 patients) combined after classification into the four stromal subtypes (i.e., stromal phenotypes).

FIGS. 6A and 6B show representative gene ontology signatures expressed as box plots in the ACRG cohort. FIG. 6A shows box plots of the median and range of values for the expression levels from the Treg signature as a function of the four stromal subtypes (i.e., stromal phenotypes) in the ACRG data. FIG. 6B shows a box plot of the median and range of values for the expression levels of an inflammatory response signature as a function of the four stromal subtypes (i.e., stromal phenotypes) in the ACRG data.

FIGS. 7A and 7B show representative gene ontology signatures in the ACRG cohort that reflect the biology of the titles of the individual plots. FIG. 7A shows that Signature 1 activation is correlated with endothelial cell signature activation. FIG. 7B shows that Signature 2 activation is correlated with inflammatory and immune cell signature activation.

FIGS. 8A and 8B show representative gene ontology signatures in the TCGA dataset that reflect the biology of the titles of the individual plots. FIG. 8A shows that Signature 1 activation is correlated with endothelial cell signature activation. FIG. 8B shows that Signature 2 activation is correlated with inflammatory and immune cell signature activation.

FIGS. 9A and 9B show representative gene ontology signatures in the Singapore cohort that reflect the biology of the titles of the individual plots. FIG. 9A shows that Signature 1 activation is correlated with endothelial cell signature activation. FIG. 9B shows that Signature 2 activation is correlated with inflammatory and immune cell signature activation.

FIG. 10 is a chart showing tumor microenvironment (TME) assignments based on the application of a classifier disclosed herein, as well as treatment classes assigned to each TME class.

FIG. 11 depicts a logistic function used in the logistic regression model.

FIG. 12A is an exemplary small decision tree.

FIG. 12B shows that predictions for new samples can be made by averaging the predictions from the individual trees.

FIG. 13 shows the parameters from the Random Forest classifier.

FIG. 14 shows part of an Artificial Neural Network (ANN) training set comprising a number of Samples, each one corresponding to a subject (column A), the TME class for the subject's cancer assigned according to the population-based classifier of the present disclosure (column B), and RNA expression levels corresponding to different genes in the selected gene panel (columns C, D, E, etc.).

FIG. 15 shows a simplified view of an ANN used as a non-population based classifier in the present disclosure. The ANN comprises an input layer with inputs corresponding to each gene in the gene panel (e.g., a 124 gene panel, 105 gene panel, 98 gene panel, or alternatively an 87 gene panel), a hidden layer comprising two neurons (or alternatively 3, 4 or 5 neurons), and an output layer that would correspond to TME class assignments (i.e., stromal phenotype assignments).

FIG. 16 is a schematic representation showing alternative ANN architectures that can be used to develop a non-population based classifier according to the present disclosure.

FIG. 17 shows that inputs to the ANN corresponding to mRNA levels (x) for genes 1 to n are fed to the hidden layer neurons, and a bias (b) is applied to the hidden layer neurons. The input to the neuron is integrated through a function (f) which incorporates the bias and the mRNA expression levels (x₁ . . . xₙ) normalized according to their respective weights (w₁ . . . wₙ).

FIG. 18 shows different activation functions that can be applied to the neurons in the hidden layer.

FIG. 19 shows the artificial neuronal network (ANN) model architecture. The “Input layer” is a vector of expressions xi, i∈G from a single sample. The “Hidden layer” comprises two neurons, each taking gene expression as input. The “Output layer” comprises four neurons, each taking activations of the two hidden neurons as input, transforming them with the tanh (hyperbolic tangent) activation function as a weighted sum to yield (y), followed by a logistic regression classifier (e.g., Softmax function) (zi) to produce probabilities of the four phenotype classes (IA, ID, A, IS). Alternative aspects of the ANN can comprise, e.g., five neurons instead of two neurons.

FIG. 20 shows the Kaplan-Meier survival curve for in a population of gastric cancer patients with known biomarker status and known outcome treated with pembrolizumab monotherapy.

FIG. 21A shows the application of machine-learning (ANN) to optimize the cut-off defining patients that are responders with respect to the non-responders, and two possible options for patient selection.

FIG. 21B illustrates that in addition to the use of linear thresholds different from the Cartesian x=0, y=0 thresholds to define patients that are responders with respect to the non-responders as exemplified in FIG. 21A, it is possible to use non-linear thresholds to define patient populations and to use such non-linear thresholds for patient selection.

FIG. 22 shows the Kaplan-Meier survival curve for Navi 1B reproductive cancer patients with known biomarker status and known outcome.

FIG. 23 shows probability contours, expressed as a percentage, of TME classes for the pembrolizumab patient data of Example 12, overlaid on a latent space plot of the activation scores 1 and 2 of the ANN model (x and y axes). The top left quadrant corresponds to the A TME stromal phenotype, the lower left quadrant corresponds to the ID TME stromal phenotype, the lower right quadrant corresponds to the IA TME stromal phenotype, and the top right quadrant corresponds to the IS TME stromal phenotype. Patient Best Objective Response outcome is represented by: Progressive Disease (PD)—circle; Stable Disease (SD)—triangle; Partial Response (PR)—square; and Complete Response (CR)—“x.” Filled shapes represent the patients with a PD-L1 status ≥1, empty shapes are PD-L1<1. Of the 73 patients of Example 12, four were missing PD-L1 status and so are omitted from the plot.

FIG. 24 shows probability of biomarker positivity informed by a logistic regression classifier based on Progression-Free Survival (PFS) greater than 5 months, of TME classes of the pembrolizumab patient data of Example 12, overlaid on a latent space plot of the activation scores 1 and 2 of the ANN model (x and y axes). The classifier was trained based on the samples using a neutrophil leukocyte ratio less than 4 (NLR<4), using PFS>5 as a positive class. The top left quadrant corresponds to the A TME stromal phenotype, the lower left quadrant corresponds to the ID TME stromal phenotype, the lower right quadrant corresponds to the IA TME stromal phenotype, and the top right quadrant corresponds to the IS TME stromal phenotype. Patient Best Objective Response outcome is represented by: Progressive Disease (PD)—circle; Stable Disease (SD)—triangle; Partial Response (PR)—square; and Complete Response (CR)—“x.” Filled shapes represent the patients with a PD-L1 status ≥1, empty shapes are PD-L1<1. Of the 73 patients of Example 12, four were missing PD-L1 status and so are omitted from the plot.

FIG. 25 shows probability of biomarker positivity informed by logistic regression classifier based on Best Objective Response of TME classes of the pembrolizumab patient data of Example 12 overlaid on a latent space plot of the activation scores 1 and 2 of the ANN model (x and y axes). The classifier was trained based on the samples using a neutrophil leukocyte ratio less than 4 (NLR<4), using Complete Responder and Partial Responders (CR+PR) as a positive class. Top left quadrant corresponds to the A TME stromal phenotype, the lower left quadrant corresponds to the ID TME stromal phenotype, the lower right quadrant corresponds to the IA TME stromal phenotype, and the top right quadrant corresponds to the IS TME stromal phenotype. Patient Best Objective Response outcome is represented by: Progressive Disease (PD)—circle; Stable Disease (SD)—triangle; Partial Response (PR)—square; and Complete Response (CR)—“x.” Filled shapes represent the patients with a PD-L1 status ≥1, empty shapes are PD-L1<1. Of the 73 patients of Example 12, four were missing PD-L1 status and so are omitted from the plot.

FIG. 26 shows probability of TME class of the bavituximab and pembrolizumab combination therapy clinical data of Example 7 overlaid on a latent space plot of activation scores 1 and 2 of the ANN model (x and y axes), for all patients (n=38). The top left quadrant corresponds to the A TME stromal phenotype, the lower left quadrant corresponds to the ID TME stromal phenotype, the lower right quadrant corresponds to the IA TME stromal phenotype, and the top right quadrant corresponds to the IS TME stromal phenotype. Patient Best Objective Response outcome is represented by: Progressive Disease (PD)—circle; Stable Disease (SD)—triangle; Partial Response (PR)—square; and Complete Response (CR)—“x.” Filled shapes represent the patients with confirmed responses, empty shapes are unconfirmed responses.

FIG. 27 shows neural net activation scores (filled circles, activation score 1 (node 1); open squares, activation score 2 (node 2)) and predicted TME class (ANN phenotype call) for tissue samples each from colorectal cancer (left, n=370), gastric cancer (center, n=337), and ovarian cancer (right, n=392). The distribution of samples between the four TME classes is similar for different disease groups.

FIG. 28A shows the presence (open cells) or absence (full cells) of 124 genes in Genesets 1 to 44.

FIG. 28B shows the presence (open cells) or absence (full cells) of 124 genes in Genesets 45 to 88.

FIG. 28C shows the presence (open cells) or absence (full cells) of 124 genes in Genesets 89 to 132.

FIG. 28D shows the presence (open cells) or absence (full cells) of 124 genes in Genesets 133 to 177.

FIG. 28E shows the presence (open cells) or absence (full cells) of 124 genes in Geneset 178 to 222.

FIG. 28F shows the presence (open cells) or absence (full cells) of 124 genes in Geneset 223 to 267.

FIG. 28G shows the presence (open cells) or absence (full cells) of 124 genes in Geneset 268 to 282.

FIG. 29A is an illustrative schematic of gene weights in a first node of an ANN model, presented as a histogram of a sample of 30 gene weights (X axis). Open bars, a subset of genes of Signature 1, closed bars, a subset of genes of Signature 2. Weights are given on the Y axis.

FIG. 29B is an illustrative schematic of gene weights in a second node of an ANN model, presented as a histogram of a sample of 30 gene weights (X axis). Open bars, a subset of genes of Signature 1, closed bars, a subset of genes of Signature 2. Weights are given on the Y axis.

### DETAILED DESCRIPTION

The present disclosure provides methods to classify patients and cancers according to population and non-population tumor microenvironment (TME) classification methods. The population methods (i.e., population-based classifiers) disclosed herein can be used not only as stand-alone classifiers, but also as means to preprocess gene expression data to be used as training sets for the generation of non-population models (i.e., non-population-based classifiers) based on the application of machine-learning techniques, e.g., predictive models based on Artificial Neural Networks (ANN).

As used herein, the term “non-population-based” method or classifier is interchangeable with the terms machine learning (ML) method or ML classifier, e.g., an ANN classifier of the present disclosure. As used herein, the term “population-based” method or classifier is interchangeable with the terms Z-score method or Z-score classifier.

In some aspects, gene sets that can represent one or more biological signatures (i.e., a Signature 1, Signature 2, Signature 3, . . . Signature N) are used according to the methods disclosed herein to compute a Z-score for Signatures 1 . . . N. This comprises a population model which can be used to reveal the dominant biologies represented by each signature and the TME phenotypes defined by the matrix of those signatures. In some aspects, a machine learning model (e.g. ANN) can be trained, e.g., using as features the geneset derived from the signatures, and as expressions a historic patient dataset, e.g., the ACRG (Asian Cancer Research Group) patient dataset.

The machine learning model (e.g., an ANN) learns the (latent) gene expression patterns that classify an individual patient into specific TME phenotypes. The machine learning model (e.g. ANN) effectively compresses the high dimensional data (gene expressions of all genes in the input geneset) into a lower dimensional (latent) space, e.g. the two hidden neurons in an ANN disclosed herein. The machine learning model (e.g. ANN) then outputs phenotype classes, e.g., four TME phenotype classes, which themselves can be used to define biomarker positivity, alone (in whole or in part) or in combination with one another (again, in whole or in part), in a drug specific manner. Alternatively, a secondary model (e.g., a logistic regression classifier) can be trained on the latent space in order to learn not the TME phenotypes, but rather to learn directly the biomarker positive versus biomarker negative decision boundary based on patient outcome labels.

In some aspects, the secondary model (e.g., a logistic regression classifier) applied to the ANN classifications according to the methods of the present disclosure can be optimized for BOR (Best Objective Response), ORR (Overall Response Rate), MSS/MSI-high (Microsatellite Stable/Microsatellite Instability-high) status, PD-1/PD-L1 status, PFS (Progression-Free Survival), NLR (Neutrophil Leukocyte Ratio), Tumor Mutation Burden (TMB) or any combination thereof.

Accordingly, in some aspects, the present disclosure provides population classifiers based on the integration of a number of signatures, i.e., global scores related to the expression of genes (e.g., those in TABLES 1 and TABLE 2) in particular gene panels (e.g., those in TABLES 3 and TABLE 4), such as Signature 1 and Signature 2 disclosed herein. These signature scores allow patients and cancers to be stratified according to TME, and treatment decisions are then guided by the presence or absence of a particular TME.

In other aspects, the present disclosure provides non-population classifiers based on the application of machine-learning techniques, e.g., logistic regression, random forests, or artificial neural networks (ANN). The ANN classifiers disclosed herein are based, e.g., on training a neural network using a dataset preprocessed according to the population-based classifiers disclosed herein.

An advantage of the non-population-based classifiers (ANN classifiers) disclosed herein over the population-based classifier also disclosed herein, is that a sample from a patient who is, e.g., part of a clinical trial or a clinical regimen, can be correctly assessed for stromal phenotype or biomarker positivity, without reference to any other current patient data. Thus, while the availability of a latent plot with the probabilities for each phenotypic class is useful, it is not required to correctly assess for stromal phenotype or biomarker positivity.

The present disclosure also provides methods for treating a subject, e.g., a human subject, afflicted with cancer comprising administering a particular therapy depending on the classification of the cancer's TME according to the population and/or non-population-based classifiers disclosed herein, for example, based on the presence (biomarker-positive) and/or absence (biomarker-negative) of one or more TME class assignments (e.g., whether the subject is A and IS biomarker-positive, and/or ID and IA biomarker-negative).

Also provided are personalized treatments that can be administered to a subject having a cancer classified into a particular TME class or group thereof (i.e., the subject is biomarker-positive for a particular TME class or group thereof), or determined not to have a cancer classified into a particular TME class or group thereof (i.e., the subject is biomarker-negative for a particular TME class or group thereof). The disclosure also provides gene panels (e.g., those disclosed in TABLE 3 and TABLE 4) that can be used for identifying a human subject afflicted with a cancer suitable for treatment with a particular therapeutic agent, e.g., a TME-specific therapy.

The application of the methods and compositions disclosed herein can improve clinical outcomes by matching patients to therapies (e.g., any of the TME-specific therapies disclosed below or a combination thereof depending on the biomarker-positive and/or biomarker-negative status of the subject) with a mechanism of action that targets one or more specific stromal subtypes (i.e., stromal phenotypes) or tumor biology.

Dominant stromal phenotypes can be directional but modified for any specific drug based on the complexity of the mechanism of action of drug, drugs, or clinical regimen. Combinations of drugs or clinical regimens (i.e., one or more TME-specific therapies disclosed below) can be applied to multiple stromal phenotypes if relevant, e.g., to a patient or group of patients that are biomarker-positive for more than one stromal phenotype or are predominantly one stromal phenotype, but there is contribution of other stromal phenotypes in the biomarker signal as seen in the probability function of the ANN model or logistic regressions applied to the latent space, as in this disclosure. Thus, the term “predominantly,” as applied to a stromal phenotype disclosed herein indicates that a patient or sample is biomarker positive for a particular stromal phenotype (e.g., IA), but other stromal phenotypes (e.g., IS, ID or A) or combinations thereof also contribute to the biomarker signal as seen in the probability function of the ML model, e.g., ANN model disclosed herein, or in logistic regressions applied to the latent space.

In some aspects, the patient can be biomarker positive for a specific part of the stromal phenotype, e.g., a patient may be considered biomarker-positive above or below a specific threshold or combination thereof (e.g., an upper and a lower threshold) within a particular stromal phenotype. Stated another way, a stromal phenotype can match the drug (e.g., the IA stromal phenotype can match the drug pembrolizumab), but when a drug or drug combination can modify multiple stromal phenotypes, the stromal phenotypes can be used as a starting point to develop a drug-specific combination, e.g. using bavituximab plus pembrolizumab. Accordingly, determining that a patient or a population of patients are biomarker-positive for two or more stromal phenotypes can be used to develop new therapies by combining two or more TME-specific therapies. For example, the clinical regimen of bavituximab and pembrolizumab targets two stromal phenotypes, IA and IS, and so a diagnostic or biomarker signature for this combination will be a synthesis and refinement based on both stromal phenotypes. Another illustrative example is the bispecific antibody navicixizumab, which is both a VEGF- and DLL4-targeting agent. While VEGF clearly targets the A stromal phenotype, there are features of the IS group that reflect the milieu of the DLL4 biology. Thus, a diagnostic biomarker signature utilizing an algorithm that integrates the A and IS stromal phenotypes (or, e.g., subsets thereof defined for example by one or more threshold values), and additional genes, as described herein can be used to bring out non-angiogenic features of the DLL4 biology.

### Terms

In order that the present disclosure can be more readily understood, certain terms are first defined. As used in this disclosure, except as otherwise expressly provided herein, each of the following terms shall have the meaning set forth below. Additional definitions are set forth throughout the disclosure.

“Administering” refers to the physical introduction of a composition comprising a therapeutic agent (e.g., a monoclonal antibody) to a subject, using any of the various methods and delivery systems known to those skilled in the art. Preferred routes of administration include intravenous, intramuscular, subcutaneous, intraperitoneal, spinal or other parenteral routes of administration, for example by injection or infusion.

The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, intraocular, intravitreal, periorbital, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “antibody” includes, by way of example, monoclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody. As used herein, the term “antibody” does not include naturally occurring antibodies or polyclonal antibodies. As used herein, the term “naturally occurring antibodies” and “polyclonal antibodies” do not include antibodies resulting from an immune reaction induced by a therapeutic intervention, e.g., a vaccine.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 (e.g., sintilimab, tislelizumab, pembrolizumab, or an antigen binding portion thereof) can, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

A “bispecific antibody” as used herein refers to an antibody comprising two antigen-binding sites, a first binding site having affinity for a first antigen or epitope and a second binding site having binding affinity for a second antigen or epitope distinct from the first.

An “anti-antigen antibody” refers to an antibody that binds specifically to the antigen. For example, an anti-PD-1 antibody (e.g., sintilimab, tislelizumab, pembrolizumab, or an antigen binding portion thereof) binds specifically to PD-1, and an anti-PD-L1 antibody binds specifically to PD-L1.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-PD-1 antibody (e.g., sintilimab, tislelizumab, pembrolizumab, or an antigen binding portion thereof) or an anti-PD-L1 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using available techniques in the art, and the fragments are screened for utility in the same manner as are intact antibodies. Antigen-binding portions can be produced by recombinant DNA techniques, or by enzymatic or chemical cleavage of intact immunoglobulins.

As used herein, the term “antibody,” when applied to a specific antigen, encompasses also antibody molecules comprising other binding moieties with different binding specificities. Accordingly, in one aspect, the term antibody also encompasses antibody drug conjugates (ADC). In another aspect, the term antibody encompasses multispecific antibodies, e.g., bispecific antibodies. Thus, for example, the term anti-PD-1 antibody would also encompass ADCs comprising an anti-PD-1 antibody or an antigen-binding portion thereof. Similarly, the term anti-PD-1 antibody would encompass bispecific antibodies comprising an antigen-binding portion capable of specifically binding to PD-1.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. The term “tumor” refers to a solid cancer. The term “carcinoma” refers to a cancer of epithelial origin.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

In the context of the present disclosure, the terms “immunosuppressed” or “immunosuppression” describe the status of the immune response to the cancer. The patient's immune response to the cancer can be dampened by immune suppressive cells in the tumor microenvironment, thus blocking, preventing, or diminishing an immune system attack on the cancer. In immunosuppression therapy the goal is to relieve immunosuppression (as opposed to causing immunosuppression, e.g., as in the context of an organ transplant) by giving patients certain drugs, so that the immune system can attack the cancer.

The term “small molecule” refers to an organic compound having a molecular weight of less than about 900 Daltons, or less than about 500 Daltons. The term includes agents having the desired pharmacological properties, and includes compounds that can be taken orally or by injection. The term includes organic compounds that modulate the activity of TGF-β, and/or other molecules associated with enhancing or inhibiting an immune response.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7. The human PD-L1 protein is encoded by the human CD274 gene (NCBI Gene ID: 29126).

As used herein, the term “subject” includes any human or nonhuman animal. The terms, “subject” and “patient” are used interchangeably herein. The term “nonhuman animal” includes, but is not limited to, vertebrates such as dogs, cats, horses, cows, pigs, boar, sheep, goat, buffalo, bison, llama, deer, elk and other large animals, as well as their young, including calves and lambs, and to mice, rats, rabbits, guinea pigs, primates such as monkeys and other experimental animals. Within animals, mammals are preferred, most preferably, valued and valuable animals such as domestic pets, race horses and animals used to directly produce (e.g., meat) or indirectly produce (e.g., milk) food for human consumption, although experimental animals are also included. In specific aspects, the subject is a human. Thus, the present disclosure is applicable to clinical, veterinary and research uses.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis). As used here, the terms “treat,” “treating,” and “treatment” refer to the administration of an effective dose or effective dosage.

The term “effective dose” or “effective dosage” is defined as an amount sufficient to achieve or at least partially achieve a desired effect.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction.

A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease.

In addition, the terms “effective” and “effectiveness” with regard to a treatment disclosed herein includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

The ability of a therapeutic agent to promote disease regression, e.g., cancer regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” or combination thereof promotes cancer regression in a subject. In some aspects, a therapeutically effective amount of the therapeutic agent promotes cancer regression to the point of eliminating the cancer.

In some aspects of the present disclosure, the anticancer agents are administered as a combination of therapies: a therapy comprising the administration of (i) an anti-PD-1 antibody (e.g., sintilimab, tislelizumab, pembrolizumab, or an antigen binding portion thereof), and (ii) an anti-phosphatidylserine (PS) targeting antibody, e.g., bavituximab.

“Promoting cancer regression” means that administering an effective amount of the drug or combination thereof (administered together as a single therapeutic composition or as separate compositions in separate treatments as discussed above), results in a reduction in cancer burden, e.g., reduction in tumor growth or size, necrosis of the tumor, a decrease in severity of at least one disease symptom, an increase in frequency and duration of disease symptom-free periods, or a prevention of impairment or disability due to the disease affliction.

Notwithstanding these ultimate measurements of therapeutic effectiveness, evaluation of immunotherapeutic drugs must also make allowance for immune-related response patterns. The ability of a therapeutic agent to inhibit cancer growth, e.g., tumor growth, can be evaluated using assays described herein and other assays known in the art. Alternatively, this property of a composition can be evaluated by examining the ability of the compound to inhibit cell growth, such inhibition can be measured in vitro by assays known to the skilled practitioner.

The terms “biological sample” or “sample” as used herein refers to biological material isolated from a subject. The biological sample can contain any biological material suitable for determining gene expression, for example, by sequencing nucleic acids.

The biological sample can be any suitable biological tissue, for example, cancer tissue. In one aspect, the sample is a tumor tissue biopsy, e.g., a formalin-fixed, paraffin-embedded (FFPE) tumor tissue or a fresh-frozen tumor tissue or the like. In another aspect, an intratumoral sample is used. In another aspect, biological fluids can be present in a tumor tissue biopsy, but the biological sample will not be a biological fluid per se.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about,” “comprising essentially of,” or “consisting essentially of,” refer to a value or composition that is within an acceptable error range for the particular value or composition as determined by one of ordinary skill in the art, which will depend in part on how the value or composition is measured or determined, i.e., the limitations of the measurement system. For example, “about,” “comprising essentially of,” or “consisting essentially of,” can mean within 1 or more than 1 standard deviation per the practice in the art. Alternatively, “about,” “comprising essentially of,” or “consisting essentially of,” can mean a range of up to 10%. Furthermore, particularly with respect to biological systems or processes, the terms can mean up to an order of magnitude or up to 5-fold of a value. When particular values or compositions are provided in the specification and claims, unless otherwise stated, the meaning of “about,” “comprising essentially of,” or “consisting essentially of,” should be assumed to be within an acceptable error range for that particular value or composition.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

### I. Tumor Microenvironment (TME) Classification

The present disclosure provides methods for the classification of the tumor microenvironment (TME) of a cancer in a subject in need thereof. These classifiers can be population-based classifiers, non-population-based classifiers, or combinations thereof.

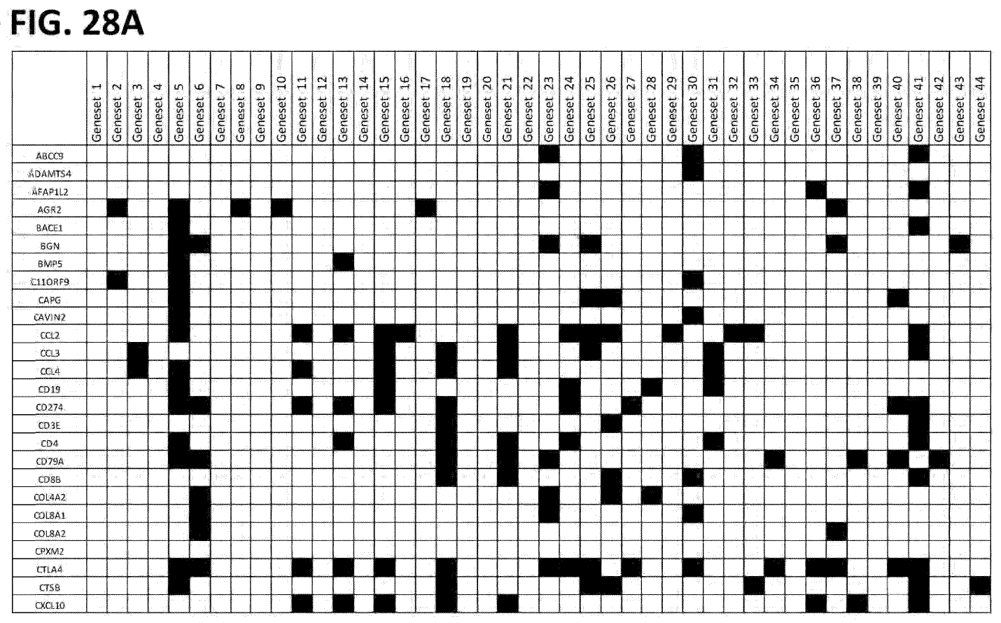

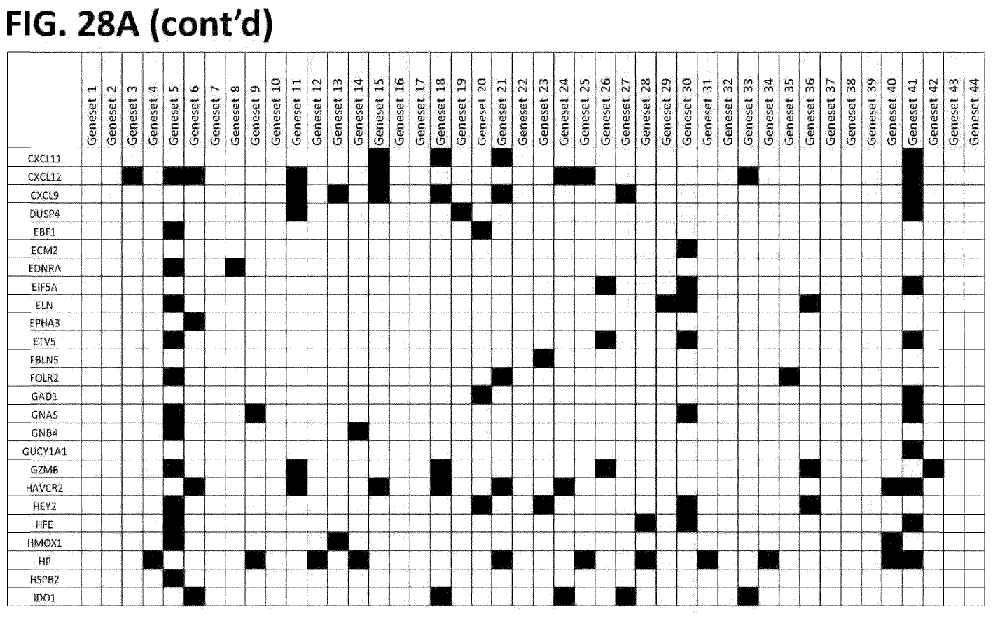

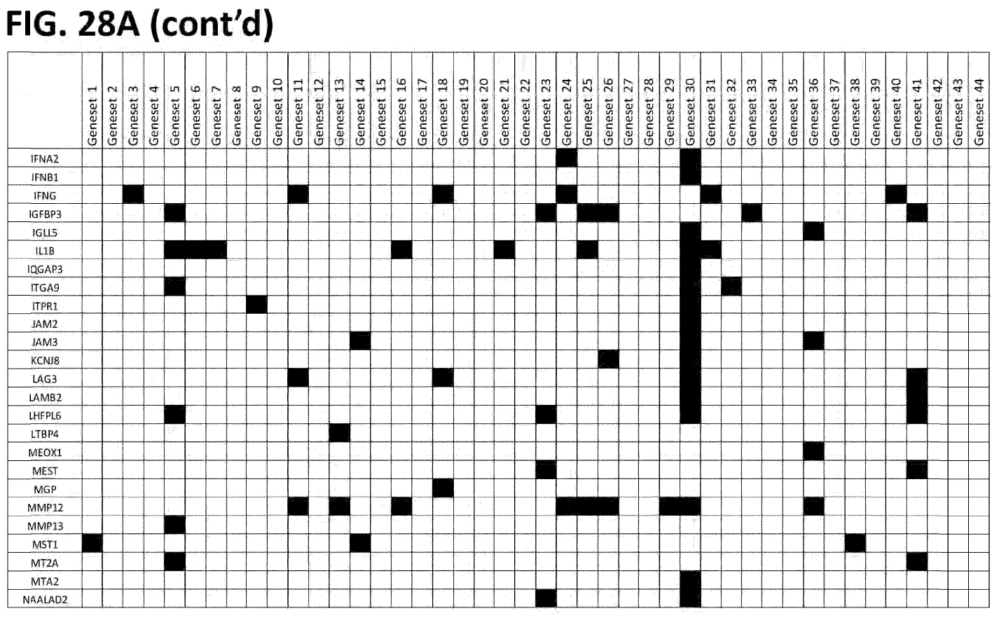

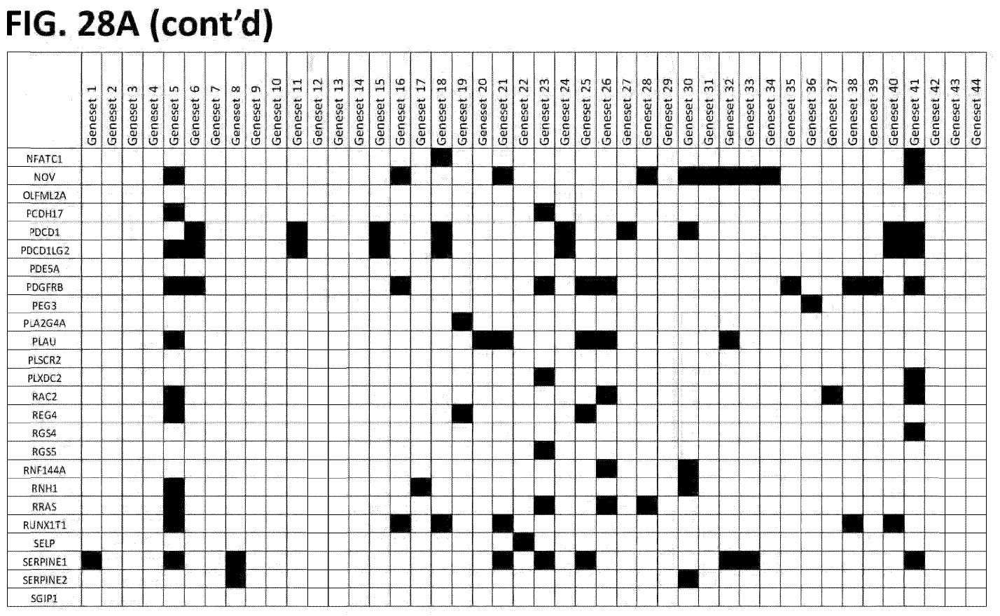

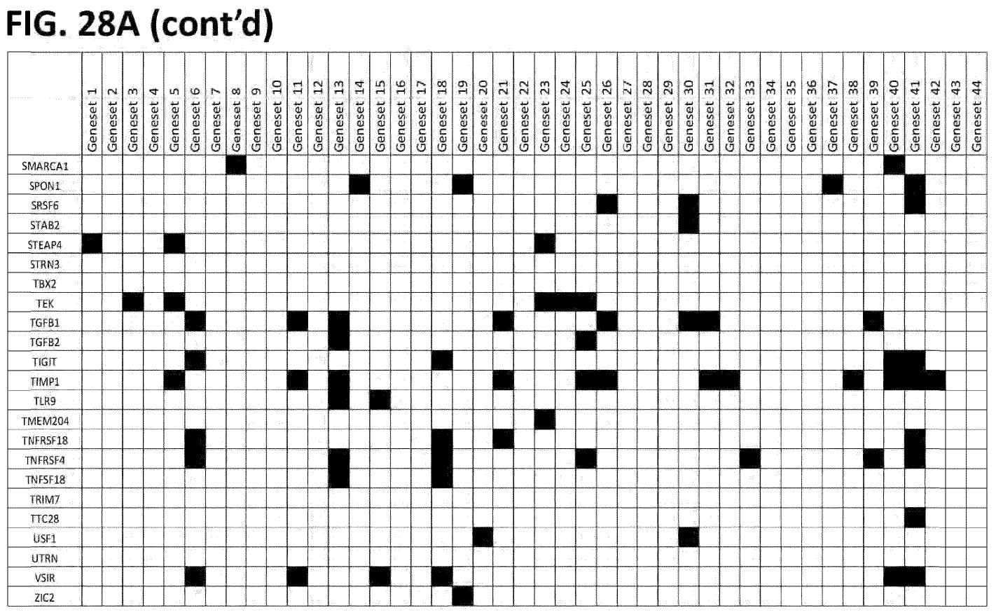

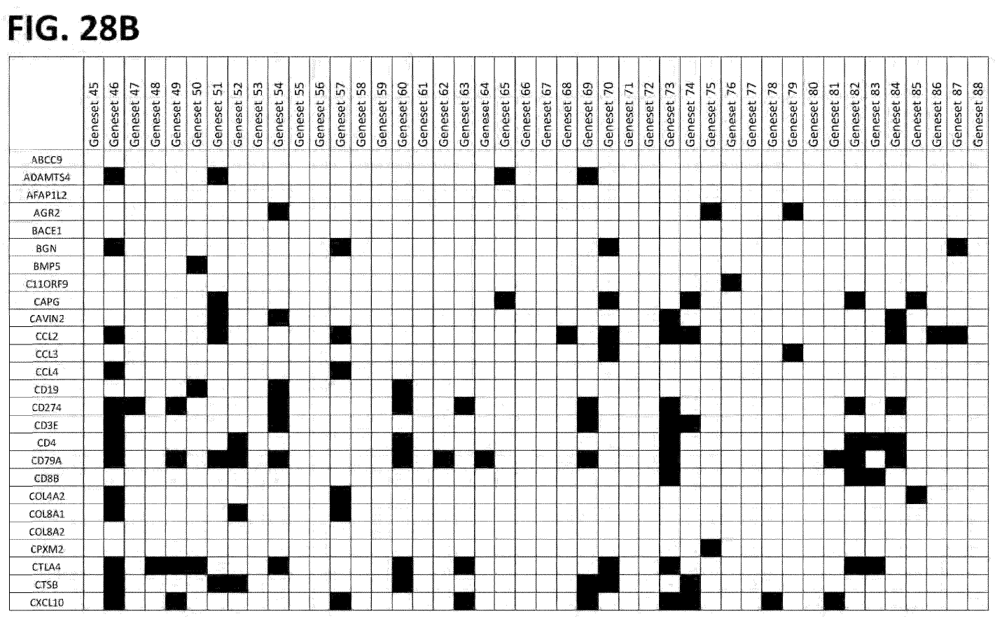

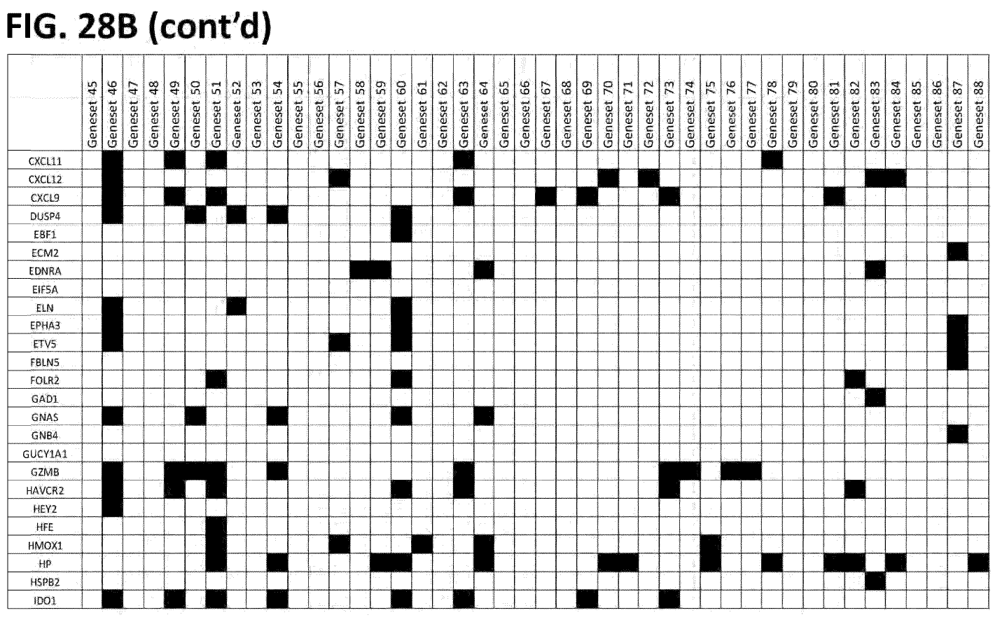

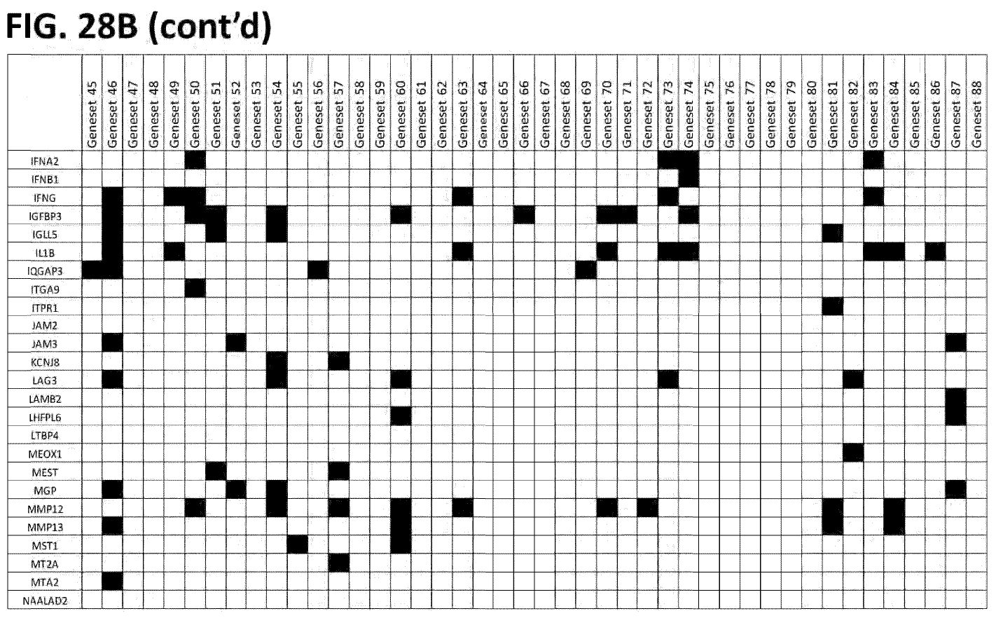

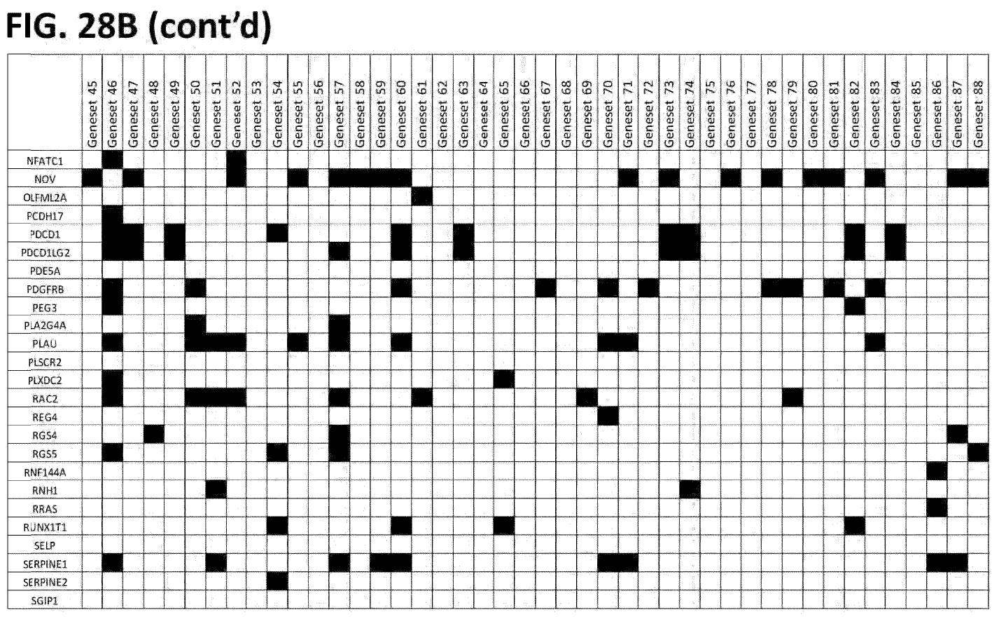

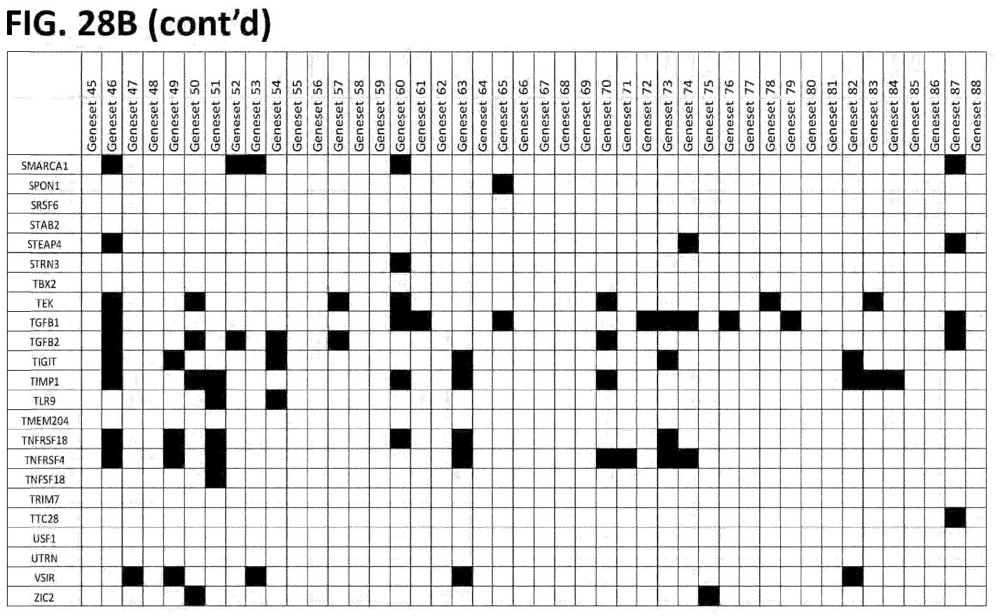

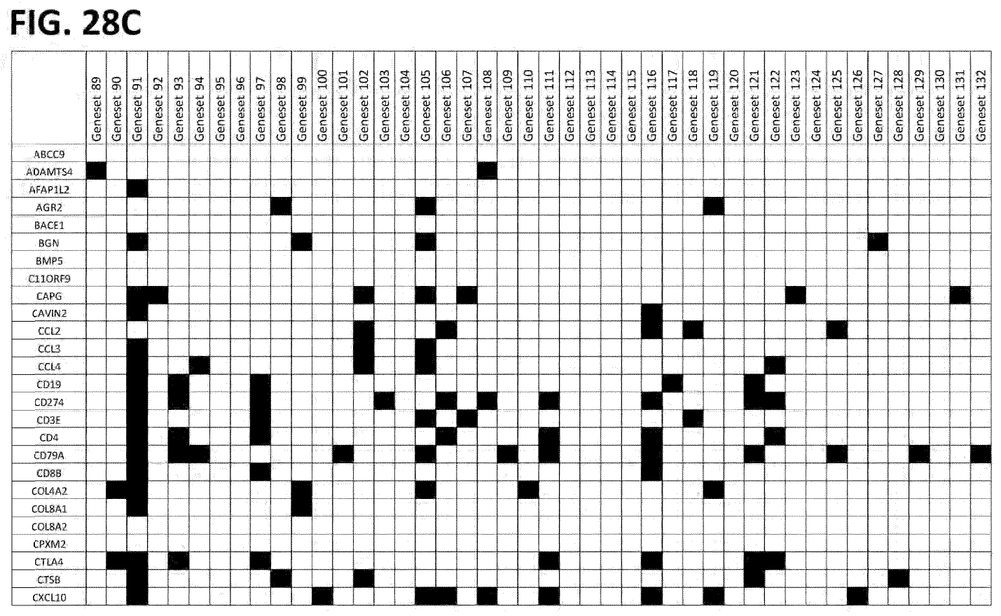

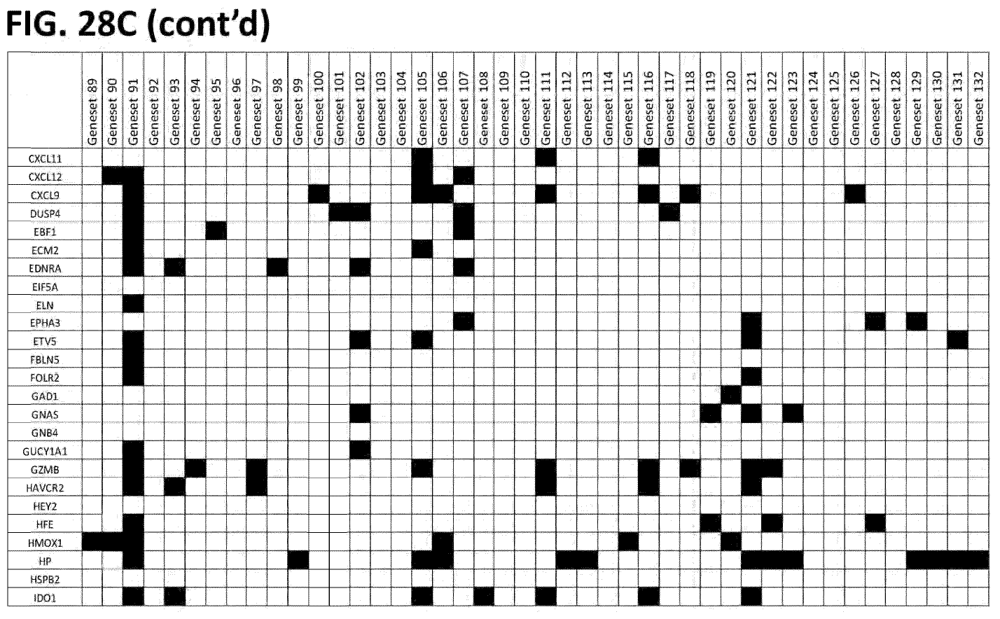

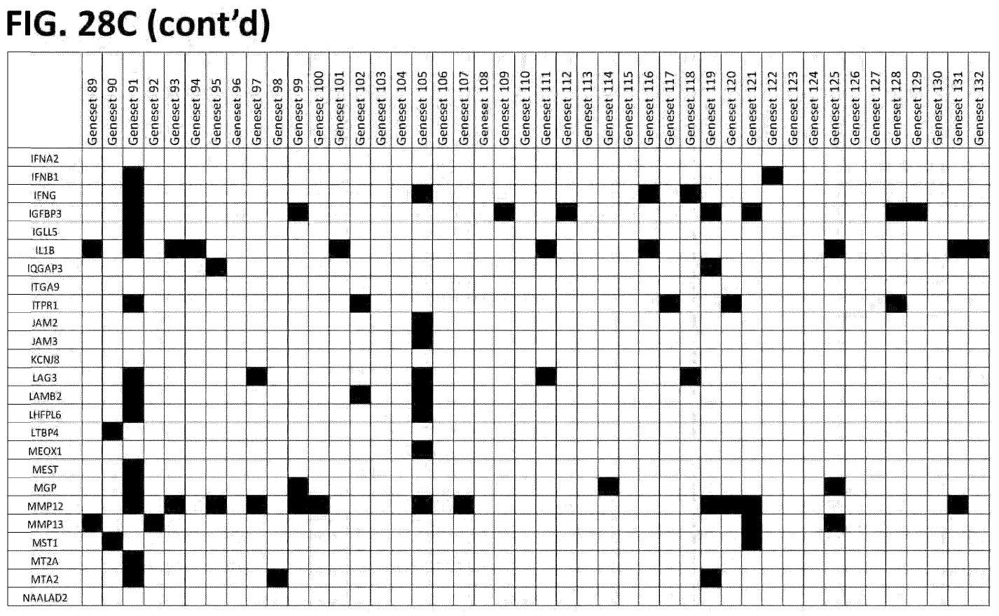

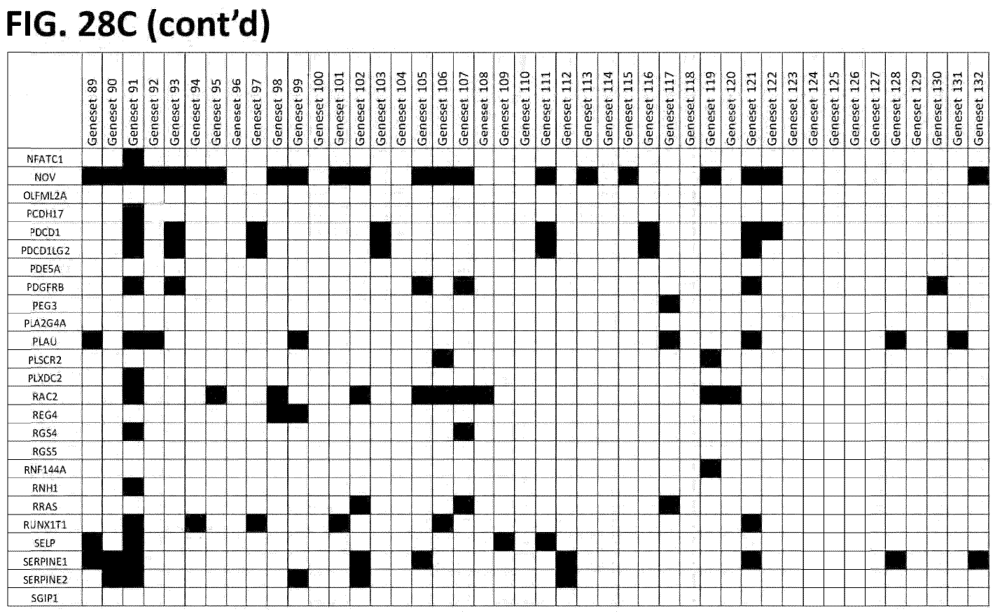

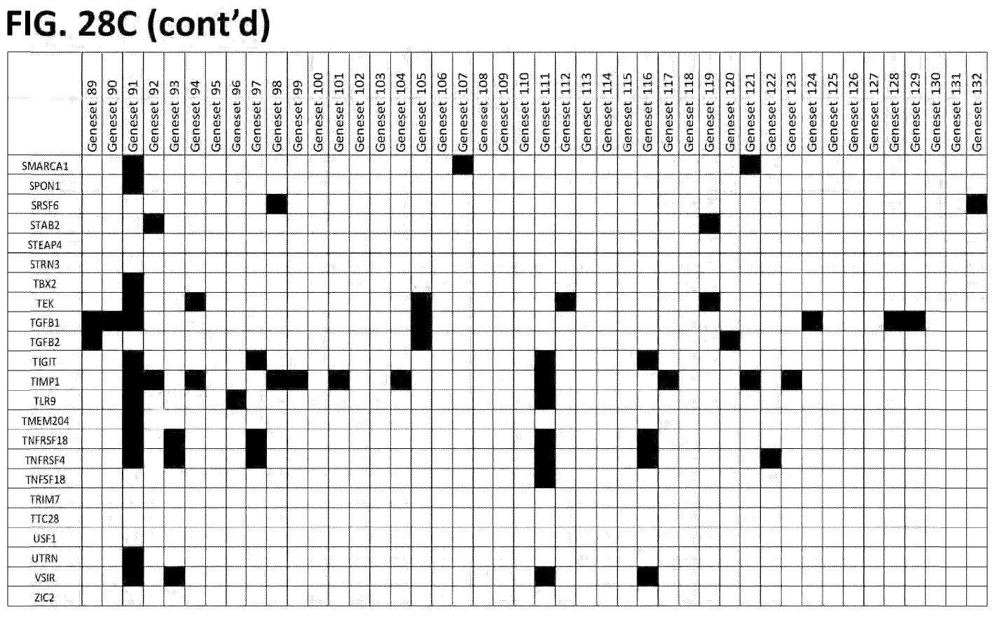

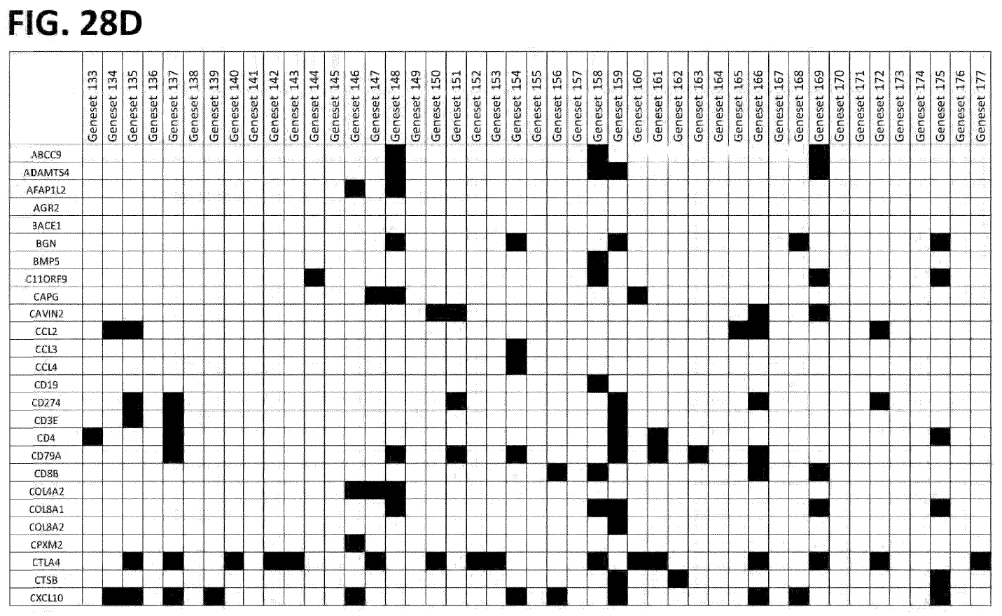

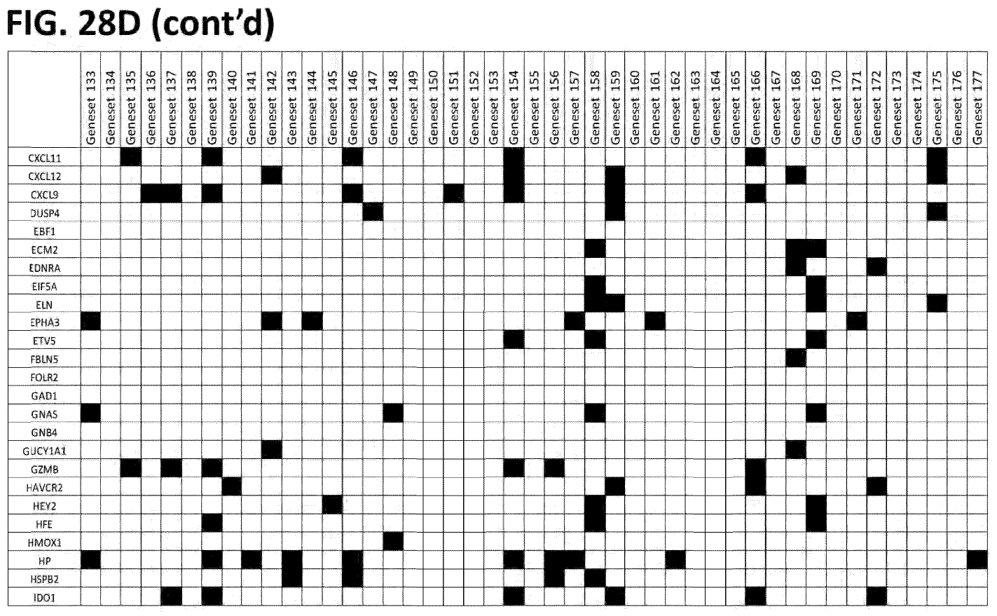

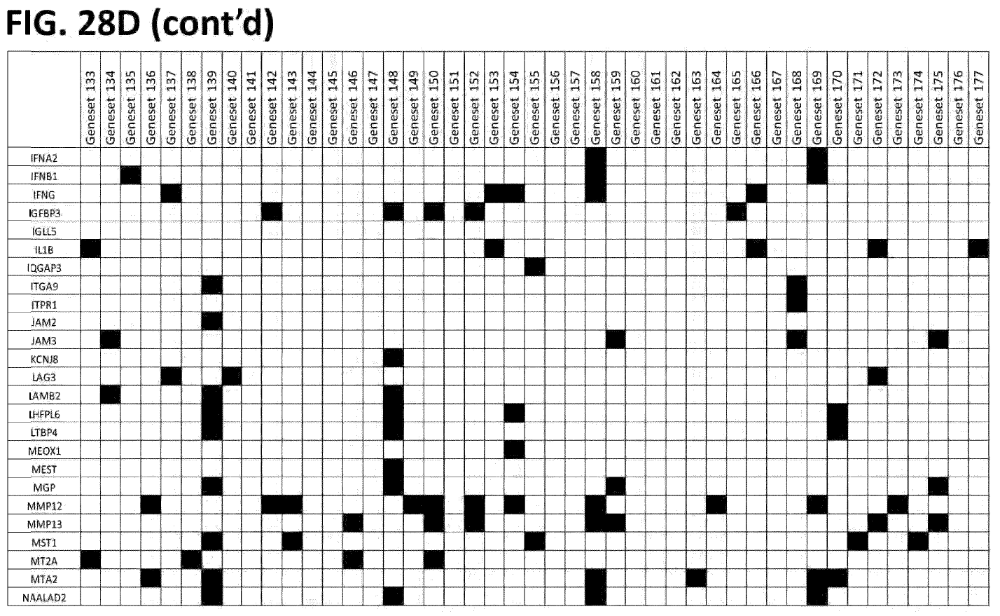

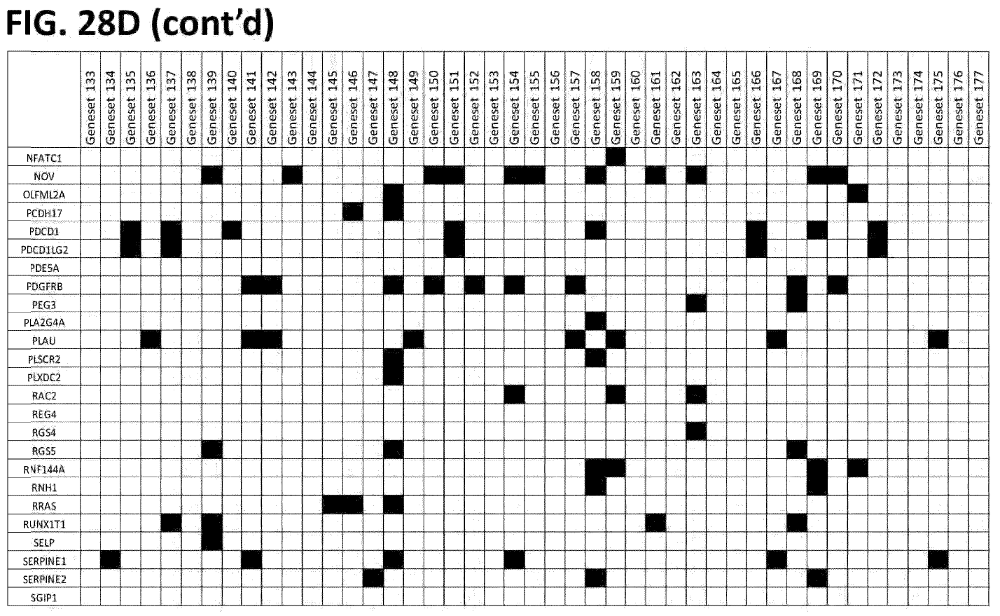

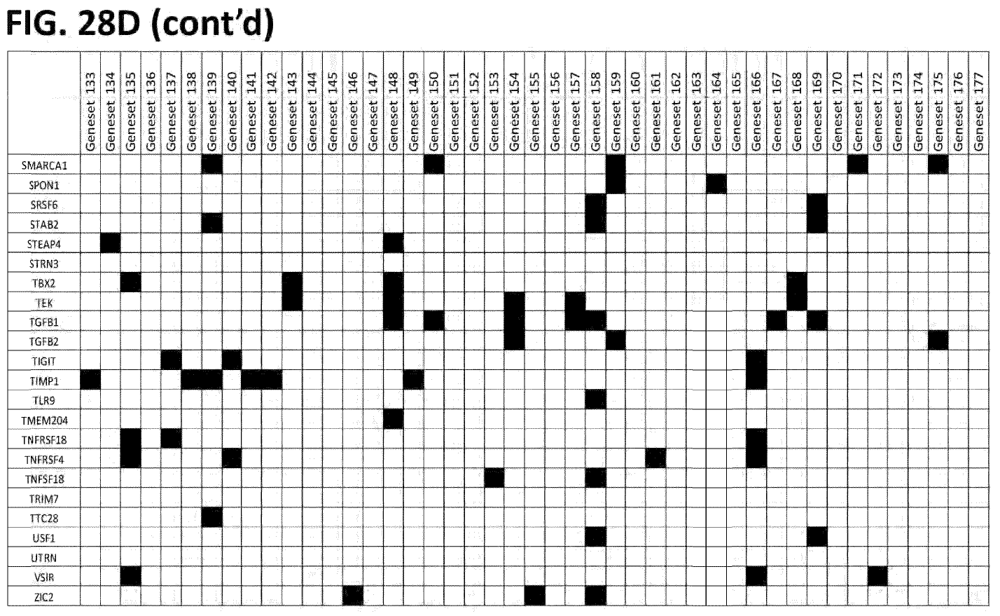

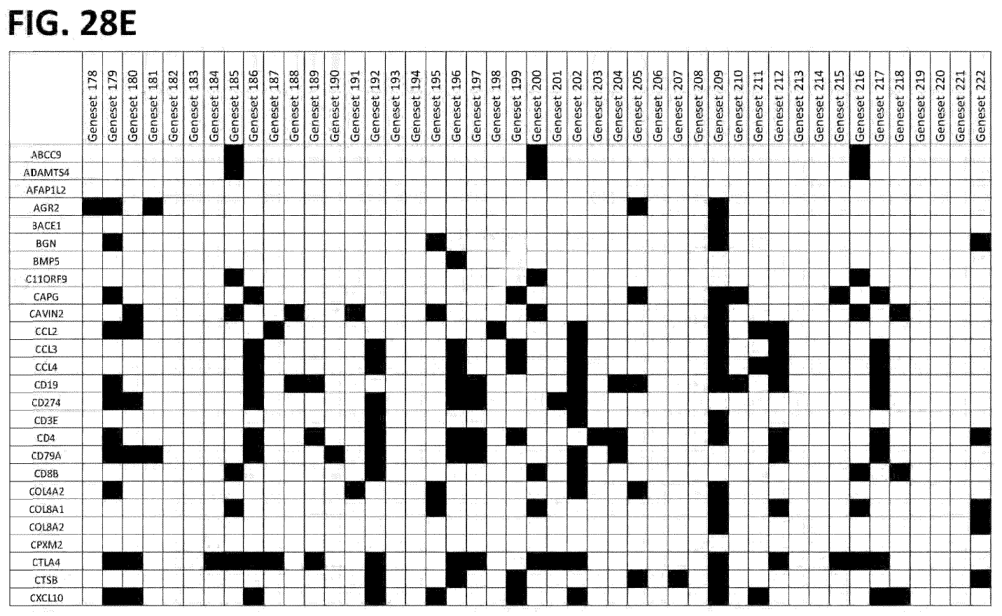

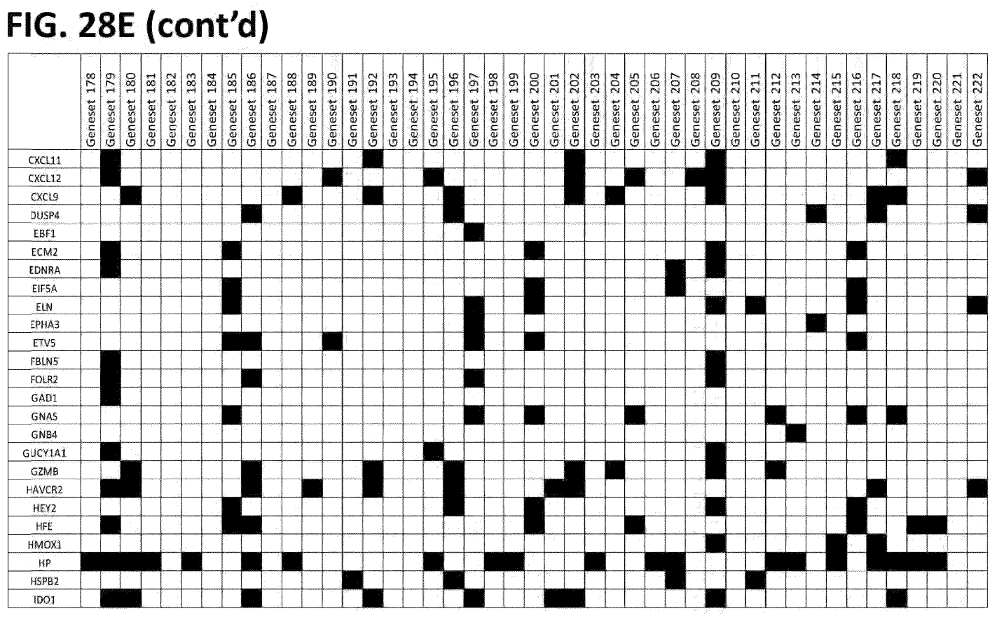

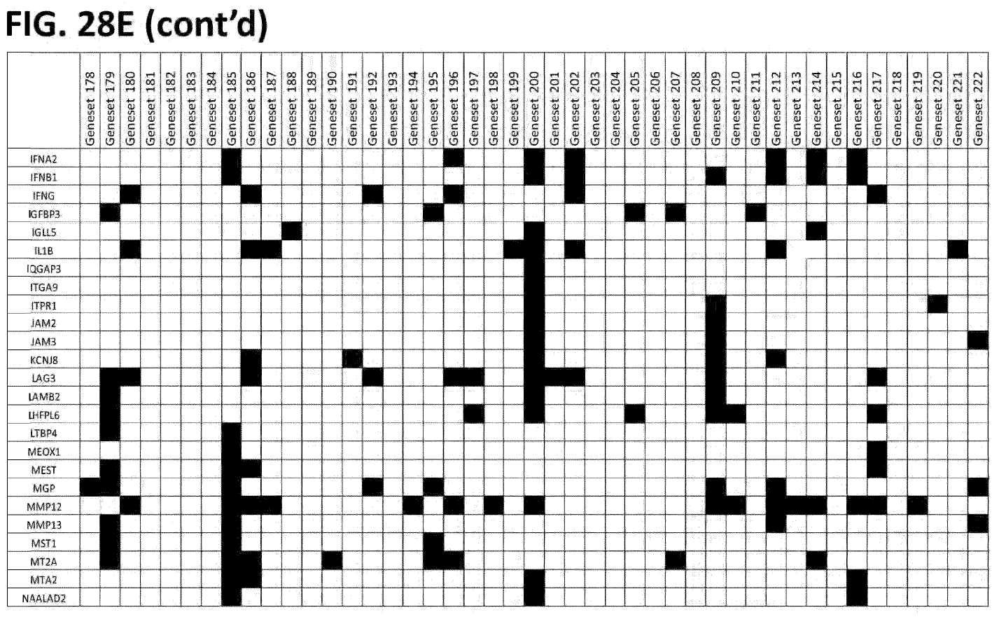

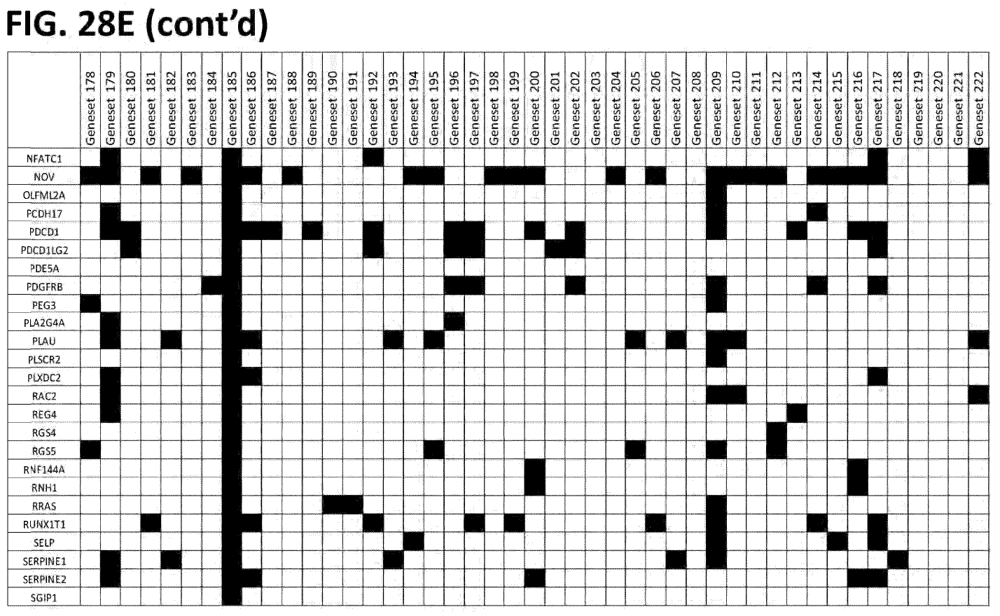

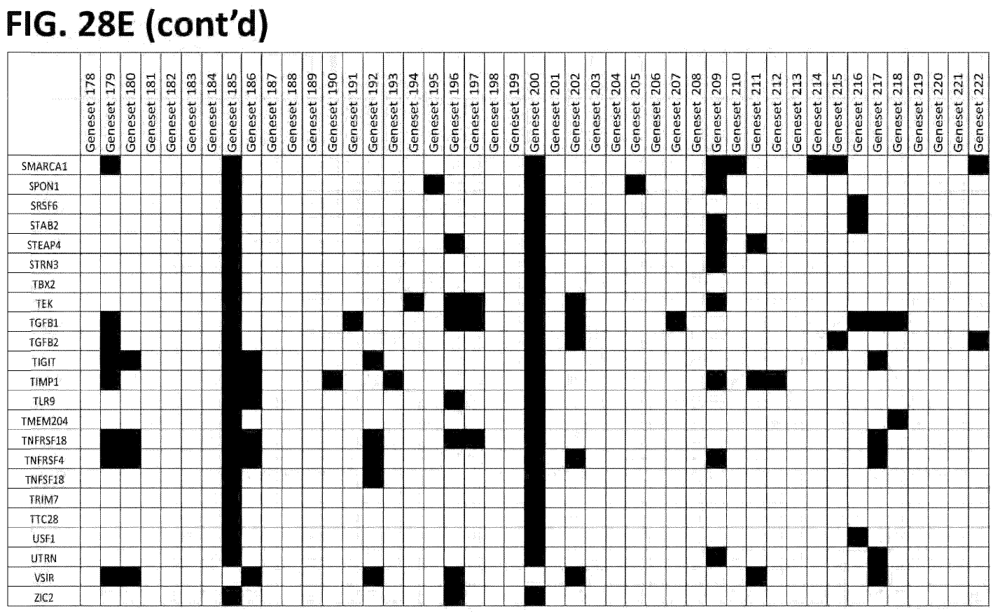

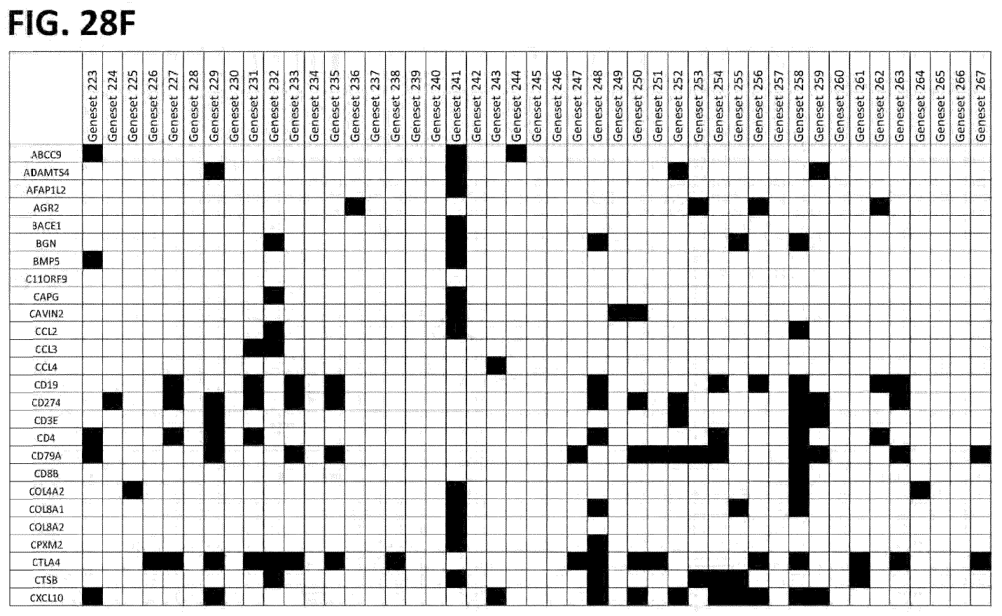

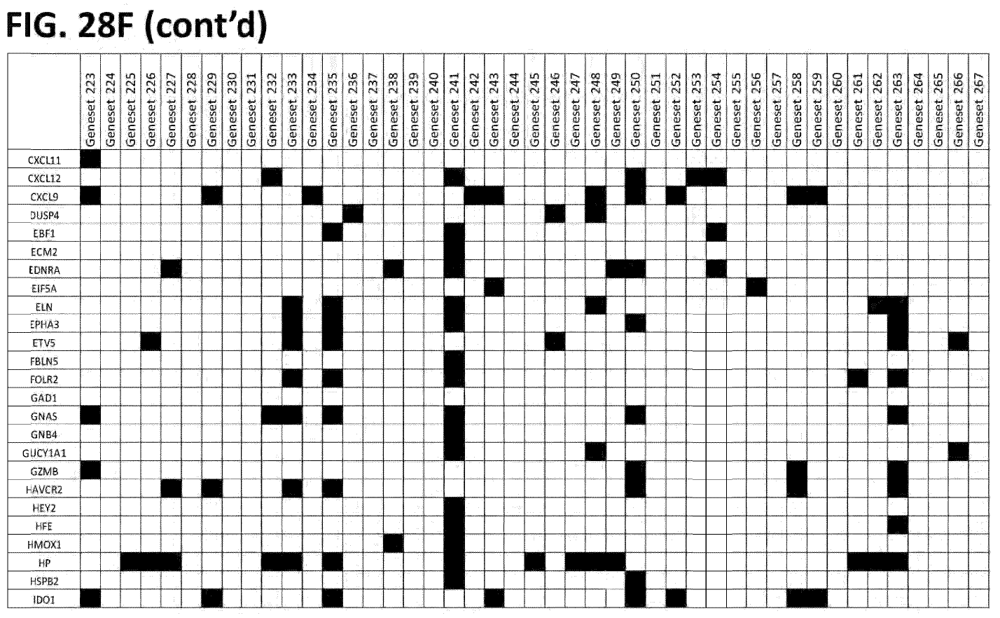

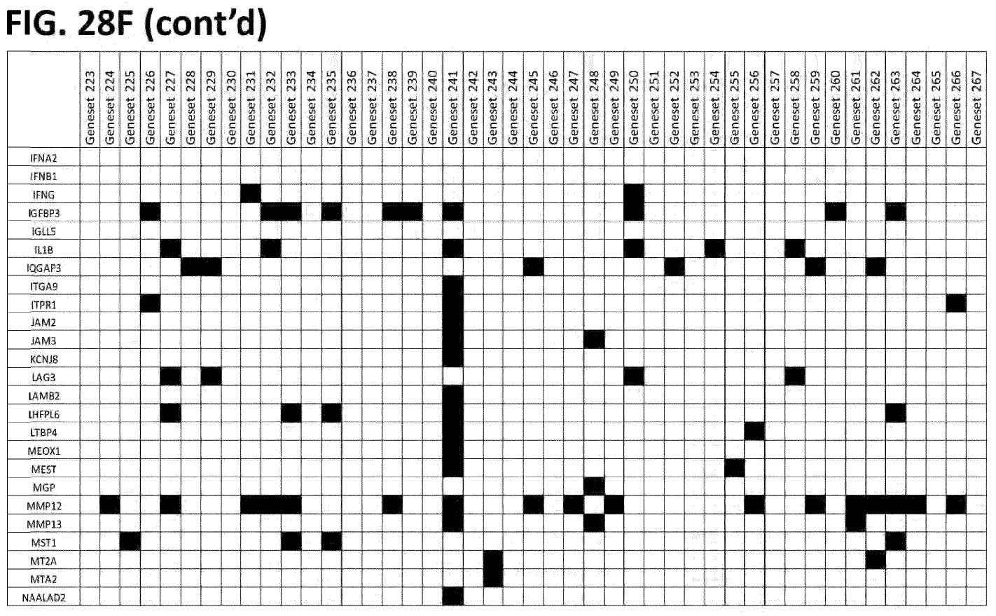

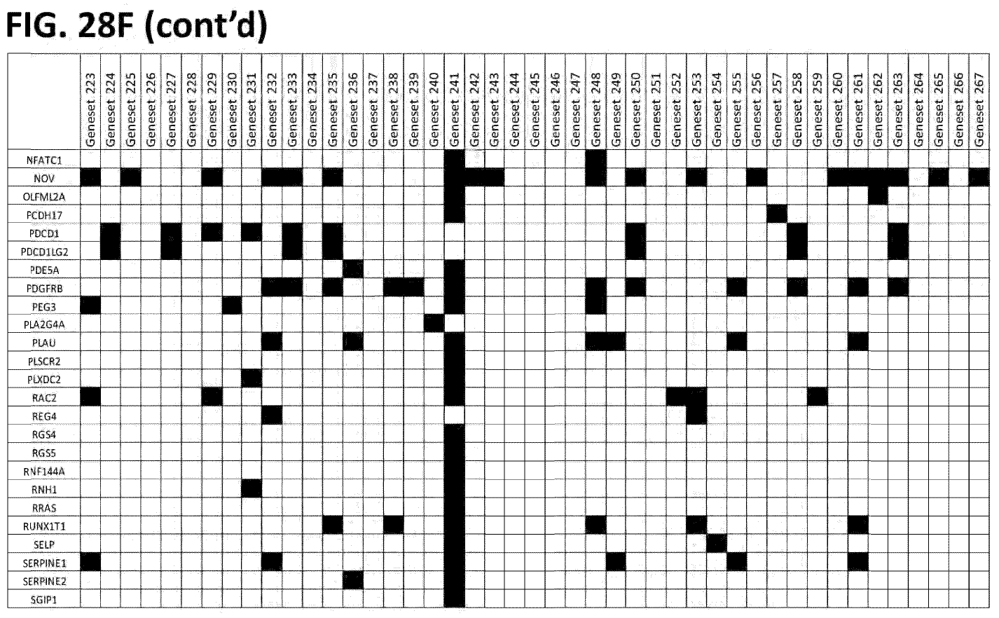

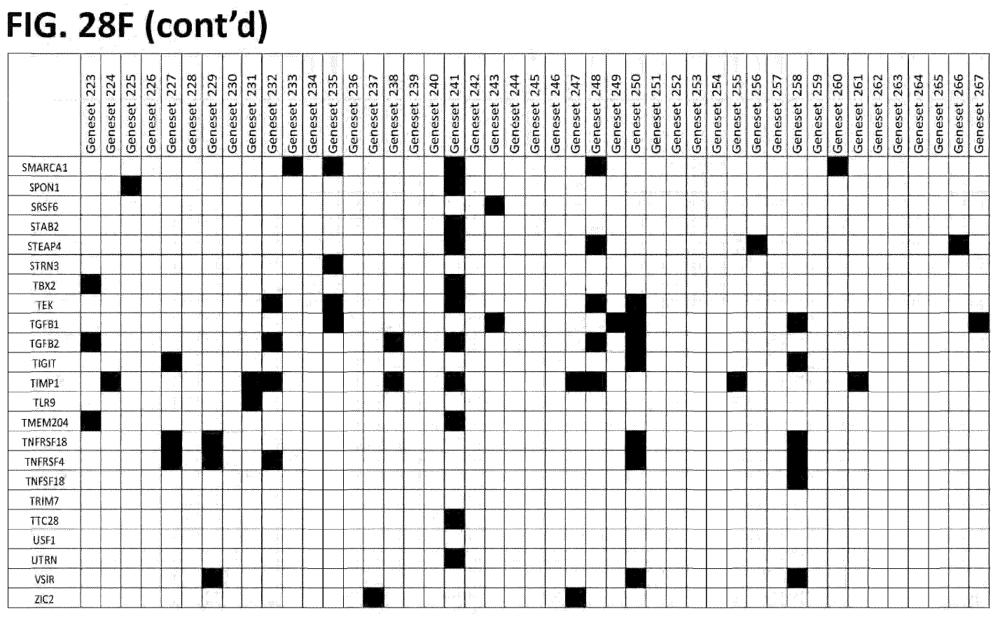

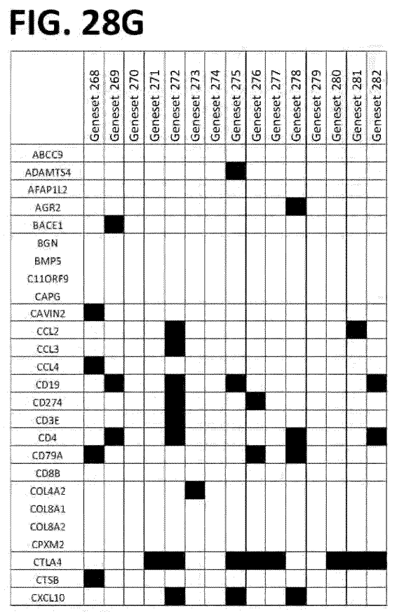

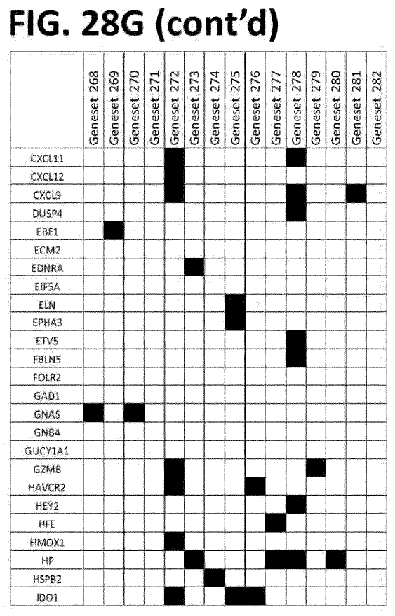

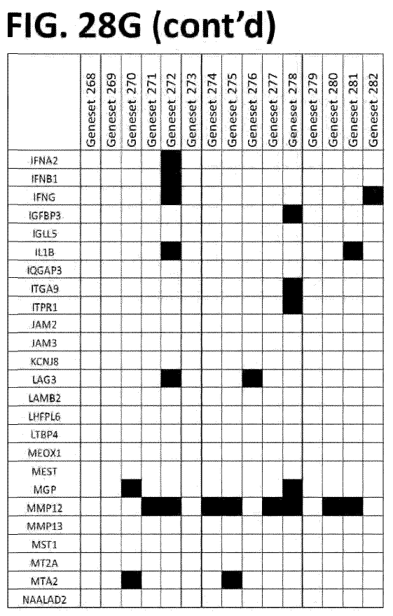

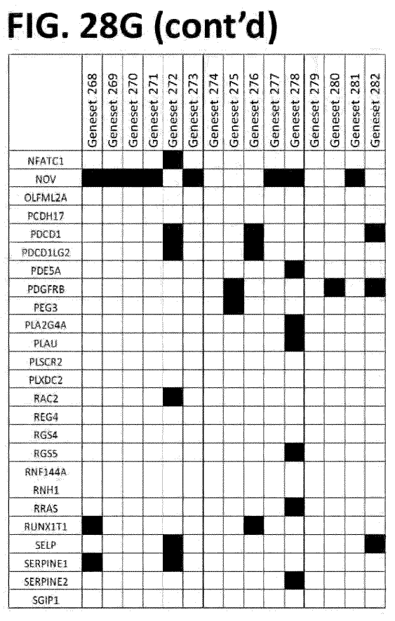

As used herein the term “population-based classifier” refers to a method of TME classification based on calculating one or more signatures corresponding to one or more characteristics (e.g., nucleic acid or protein expression levels) of a population of biomarkers (e.g., a population of biomarker genes disclosed herein). In some aspects, each signature is calculated using gene expression data (e.g., RNA expression data) obtained for a set of genes from a gene panel disclosed herein, e.g., a subset of the genes disclosed in TABLE 1 or TABLE 2, or any of the gene panels (Genesets) disclosed in FIGS. 28A-G.

As used herein, the term “non-population-based classifier” refers to a method of TME classification based on the application of a predictive model generated by machine-learning, e.g., ANN. In some aspects, the non-population-based classifier is generated using, for example, a training set comprising expression data (e.g., RNA expression data) preprocessed according to a population-based classifier disclosed herein as training set.

In some aspects, there is no difference in the results of the application of either the population-based methods or non-population-based methods as disclosed herein when archival samples are used as compared to fresh samples (non-archival samples). Example 7 discloses an application of an ANN method to fresh samples (non-archival samples). Example 12 discloses an application of an ANN method to archival samples.

In some aspects, fresh samples are preferred to archival samples. As used herein, the terms “fresh sample,” “non-archival sample,” and grammatical variants thereof refer to a sample (e.g., a tumor sample) which has been processed (e.g., to determine RNA or protein expression) before a predetermined period of time, e.g., one week, after extraction from a subject. In some aspects, a fresh sample has not been frozen. In some aspects, a fresh sample has not been fixed. In some aspects, a fresh sample has been stored for less than about two weeks, less than about one week, or less than six, five, four, three, or two days before processing. As used herein, the term “archival sample” and grammatical variants thereof refers to a sample (e.g., a tumor sample) which has been processed (e.g., to determine RNA or protein expression) after a predetermined period of time, e.g., a week, after extraction from a subject. In some aspects, an archival sample has been frozen. In some aspects, an archival sample has been fixed. In some aspect, an archival sample has a known diagnostic and/or a treatment history. In some aspects, an archival sample has been stored for at least one week, at least one month, at least six months, or at least one year, before processing.

In some aspects, a population-based classifier of the present disclosure comprises, e.g., determining a combined biomarker comprising at least a signature score determined by measuring the expression levels of a gene panel (e.g., a gene panel comprising at least one gene from TABLE 1 or TABLE 2, or any of the gene panels (Genesets) disclosed in FIGS. 28A-G, or a combination thereof) in a sample obtained from the subject; wherein the at least one signature score allows assignment of the subject's cancer to a particular TME class or a combination thereof.

In some aspects, a non-population-based classifier of the present disclosure comprises measuring the expression levels of a gene panel (e.g., a gene panel comprising at least one gene from TABLE 1 or TABLE 2, or any of the gene panels (Genesets) disclosed in FIGS. 28A-G, or a combination thereof) in a sample obtained from the subject; and applying a predictive model generated via machine-learning (e.g., a logistic regression, a random forest, an artificial neural network, or a support vector machine model), which assigns the subject's cancer to a particular TME class or a combination thereof. In some aspects, the machine-learning model output (e.g., the output from an ANN disclosed herein) is post-processed using a statistical function which assigns the machine-learning model output to a particular TME class or a combination thereof.

Afterwards, the classifier output (e.g., from a population-based classifier, a non-population-based classifier, or a combination thereof) assigning the subject's cancer to a particular TME or a combination thereof would guide the selection and administration of a specific treatment or treatments which have been determined to be effective to treat the same type of cancer in other subjects having the same TME, i.e., a TME-class therapy disclosed below or a combination thereof.

As used herein, the terms “tumor microenvironment” and “TME” refer to the environment surrounding tumor cells, including, e.g., blood vessels, immune cells, endothelial cells, fibroblasts, other stromal cells, signaling molecules, and the extracellular matrix. In some aspects, the terms “stromal subtype,” “stromal phenotype,” and grammatical variants thereof are used interchangeably with the term “TME.”

The tumor cells and the surrounding microenvironment are closely related and interact constantly. In general, tumor microenvironment (also known as, e.g., stromal phenotype) encompasses any structural and/or functional characteristic of the stroma of a tumor and tumoral environment. Numerous non-tumoral cell types can exist in a TME, e.g., carcinoma associated fibroblasts, myeloid-derived suppressor cells, tumor-associated macrophages, neutrophils, or tumor infiltrating lymphocytes. In some aspects, the classification of a particular TME can include the analysis of the cell types present in the stroma. A TME can also be characterized by specific functional characteristics, e.g., by abnormal oxygenation levels, abnormal blood vessel permeability, or abnormal levels of particular proteins such as collagens, elastin, glycosaminoglycans, proteoglycans, or glycoproteins.

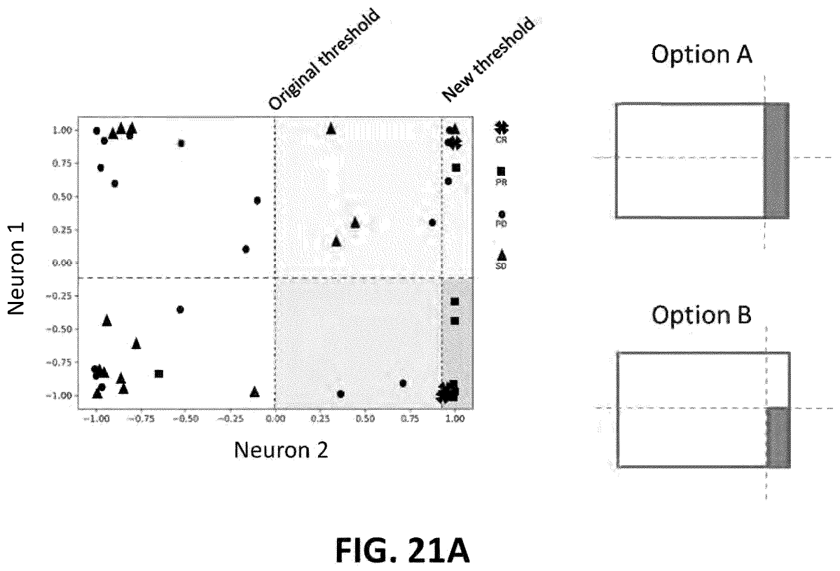

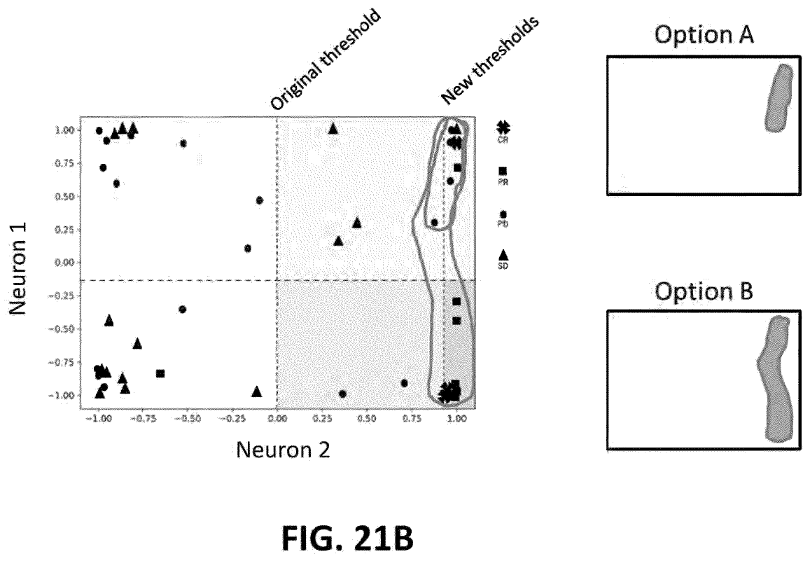

The population-based and non-population-based classifiers disclosed herein can be used to assign a patient or a cancer sample to a specific TME class (e.g., ID, IA, IS, or A) or to a combination thereof (e.g., ID and IA, ID and IS, ID and A, and so on). Specific subpopulations of patients within a specific TME class can be further classified based on the application of thresholds (e.g., by using a linear threshold or combination thereof, as exemplified in FIG. 21A, or by using non-linear thresholds as exemplified in FIG. 21B, or combinations thereof).

This classification functions as a combined biomarker, i.e., it is a biomarker derived from discrete biomarkers (e.g., a TME class or a subset within a specific TME defined, e.g., according to linear or non-linear threshold, or a combination thereof) integrated into a single score or a combination thereof in the case of a population-based classifier, or into a model in a non-population-based classifier. Accordingly, a patient or cancer sample can be “biomarker-positive” for a single TME class, e.g., ID, IA, IS or A, in which the patient or sample would be described as being, e.g., ID biomarker-positive, IA biomarker-positive, IS biomarker-positive, or A biomarker-positive. In some aspects, a patient or cancer sample can be biomarker-positive for more than one TME class. Thus, in some aspects, a patient or cancer sample can be biomarker-positive for 2, 3, 4 or more TME classes. In some aspects, a patient or cancer sample can be, e.g., ID and IA biomarker-positive; ID and IS biomarker-positive; ID and A biomarker-positive; IA and IS biomarker-positive; IA and A biomarker-positive; or IS and A biomarker-positive. In some aspects, a patient or cancer sample can be, e.g., ID, IA, and IS biomarker-positive; ID, IA, and A biomarker-positive; or ID, IS, and A biomarker-positive.

In some aspects, a combined probability for biomarker positive status (i.e., a combination of one or more probabilities coming from the stromal phenotype classifier) is used. The combined probability for biomarker positive status can be calculated using mathematical techniques known in the art.

A patient or cancer sample can also be defined as “biomarker-negative” for a single TME class, e.g., ID, IA, IS, or A. Thus, the patient or sample would be described as being, e.g., ID biomarker-negative, IA biomarker-negative, IS biomarker-negative, or A biomarker-negative. In some aspects, a patient or cancer sample can be biomarker-negative for more than one TME class. Thus, in some aspects, a patient or cancer sample can be biomarker-negative for 2, 3, 4 or more TME classes. In some aspects, a patient or cancer sample can be, e.g., ID and IA biomarker-negative; ID and IS biomarker-negative; ID and A biomarker-negative; IA and IS biomarker-negative; IA and A biomarker-negative; or IS and A biomarker-negative. In some aspects, a patient or cancer sample can be, e.g., ID, IA, and IS biomarker-negative; ID, IA, and A biomarker-negative; or ID, IS, and A biomarker-negative.

In some aspects, a combined probability for biomarker negative status (i.e., a combination of one or more probabilities coming from the stromal phenotype classifier) is used. The combined probability for biomarker negative status can be calculated using mathematical techniques known in the art.

In some aspects, assignment of a TME-class specific therapy is based on the presence of a specific stromal phenotype, i.e., if a subject presents an IA stromal phenotype (and therefore the subject is IA biomarker-positive), an IA-class TME therapy would be administered. In some aspects, assignment of a TME-class specific therapy is based on the absence of a specific stromal phenotype, i.e., if a subject does not present an IA stromal phenotype (and therefore the subject is IA biomarker-negative), an IA-class TME therapy would not be administered.

In some aspects, the classification of a patient or cancer sample to a TME class, and assignment of a TME class therapy to the patient or cancer is not biunivocal. In other words, a patient or cancer sample can be classified as biomarker-positive and/or biomarker-negative for more than one TME class, and more than one TME class therapy or a combination thereof can be used to treat that patient. For example, the classification of a patient or cancer sample as biomarker-positive for two different TME classes (i.e., two stromal phenotypes) could be used to select a treatment comprising a combination of pharmacological approaches in the TME class therapies corresponding to the TME classes for which the patient or cancer sample is biomarker-positive. Furthermore, if the patient or cancer sample is biomarker-negative for a particular TME class, such knowledge can be used to exclude specific pharmacological approaches in the TME class therapy corresponding to the TME class for which the patient or cancer sample is biomarker-negative. Thus, drugs or combinations thereof, treatments or combinations thereof, and/or clinical regimens or combinations that are useful to treat a cancer sample classified as biomarker-positive for a particular TME class, can be combined to treat patients having more than one biomarker-positive signal (i.e., having a cancer sample classified as biomarker-positive for more than one stromal phenotype).

In some aspects, depending on the mechanism of action of a drug or a clinical regimen, different classification parameters, e.g., different gene panel subsets, different thresholds, different ANN architectures, different activation functions, or different post-processing functions, can be used to yield different TME classes, which in turn would be used to select appropriate TME class therapies. Accordingly, each drug or drug regimen may have different diagnostic gene panels and differently configured population based or non-population based classifiers to inform the clinician (such as a medical doctor), e.g., to decide whether a patient should be selected for treatment, whether treatment should be initiated, whether treatment should be suspended, or whether treatment should be modified.

In some aspects, a clinician can account for co-variates of biomarker status of a patient, and combine the probability of the stromal phenotype or biomarker status with MSI/MSS (Microsatellite Instability/Microsatellite Stability-high) status, EBV (Epstein-Barr virus) status, PD-1/PD-L1 status (such as CPS, i.e., combined positive score), neutrophil-leukocyte ratio (NLR), or confounding variables such as prior treatment history.

In some aspects, the clinician is given a binary result from the algorithm, and the decision to treat or not treat as described herein is made. In one aspect, the clinician is given, e.g., a plot of the patient's result superimposed on a latent space and interpreted with probability thresholds, or a linear or polynomial logistic regression.

### I.A. Gene Panels

The population- and non-population-based classifiers of the present disclosure rely on the selection of a specific gene panel as the source of the input data used by the classifier. In some aspects, each one of the genes in a gene panel of the present disclosure is referred to as a “biomarker.” The terms “geneset” and “gene panel” are used interchangeably.

In some aspects, the biomarker is a nucleic acid biomarker. The term “nucleic acid biomarker,” as used herein, refers to a nucleic acid (e.g., a gene in a gene panel disclosed herein) that can be detected (e.g., quantified) in a subject or a sample therefrom, e.g., a sample comprising tissues, cells, stroma, cell lysates, and/or constituents thereof, e.g., from a tumor. In some aspects, the term nucleic acid biomarker refers to the presence or absence of a specific sequence of interest (e.g., a nucleic acid variant or a single nucleotide polymorphism) in a nucleic acid (e.g., a gene in a gene panel disclosed herein) that can be detected (e.g., quantified) in a subject or a sample therefrom, e.g., a sample comprising tissues, cells, stroma, cell lysates, and/or constituents thereof, e.g., from a tumor.

The “level” of a nucleic acid biomarker can, in some aspects, refer to the “expression level” of the biomarker, e.g., the level of an RNA or DNA encoded by the nucleic acid sequence of the nucleic acid biomarker in a sample. For example, in some aspects, the expression level of a particular gene disclosed in TABLE 1 or TABLE 2, or any of the gene panels (Genesets) disclosed in FIGS. 28A-G, refers to the amount of mRNA encoding such gene present in a sample obtained from a subject.

In some aspects, the “level” of a nucleic acid biomarker, e.g., an RNA biomarker, can be determined by measuring a downstream output (e.g., an activity level of a target molecule or an expression level of an effector molecule that is modulated, e.g., activated or inhibited, by the nucleic acid biomarker or an expression product, e.g., RNA or DNA, thereof).

In some aspects, the nucleic acid biomarker is an RNA biomarker. An “RNA biomarker,” as used herein, refers to an RNA comprising the nucleic acid sequence of a nucleic acid biomarker of interest, e.g., RNA encoding a particular gene disclosed in TABLE 1 or TABLE 2, or any of the gene panels (Genesets) disclosed in FIGS. 28A-G.

The “expression level” of an RNA biomarker generally refers to a detected quantity of RNA molecules comprising the nucleic acid sequence of interest present in the subject or sample therefrom, e.g., the quantity of RNA molecules expressed from a DNA molecule (e.g., the genome of the subject or the subject's cancer) comprising the nucleic acid sequence.

In some aspects, the expression level of an RNA biomarker is the quantity of the RNA biomarker in a tumor stromal sample. In some aspects, an RNA biomarker is quantified using PCR (e.g., real-time PCR), sequencing (e.g., deep sequencing or next generation sequencing, e.g., RNA-Seq), or microarray expression profiling or other technologies that utilize RNAse protection in combination with amplification or amplification and new quantitation methods such as RNA-Seq or other methods.

In some aspects, a population-based classifier disclosed herein comprises signatures calculated using expression levels of a gene disclosed in TABLE 1 and TABLE 2 (or in any of the gene panels (Genesets) disclosed in FIGS. 28A-G). For example, a population-based classifier comprising two signatures can comprise a Signature 1 obtained from expression levels corresponding to the genes disclosed in TABLE 1 or a subset thereof, and a Signature 2 obtained from expression levels corresponding to the genes disclosed in TABLE 2 or a subset thereof. In some specific aspects, the population-based classifier can use subsets (gene panels) disclosed in TABLE 3 and TABLE 4. For example, a population-based classifier comprising two signatures can comprise a Signature 1 obtained from expression levels corresponding to genes in a gene panel disclosed in TABLE 3, and a Signature 2 obtained from expression levels corresponding to genes in a gene panel disclosed in TABLE 4 or a subset thereof.

In the population-based classifiers disclosed herein, expression levels for genes in a gene panel acquired from a population of samples (e.g., samples from a clinical study) can be used to classify groups of samples in the population as belonging to a TME class (or a combination thereof, i.e., a sample can be classified not only as biomarker-positive for a single TME class, but also can be classified as biomarker-positive for two or more TME classes) according to whether calculated signature levels are above or below certain threshold values. Subsequently, expression levels for genes in a gene panel obtained from a sample or samples from a test subject can be used to classify the subject's TME into one of the TME classes identified in the population.

In the non-population-based classifiers disclosed herein, expression levels for genes in a gene panel acquired from a population of samples (e.g., samples from a clinical study) and their assignments to a TME class (or a combination thereof, i.e., a sample can be classified not only as biomarker-positive for a single TME class, but also can be classified as biomarker-positive for two or more TME classes) obtained according to the populations classifiers disclosed herein can be used as a training set for machine-learning, e.g., using an ANN. The machine-learning process would yield a model, e.g., an ANN model. Subsequently, expression levels for genes in a gene panel obtained from a sample or samples from a test subject would be used as input for the model, which would classify the subject's TME into a particular TME class (or a combination thereof, i.e., a sample can be classified not only as biomarker-positive for a single TME class, but also can be classified as biomarker-positive for two or more TME classes).

Standard names, aliases, etc. of proteins and genes designated by identifiers used throughout this disclosure can be identified, for example, via Genecards (www.genecards.org) or Uniprot (www.uniprot.org).

TABLE 1
Signature 1 genes and accession numbers (n = 63)

| Gene | | RefSeq RNA (NM_xxxxxx) and Transcript variants |
|---|---|---|
| Symbol | Gene Description | (XM_xxxxxx) |
| ABCC9 | ATP binding cassette | NM_005691.3, NM_020297.3, NM_020298.2, |
| | subfamily C member 9 | XM_005253284.3, XM_005253286.3 XM_005253287.4, |
| | | XM_005253288.3, XM_005253289.3, XM_005253290.3, |
| | | XM_006719025.3, XM_011520545.2 |
| AFAP1L2 | actin filament | NM_001146337.2, NM_001323062.1, NM_001323063.1, |
| | associated protein 1 | NM_152406.3, XM_011537558.1, XM_017009036.1 |
| | like 2 | |
| BACE1 | beta-secretase 1 | NM_001207048.1, NM_001207049.1, NM_012104.4, |
| | | NM_138971.3, NM_138972.3, NM_138973.3 |
| BGN | Biglycan | NM_001711.5, XM_017029724.1 |
| BMP5 | bone morphogenetic | NM_001329754.1, NM_001329756.1, NM_021073.3, |
| | protein 5 | XM_005249304.3, XM_011514816.2, XM_011514817.2, |
| | | XM_017011198.1 |
| COL4A2 | collagen type IV alpha | NM_001846.3 |
| | 2 chain | |
| COL8A1 | collagen type VIII | NM_001850.4, NM_020351.3 |
| | alpha 1 chain | |
| COL8A2 | collagen type VIII | NM_001294347.1, NM_005202.3, XM_005270477.3 |
| | alpha 2 chain | |
| CPXM2 | carboxypeptidase X, | NM_198148.2, XM_005269528.3, XM_011539283.2, |
| | M14 family member 2 | XM_011539285.2, XM_011539286.1, XM_017015673.1, |
| | | XM_017015674.1 |
| CXCL12 | C—X—C motif | NM_000609.6, NM_001033886.2, NM_001178134.1, |
| | chemokine ligand 12 | NM_001277990.1, NM_199168.3 |
| EBF1 | early B cell factor 1 | NM_001290360.2, NM_001324101.1, NM_001324103.1, |
| | | NM_001324106.1, NM_001324107.1, NM_001324108.1, |
| | | NM_001324109.1, NM_001324111.1, NM_024007.4, |
| | | NM_182708.2, XM_017009192.1, XM_017009193.1, |
| | | XM_017009194.1, XM_017009195.1, XM_017009196.1, |
| | | XM_017009197.1, XM_017009198.1, XM_017009199.1, |
| | | XM_017009200.1, XM 017009201.1, XM_017009202.1, |
| | | XM_017009203.1, XM_017009204.1 |
| ECM2 | extracellular matrix | NM_001197295.1, NM_001197296.1, NM_001393.3, |
| | protein 2 | XM_017014376.1, XM_017014377.1 |
| EDNRA | endothelin receptor | NM_001166055.1, NM_001354797.1, NM_001957.3, |
| | type A | NM_001256283.1 |
| ELN | Elastin | NM_000501.3, NM_001081752.2, NM_001081753.2, |
| | | NM_001081754.2, NM_001081755.2, NM_001278912.1, |
| | | NM_001278913.1, NM_001278914.1, NM_001278915.1, |
| | | NM_001278916.1, NM_001278917.1, NM_001278918.1, |
| | | NM_001278939.1, XM_005250187.1, XM_005250188.1, |
| | | XM_011515868.1, XM_011515869.1, XM_011515870.1, |
| | | XM_011515871.1, XM_011515872.1, XM_011515873.1, |
| | | XM_011515874.1, XM_011515875.1, XM_011515876.1, |
| | | XM_011515877.1, XM_017011813.1, XM_017011814.1 |
| EPHA3 | EPH receptor A3 | NM_005233.5, NM_182644.2, XM_005264715.2, |
| | | XM_005264716.2 |
| FBLN5 | fibulin 5 | NM_006329.3 XM_005267267.3 XM_011536356.1 |
| | | XM_011536357.1 XM_011536358.1 XM_017020929.1 |
| GNAS | GNAS complex locus | NM_000516.5, NM_001077488.3, NM_001077489.3, |
| | | NM_001077490.2, NM_001309840.1, NM_001309842.1, |
| | | NM_001309861.1, NM_001309883.1, NM_016592.3, |
| | | NM_080425.3, NM_080426.3, XM_017027812.1, |
| | | XM_017027813.1, XM_017027814.1, XM_017027815.1, |
| | | XM_017027816.1, XM_017027817.1, XM_017027818.1, |
| | | XM_017027819.1, XM_017027820.1, XM_017027821.1, |
| | | XM_017027822.1 |
| GNB4 | G protein subunit beta | NM_021629.3, XM_005247692.2, XM_006713721.2 |
| | 4 | |
| GUCY1A3 | guanylate cyclase 1 | NM_000856.5, NM_001130682.2, NM_001130683.3, |
| | soluble subunit alpha | NM_001130684.2, NM_001130685.2, NM_001130687.2, |
| | 1 | NM_001256449.1, NM_001130686.1, XM_005262955.2, |
| | | XM_005262956.2, XM_005262957.2, XM_006714196.2, |
| | | XM_006714197.2, XM_006714198.2, XM_011531900.2 |
| HEY2 | HES related family | NM_012259.2, XM_017010627.1, XM_017010628.1, |
| | bHLH transcription | XM_017010629.1 |
| | factor with YRPW | |
| | motif 2 | |
| HSPB2 | heat shock protein | NM_001541.3 |
| | family B (small) | |
| | member 2 | |
| IL1B | interleukin 1 beta | NM_000576.2, XM_017003988.1 |
| ITGA9 | integrin subunit alpha | NM_002207.2 |
| | 9 | |
| ITPR1 | inositol 1,4,5- | NM_001099952.2, NM_002222.5, NM_001168272.1, |
| | trisphosphate receptor | XM_005265109.2, XM_005265110.2, XM_006713131.2, |
| | type 1 | XM_011533681.1, XM_011533682.2, XM_011533683.2, |
| | | XM_011533684.1, XM_011533685.1, XM_011533686.1, |
| | | XM_011533687.1, XM_011533688.1, XM_011533690.1, |
| | | XM 011533691.1, XM_011533692.2, XM_017006357.1, |
| | | XM 017006358.1 |
| JAM2 | junctional adhesion | NM_001270408.1, NM_021219.3, NM_001270407.1 |
| | molecule 2 | |
| JAM3 | junctional adhesion | NM_001205329.1, NM_032801.4 |
| | molecule 3 | |
| KCNJ8 | potassium voltage- | NM_004982.3, XM_005253358.4, XM_017019283.1, |
| | gated channel | XM_017019284.1 |
| | subfamily J member 8 | |
| LAMB2 | laminin subunit beta 2 | NM_002292.3, XM_005265127.3 |
| LHFP | LHFPL tetraspan | NM_005780.2, XM_011534861.1 |
| | subfamily member 6 | |
| LTBP4 | latent transforming | NM_001042544.1, NM_001042545.1, NM_003573.2, |
| | growth factor beta | XM_011527376.2, XM_011527377.2, XM_011527378.2, |
| | binding protein 4 | XM_011527379.1, XM_011527380.2, XM_011527381.2, |
| | | XM_011527382.2, XM_011527383.2, XM_011527384.2, |
| | | XM_011527385.2, XM_011527386.2, XM_011527387.1, |
| | | XM_017027352.1, XM_017027353.1, XM_017027354.1 |
| MEOX1 | mesenchyme | NM_001040002.1, NM_004527.3, NM_013999.3, |
| | homeobox 1 | XM_011524818.1 |
| MGP | matrix Gla protein | NM_000900.4, NM_001190839.2 |
| MMP12 | matrix | NM_002426.5 |
| | metallopeptidase 12 | |
| MMP13 | matrix | NM_002427.3 |
| | metallopeptidase 13 | |
| NAALAD2 | N-acetylated alpha- | NM_001300930.1, NM_005467.3, XM_017017043.1, |
| | linked acidic | XM_017017044.1, XM_017017045.1, XM_017017046.1 |
| | dipeptidase 2 | |
| NFATC1 | nuclear factor of | NM_001278669.1, NM_001278670.1, NM_001278672.1, |
| | activated T cells 1 | NM_001278673.1, NM_001278675.1, NM_006162.4, |
| | | NM_172387.2, NM_172388.2, NM_172389.2, NM_172390.2, |
| | | XM_017025783.1 |
| NOV | nephroblastoma | NM_002514.3 |
| | overexpressed | |
| OLFML2A | olfactomedin like 2A | NM_001282715.1, NM_182487.3, XM_005251760.4, |
| | | XM_006716989.2 |
| PCDH17 | protocadherin 17 | NM_001040429.2, NM_014459.2, XM_005266357.2, |
| | | XM_005266358.2, XM_017020547.1 |
| PDE5A | phosphodiesterase 5A | NM_001083.3, NM_033430.2, NM_033437.3, |
| | | XM_017008791.1 |
| PDGFRB | platelet derived | XM_011537659.1, XM_011537658.1, XM_005268464.2, |
| | growth factor receptor | NM_002609.3, NM_001355017.1, NM_001355016.1 |
| | beta | |
| PEG3 | paternally expressed 3 | NM_001146184.1, NM_001146185.1, NM_001146186.1, |
| | | NM_001146187.1, NM_006210.2 |
| PLSCR2 | phospholipid | NM_001199978.1, NM_001199979.1, NM_020359.2, |
| | scramblase 2 | XM_011513013.2, XM_011513019.2, XM_011513020.2, |
| | | XM_011513021.2, XM_011513022.2, XM_011513023.2, |
| | | XM_017006898.1, XM_017006899.1, XM_017006900.1, |
| | | XM_017006901.1, XM_017006902.1, XM_017006903.1, |
| | | XM_017006904.1, XM_017006905.1, XM_017006906.1, |
| | | XM_017006907.1, XM_017006908.1, XM_017006909.1, |
| | | XM_017006910.1, XM_017006911.1, XM_017006912.1, |
| | | XM_017006913.1, XM_017006914.1, XM_017006915.1 |
| PLXDC2 | plexin domain | NM_001282736.1, NM_032812.8, XM_011519750.2 |
| | containing 2 | |
| RGS4 | regulator of G protein | NM_001102445.2, NM_001113380.1, NM_001113381.1, |
| | signaling 4 | NM_005613.5 |
| RGS5 | regulator of G protein | NM_001195303.2, NM_001254748.1, NM_001254749.1, |
| | signaling 5 | NM_003617.3, NM_025226.1 |
| RNF144A | ring finger protein | NM_001349181.1, NM_001349182.1, NM_001349183.1, |
| | 144A | NM_001349184.1, NM_001349185.1, NM_001349186.1, |
| | | NM_014746.5, XM_005246200.3, XM_005246202.4, |
| | | XM_017005396.1, XM_017005397.1, XM_017005398.1, |
| | | XM_017005399.1, XM_017005400.1, XM_017005401.1, |
| | | XM_017005402.1, XM_017005403.1, XM_017005404.1 |
| RRAS | RAS related | NM_006270.4 |
| RUNX1T1 | RUNX1 translocation | NM_001198625.1, NM_001198626.1, NM_001198627.1, |
| | partner 1 | NM_001198628.1, NM_001198629.1, NM_001198630.1, |
| | | NM_001198631.1, NM_001198632.1, NM_001198633.1, |
| | | NM_001198634.1, NM_001198679.1, NM_004349.3, |
| | | NM_175634.2, NM_175635.2, NM_175636.2, |
| | | XM_006716676.3, XM_011517351.2, XM_011517352.2, |
| | | XM_011517353.2, XM_017013930.1, XM_017013931.1, |
| | | XM_017013932.1, XM_017013933.1, XM_017013934.1, |
| | | XM_017013935.1, XM_017013936.1, XM_017013937.1, |
| | | XM_017013938.1, XM_017013939.1, XM_017013940.1, |
| | | XM_017013941.1 |
| CAV2 | caveolae associated | NM_004657.5 |
| | protein 2 | |
| SELP | selectin P | NM_003005.3, XM_005245435.1, XM_005245436.3, |
| | | XM_005245438.1, XM_005245439.1, XM_005245440.1 |
| SERPINE2 | serpin family E | NM_001136528.1, NM_001136530.1, NM_006216.3, |
| | member 2 | XM_005246641.2, XM_017004329.1, XM_017004330.1, |
| | | XM_017004331.1, XM_017004332.1 |
| SGIP1 | SH3 domain GRB2 | NM_001308203.1, NM_001350217.1, NM_001350218.1, |
| | like endophilin | NM_032291.3, XM_005271264.3, XM_005271268.3, |
| | interacting protein 1 | XM_005271270.4, XM_006710961.2, XM_006710966.2, |
| | | XM_006710967.2, XM_006710969., XM_006710971.2, |
| | | XM_006710972.2, XM_006710973.2, XM_006710974.2, |
| | | XM_011542291.1, XM_011542292.1, XM_011542293.1, |
| | | XM_017002505.1, XM_017002506.1, XM_017002507.1, |
| | | XM_017002508.1, XM_017002509.1, XM_017002510.1, |
| | | XM_017002511.1, XM_017002512.1, XM_017002513.1, |
| | | XM_017002514.1, XM_017002515.1, XM_017002516.1, |
| | | XM_017002517.1, XM_017002518.1, XM_017002519.1, |
| | | XM_017002520.1, XM_017002521.1, XM_017002522.1, |
| | | XM_017002523.1, XM_017002524.1, XM_017002525.1, |
| | | XM_017002526.1, XM_017002527.1, XM_017002528.1, |
| | | XM_017002529.1, XM_017002530.1, XM_017002531.1, |
| | | XM_017002532.1, XM_017002533.1, XM_017002534.1, |
| | | XM_017002535.1, XM_017002536.1, XM_017002537.1 |
| SMARCA1 | SWI/SNF related, | NM_001282874.1, NM_001282875.1, NM_003069.4, |
| | matrix associated, | NM_139035.2, XM_005262461.2, XM_005262462.2, |
| | actin dependent | XM_006724782.2, XM_017029750.1, XM_017029751.1 |
| | regulator of | |
| | chromatin, subfamily | |
| | a, member 1 | |
| SPON1 | spondin 1 | NM_006108.3 |
| STAB2 | stabilin 2 | NM_017564.9, XM_011538537.2, XM_011538538.2, |
| | | XM_011538539.2, XM_011538541.2, XM_011538542.2, |
| | | XM_017019585.1 |
| STEAP4 | STEAP4 | NM_001205315.1, NM_001205316.1, NM_024636.3 |
| | metalloreductase | |
| TBX2 | T-box 2 | NM_005994.3 |
| TEK | TEK receptor tyrosine | NM_000459.4, NM_001290077.1, NM_001290078.1, |
| | kinase | XM_005251561.2, XM_005251563.2 |
| TGFB2 | transforming growth | NM_001135599.3, NM_003238.4 |
| | factor beta 2 | |
| TMEM204 | transmembrane | NM_001256541.1, NM_024600.5 |
| | protein 204 | |
| TTC28 | tetratricopeptide | NM_015281.1, NM_001145418.1, XM_005261405.2, |
| | repeat domain 28 | XM_006724171.4, XM_011530018.3, XM_011530019.2, |
| | | XM_011530020.1, XM_011530021.3, XM_011530022.1, |
| | | XM_017028673.2 |
| UTRN | Utrophin | NM_007124.2, XM_005267127.5, XM_005267130.2, |
| | | XM_005267133.3, XM_006715560.4, XM_011536101.3, |
| | | XM_011536102.2, XM_011536106.2, XM_011536109.3, |
| | | XM_017011243.2, XM_017011244.1, XM_017011245.1, |
| | | XM_024446536.1 |

TABLE 2
Signature 2 genes and accession numbers (n = 61)

| Gene | | RefSeq RNA (NM_xxxxxx) and Transcript variants |
|---|---|---|
| Symbol | Gene Description | (XM_xxxxxx) |
| AGR2 | anterior gradient 2, | NM_006408.3, XM_005249581.4 |
| | protein disulphide | |
| | isomerase family | |
| | member | |
| C11orf9 | myelin regulatory factor | NM_001127392.2, NM_013279.3, XM_005274222.1, |
| | | XM_005274223.1, XM_005274224.1, |
| | | XM_005274225.1, XM_005274226.1, XM_005274227.1, |
| | | XM_005274228.1, XM_011545234.2 |
| DUSP4 | dual specificity | NM_001394.6, NM_057158.3, XM_011544428.2 |
| | phosphatase 4 | |
| EIF5A | eukaryotic translation | NM_001143760.1, NM_001143761.1, NM_001143762.1, |
| | initiation factor 5A | NM_001970.4, XM_005256509.2, XM_011523710.2, |
| | | XM_011523711.2, XM_011523712.2, XM_011523713.2, |
| | | XM_017024300.1, XM_017024301.1 |
| ETV5 | ETS variant 5 | NM_004454.2 |
| GAD1 | glutamate | NM_000817.2, NM_013445.3, XM_005246444.2, |
| | decarboxylase 1 | XM_011510922.1, XM_017003756.1, XM_017003757.1, |
| | | XM_017003758.1 |
| IQGAP3 | IQ motif containing | NM_178229.4, XM_011509198.2, XM_011509200.2, |
| | GTPase activating | XM_011509201.2, XM_017000317.1, XM_017000318.1 |
| | protein 3 | |
| MST1 | macrophage stimulating | NM_020998.3, XM_006713166.1, XM_011533732.1, |
| | 1 | XM_011533737.2 , XM_011533738.2, XM_017006460.1, |
| | | XM_017006461.1, XM 017006462.1, XM_017006463.1, |
| | | XM_017006464.1, XM_017006465.1, XM_017006466.1, |
| | | XM_017006467.1, XM_017006468.1 |
| MT2A | metallothionein 2A | NM_005953.4 |
| MTA2 | metastasis associated 1 | NM_001330292.1, NM_004739.3, XM_017018561.1 |
| | family member 2 | |
| PLA2G4A | phospholipase A2 | NM_001311193.1, NM_024420.2, XM_005245267.3, |
| | group IVA | XM_011509642.2 |
| REG4 | regenerating family | NM_001159352.1, NM_001159353.1, NM_032044.3 |
| | member 4 | |
| SRSF6 | serine and arginine rich | NM_006275.5 |
| | splicing factor 6 | |
| STRN3 | striatin 3 | NM_001083893.1, NM_014574.3, XM_005267569.3, |
| | | XM_005267570.3 |
| TRIM7 | tripartite motif | NM_033342.3, NM_203293.2, NM_203294.1, NM_203295.1, |
| | containing 7 | NM_203296.1, NM_203297.1, XM_017009903.1, |
| | | XM_017009904.1 |
| USF1 | upstream transcription | NM_001276373.1, NM_007122.4, NM_207005.2 |
| | factor 1 | |
| ZIC2 | Zic family member 2 | NM_007129.4, XM_011521110.2 |
| C10orf54 | V-set | NM_022153.1 |
| | immunoregulatory | |
| | receptor | |
| CCL3 | C—C motif chemokine | NM_002983.2 |
| | ligand 3 | |
| CCL4 | C—C motif chemokine | NM_002984.3 |
| | ligand 4 | |
| CD19 | CD19 molecule | NM_001178098.1, NM_001770.5, XM_006721103.3, |
| | | XM_011545981.1, XM_017023893.1 |
| CD274 | CD274 molecule | NM_001267706.1, NM_001314029.1, NM_014143.3 |
| CD3E | CD3e molecule | NM_000733.3 |
| CD4 | CD4 molecule | NM_000616.4, NM_001195014.2, NM_001195015.2, |
| | | NM_001195016.2, NM_001195017.2, XM_017020228.1 |
| CD8B | CD8b molecule | NM_001178100.1, NM_004931.4, NM_172101.3, |
| | | NM_172102.3, NM_172213.3, NM_172099.2, XM_011533164.2 |
| CTLA4 | cytotoxic T-lymphocyte | NM_001037631.2, NM_005214.4 |
| | associated protein 4 | |
| CXCL10 | C—X—C motif | NM_001565.3 |
| | chemokine ligand 10 | |
| IFNA2 | interferon alpha 2 | NM_000605.3 |
| IFNB1 | interferon beta 1 | NM_002176.3 |
| IFNG | interferon gamma | NM_000619.2 |
| LAG3 | lymphocyte activating 3 | NM_002286.5, XM_011520956.1 |
| PDCD1 | programmed cell death | NM_005018.2, XM_006712573.2, XM_017004293.1 |
| | 1 | |
| PDCD1LG2 | programmed cell death | NM_025239.3, XM_005251600.3 |
| | 1 ligand 2 | |
| TGFB1 | transforming growth | NM_000660.6, XM_011527242.1 |
| | factor beta 1 | |
| TIGIT | T cell immunoreceptor | NM_173799.3, XM_011512538.1, XM_017005865.1 |
| | with Ig and ITIM | |
| | domains | |
| TNFRSF18 | TNF receptor | NM_004195.2, NM_148901.1, NM_148902.1, XM_017002722.1 |
| | superfamily member 18 | |
| TNFRSF4 | TNF receptor | NM_003327.3, XM_011542074.2, XM_011542075.2, |
| | superfamily member 4 | XM_011542076.2, XM_011542077.2, M_017002231.1, |
| | | XM_017002232.1 |
| TNFSF18 | TNF superfamily | NM_005092.3 |
| | member 18 | |
| TLR9 | toll like receptor 9 | NM_017442.3, NM_138688.1 |
| HAVCR2 | hepatitis A virus | NM_032782.4 |
| | cellular receptor 2 | |
| CD79A | CD79a molecule | NM_001783.3, NM_021601.3 |
| CXCL11 | C—X—C motif | NM_001302123.1, NM_005409.4 |
| | chemokine ligand 11 | |
| CXCL9 | C—X—C motif | NM_002416.2 |
| | chemokine ligand 9 | |
| GZMB | granzyme B | NM_001346011.1, NM_004131.5, XM_011536685.2 |
| IDO1 | indoleamine 2,3- | NM_002164.5 |
| | dioxygenase 1 | |
| IGLL5 | immunoglobulin | NM_001178126.1, NM_001256296.1 |
| | lambda like polypeptide | |
| | 5 | |
| ADAMTS4 | ADAM | NM_001320336.1, NM_005099.5 |
| | metallopeptidase with | |
| | thrombospondin type 1 | |
| | motif 4 | |
| CAPG | capping actin protein, | NM_001256139.1, NM_001256140.1, NM_001320732.1, |
| | gelsolin like | NM_001320733.1, NM_001320734.1, NM_001747.3, |
| | | XM_011533122.1, XM_011533123.1 |
| CCL2 | C—C motif chemokine | NM_002982.3 |
| | ligand 2 | |
| CTSB | cathepsin B | NM_001317237.1, NM_001908.4, NM_147780.3, |
| | | NM_147781.3, NM_147782.3, NM_147783.3, |
| | | XM_006716244.2, XM_006716245.2, XM_011543812.2, |
| | | XM_017013097.1, XM_017013098.1, XM_017013099.1, |
| | | XM_017013100.1, XM_017013101.1 |
| FOLR2 | folate receptor beta | NM_000803.4, NM_001113534.1, NM_001113535.1, |
| | | NM_001113536.1, XM_005273856.3 |
| HFE | homeostatic iron | NM_000410.3, NM_001300749.1, NM_139003.2, |
| | regulator | NM_139004.2, NM_139006.2, NM_139007.2, NM_139008.2, |
| | | NM_139009.2, NM_139010.2, NM_139011.2, NM_139002.2, |
| | | NM_139005.2, XM_011514543.2 |
| HMOX1 | heme oxygenase 1 | NM_002133.2 |
| HP | Haptoglobin | NM_001126102.2, NM_001318138.1, NM_005143.4 |
| IGFBP3 | insulin like growth | NM_000598.4, NM_001013398.1, XM_017012152.1 |
| | factor binding protein 3 | |
| MEST | mesoderm specific | NM_001253900.1, NM_001253901.1, NM_001253902.1, |
| | transcript | NM_002402.3, NM_177524.2, NM_177525.2, |
| | | XM_011516222.1, XM_017012218.1 |
| PLAU | plasminogen activator, | NM_001145031.2, NM_001319191.1, NM_002658.4, |
| | urokinase | XM_011539866.2 |
| RAC2 | Rac family small | NM_002872.4, XM_006724286.3 |
| | GTPase 2 | |
| RNH1 | ribonuclease/angiogenin | NM_002939.3, NM_203383.1, NM_203384.1, NM_203385.1, |
| | inhibitor 1 | NM_203386.2, NM_203387.2, NM_203388.2, NM_203389.2, |
| | | XM_011520255.1, XM_011520257.2, XM_011520258.2, |
| | | XM_011520259.2, XM_011520260.2, XM_011520261.2, |
| | | XM_011520262.2, XM_011520263.1, XM_017018106.1 |
| SERPINE1 | serpin family E member | NM_000602.4, NM_001165413.2, XM_017012260.1 |
| | 1 | |
| TIMP1 | TIMP metallopeptidase | NM_003254.2, XM_017029766.1 |
| | inhibitor 1 | |

TABLE 3
Signature 1 Gene Panels

| Panel | N | Gene Symbols |
|---|---|---|
| S1A | 63 | ABCC9, AFAP1L2, BACE1, BGN, BMP5, COL4A2, COL8A1, COL8A2, |
| | | CPXM2, CXCL12, EBF1, ECM2, EDNRA, ELN, EPHA3, FBLN5, GNAS, |
| | | GNB4, GUCY1A3, HEY2, HSPB2, IL1B, ITGA9, ITPR1, JAM2, JAM3, |
| | | KCNJ8, LAMB2, LHFP, LTBP4, MEOX1, MGP, MMP12, MMP13, |
| | | NAALAD2, NFATC1, NOV, OLFML2A, PCDH17, PDE5A, PDGFRB, PEG3, |
| | | PLSCR2, PLXDC2, RGS4, RGS5, RNF144A, RRAS, RUNX1T1, CAV2, |
| | | SELP, SERPINE2, SGIP1, SMARCA1, SPON1, STAB2, STEAP4, TBX2, |
| | | TEK, TGFB2, TMEM204, TTC28, UTRN |
| S1B | 50 | ELN, EPHA3, FBLN5, GNAS, GNB4, GUCY1A3, HEY2, HSPB2, IL1B, |
| | | ITGA9, ITPR1, JAM2, JAM3, KCNJ8, LAMB2, LHFP, LTBP4, MEOX1, |
| | | MGP, MMP12, MMP13, NAALAD2, NFATC1, NOV, OLFML2A, PCDH17, |
| | | PDE5A, PDGFRB, PEG3, PLSCR2, PLXDC2, RGS4, RGS5, RNF144A, |
| | | RRAS, RUNX1T1, CAV2, SELP, SERPINE2, SGIP1, SMARCA1, SPON1, |
| | | STAB2, STEAP4, TBX2, TEK, TGFB2, TMEM204, TTC28, UTRN |
| S1C | 40 | ITPR1, JAM2, JAM3, KCNJ8, LAMB2, LHFP, LTBP4, MEOX1, MGP, |
| | | MMP12, MMP13, NAALAD2, NFATC1, NOV, OLFML2A, PCDH17, |
| | | PDE5A, PDGFRB, PEG3, PLSCR2, PLXDC2, RGS4, RGS5, RNF144A, |
| | | RRAS, RUNX1T1, CAV2, SELP, SERPINE2, SGIP1, SMARCA1, SPON1, |
| | | STAB2, STEAP4, TBX2, TEK, TGFB2, TMEM204, TTC28, UTRN |
| S1D | 30 | MMP13, NAALAD2, NFATC1, NOV, OLFML2A, PCDH17, PDE5A, |
| | | PDGFRB, PEG3, PLSCR2, PLXDC2, RGS4, RGS5, RNF144A, RRAS, |
| | | RUNX1T1, CAV2, SELP, SERPINE2, SGIP1, SMARCA1, SPON1, STAB2, |
| | | STEAP4, TBX2, TEK, TGFB2, TMEM204, TTC28, UTRN |
| S1E | 20 | PLXDC2, RGS4, RGS5, RNF144A, RRAS, RUNX1T1, CAV2, SELP, |
| | | SERPINE2, SGIP1, SMARCA1, SPON1, STAB2, STEAP4, TBX2, TEK, |
| | | TGFB2, TMEM204, TTC28, UTRN |
| S1F | 10 | SMARCA1, SPON1, STAB2, STEAP4, TBX2, TEK, TGFB2, TMEM204, |
| | | TTC28, UTRN |

TABLE 4
Signature 2 gene panels

| Panel | N | Gene Symbols |
|---|---|---|
| S2A | 61 | AGR2, C11orf9, DUSP4, EIF5A, ETV5, GAD1, IQGAP3, MST1, MT2A, |
| | | MTA2, PLA2G4A, REG4, SRSF6, STRN3, TRIM7, USF1, ZIC2, C10orf54, |
| | | CCL3, CCL4, CD19, CD274, CD3E, CD4, CD8B, CTLA4, CXCL10, IFNA2, |
| | | IFNB1, IFNG, LAG3, PDCD1, PDCD1LG2, TGFB1, TIGIT, TNFRSF18, |
| | | TNFRSF4, TNFSF18, TLR9, HAVCR2, CD79A, CXCL11, CXCL9, GZMB, |
| | | IDO1, IGLL5, ADAMTS4, CAPG, CCL2, CTSB, FOLR2, HFE, HMOX1, HP, |
| | | IGFBP3, MEST, PLAU, RAC2, RNH1, SERPINE1, TIMP1 |
| S2B | 50 | REG4, SRSF6, STRN3, TRIM7, USF1, ZIC2, C10orf54, CCL3, CCL4, CD19, |
| | | CD274, CD3E, CD4, CD8B, CTLA4, CXCL10, IFNA2, IFNB1, IFNG, LAG3, |
| | | PDCD1, PDCD1LG2, TGFB1, TIGIT, TNFRSF18, TNFRSF4, TNFSF18, |
| | | TLR9, HAVCR2, CD79A, CXCL11, CXCL9, GZMB, IDO1, IGLL5, |
| | | ADAMTS4, CAPG, CCL2, CTSB, FOLR2, HFE, HMOX1, HP, IGFBP3, |
| | | MEST, PLAU, RAC2, RNH1, SERPINE1, TIMP1 |
| S2C | 40 | CD274, CD3E, CD4, CD8B, CTLA4, CXCL10, IFNA2, IFNB1, IFNG, LAG3, |
| | | PDCD1, PDCD1LG2, TGFB1, TIGIT, TNFRSF18, TNFRSF4, TNFSF18, |
| | | TLR9, HAVCR2, CD79A, CXCL11, CXCL9, GZMB, IDO1, IGLL5, |
| | | ADAMTS4, CAPG, CCL2, CTSB, FOLR2, HFE, HMOX1, HP, IGFBP3, |
| | | MEST, PLAU, RAC2, RNH1, SERPINE1, TIMP1 |
| S2D | 30 | PDCD1, PDCD1LG2, TGFB1, TIGIT, TNFRSF18, TNFRSF4, TNFSF18, |
| | | TLR9, HAVCR2, CD79A, CXCL11, CXCL9, GZMB, IDO1, IGLL5, |
| | | ADAMTS4, CAPG, CCL2, CTSB, FOLR2, HFE, HMOX1, HP, IGFBP3, |
| | | MEST, PLAU, RAC2, RNH1, SERPINE1, TIMP1 |
| S2E | 20 | CXCL11, CXCL9, GZMB, IDO1, IGLL5, ADAMTS4, CAPG, CCL2, CTSB, |
| | | FOLR2, HFE, HMOX1, HP, IGFBP3, MEST, PLAU, RAC2, RNH1, |
| | | SERPINE1, TIMP1 |
| S2F | 10 | HFE, HMOX1, HP, IGFBP3, MEST, PLAU, RAC2, RNH1, SERPINE1, |
| | | TIMP1 |

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise ABCC9, AFAP1L2, BGN, COL4A2, COL8A1, FBLN5, HEY2, IGFBP3, LHFP, NAALAD2, PCDH17, PDGFRB, PLXDC2, RGS5, RRAS, SERPINE1, STEAP4, TEK, TMEM204, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of ABCC9, AFAP1L2, BGN, COL4A2, COL8A1, FBLN5, HEY2, IGFBP3, LHFP, NAALAD2, PCDH17, PDGFRB, PLXDC2, RGS5, RRAS, SERPINE1, STEAP4, TEK, and TMEM204.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise ABCC9, COL4A2, MEST, OLFML2A, PCDH17, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of ABCC9, COL4A2, MEST, OLFML2A, and PCDH17.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise ADAMTS4, CD274, CXCL10, IDO1, RAC2, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of ADAMTS4, CD274, CXCL10, IDO1, and RAC2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise BGN, CCL2, CD19, CD274, CD3E, CD4, CD79A, COL4A2, COL8A1, CTLA4, CXCL9, GZMB, HAVCR2, IDO1, IL1B, LAG3, PDCD1, PDGFRB, TIGIT, TNFRSF18, TNFRSF4, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of BGN, CCL2, CD19, CD274, CD3E, CD4, CD79A, COL4A2, COL8A1, CTLA4, CXCL9, GZMB, HAVCR2, IDO1, IL1B, LAG3, PDCD1, PDGFRB, TIGIT, TNFRSF18, and TNFRSF4.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise BGN, CCL2, COL4A2, COL8A1, CTLA4, CXCL10, CXCL9, GZMB, HAVCR2, IL1B, LAG3, TIGIT, TNFRSF18, TNFRSF4, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of BGN, CCL2, COL4A2, COL8A1, CTLA4, CXCL10, CXCL9, GZMB, HAVCR2, IL1B, LAG3, TIGIT, TNFRSF18, and TNFRSF4.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise BGN, CD19, CD274, CD3E, CD4, CD79A, COL4A2, COL8A1, CTLA4, CXCL10, CXCL9, GZMB, HAVCR2, IDO1, IL1B, LAG3, PDCD1, PDGFRB, TIGIT, TNFRSF18, TNFRSF4, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of BGN, CD19, CD274, CD3E, CD4, CD79A, COL4A2, COL8A1, CTLA4, CXCL10, CXCL9, GZMB, HAVCR2, IDO1, IL1B, LAG3, PDCD1, PDGFRB, TIGIT, TNFRSF18, and TNFRSF4.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise BGN, PDGFRB, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of BGN and PDGFRB.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise C10orf54, NFATC1, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of C10orf54 and NFATC1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CAPG, DUSP4, LAG3, PLXDC2, TNFRSF18, TNFRSF4, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CAPG, DUSP4, LAG3, PLXDC2, TNFRSF18, and TNFRSF4.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL2, CCL4, CXCL9, GZMB, MGP, MMP12, RAC2, TIMP1, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL2, CCL4, CXCL9, GZMB, MGP, MMP12, RAC2, and TIMP1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL2, CD3E, CXCL10, CXCL11, GZMB, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL2, CD3E, CXCL10, CXCL11, and GZMB.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL2, CD4, CXCL10, MMP13, TIMP1, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL2, CD4, CXCL10, MMP13, and TIMP1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL3, CCL4, CTLA4, ETV5, HAVCR2, IFNG, LAG3, MTA2, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL3, CCL4, CTLA4, ETV5, HAVCR2, IFNG, LAG3, and MTA2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL4, CD3E, CXCL10, CXCL11, CXCL9, GZMB, HAVCR2, IDO1, IFNG, LAG3, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL4, CD3E, CXCL10, CXCL11, CXCL9, GZMB, HAVCR2, IDO1, IFNG, and LAG3.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL4, CD3E, CXCL10, CXCL11, CXCL9, GZMB, HAVCR2, IFNG, LAG3, PDCD1, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL4, CD3E, CXCL10, CXCL11, CXCL9, GZMB, HAVCR2, IFNG, LAG3, and PDCD1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL4, CXCL10, CXCL11, CXCL9, IDO1, IFNG CCL4, CXCL10, CXCL11, CXCL9, IFNG, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL4, CXCL10, CXCL11, CXCL9, IDO1, IFNG CCL4, CXCL10, CXCL11, CXCL9, and IFNG.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CCL4, GZMB, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CCL4 and GZMB.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CD274, CD3E, CD4, CXCL9, GZMB, IDO1, IFNG, LAG3, PDCD1LG2, TIGIT, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CD274, CD3E, CD4, CXCL9, GZMB, IDO1, IFNG, LAG3, PDCD1LG2, and TIGIT.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CD274, CD3E, CD79A, CXCL10, CXCL9, IDO1, IQGAP3, RAC2, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CD274, CD3E, CD79A, CXCL10, CXCL9, IDO1, IQGAP3, and RAC2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CD274, CTLA4, CXCL10, CXCL9, GZMB, HAVCR2, IFNG, IGFBP3, LAG3, PDCD1, PDGFRB, TEK, TGFB1, TGFB2, TIGIT, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CD274, CTLA4, CXCL10, CXCL9, GZMB, HAVCR2, IFNG, IGFBP3, LAG3, PDCD1, PDGFRB, TEK, TGFB1, TGFB2, and TIGIT.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CD3E, CTLA4, GZMB, LAG3, TGFB2, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CD3E, CTLA4, GZMB, LAG3, and TGFB2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CD4, CD79A, CXCL9, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CD4, CD79A, and CXCL9.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CD79A, CTLA4, EBF1, EPHA3, ETV5, GNAS, PDCD1, PDCD1LG2, PDGFRB, RUNX1T1, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CD79A, CTLA4, EBF1, EPHA3, ETV5, GNAS, PDCD1, PDCD1LG2, PDGFRB, and RUNX1T1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CD8B, CXCL10, CXCL11, GZMB, IFNG, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CD8B, CXCL10, CXCL11, GZMB, and IFNG.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise COL4A2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of COL4A2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CTLA4, CXCL10, CXCL11, CXCL9, GZMB, IDO1, IFNG, TIGIT, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CTLA4, CXCL10, CXCL11, CXCL9, GZMB, IDO1, IFNG, and TIGIT.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CTLA4, CXCL10, CXCL11, CXCL9, GZMB, IFNG, TIGIT, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CTLA4, CXCL10, CXCL11, CXCL9, GZMB, IFNG, and TIGIT.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CTLA4, CXCL10, CXCL11, TIGIT, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CTLA4, CXCL10, CXCL11, and TIGIT.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CTSB, DUSP4, MT2A, SERPINE2, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CTSB, DUSP4, MT2A, and SERPINE2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CXCL10, CXCL12, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CXCL10, and CXCL12.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CXCL10, CXCL9, GZMB, IFNG, IGFBP3, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CXCL10, CXCL9, GZMB, IFNG, and IGFBP3.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CXCL10, LAG3, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CXCL10, and LAG3.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CXCL12, PDGFRB, STEAP4, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CXCL12, PDGFRB, and STEAP4.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CXCL9, GZMB, IFNG, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CXCL9, GZMB, and IFNG.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CXCL9, IFNG, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CXCL9 and IFNG.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise CXCL9, MGP, RAC2, TIMP1, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of CXCL9, MGP, RAC2, and TIMP1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise EDNRA, IFNG, PDGFRB, TGFB1, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of EDNRA, IFNG, PDGFRB, and TGFB1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise ELN.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of ELN.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise NOV.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of NOV.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise EPHA3, GNAS, or a combination thereof.

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of EPHA3 and GNAS.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise GNAS. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of GNAS.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise HAVCR2, PDCD1, TIGIT, or a combination thereof. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of HAVCR2, PDCD1, and TIGIT.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise HAVCR2, TIGIT, or a combination thereof. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of HAVCR2 and TIGIT.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise IGFBP3, TGFB1, or a combination thereof. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of IGFBP3 and TGFB1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise IGFBP3. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of IGFBP3.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise PDCD1. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of PDCD1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise PDGFRB. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of PDGFRB.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise RGS5. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of RGS5.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise TGFB1. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of TGFB1.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise TIGIT. In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of TIGIT.

In some aspects, a gene panel to determine a Signature 1 score in a population-based classifier or a gene panel to be used as part of the training set or model input in a non-population-based classifier does not include BMP5, GNAS, IL1B, MMP12, NAALAD2, and STAB2. In some aspects, a gene panel to determine a Signature 1 score in a population-based classifier or a gene panel to be used as part of the training set or model input in a non-population-based classifier does not include 1, 2, 3, 4, 5, or 6 genes selected from the group consisting of BMP5, GNAS, IL1B, MMP12, NAALAD2, and STAB2. In some aspects, a gene panel to determine a Signature 1 score in a population-based classifier or a gene panel to be used as part of the training set or model input in a non-population-based classifier does not consist of BMP5, GNAS, IL1B, MMP12, NAALAD2, and STAB2.

In some aspects, a gene panel to determine a Signature 2 score in a population-based classifier or a gene panel to be used as part of the training set or model input in a non-population-based classifier does not include AGR2, C11orf9, CD79A, EIF5A, HFE, HP, MEST, MST1, MT2A, PLA2G4A, PLAU, STRN3, TNFSF18, TRIM7, USF1, and ZIC2. In some aspects, a gene panel to determine a Signature 2 score in a population-based classifier or a gene panel to be used as part of the training set or model input in a non-population-based classifier does not include 1, 2, 3, 4, 5, 6, 7, 8, 9, 10, 11, 12, 13, 14, 15 or 16 genes selected from the group consisting of AGR2, C11orf9, CD79A, EIF5A, HFE, HP, MEST, MST1, MT2A, PLA2G4A, PLAU, STRN3, TNFSF18, TRIM7, USF1, and ZIC2. In some aspects, a gene panel to determine a Signature 2 score in a population-based classifier or a gene panel to be used as part of the training set or model input in a non-population-based classifier does not consist of AGR2, C11orf9, CD79A, EIF5A, HFE, HP, MEST, MST1, MT2A, PLA2G4A, PLAU, STRN3, TNFSF18, TRIM7, USF1, and ZIC2.

Gene and Genesets that can be used according to the methods disclosed herein are presented in FIG. 28A, FIG. 28B, FIG. 28C, FIG. 28D, FIG. 28E, FIG. 28F, or FIG. 28G. Presence of a particular gene in a geneset presented in FIG. 28A-FIG. 28G is indicated by an open cell (white), whereas the absence of a particular gene in a geneset presented in FIG. 28A-FIG. 28G is indicated by a full cell (black).

In some aspects, a gene panel to determine a Signature 1 or a Signature 2 in a population-based classifier, or a gene panel to be used as part of the training set or model input in a non-population based classifier disclosed herein comprises ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2. In some aspects, a gene panel to determine a Signature 1 or a Signature 2 in a population-based classifier, or a gene panel to be used as part of the training set or model input in a non-population based classifier disclosed herein consists of ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2.

In some aspects, a gene panel to determine a Signature 1 or a Signature 2 in a population-based classifier or a gene panel to be used as part of the training set or model input in a non-population based classifier disclosed herein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 genes selected from the group consisting of ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2.

In some aspects, a gene panel to determine a Signature 1 or a Signature 2 in a population-based classifier or a gene panel to be used as part of the training set or model input in a non-population based classifier disclosed herein consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124, genes selected from the group consisting of ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2.

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise the genes present in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by black cells in FIGS. 28A-G).

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of the genes present in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by black cells in FIGS. 28A-G).

In some aspects, a gene panel disclosed herein (e.g., a gene panel to determine a Signature 1 score or a Signature 2 score in a population-based classifier, or a gene panel to be used as part of the training set or model input in a non-population-based classifier) comprises the genes present in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by black cells in FIGS. 28A-G).

In some aspects, a gene panel disclosed herein (e.g., a gene panel to determine a Signature 1 score or a Signature 2 score in a population-based classifier, or a gene panel to be used as part of the training set or model input in a non-population-based classifier) consists of the genes present in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by black cells in FIGS. 28A-G).

In some aspects, a gene panel disclosed herein (e.g., a gene panel to determine a Signature 1 score or a Signature 2 score in a population-based classifier, or a gene panel to be used as part of the training set or model input in a non-population-based classifier) does not comprise the genes absent in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by empty cells in FIGS. 28A-G).

In some aspects, a gene panel disclosed herein (e.g., a gene panel to determine a Signature 1 score or a Signature 2 score in a population-based classifier, or a gene panel to be used as part of the training set or model input in a non-population-based classifier) does not consist of the genes absent in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by empty cells in FIGS. 28A-G).

In some aspects, a gene panel disclosed herein (e.g., a gene panel to determine a Signature 1 score or a Signature 2 score in a population-based classifier, or a gene panel to be used as part of the training set or model input in a non-population-based classifier) comprises the genes absent in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by empty cells in FIGS. 28A-G).

In some aspects, a gene panel disclosed herein (e.g., a gene panel to determine a Signature 1 score or a Signature 2 score in a population-based classifier, or a gene panel to be used as part of the training set or model input in a non-population-based classifier) consists of the genes absent in Geneset 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281 or 282 (the genes indicated by empty cells in FIGS. 28A-G).

### I.B. Samples and Sample Processing

The methods disclosed herein comprise measuring the expression levels of a gene panel selected from a sample, e.g., a biological sample obtained from a subject. In some aspects, e.g., when two signature scores are determined (e.g., a Signature 1 score and a Signature 2 score as disclosed herein), each sample can be the same or it can be different. Thus, in some aspects, the first sample and the second sample used respectively to determine a first score and a second score are the same sample. In other aspects, the first sample and the second sample used respectively to determine a first score and a second score are different samples. In some aspects, the sample comprises intratumoral tissue. In some aspects, the first sample and/or the second sample comprises intratumoral tissue. In some aspects, the first sample and/or the second sample can incidentally include peritumoral tissue and/or healthy tissue that has infiltrated a regularly or irregularly shaped tumor. Biomarker levels (e.g., expression levels of genes in a gene panel of the present disclosure) can be measured in any biological sample that contains or is suspected to contain one or more of the biomarkers (e.g., RNA biomarkers) disclosed herein, including any tissue sample or biopsy from an animal, subject or patient, e.g., cancer tissue, tumor, and/or stroma of a subject. In some aspects, biomarker levels are derived from tumor tissue (e.g., fresh tissue, frozen tissue, or preserved tissue). The source of the tissue sample can be solid tissue, e.g., from a fresh, frozen and/or preserved organ, tissue sample, biopsy, or aspirate. In some aspects, the sample is a cell-free sample, e.g., comprising cell-free nucleic acids (e.g., DNA or RNA). A sample can, in some aspects, comprise compounds that are not naturally intermixed with the tissue in nature such as preservatives, anticoagulants, buffers, fixatives, nutrients, antibiotics or the like.

Biomarker levels can, in some instances, be derived from fixed tumor tissue. In some aspects, the sample is preserved as a frozen sample or as formalin-, formaldehyde-, or paraformaldehyde-fixed paraffin-embedded (FFPE) tissue preparation. For example, the sample can be embedded in a matrix, e.g., an FFPE block or a frozen sample. In some aspects, a sample can comprise bone marrow; aspirates; scrapings; bone marrow specimens; tissue biopsy specimens; surgical specimens; etc. In some aspects, a sample is or comprises cells obtained from an individual, e.g., from an individual from whom the sample is obtained.

In some aspects, the sample can be obtained, e.g., from surgical material or from biopsy (e.g., a recent biopsy, a recent biopsy since last progression, or a recent biopsy since the last failed therapy). In some aspects, the biopsy can be archival tissue from a previous line of therapy. In some aspects, the biopsy can be from tissue that is therapy naïve. In some aspects, biological fluids are not used as samples.

I.B.1 Expression Levels and their Measurements

The level of expression of the genes in the gene panels described herein can be determined using any method in the art. For example, expression levels can be determined by detecting expression of nucleic acids (e.g., RNA or mRNA) or proteins encoded by the gene. Thus, in some aspects, the expression levels are transcribed RNA levels and/or expressed protein levels.

In some aspects, the RNA levels are determined using sequencing methods, e.g., Next Generation Sequencing (NGS). In some aspects, the NGS is RNA-Seq, EdgeSeq, PCR, Nanostring, or combinations thereof, or any technologies that measure RNA. In some aspects, the RNA measurement methods comprise nuclease protection.

In some aspects, the RNA levels are determined using fluorescence. In some aspects, the RNA levels are determined using an Affymetrix microarray or a microarray such as sold by Agilent. More detailed description of methods suitable for the determination of nucleic acid expression levels (generally mRNA levels) and protein expression levels are provided below.

### I.B.1.a Nucleic Acid Expression Levels

Nucleic acid expression levels can be determined, in some instances, using methods of sequencing nucleic acids. Any method of sequencing known in the art can be used. Sequencing of nucleic acids isolated by selection methods are typically carried out using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10 molecules are sequenced simultaneously). In one aspect, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46; Eastel et al. (2019) Expert Rev. Mol. Diag. 19:591-98; and, McCombie et al. (2019) Cold Spring Harb. Perspect. Med. 9:a036798; which are herein incorporated by reference in their entireties.

In some aspects, next-generation sequencing allows for the determination of the nucleotide sequence of an individual nucleic acid biomarker (e.g., Helicos BioSciences' HeliScope Gene Sequencing system, and Pacific Biosciences' PacBio RS system). In other aspects, the sequencing method determines the nucleotide sequence of clonally expanded proxies for individual nucleic acid biomarkers and/or quantification of the level (e.g., relative quantity of copies) of individual nucleic acid biomarkers, e.g., RNA biomarkers, e.g., as listed in any of Tables 1-4 (e.g., the Solexa sequencer, Illumina Inc., San Diego, Calif; 454 Life Sciences (Branford, Conn.), and Ion Torrent), e.g., massively parallel short-read sequencing (e.g., the Solexa sequencer, Illumina Inc., San Diego, Calif.), which generates more bases of sequence per sequencing unit than other sequencing methods that generate fewer but longer reads. Other methods or machines for next-generation sequencing include, but are not limited to, the sequencers provided by 454 Life Sciences (Branford, Conn.), Applied Biosystems (Foster City, Calif.; SOLiD sequencer), Helicos BioSciences Corporation (Cambridge, Mass.), and emulsion and microfluidic sequencing technology nanodroplets (e.g., GnuBio droplets).

Platforms for next-generation sequencing include, but are not limited to, Roche/454's Genome Sequencer (GS) FLX System, Illumina/Solexa's Genome Analyzer (GA), Life/APG's Support Oligonucleotide Ligation Detection (SOLiD) system, Polonator's G.007 system, Helicos BioSciences' HeliScope Gene Sequencing system, and Pacific Biosciences' PacBio RS system, HTG Molecular Diagnostics' EdgeSeq, and Nanostring Technology's Hyb & Seq NGS Technology.

NGS technologies can include one or more of steps, e.g., template preparation, sequencing and imaging, and data analysis, which are disclosed more in detail below.

It is noted that template amplification methods, such as PCR methods known in the art, can also be used to quantify biomarker levels. Exemplary template enrichment methods include, e.g., microdroplet PCR technology (Tewhey R. et al., Nature Biotech. 2009, 27:1025-1031), custom-designed oligonucleotide microarrays (e.g., Roche/NimbleGen oligonucleotide microarrays), and solution-based hybridization methods (e.g., molecular inversion probes (MIPs) (Porreca G. J. et al., Nature Methods, 2007, 4:931-936; Krishnakumar S. et al., Proc. Natl. Acad. Sci. USA, 2008, 105:9296-9310; Turner E. H. et al., Nature Methods, 2009, 6:315-316), and biotinylated RNA capture sequences (Gnirke A. et al., Nat. Biotechnol. 2009; 27(2):182-9).

(a) Template preparation. Methods for template preparation can include steps such as randomly breaking nucleic acids (e.g., RNA) into smaller sizes and generating sequencing templates (e.g., fragment templates or mate-pair templates). The spatially separated templates can be attached or immobilized to a solid surface or support, allowing massive amount of sequencing reactions to be performed simultaneously. Types of templates that can be used for NGS reactions include, e.g., clonally amplified templates originating from single DNA molecules, and single DNA molecule templates. Methods for preparing clonally amplified templates include, e.g., emulsion PCR (emPCR) and solid-phase amplification.

EmPCR can be used to prepare templates for NGS. Typically, a library of nucleic acid fragments is generated, and adaptors containing universal priming sites are ligated to the ends of the fragment. The fragments are then denatured into single strands and captured by beads. Each bead captures a single nucleic acid molecule. After amplification and enrichment of emPCR beads, a large amount of templates can be attached or immobilized in a polyacrylamide gel on a standard microscope slide (e.g., Polonator), chemically crosslinked to an amino-coated glass surface (e.g., Life/APG; Polonator), or deposited into individual PicoTiterPlate (PTP) wells (e.g., Roche/454), in which the NGS reaction can be performed.

Solid-phase amplification can also be used to produce templates for NGS.

Typically, forward and reverse primers are covalently attached to a solid support. The surface density of the amplified fragments is defined by the ratio of the primers to the templates on the support. Solid-phase amplification can produce hundreds of millions spatially separated template clusters (e.g., Illumina/Solexa). The ends of the template clusters can be hybridized to universal sequencing primers for NGS reactions.

Other methods for preparing clonally amplified templates also include, e.g., Multiple Displacement Amplification (MDA) (Lasken R. S. Curr Opin Microbiol. 2007; 10(5):510-6). MDA is a non-PCR based DNA amplification technique. The reaction involves annealing random hexamer primers to the template and DNA synthesis by high fidelity enzyme, typically bacteriophage D29 DNA polymerase at a constant temperature. MDA can generate large sized products with lower error frequency.

Single-molecule templates are another type of templates that can be used for NGS reaction. Spatially separated single molecule templates can be immobilized on solid supports by various methods. In one approach, individual primer molecules are covalently attached to the solid support. Adaptors are added to the templates and templates are then hybridized to the immobilized primers. In another approach, single-molecule templates are covalently attached to the solid support by priming and extending single-stranded, single-molecule templates from immobilized primers. Universal primers are then hybridized to the templates. In yet another approach, single polymerase molecules are attached to the solid support, to which primed templates are bound.

(b) Sequencing and imaging. Exemplary sequencing and imaging methods for NGS include, but are not limited to, cyclic reversible termination (CRT), sequencing by ligation (SBL), single-molecule addition (pyrosequencing), and real-time sequencing.

CRT uses reversible terminators in a cyclic method that minimally includes the steps of nucleotide incorporation, fluorescence imaging, and cleavage. Typically, a DNA polymerase incorporates a single fluorescently modified nucleotide corresponding to the complementary nucleotide of the template base to the primer. DNA synthesis is terminated after the addition of a single nucleotide and the unincorporated nucleotides are washed away. Imaging is performed to determine the identity of the incorporated labeled nucleotide. Then in the cleavage step, the terminating/inhibiting group and the fluorescent dye are removed. Exemplary NGS platforms using the CRT method include, but are not limited to, Illumina/Solexa Genome Analyzer (GA), which uses the clonally amplified template method coupled with the four-color CRT method detected by total internal reflection fluorescence (TIRF); and Helicos BioSciences/HeliScope, which uses the single-molecule template method coupled with the one-color CRT method detected by TIRF.

SBL uses DNA ligase and either one-base-encoded probes or two-base-encoded probes for sequencing. Typically, a fluorescently labeled probe is hybridized to its complementary sequence adjacent to the primed template. DNA ligase is used to ligate the dye-labeled probe to the primer. Fluorescence imaging is performed to determine the identity of the ligated probe after non-ligated probes are washed away. The fluorescent dye can be removed by using cleavable probes to regenerate a 5′-PO₄ group for subsequent ligation cycles. Alternatively, a new primer can be hybridized to the template after the old primer is removed. Exemplary SBL platforms include, but are not limited to, Life/APG/SOLiD (support oligonucleotide ligation detection), which uses two-base-encoded probes.

Pyrosequencing method is based on detecting the activity of DNA polymerase with another chemiluminescent enzyme. Typically, the method allows sequencing of a single strand of DNA by synthesizing the complementary strand along it, one base pair at a time, and detecting which base was actually added at each step. The template DNA is immobile, and solutions of A, C, G, and T nucleotides are sequentially added and removed from the reaction. Light is produced only when the nucleotide solution complements the first unpaired base of the template. The sequence of solutions which produce chemiluminescent signals allows the determination of the sequence of the template. Exemplary pyrosequencing platforms include, but are not limited to, Roche/454, which uses DNA templates prepared by emPCR with 1-2 million beads deposited into PTP wells.

Real-time sequencing involves imaging the continuous incorporation of dye-labeled nucleotides during DNA synthesis. Exemplary real-time sequencing platforms include, but are not limited to, Pacific Biosciences platform, which uses DNA polymerase molecules attached to the surface of individual zero-mode waveguide (ZMW) detectors to obtain sequence information when phospholinked nucleotides are being incorporated into the growing primer strand; Life/VisiGen platform, which uses an engineered DNA polymerase with an attached fluorescent dye to generate an enhanced signal after nucleotide incorporation by fluorescence resonance energy transfer (FRET); and LI-COR Biosciences platform, which uses dye-quencher nucleotides in the sequencing reaction.

Other sequencing methods for NGS include, but are not limited to, nanopore sequencing, sequencing by hybridization, nano-transistor array based sequencing, polony sequencing, scanning tunneling microscopy (STM) based sequencing, and nanowire-molecule sensor based sequencing.

Nanopore sequencing involves electrophoresis of nucleic acid molecules in solution through a nano-scale pore which provides a highly confined space within which single-nucleic acid polymers can be analyzed. Exemplary methods of nanopore sequencing are described, e.g., in Branton D. et al., Nat Biotechnol. 2008; 26(10):1146-53.

Sequencing by hybridization is a non-enzymatic method that uses a DNA microarray. Typically, a single pool of DNA is fluorescently labeled and hybridized to an array containing known sequences. Hybridization signals from a given spot on the array can identify the DNA sequence. The binding of one strand of DNA to its complementary strand in the DNA double-helix is sensitive to even single-base mismatches when the hybrid region is short or if specialized mismatch detection proteins are present. Exemplary methods of sequencing by hybridization are described, e.g., in Hanna G. J. et al., J. Clin. Microbiol. 2000; 38 (7): 2715-21; and Edwards J. R. et al., Mut. Res. 2005; 573 (1-2): 3-12.

Polony sequencing is based on polony amplification and sequencing-by-synthesis via multiple single-base-extensions (FISSEQ). Polony amplification is a method to amplify DNA in situ on a polyacrylamide film. Exemplary polony sequencing methods are described, e.g., in US Patent Application Publication No. 2007/0087362.

Nano-transistor array based devices, such as Carbon NanoTube Field Effect Transistor (CNTFET), can also be used for NGS. For example, DNA molecules are stretched and driven over nanotubes by micro-fabricated electrodes. DNA molecules sequentially come into contact with the carbon nanotube surface, and the difference in current flow from each base is produced due to charge transfer between the DNA molecule and the nanotubes. DNA is sequenced by recording these differences. Exemplary Nano-transistor array based sequencing methods are described, e.g., in U.S. Patent Application Publication No. 2006/0246497.

Scanning tunneling microscopy (STM) can also be used for NGS. STM uses a piezo-electric-controlled probe that performs a raster scan of a specimen to form images of its surface. STM can be used to image the physical properties of single DNA molecules, e.g., generating coherent electron tunneling imaging and spectroscopy by integrating scanning tunneling microscope with an actuator-driven flexible gap. Exemplary sequencing methods using STM are described, e.g., in U.S. Patent Application Publication No. 2007/0194225.

A molecular-analysis device which is comprised of a nanowire-molecule sensor can also be used for NGS. Such device can detect the interactions of the nitrogenous material disposed on the nanowires and nucleic acid molecules such as DNA. A molecule guide is configured for guiding a molecule near the molecule sensor, allowing an interaction and subsequent detection. Exemplary sequencing methods using nanowire-molecule sensor are described, e.g., in U.S. Patent Application Publication No. 2006/0275779.

Double-ended sequencing methods can be used for NGS. Double-ended sequencing uses blocked and unblocked primers to sequence both the sense and antisense strands of DNA. Typically, these methods include the steps of annealing an unblocked primer to a first strand of nucleic acid; annealing a second blocked primer to a second strand of nucleic acid; elongating the nucleic acid along the first strand with a polymerase; terminating the first sequencing primer; deblocking the second primer; and elongating the nucleic acid along the second strand. Exemplary double ended sequencing methods are described, e.g., in U.S. Pat. No. 7,244,567. In an aspect, only the exome is sequenced, e.g., whole exome sequencing (WES).

(c) Data analysis. After NGS reads have been generated, they can be aligned to a known reference sequence or assembled de novo. For example, identifying and quantifying copies of nucleic acids (e.g., RNAs) can be accomplished by aligning NGS reads to a reference sequence (e.g., a wild-type sequence). Methods of sequence alignment for NGS are described e.g., in Trapnell C. and Salzberg S. L. Nature Biotech., 2009, 27:455-457; snd Saeed & Usman “Biological Sequence Analysis” in Husi H, editor. Computational Biology. Brisbane (AU): Codon Publications; 2019 Nov. 21. Chapter 4; or Mielczarek & Szyka (2016) J. Appl. Genet. 57:71-9; Conesa et al. (2016) Genome Biol. 17:13, which are herein incorporated by reference in their entireties. Sequence alignment or assembly can be performed using read data from one or more NGS platforms, e.g., mixing Roche/454 and Illumina/Solexa read data.

As disclosed above, various technologies exist for measuring gene expression where each platform technology requires specific preprocessing of the raw data. The population-based classifier described in the Examples section supports, e.g., Affymetrix DNA microarray, and high throughput next generation RNA sequencing (NGS). However, the methodologies used can be extended to other technologies.

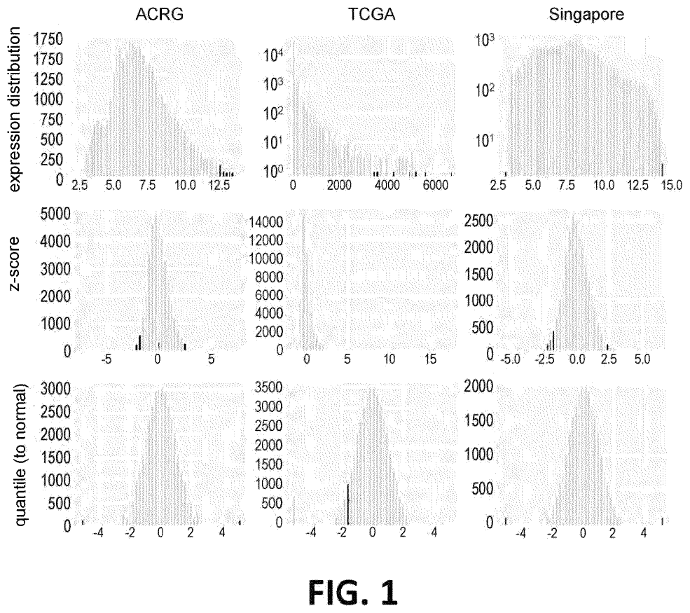

For microarray data, the Affymetrix chip procedure measures the intensity pixel values per cell (each containing a unique probe) which are stored in a CEL file. In some aspects, CEL files are processed using the Affy R package. In some aspects, the expresso function is applied using the following parameters: RMA (Robust Multichip Average) background correction method, quantile normalization, no probe-specific correction, and medianpolish summarization (J. W. Tukey, Exploratory Data Analysis, Addison-Wesley, 1977). In some aspects, the expression values returned by the expresso function are log₂-transformed, and expressions are quantile transformed to normal output distribution, binning input values into, e.g., 100 quantiles (see FIG. 1).

In some aspects, Illumina RNA-Seq sequencing reads are processed by cleaning up reads, aligning them to a reference genome and quantifying gene expression. Thus, in some aspects, the analysis steps include three key steps: trimming (e.g., using BBDuk; jgi.doe.gov/data-and-tools/bbtools/bb-tools-user-guide/bbduk-guide/), mapping (e.g., using STAR; see Dobin & Gingeras (2015) Curr. Protoc. Bioinformatics 51:11.14.1-11.14.19), and expression quantification (e.g., using featureCounts; Liao et al. (2014) Bioinformatics 30:923-930). In some aspects, the current reference human genome is Ensembl, version 92, extended with references for common spike-in standards such as ERCC (External RNA Controls Consortium) external RNA controls and SIRV (Spike-In RNA Variants). In other aspects, a more recent reference human genome is used. In some aspects, as an additional quality control step, a sample of a million reads (processed, e.g., with Seqtk tool; arc.vt.edu/userguide/seqtk/) is mapped to rRNA and globin sequences of the selected species to determine the overall proportion of these kinds of reads in the sample. Results can be reported, e.g., in the summary table of a report tool such as MultiQC. In some aspects, raw and normalized (e.g., TPM, Transcripts Per Kilobase Million; or FPKM, Fragments Per Kilobase Million) expression values are provided by software.

In some specific aspects of the methods disclosed herein, prior to stratifying the samples with the Z-score-based model, TPM normalized expressions can be quantile transformed to normal output distribution, binning input values into, e.g., 100 quantiles (see FIG. 1).

In some aspects aspect, different batches of expression data can be independently normalized in order to train a machine learning model. Independent normalization can be utilized when there is a pronounced batch effect. In some aspects, principal component analysis, as known in the art, can reveal batch effects, including those that might arise, in a non-limiting example, when sequencing expression values obtained from one source (e.g., RNA Exome (WES)) are used to train a machine learning model in addition to sequencing expression values obtained from a different source (e.g., RNA-Seq). In some aspects, asynchronicity of sample collection is not a source of batch effects. In some aspects, asynchronicity of sample collection is a source of batch effects, which can be addressed, e.g., with normalization techniques.

For all platform technologies disclosed herein, quantile normalization can be used for cross-platform harmonization, for example when utilizing Illumina and EdgeSeq (HTG Molecular Diagnostics, Inc.) data. Another example is the use of quantile normalization to harmonize microarray and RNA-Seq data, e.g., a model can be trained on microarray data (e.g., from the ACRG patient dataset) and then applied to a total-RNA platform (e.g., RNA-Seq).

Input values can be binned into, e.g., 10, 15, 20, 25, 30, 35, 40, 45, 50, 55, 60, 65, 70, 75, 80, 85, 90, 95, 100 or more quantiles and applying a normal or uniform output distribution function. In some aspects, quantile normalization can be applied to the normal distribution for a Z-Score classifier disclosed herein. In some aspects, quantile normalization can be applied to the uniform distribution of an ANN classifier disclosed herein. In some aspects, the number of quantiles is above, below, or between any of the values provided above.

### I.B.1.b Protein Expression Levels

Exemplary methods for detecting expression levels of proteins (e.g., polypeptides) include, but are not limited to, immunohistochemical methods, ELISA, Western analysis, HPLC, and proteomics assays. In some aspects, the protein expression level is determined by an immunohistochemical method. For example, formalin fixed paraffin embedded tissue is contacted with an antibody that specifically binds a biomarker described herein. Bound antibody is detected using a secondary antibody coupled to a detectable label or a detectable label such as a colorimetric label (e.g., an enzyme substrate product with HRP or AP). Antibody positive signals are scored by estimating the ratio of positive tumor cells and the average staining intensity of positive tumor cells. Both the ratio and the intensity score are combined into a total score comparing both factors.

In some aspects, protein expression levels are determined by digital pathological methods. Digital pathological methods include scanned images of tissue on a solid support such as a glass slide. The glass slide is scanned into a full slide image using a scanning device. The scanned image is typically stored in an information management system for archival recording and retrieval. An image analysis tool can be used to obtain objective quantitative measurement results from digital slides. For example, the area and intensity of immunohistochemical staining can be analyzed using an appropriate image analysis tool. Digital pathology systems can include scanners, analysis tools (visualization software, information management systems and image analysis platforms), storage and communication (shared services, software). Digital pathology systems are available from a number of commercial sources, such as Aperio Technologies, Inc. (a subsidiary of Leica Microsystems GmbH), and Ventana Medical Systems, Inc. (now part of Roche) available. Expression levels by can be quantified by a commercial service provider, including Flagship Biosciences (Colorado), Pathology, Inc. (California), Quest Diagnostics (New Jersey), and Premier Laboratory LLC (Colorado).

### I.C Population-Based Classifiers

The population-based classifiers disclosed herein rely on the integration of expression levels of a plurality of genes related, e.g., to structural and functional aspects of the TME, to derive a score which is correlated with responses to particular anticancer therapies. Thus, the determination that a cancer's particular TME or combination has a particular score (or combination of scores if multiple gene panels are used) allows the selection of the appropriate TME-class treatment or combination thereof. Thus, in one aspect, the present disclosure provides methods for determining the tumor microenvironment (TME) of a cancer in a subject in need thereof, wherein the method comprises determining a combined biomarker which comprises
(a) a Signature 1 score (e.g., a signature in which gene activation is correlated with endothelial cell signature activation); and,
(b) a Signature 2 score (e.g., a signature in which activation is correlated with inflammatory and immune cell signature activation),
wherein
(i) the Signature 1 score is determined by measuring the expression levels of a gene panel selected from TABLE 3 in a first sample obtained from the subject; and,
(ii) the Signature 2 score is determined by measuring the expression levels of a gene panel selected from TABLE 4 in a second sample obtained from the subject.

In some aspects, the Signature 1 score is determined using a gene panel selected from TABLE 3, wherein the gene panel comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 genes selected from TABLE 1.

In some aspects, the gene panel selected from TABLE 3 comprises ABCC9, AFAP1L2, BACE1, BGN, BMP5, COL4A2, COL8A1, COL8A2, CPXM2, CXCL12, EBF1, ECM2, EDNRA, ELN, EPHA3, FBLN5, GNAS, GNB4, GUCY1A3, HEY2, HSPB2, IL1B, ITGA9, ITPR1, JAM2, JAM3, KCNJ8, LAMB2, LHFP, LTBP4, MEOX1, MGP, MMP12, MMP13, NAALAD2, NFATC1, NOV, OLFML2A, PCDH17, PDE5A, PDGFRB, PEG3, PLSCR2, PLXDC2, RGS4, RGS5, RNF144A, RRAS, RUNX1T1, CAV2, SELP, SERPINE2, SGIP1, SMARCA1, SPON1, STAB2, STEAP4, TBX2, TEK, TGFB2, TMEM204, TTC28, and UTRN; or any combination thereof.

In some aspects, the gene panel selected from TABLE 3 consists of ABCC9, AFAP1L2, BACE1, BGN, BMP5, COL4A2, COL8A1, COL8A2, CPXM2, CXCL12, EBF1, ECM2, EDNRA, ELN, EPHA3, FBLN5, GNAS, GNB4, GUCY1A3, HEY2, HSPB2, IL1B, ITGA9, ITPR1, JAM2, JAM3, KCNJ8, LAMB2, LHFP, LTBP4, MEOX1, MGP, MMP12, MMP13, NAALAD2, NFATC1, NOV, OLFML2A, PCDH17, PDE5A, PDGFRB, PEG3, PLSCR2, PLXDC2, RGS4, RGS5, RNF144A, RRAS, RUNX1T1, CAV2, SELP, SERPINE2, SGIP1, SMARCA1, SPON1, STAB2, STEAP4, TBX2, TEK, TGFB2, TMEM204, TTC28, and UTRN.

In some aspects, the Signature 2 score is determined using a gene panel selected from TABLE 4, wherein the gene panel comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes selected from TABLE 2.

In some aspects, the gene panel selected from TABLE 4 comprises, e.g., AGR2, C11orf9, DUSP4, EIF5A, ETV5, GAD1, IQGAP3, MST1, MT2A, MTA2, PLA2G4A, REG4, SRSF6, STRN3, TRIM7, USF1, ZIC2, C10orf54, CCL3, CCL4, CD19, CD274, CD3E, CD4, CD8B, CTLA4, CXCL10, IFNA2, IFNB1, IFNG, LAG3, PDCD1, PDCD1LG2, TGFB1, TIGIT, TNFRSF18, TNFRSF4, TNFSF18, TLR9, HAVCR2, CD79A, CXCL11, CXCL9, GZMB, IDO1, IGLL5, ADAMTS4, CAPG, CCL2, CTSB, FOLR2, HFE, HMOX1, HP, IGFBP3, MEST, PLAU, RAC2, RNH1, SERPINE1, and TIMP1; or, any combination thereof.

In some aspects, the gene panel selected from TABLE 4 consists of AGR2, C11orf9, DUSP4, EIF5A, ETV5, GAD1, IQGAP3, MST1, MT2A, MTA2, PLA2G4A, REG4, SRSF6, STRN3, TRIM7, USF1, ZIC2, C10orf54, CCL3, CCL4, CD19, CD274, CD3E, CD4, CD8B, CTLA4, CXCL10, IFNA2, IFNB1, IFNG, LAG3, PDCD1, PDCD1LG2, TGFB1, TIGIT, TNFRSF18, TNFRSF4, TNFSF18, TLR9, HAVCR2, CD79A, CXCL11, CXCL9, GZMB, IDO1, IGLL5, ADAMTS4, CAPG, CCL2, CTSB, FOLR2, HFE, HMOX1, HP, IGFBP3, MEST, PLAU, RAC2, RNH1, SERPINE1, and TIMP1.

In some aspects, a Signature 1 gene can be an angiogenic biomarker. The term “angiogenic biomarker,” as used herein, refers to a biomarker (e.g., nucleic acid biomarker, e.g., RNA biomarker) that is differentially expressed in a tumor, or stroma thereof, comprising pathological levels of angiogenesis relative to a comparable non-cancerous tissue or reference sample. Exemplary angiogenic biomarkers are listed in TABLE 1. In some aspects, a tumor, or stroma thereof, can exhibit a substantial elevation or decrease of expression levels of a plurality of biomarkers listed in TABLE 1.

In some aspects, a tumor, or stroma thereof, exhibits substantial elevation or decrease of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of the biomarkers listed in TABLE 1, e.g., relative to the median level of a population of patients with cancer.

In some aspects, a Signature 2 gene can be an immune biomarker. The term “immune biomarker” as used herein, refers to a biomarker (e.g., nucleic acid biomarker, e.g., RNA biomarker) that is differentially expressed in a tumor, or stroma thereof, comprising increased immune infiltration relative to a comparable reference sample or samples, such that an immune response can be induced if the tumor is treated with an immunotherapy. Exemplary immune biomarkers are listed in TABLE 2. In some aspects, a tumor, or stroma thereof, can exhibit a substantial elevation or decrease of expression levels of a plurality of biomarkers listed in TABLE 2.

In some aspects, a tumor, or stroma thereof, exhibits substantial elevation or decrease of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of the biomarkers listed in TABLE 2, e.g., relative to the median level of a population of patients with cancer.

In particular aspects disclosed herein, two classifiers are used: a Signature 1 score (derived from measuring expression levels corresponding to biomarker genes of TABLE 1 or a subset thereof); and, a Signature 2 score (derived from measuring expression levels corresponding to biomarkers genes of TABLE 2 or a subset thereof). Two different states are considered for each of the classifiers (i.e., a positive or negative score depending on whether the score integrating the expression values for the genes in a gene panel is above or below a certain threshold). This approach allows the stratification of cancer samples into four different TMEs.

If additional gene panels are incorporated to the population-based classifiers of the present disclosure, the granularity of the TME classification increases. For example, the use of three Signature scores, each one with a possible positive or negative value allows to stratify a population of samples into eight different TMEs. Alternatively, if the same Signature scores used herein have not just a positive or negative state, but additional states falling within, e.g., 3 ranges, based on two thresholds, granularity would also be increased. In addition to using a plurality of thresholds, Signature score values could be grouped based on other criteria, e.g., assigning a score to a certain tercile, quartile, or quintile, based on the observed distribution of score values.

It should be appreciated that while the genes of Signature 1 and Signature 2, as utilized by the ANN method, have proven predictive, the ANN method has the capability to be used with other gene signatures (each one defined by a gene panel comprising a subset of the genes disclosed in TABLE 1 and/or TABLE 2) for other TMEs, e.g., the four TMEs disclosed herein, combinations thereof, or other TMEs resulting from the application of different thresholds to the ANN output, or, e.g., the use of different ANN architectures, weights, or activation functions. The ANN method also has the capability to be used in combination with Signatures 1 and 2, optionally with gene signatures for other TMEs as described above, and/or with one or more simplified measurements of gene activity (e.g., expression activity and/or expression levels of molecular biomarkers).

Increasing the granularity of the population-based classifiers can result in increased precision and increased efficacy of the selected therapies. For example, using the classifiers disclosed herein (Signature 1 and Signature 2) but having three states (e.g., three ranges determined by two different thresholds) would allow to stratify a population of cancer samples into nine different TMEs. Such increase in granularity of the TME population classification would also be associated with an increase in the granularity of the treatment options; in other words, the TME classification of cancer samples into a larger number of TMEs would allow a more precise determination of an optimal treatment. For example, a TME classification into four TMEs can be sufficient to determine that anti-PD-1 antibodies (e.g., sintilimab, tislelizumab, pembrolizumab, or an antigen binding portion thereof) in general are the best treatment option, but a TME classification into a larger number of TMEs could be sufficient to pinpoint a certain anti-PD1 antibody (e.g., sintilimab, tislelizumab, pembrolizumab, or an antigen binding portion thereof), or a certain anti-angiogenic, such as a TKI inhibitor, as the best treatment option. Thus, in some aspects, the granularity of the classification can be incremented by increasing the number of TME classes. In some aspects, the granularity of the classification can also be incremented by including combinations of TME classes, e.g., classifying a cancer sample as biomarker-positive for 2 (e.g., ID and IS biomarker-positive), 3 (e.g., ID, IA, and IS biomarker-positive), or more TME classes.

### I.C.1 Score Calculation and Classification

The present disclosure provides the methodology to create a population-based Z-score classifier (or set of classifiers) that is able to stratify (or classify) gene expression samples into several TME classes or combinations thereof. The term “Z-score,” also referred to in the art as a standard score, Z-value, or normal score, among other terms, is a dimensionless quantity that is used to indicate the signed, fractional, number of standard deviations by which an event is above the mean value being measured. Values above the mean have positive Z-scores, while values below the mean have negative Z-scores.

In a particular aspect, the population-based classifier of the present disclosure comprises two classifiers (Signature 1 and Signature 2), each one with two possible states (positive or negative), which can stratify a population of gene expression samples into four different TME classes. The population-based Z-score classifier of the present disclosure also is able to classify a test sample with a subject with cancer into one specific TME class, or a combination thereof. Based on the assignment of the subject's sample to a specific TME class or a combination thereof, it is possible to select a personalized treatment known to have a high probability of being effective to treat the subject's cancer. As used herein, the TME classifications can also be referred to as stromal types, stromal subtypes, stromal phenotypes, or variations thereof. In some aspects, the application of different weights and parameters to the calculation of Z-scores and/or the application of different thresholds, can assign the subject's sample to two or more TMEs. Thus, in some aspects, depending on whether assignments to two or more TME classes are considered, a population of gene expression samples can be stratified into more than four different TME classes, e.g., into the four different TME classes disclosed (A, IS, ID, and IA) and/or combinations thereof.

### I.C.1.a Sample Classification.

The classification or stratification of samples into specific TME can be effected using a population-based classifier, i.e., a classification system based on data (e.g., parameters related to the specific cancer, biomarker expression levels, treatments, and outcomes of those treatments). In some aspects, the population-based classifier (or population-based method) disclosed herein assumes a zero-centered normal distribution (μ=0) of gene expression levels.

In a particular aspect of the population-based classifiers disclosed herein, the expression levels for a gene panel obtained from TABLE 1 or TABLE 2, or any of the gene panels (Genesets) disclosed in FIGS. 28A-G, are determined as disclosed above across an entire patient population. Across the whole patient population, the mean and standard deviation per gene are calculated from the expression levels of that gene. These values can be stored for future use as reference values for each gene in a gene panel.

From an individual patient sample (test sample), the patient's standardized expression level can be determined per each of the genes in the gene panel. The population mean value is subtracted from the patient's expression level for each gene in the gene panel. The resulting value is then divided by that particular gene standard deviation, to yield the Z-score for that gene in the panel. In some aspects, there is no correction for degrees of freedom. In other aspects, there is correction for degrees of freedom.

All the Z-scores corresponding to the genes in the gene panel are added, and then divided by the square root of the number of genes. The result is the Activation Score, zₛ, (Signature value) according to Equation 1:

$${z}_{s} = {\sum }_{g ∈ G} {z}_{s , g} / \sqrt{\vert G \vert} \qquad ( Equation 1 )$$

wherein z refers to Z-score, s to a sample (patient), g to gene, and G to the Signature geneset (i.e., the gene panel). |G| indicates the size of geneset G (i.e., the gene panel). z_{s,g} is a vector that describes the magnitude and direction away from the mean of population, and is unitless; the Activation Score zs is also unitless.

When the Activation Score (i.e., the Signature value) is equal to or greater than zero, i.e., zₛ>=0, then that Signature is said to be positive. When the Activation Score (i.e., the Signature value) is lower than zero, i.e., zₛ<0, then that Signature is said to be negative.

In some aspects, the calculation of a signature score, e.g., a Signature 1 or Signature 2, comprises
(i) measuring the expression level (e.g., mRNA expression level) for each gene in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and,
(iv) adding all the values obtained in step (iii) and dividing the resulting number by the square root of the number of genes in the gene panel,
wherein if the value obtained in (iv) is above zero, the signature score is a positive signature score, and wherein if the value obtained in (iv) is below zero, the signature score is a negative signature score.

In some aspects, the expression level for each gene in the gene panel in a test sample from the subject is merged with population data, e.g., expression data from the public datasets disclosed in the Examples section of the present disclosure.

It is to be understood that variations of the formula above are possible, for example, by grouping the expression levels of several genes (e.g., by gene family, of by common functional attributes such as several genes encoding ligands that bind to the same receptor) and/or assigning weights to the expression values or the Z-scores, and/or applying gene-specific thresholds.

A generalization of this population-based classifier is to compare patient Z-scores not to zero but to a signature-specific threshold (“threshold”), where zₛ>=threshold means positive (+) for the Signature, and zₛ<threshold means negative (−) for the Signature. The threshold is a hyperparameter of the classifier and depends on the disease being modeled. The threshold affects sensitivity and specificity of the population-based classifier.

Accordingly, in some aspects the Activation Score, zₛ, (Signature value) is calculated according to Equation 2, wherein T is a threshold value which would apply to the Activation Score.

$${z}_{s} = [ {\sum }_{g ∈ G} {z}_{s , g} / \sqrt{\vert G \vert} ] + T \qquad ( Equation 2 )$$

In some aspects, the Activation Score threshold value is about +0.01, about +0.02, about +0.03, about +0.04, about +0.05, about +0.06, about +0.07, about +0.08, about +0.09, about +0.10, about +0.15, about +0.20, about +0.25, about +0.30, about +0.35, about +0.40, about +0.45, about +0.50, about +0.55, about +0.60, about +0.65, about +0.70, about +0.75, about +0.80, about +0.85, about +0.90, about +0.95, about +1, about +2, about +3, about +4, about +5, about +6, about +7, about +8, about +9, about +10, or higher than +10.

In some aspects, the Activation Score threshold value is about −0.01, about −0.02, about −0.03, about −0.04, about −0.05, about −0.06, about −0.07, about −0.08, about −0.09, about −0.10, about −0.15, about −0.20, about −0.25, about −0.30, about −0.35, about −0.40, about −0.45, about −0.50, about −0.55, about −0.60, about −0.65, about −0.70, about −0.75, about −0.80, about −0.85, about −0.90, about −0.95, about −1, about −2, about −3, about −4, about −5, about −6, about −7, about −8, about −9, about −10, or lower than −10.

Accordingly, in some aspects the Activation Score, zₛ, (Signature value) is calculated according to Equation 3, wherein T is an independent threshold value which would apply to each gene in the panel.

$${z}_{s} = [ {\sum }_{g ∈ G} ( {z}_{s , g} + T ) / \sqrt{\vert G \vert} ] \qquad ( Equation 3 )$$

In some aspects, the gene-specific threshold can be at least about 5%, at least about 10%, at least about 15%, at least about 20%, at least about 25%, at least about 30%, at least about 35%, at least about 40%, or at least about 45% more than the mean, or zero.

In some aspects, the gene-specific threshold can also be at least about 5%, at least about 10%, at least about 15%, at least about 20%, at least about 25%, at least about 30%, at least about 35%, at least about 40%, or at least about 45% less than the mean, or zero.

In some aspects, the gene-specific threshold, which is unitless, can be about 0.05, about 0.10, about 0.15, about 0.20, about 0.25, about 0.30, about 0.35, about 0.40, about 0.45, about 0.50, about 0.55, about 0.60, about 0.65, about 0.70, about 0.75, about 0.80, about 0.85, about 0.90, about 0.95 or about 1.00 or more than the mean, or zero.

In some aspects, the gene-specific threshold, which is unitless, can be about 0.05, about 0.10, about 0.15, about 0.20, about 0.25, about 0.30, about 0.35, about 0.40, about 0.45, about 0.50, about 0.55, about 0.60, about 0.65, about 0.70, about 0.75, about 0.80, about 0.85, about 0.90, about 0.95 or about 1.00 or less than the mean, or zero.

In yet other aspects, the Activation Score, zₛ, (Signature value) is calculated according to Equation 4, wherein T₁ is an independent threshold value which would apply to each gene in the panel, and T₂ is a second threshold that would apply to the Activation Score.

$${z}_{s} = [ {\sum }_{g ∈ G} ( {z}_{s , g} + {T}_{1} ) / \sqrt{\vert G \vert} ] + {T}_{2} \qquad ( Equation 4 )$$

In some aspects, the same threshold can be applied to each Signature in the population-based classifier, e.g., Signature 1 and Signature 2. In other aspects, a different threshold can be applied to each Signature in the population-based classifier, e.g., Signature 1 and Signature 2. Thus, in a particular aspect of the present disclosure, the threshold can be different for Signature 1 and Signature 2.

In some aspects, Signature scores can be calculated according to alternative methods such as:
Signature score=SUM (test expression values—reference expression values), which could >0 or <0.
Signature score=Mean in distribution of (test expression values—reference expression values) with respect to threshold. If above threshold, positive. If below threshold, negative.
Signature score=Median in distribution of (test expression values—reference expression values) with respect to threshold. If above threshold, positive. If below threshold, negative.

In all these alternative methods, a normal distribution of RNA expression level values is required.

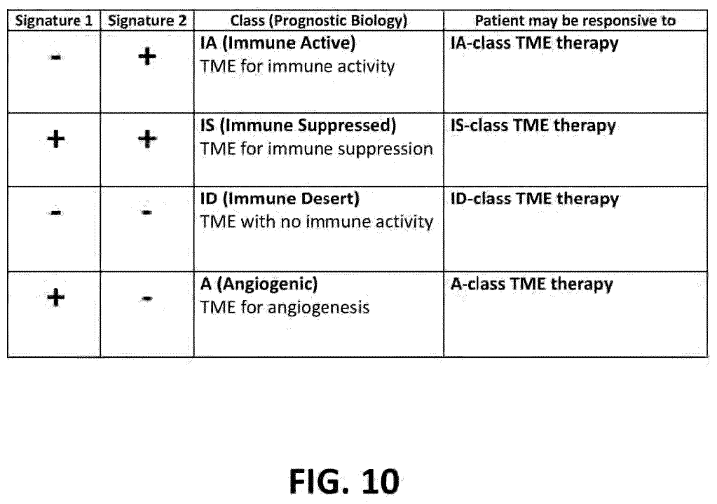

Prognostications or predictions based on a two Signature population-based classifier as disclosed herein, which would provide four TMEs (stromal phenotypes), can be made by correlating the Activation Score obtained from a patient's sample with the table in FIG. 10. In other words, based on the sign of patient Z-scores, and the thresholds used (e.g., positive or negative zₛ), the patients can be classified into one of the four TMEs, by applying the rules in FIG. 10 (patient classification rules based on the sign of the summed Signature 1 and Signature 2 Z-scores). These four TMEs are:
(a) IA (immune active): Defined by a negative Signature 1 and a positive Signature 2.
(b) IS (immune suppressed): Defined by a positive Signature 1 and a positive Signature 2.
(c) ID (immune desert): Defined by a negative Signature 1 and a negative Signature 2.
(d) A (angiogenic): Defined by a positive Signature 1 and a negative Signature 2.

The IS TME (stromal phenotype) generally does not include EBV (Epstein-Barr virus)-positive patients, MSI-H (microsatellite instability biomarker high) patients, or PD-L1-high patients. Those patients are generally found in the IA TME (stromal phenotype). Generalizations are illustrative, not definitive. Accordingly, in some aspects, the IS patient is not an EBV-positive patient. In some aspects, the IS patient is not an MSI-H patient. In some aspects, the IS patient is not a PD-L1 high patient. In some aspects, the IA patient is an EBV-positive patient. In some aspects, the IA patient is an MSI-H patient. In some aspects, the IA patient is a PD-L1 high patient.

In some aspects, a patient receiving an IS-class TME therapy is not an EBV-positive patient. In some aspects, a patient receiving an IS-class TME therapy not an MSI-H patient. In some aspects, a patient receiving an IS-class TME therapy is not a PD-L1 high patient.

In some aspects, a patient receiving an IA-class TME therapy is an EBV-positive patient. In some aspects, a patient receiving an IA-class TME therapy is an MSI-H patient. In some aspects, a patient receiving an IA-class TME therapy a PD-L1 high patient.

In some aspects, depending on the application of different weights and parameters to the calculation of Z-scores and the application of different thresholds, a tumor sample can be classified in two or more TMEs. In these aspects, the tumor sample or patient would be biomarker-positive for two or more TMEs, e.g., A and IS biomarker-positive. Consequently, such tumor or patient could be treated with two or more TME-class therapies disclosed herein, e.g., as a combination therapy, wherein each TME-class therapy would correspond to one of the TMEs for which the tumor sample or patient is biomarker-positive.

For the TME that is dominated by immune activity, such as the IA (Immune Active) phenotype, a patient with this biology might be responsive to anti-PD-1 (e.g., sintilimab, tislelizumab, pembrolizumab, or an antigen binding portion thereof), anti-PD-L1, anti-CTLA4 (the checkpoint inhibitors, or CPIs), or RORγ agonist therapeutics (all therapeutics for all stromal subtypes described more thoroughly below).

For the TME that is dominated by angiogenic activity, such as a patient classified as the A (Angiogenic) phenotype, a patient with this biology might be responsive to VEGF-targeted therapies, DLL4-targeted therapies, angiopoietin/TIE2-targeted therapies, anti-VEGF/anti-DLL4 bispecific antibodies, such as navicixizumab, as well as anti-VEGF antibodies such as varisacumab or bevacizumab.

For the TME that is dominated by immune suppression, such a patient classified as the IS (Immune Suppressed) phenotype might be resistant to checkpoint inhibitors unless also given a drug to reverse immunosuppression such as anti-phosphatidylserine (anti-PS) therapeutics, PI3Kγ inhibitors, adenosine pathway inhibitors, IDO, TIMs, LAG3, TGFβ, and CD47 inhibitors. Bavituximab is a preferred anti-PS therapeutic. A patient with this biology also has underlying angiogesis and can also get benefit from anti-angiogenics, such as those used for the A stromal subtype.

For the TME with no immune activity, such as a patient classified as the ID (Immune Desert) phenotype, a patient with this biology would not respond to checkpoint inhibitors, anti-angiogenics or other TME targeted therapies, and so should not be treated anti-PD-1s (e.g., sintilimab, tislelizumab, pembrolizumab, or an antigen binding portion thereof), anti-PD-L1s, anti-CTLA-4s, or RORγ agonists as monotherapies. A patient with this biology might be treated with therapies that induce immune activity allowing them to then get benefit from checkpoint inhibitors. Therapies that might induce immune activity for these patients include vaccines, CAR-Ts, neo-epitope vaccines, including personalized vaccines, and TLR-based therapies.

In one aspect, different subsets of genes within a Signature can be equally predictive because such genes represent numerous facets of a wide biology. Thus, a four TME classifier as disclosed herein can be generated using the entire genesets of TABLE 1 and TABLE 2 (or any of the genesets disclosed in FIGS. 28A-G), or use subsets of genes from TABLE 1 and TABLE 2 (or subsets of genes from any of the genesets disclosed in FIGS. 28A-G), e.g., the subsets disclosed in TABLE 3 and TABLE 4.

In some aspects, the population-based classifiers disclosed herein are used prognostically. In some aspects, the population-based classifiers disclosed here are used predictively in a clinical setting, i.e., as predictive biomarkers.

In some aspects, a population can be stratified into more than four classes if the classifier determines that samples or patients are biomarker-positive for two more TME classes disclosed herein. For example, a population could be stratified as being IA biomarker-positive, ID biomarker-positive, A biomarker-positive, IS biomarker-positive, IA and ID biomarker-positive, IA and A biomarker-positive, and so forth. Conversely, a population could be stratified as being IA biomarker-negative, ID biomarker-negative, A biomarker-negative, IS biomarker-negative, IA and ID biomarker-negative, IA and A biomarker-negative, and so forth.

I.D Non-population-based classifiers

In some aspects, the present disclosure provides the methodology to create non-population-based classifiers (or sets of classifiers) that are able to stratify (or classify) gene expression samples into several TME classes. The underlying tumor biology of the four TMEs (i.e., stromal subtypes or phenotypes): IA (immune active), ID (immune desert), A (angiogenic) and IS (immune suppressed), discussed above, can be revealed by application of artificial neural network (ANN) methods and other machine-learning techniques. In some aspects, application of the methods disclosed herein can classify a tumor sample or patient into more than one of the TMEs disclosed herein, e.g., a patient or sample can be biomarker positive for two or more TMEs.

In the context of the present disclosure, it is to be understood that the term classifier includes one or more classifiers, or combinations of classifiers, which can belong to the same or different classes (e.g., population and/or non-population classifiers, or a combination of non-population classifiers) wherein the term classifier is used to describe the output of a mathematical model assigning, e.g., a test sample to a specific TME class.

While the population-based classifiers disclosed herein rely on datasets that have RNA expression values for many patients to then classify those patients, the machine-learning methods (e.g., ANN, logistic regression, or random forests) replicate, recapitulate, reproduce, and/or closely estimate the output of the population-based classifiers.

For example, the ANN method takes as input the gene expression values of the genes or subset thereof disclosed herein (i.e. features), and based on the pattern of expression, identifies patient samples (i.e., patients) with either predominantly angiogenic expression, predominantly activated immune gene expression, a mixture of both or neither of these expression patterns. These four phenotypic types are predictive of the response to certain types of treatment.

Thus, in some aspects of the current disclosure, a classification of the TME as IS (immuno suppressed), as assigned to a patient sample (i.e., a patient) by a machine-learning method disclosed herein (e.g., an ANN), means that the patient has both activated immune gene expression and angiogenic gene expression.

The A (angiogenic) TME classification, as assigned to a patient sample by a non-population-based classifier disclosed herein, e.g., an ANN, means that the patient sample has predominantly angiogenic gene expression. The IA (immune active) TME classification, as assigned to a patient sample by a non-population-based classifier disclosed herein, e.g., an ANN, means that the patient sample has predominantly activated immune gene expression. The ID (immune desert) TME, as assigned to a patient sample by a non-population-based classifier disclosed herein, e.g., an ANN, means that the patient sample has no, highly reduced, low, or very low immune gene expression and angiogenic immune gene expression.

In some aspects, the non-population-based classifier disclosed herein is a classifier obtained by the application of machine-learning techniques. In some aspects, the machine-learning technique is selected from the group consisting of Logistic Regression, Random Forest, Artificial Neural Network (ANN), Support Vector Machine (SVM), XGBoost (XGB; an implementation of gradient boosted decision trees designed for speed and performance), Glmnet (a package that fits a generalized linear model via penalized maximum likelihood), cforest (implementation of the random forest and bagging ensemble algorithms utilizing conditional inference trees as base learner), Classification and Regression Trees for Machine-learning (CART), Treebag (bagging, i.e., bootstrap aggregating, algorithm to improve model accuracy in regression and classification problems which building multiple models from separated subsets of train data, and constructs a final aggregated model), K-Nearest Neighbors (kNN), or a combination thereof.

Logistic Regression often is regarded as one of the best predictors on small datasets. However, Tree-based models (e.g., Random Forest, ExtraTrees) and ANNs can uncover latent interactions among features. When there is little interaction, though, Logistic Regression and more complex models have similar performance.

The non-population based classifiers disclosed herein can be trained with data corresponding to a set of samples for which gene expression data, e.g., mRNA expression data, corresponding to a gene panel has been obtained. For example, the training set comprises expression data from the genes presented in TABLE 1 and TABLE 2 (or in any of the gene panels (Genesets) disclosed in FIGS. 28A-G), and any combination thereof. In some aspects, the gene panel comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, or 100 genes. In some aspects, the gene panel comprises more than 100 genes. In some aspects, the gene panel comprises between about 10 and about 20, about 20 and about 30, about 30 and about 40, about 40 and about 50, about 50 and about 60, about 60 and about 70, about 70 and about 80, about 80 and about 90, or about 90 and about 100 genes selected from TABLE 1 and TABLE 2 (or from any of the gene panels (Genesets) disclosed in FIGS. 28A-G).

In some aspects, the training dataset comprises further variables for each sample, for example the sample classification according to a population-based classifier disclosed herein. In other aspects, the training data comprises data about the sample such as type of treatment administered to the subject, dosage, dose regimen, administration route, presence or absence of co-therapies, response to the therapy (e.g., complete response, partial response or lack of response), age, body weight, gender, ethnicity, tumor size, tumor stage, presence or absence of biomarkers, etc.

In some aspects, it is helpful to select genes for the training dataset on the basis of a combination of factors including p value, fold change, and coefficient of variation as would be understood by a person skilled in the art. In some aspects, the use of one or more selection criteria and subsequent rankings permits the selection of the top 2.5%, 5%, 7.5%, 10%, 12.5%, 15%, 17.5%, 20%, 30%, 40%, 50% or more of the ranked genes in a gene panel for input into the model. As would be understood, one can select therefore all of the individually identified gene or subsets of the genes in TABLES 1 and 2, and test all possible combinations of the selected genes to identify useful combinations of genes to generate a predictive model. A selection criterion to determine the number of selected individual genes to test in combination, and to select the number of possible combinations of genes will depend upon the resources available for obtaining the gene data and/or the computer resources available for calculating and evaluating classifiers resulting from the model.

In some aspects, genes can appear to be driver genes, based on the results of the training of the machine learning model. The term “driver gene” as used herein, refers to a gene which includes a driver gene mutation. In some aspects, a driver gene is a gene in which one or more acquired mutations, e.g., driver gene mutations, can be causally linked to cancer progression. In some aspects, a driver gene can modulate one or more cellular processes including: cell fate determination, cell survival and genome maintenance. A driver gene can be associated with (e.g., can modulate) one or more signaling pathways, e.g., a TGF-beta pathway, a MAPK pathway, a STAT pathway, a PI3K pathway, a RAS pathway, a cell cycle pathway, an apoptosis pathway, a NOTCH pathway, a Hedgehog (HH) pathway, a APC pathway, a chromatin modification pathway, a transcriptional regulation pathway, a DNA damage control pathway, or a combination thereof. Exemplary driver genes include oncogenes and tumor suppressors. In some aspects, a driver gene provides a selective growth advantage to the cell in which it occurs. In some aspects, a driver gene provides a proliferative capacity to the cell in which it occurs, e.g., allows for cell expansion, e.g., clonal expansion. In some aspects, a driver gene is an oncogene. In some aspects, a driver gene is a tumor suppressor gene (TSG).

The presence of noisy, low-expression genes in a geneset can decrease the sensitivity of the model. Accordingly, in some aspects, low-expression genes can be down-weighted or filtered (eliminated) from the machine learning model. In some aspects, low-expression gene filtering is based on a statistic calculated from gene expression (e.g., RNA levels). In some aspects, low-expression gene filtering is based on minimum (min), maximum (max), average (mean), variance (sd), or combinations thereof of, e.g., raw read counts for each gene in the geneset. For each geneset, an optimal filtering threshold can be determined. In some aspects, the filtering threshold is optimized to maximize the number of differentially expressed genes in the geneset

The non-population based classifiers generated by the machine-learning methods disclosed herein (e.g., ANN) can be subsequently evaluated by determining the ability of the classifier to correctly call each test subject. In some aspects, the subjects of the training population used to derive the model are different from the subjects of the testing population used to test the model. As would be understood by a person skilled in the art, this allows one to predict the ability of the geneset used to train the classifier as to their ability to properly characterize a subject whose stromal phenotype trait characterization (e.g., TME class) is unknown.

The data which is input into the mathematical model can be any data which is representative of the expression level of the product of the gene being evaluated, e.g., mRNA. Mathematical models useful in accordance with the present disclosure include those using supervised and/or unsupervised learning techniques. In some aspect of the disclosure, the mathematical model chosen uses supervised learning in conjunction with a “training population” to evaluate each of the possible combinations of biomarkers. In one aspect, the mathematical model used is selected from the following: a regression model, a logistic regression model, a neural network, a clustering model, principal component analysis, nearest-neighbor classifier analysis, linear discriminant analysis, quadratic discriminant analysis, a support vector machine, a decision tree, a genetic algorithm, classifier optimization using bagging, classifier optimization using boosting, classifier optimization using the Random Subspace Method, a projection pursuit, genetic programming and weighted voting. In some aspects, a logistic regression model is used. In other aspects, a decision tree model if used. In some aspects, a neural network model is used.

The results of applying a mathematical model of the present disclosure, e.g., an ANN model, to the data will generate one or more classifiers using one or more gene panels. In some aspects, multiple classifiers are created which are satisfactory for the given purpose (e.g., to correctly classify a TME, i.e., a stromal phenotype). In this instance, in some aspects, a formula is generated which utilizes more than one classifier. For example, a formula can be generated which utilizes classifiers in series (e.g. first obtains results of classifier A, then classifier B; e.g., classifier A differentiates TMEs; and classifier B then determines whether a particular treatment would be assigned to such TME). In another aspect, a formula can be generated which results from weighting the results of more than one classifier. Other possible combinations and weightings of classifiers would be understood and are encompassed herein. In some aspects, different cut-offs applied to the same classifier or different classifiers applied to the same sample can result in the classification of the sample into different stromal phenotypes. In other words, depending on the combination of threshold and/or classifiers, a sample can be classified in two or more stromal phenotypes (TMEs) and accordingly the sample can be biomarker-positive and/or biomarker-negative for the IA, ID, IS or A TME classes disclosed herein or any combination thereof (e.g., the subject can be A and IS biomarker-positive and ID and IA biomarker-negative).

Classifiers, e.g., non-population based classifiers (e.g., ANN models) generated according to the methods disclosed herein can be used to test an unknown or test subject. In one aspect, the model generated by a machine-learning method, e.g., an ANN, identified herein can detect whether an individual has a particular TME. In some aspects, the model can predict whether a subject will respond to a particular therapy. In other aspects, the model can select or be used to select a subject for administration of a particular therapy.

In one aspect of the disclosure, each classifier is evaluated for its ability to properly characterize each subject of the training population using methods known to a person skilled in the art. For example, one can evaluate the classifier using cross validation, Leave One Out Cross Validation (LOOCV), n-fold cross validation, or jackknife analysis using standard statistical methods. In another aspect, each classifier is evaluated for its ability to properly characterize those subjects of the training population which were not used to generate the classifier.

In some aspects, one can train the classifier using one dataset, and evaluate the classifier on another distinct dataset. Accordingly, since the testing dataset is distinct from the training dataset, there is no need for cross validation.

In one aspect, the method used to evaluate the classifier for its ability to properly characterize each subject of the training population is a method which evaluates the classifier's sensitivity (TPF, true positive fraction) and 1-specificity (FPF, false positive fraction). In one aspect, the method used to test the classifier is Receiver Operating Characteristic (“ROC”) which provides several parameters to evaluate both the sensitivity and specificity of the result of the model generated, e.g., a model derived from the application of an ANN.

In some aspects, the metrics used to evaluate the classifier for its ability to properly characterize each subject of the training population comprise classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), Negative Predicted Value (NPV), or any combination thereof. In one specific aspect, the metrics used to evaluate the classifier for its ability to properly characterize each subject of the training population are classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), and Negative Predicted Value (NPV).

In some aspects, the training set includes a reference population of at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, or at least about 1000 subjects.

In some aspects, the expression data, e.g., mRNA expression data, for some or all of the genes identified in the present disclosure (e.g., those presented in TABLE 1 and TABLE 2; or FIGS. 28A-G) are used in a regression model, such as but not limited to a logistic regression model or a linear regression model, so as to identify classifiers useful in classifying TMEs (i.e., stromal phenotypes). The model is used to test various combinations of two or more of the biomarker genes identified in TABLE 1 and TABLE 2 (or FIGS. 28A-G) to generate classifiers. In the case of logistic regression models, the classifiers which result are in the form of equations which provide a dependent variable Y, which represents the presence or absence of a given phenotype (e.g., TME class) where the data representing the expression of each of the biomarker genes in the equation is multiplied by a weighted coefficient as generated by the regression model. The classifiers generated can be used to analyze expression data from a test subject and provide a result indicative of the probability of a test subject having a particular TME.

In general, a multiple regression equation of interest can be written as

$$Y = α + {β}_{1} {X}_{1} + {β}_{2} {X}_{2} + … + {β}_{k} {X}_{k} + ε$$

wherein Y, the dependent variable, indicates presence (when Y is positive) or absence (when Y is negative) of the biological feature (e.g., absence or presence of one or more pathologies) associated with the first subgroup. This model says that the dependent variable Y depends on k explanatory variables (the measured characteristic values for the k select genes (e.g., the biomarker genes) from subjects in the first and second subgroups in the reference population), plus an error term that encompasses various unspecified omitted factors. In the above-identified model, the parameter β₁ gauges the effect of the first explanatory variable X₁ on the dependent variable Y (e.g., a weighting factor), holding the other explanatory variables constant. Similarly, β₂ gives the effect of the explanatory variable X₂ on Y, holding the remaining explanatory variables constant.

A logistic regression model is a non-linear transformation of the linear regression. The logistic regression model is often referred to as the “logit” model and can be expressed as

$$ln [ p / ( 1 - p ) ] = α + {β}_{1} {X}_{1} + {β}_{2} {X}_{2} + … + {β}_{k} {X}_{k} + ε$$

$$[ p / ( 1 - p ) ] = {exp}^{α} {exp}^{{β}_{1} {X}_{1}} {exp}^{{β}_{2} {X}_{2}} \times … \times {exp}^{{β}_{k} {x}_{k}} {exp}^{ε}$$

wherein,
α and ε are constants
ln is the natural logarithm, logₑ, where e=2.71828 . . . ,
p is the probability that the event Y occurs, p(Y=1),
p/(1−p) is the “odds ratio”,
ln[p/(1−p)] is the log odds ratio, or “logit”, and all other components of the model are the same as the general linear regression equation described above. The term for a and F can be folded into a single constant. In some aspects, a single term is used to represent a and F. The “logistic” distribution is an S-shaped distribution function. The logit distribution constrains the estimated probabilities (p) to lie between 0 and 1.

In some aspects, the logistic regression model is fit by maximum likelihood estimation (MLE). In other words, the coefficients (e.g., α, β₁, β₂, . . . ) are determined by maximum likelihood. A likelihood is a conditional probability (e.g., P(Y|X), the probability of Y given X). The likelihood function (L) measures the probability of observing the particular set of dependent variable values (Y₁, Y₂, . . . , Yₙ) that occur in the sample dataset. It is written as the probability of the product of the dependent variables:

$$L = Prob ( {Y}_{1} * {Y}_{2} ** * {Y}_{n} )$$

The higher the likelihood function, the higher the probability of observing the Ys in the sample. MLE involves finding the coefficients (α, β₁, β₂, . . . ) that makes the log of the likelihood function (LL<0) as large as possible or −2 times the log of the likelihood function (−2LL) as small as possible. In MLE, some initial estimates of the parameters α, β₁, β₂, . . . are made. Then the likelihood of the data given these parameter estimates is computed. The parameter estimates are improved and the likelihood of the data is recalculated. This process is repeated until the parameter estimates do not change much (for example, a change of less than 0.01 or 0.001 in the probability). Examples of logistic regression and fitting logistic regression models are found in Hastie, The Elements of Statistical Learning, Springer, New York, 2001, pp. 95-100.

In another aspect, the expression, e.g., mRNA levels, measured for each of the biomarker genes in a gene panel of the present disclosure can be used to train a neural network. A neural network is a two-stage regression or classification model. A neural network can be binary or non-binary. A neural network has a layered structure that includes a layer of input units (and the bias) connected by a layer of weights to a layer of output units. For regression, the layer of output units typically includes just one output unit. However, neural networks can handle multiple quantitative responses in a seamless fashion. As such a neural network can be applied to allow identification of biomarkers which differentiate as between more than two populations (i.e., more than two phenotypic traits), e.g., the four TME class disclosed herein.

In one specific example, a neural network can be trained using expression data from the products, e.g., mRNA, of the biomarker genes disclosed in TABLE 1 and TABLE 2 (or FIGS. 28A-G) for a set of samples obtained from a population of subjects to identify those combinations of biomarkers which are specific for a particular TME. Neural networks are described in Duda et al., 2001, Pattern Classification, Second Edition, John Wiley & Sons, Inc., New York; and Hastie et al., 2001, The Elements of Statistical Learning, Springer-Verlag, New York.

In some aspects, a neural network disclosed herein, e.g., a back-propagation neural network (see, for example Abdi, 1994, “A neural network primer”, J. Biol System. 2, 247-283) containing a single input layer with, e.g., 98 or 87 genes from TABLES 1 and 2 (or from FIGS. 28A-G), a single hidden layer of 2 neurons, and 4 outputs in a single output layer can be implemented using the EasyNN-Plus version 4.0g software package (Neural Planner Software Inc.), scikit-learn (scikit-learn.org), or any other machine learning package or program known in the art.

The pattern classification and statistical techniques described above are merely examples of the types of models that can be used to construct classifiers useful for diagnosing or detecting, e.g., one or more pathologies, for example, Clustering as described, e.g., on pages 211-256 of Duda and Hart, Pattern Classification and Scene Analysis, 1973, John Wiley & Sons, Inc., New York; Principal Component Analysis, as described, e.g., in Jolliffe, 1986, Principal Component Analysis, Springer, New York; Nearest Neighbour Classifier Analysis, as decribed, for example, in Duda, Pattern Classification, Second Edition, 2001, John Wiley & Sons, Inc, and inHastie, 2001, The Elements of Statistical Learning, Springer, New York); Linear Discriminant Analysis, as described for example in Duda, Pattern Classification, Second Edition, 2001, John Wiley & Sons, Inc; in Hastie, 2001, The Elements of Statistical Learning, Springer, New York; or in Venables & Ripley, 1997, Modern Applied Statistics with s-plus, Springer, New York); Support Vector Machines, as described, for example, in Cristianini and Shawe-Taylor, 2000, An Introduction to Support Vector Machines, Cambridge University Press, Cambridge, in Boser et al., 1992, “A training algorithm for optimal margin classifiers, in Proceedings of the 5th Annual ACM Workshop on Computational Learning Theory, ACM Press, Pittsburgh, PA, pp. 142-152; or in Vapnik, 1998, Statistical Learning Theory, Wiley, New York.

In some aspects, the non-population-based classifier comprises a model derived from an ANN. In some aspects, the ANN is a feed-forward neural network. A feed-forward neural network is an artificial network wherein connection between the input and output nodes do not form a cycle. As used here in the context of an ANN, the terms “node” and “neuron” are used interchangeably. Thus, it is different from recurrent neural networks. In this network, the information moves in only one direction, forward, from the input nodes, through the hidden nodes (if any) and to the output nodes. There are no cycles or loops in the network. Except for the input nodes, each node is a neuron that uses a nonlinear activation function, which is developed to model the frequency of action potential, or firing, of biological neurons.

In some aspects, the ANN is a single-layer perceptron network, which consists of a single layer of output nodes; the inputs are fed directly to the outputs via a series of weights. The sum of the products of the weights and the inputs is calculated in each node, and if the value is above some threshold (typically 0) the neuron fires and takes the activated value (typically 1).

In some aspects, the ANN is a multi-layer perceptron (MLP). This class of networks consists of multiple layers of computational units, usually interconnected in a feed-forward way. Each neuron in one layer has directed connections to the neurons of the subsequent layer. In many applications, the units of these networks apply an activation function, e.g., a sigmoid function. An MLP comprises at least three layers of nodes: an input layer, a hidden layer and an output layer.

In some aspects, the activation function is a sigmoid function described according to the formula y(vᵢ)=tanh(vi), i.e., a hyperbolic tangent that ranges from −1 to +1. In some aspects, the activation function is a sigmoid function described according to the formula y(vᵢ)=(1+e^{-vi})⁻¹, i.e., a logistic function similar in shape to the tanh function but ranges from 0 to +1. In these formulas, yᵢ is the output of the ith node (neuron) and vᵢ is the weighted sum of the input connections.

In some aspects, the activation function is a rectifier linear unit (ReLU) or a variant thereof, e.g., a noisy ReLU, a leaky ReLU, a parametric ReLU, or an exponential LU. In some aspects, the ReLU is defined by the formula f(x)=x+=max (0, x), wherein x is the input to a neuron. The ReLU activation function enables better training of deep neural networks (DNN) compared to the hyperbolic tangent or the logistic sigmoid. A DNN is an ANN with multiple layers between the input and output layers. DNNs are typically feed-forward networks in which data flows from the input layer to the output layer without looping back. DNNs are prone to over-fitting because of the added layers of abstraction, which allow them to model rare dependencies in the training data. In some aspects, the activation function is the softplus or smoothReLU function, a smooth approximation of the ReLU, which is described by the formula f(x)=ln(1+e^{x}). The derivative of softplus is the logistic function.

In some aspects, the MLP comprises three or more layers (an input and an output layer with one or more hidden layers) of nonlinearly-activating nodes. Its multiple layers and non-linear activation distinguish MLP from a linear perceptron. It can distinguish data that is not linearly separable. Since MLPs are fully connected, each node in one layer connects with a certain weight wᵢⱼ to every node in the following layer. Learning occurs in the perceptron by changing connection weights after each piece of data is processed, based on the amount of error in the output compared to the expected result. This is an example of supervised learning, and is carried out through backpropagation.

In some aspects, the MLP has 3 layers. In other aspects, the MLP has more than 3 layers. In some aspects, the MLP has a single hidden layer. In other aspects, the MLP has more than one hidden layer.

In some aspects, the input layer comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 neurons.

In some aspects, the input layer comprises between 70 and 100 neurons. In some aspects, the input layer comprises between 70 and 80 neurons. In some aspects, the input layer comprises between 80 and 90 neurons. In some aspects, the input layer comprises between 90 and 100 neurons. In some aspects, the input layer comprises between 70 and 75 neurons. In some aspects, the input layer comprises between 75 and 80 neurons. In some aspects, the input layer comprises between 80 and 85 neurons. In some aspects, the input layer comprises between 85 and 90 neurons. In some aspects, the input layer comprises between 90 and 95 neurons. In some aspects, the input layer comprises between 95 and 100 neurons.

In some aspects, the input layer comprises between at least about 1 to at least about 5, between at least about 5 and at least about 10, between at least about 10 and at least about 15, between at least about 15 and at least about 20, between at least about 20 and at least about 25, between at least about 25 and at least about 30, between at least about 30 and at least about 35, between at least about 35 and at least about 40, between at least about 40 and at least about 45, between at least about 45 and at least about 50, between at least about 50 and at least about 55, between at least about 55 and at least about 60, between at least about 60 and at least about 65, between at least about 65 and at least about 70, between at least about 70 and at least about 75, between at least about 75 and at least about 80, between at least about 80 and at least about 85, between at least about 85 and at least about 90, between at least about 90 and at least about 95, between at least about 95 and at least about 100, between at least about 100 and at least about 105, between at least about 105 and at least about 110, between at least about 110 and at least about 115, between at least about 115 and at least about 120, between at least about 120 and at least about 125, between at least about 125 and at least about 130, between at least about 130 and at least about 135, between at least about 135 and at least about 140, between at least about 140 and at least about 145, or between at least about 145 and at least about 150 neurons.

In some aspects, the input layer comprises between at least about 1 and at least about 10, between at least about 10 and at least about 20, between at least about 20 and at least about 30, between at least about 30 and at least about 40, between at least about 40 and at least about 50, between at least about 50 and at least about 60, between at least about 60 and at least about 70, between at least about 70 and at least about 80, between at least about 80 and at least about 90, between at least about 90 and at least about 100, between at least about 100 and at least about 110, between at least about 110 and at least about 120, between at least about 120 and at least about 130, between at least about 130 and at least about 140, or between at least about 140 and at least about 150 neurons.

In some aspects, the input layer comprises between at least about 1 and at least about 20, between at least about 20 and at least about 40, between at least about 40 and at least about 60, between at least about 60 and at least about 80, between at least about 80 and at least about 100, between at least about 100 and at least about 120, between at least about 120 and at least about 140, between at least about 10 and at least about 30, between at least about 30 and at least about 50, between at least about 50 and at least about 70, between at least about 70 and at least about 90, between at least about 90 and at least about 110, between at least about 110 and at least about 130, or between at least about 130 and at least about 150 neurons.

In some aspects, the input layer comprises more than about 1, more than about 5, more than about 10, more than about 15, more than about 20, more than about 25, more than about 30, more than about 35, more than about 40, more than about 45, more than about 50, more than about 55, more than about 60, more than about 65, more than about 70, more than about 75, more than about 80, more than about 85, more than about 90, more than about 95, more than about 100, more than about 105, more than about 110, more than about 115, more than about 120, more than about 125, more than about 130, more than about 135, more than about 140, more than about 145, or more than about 150 neurons.

In some aspects, the input layer comprises less than about 1, less than about 5, less than about 10, less than about 15, less than about 20, less than about 25, less than about 30, less than about 35, less than about 40, less than about 45, less than about 50, less than about 55, less than about 60, less than about 65, less than about 70, less than about 75, less than about 80, less than about 85, less than about 90, less than about 95, less than about 100, less than about 105, less than about 110, less than about 115, less than about 120, less than about 125, less than about 130, less than about 135, less than about 140, less than about 145, or less than about 150 neurons.

In some aspects, a weight is applied to the input of each one of the neurons in the input layer.

In some aspects, the ANN comprises a single hidden layer. In some aspects, the ANN comprises 1, 2, 3, 4, 5, 6, 7, 8, 9 or 10 hidden layers. In some aspects, the single hidden layer comprises 1, 2, 3, 4, 5, 6, 7, 8, 9, or 10 neurons. In some aspects, the single hidden layer comprises at least 1, at least 2, at least 3, at least 4, at least 5, at least 6, at least 7, at least 8, at least 9, or at least 10 neurons. In some aspects, the single hidden layer comprises less than 10, less than 9, less than 8, less than 7, less than 6, less than 5, less than 4, or less than 3 neurons. In some aspects, the single hidden layer comprises 2 neurons. In some aspects, the single hidden layer comprises 3 neurons. In some aspects, the single hidden layer comprises 4 neurons. In some aspects, the single hidden layer comprises 5 neurons. In some aspects, a bias is applied to the neurons in the hidden layer.

In some aspects, the ANN comprises four neurons in the output layer corresponding to different TMEs. In some aspects, the four neurons in the output layer correspond to the four TMEs disclosed above, IA (immune active), IS (immune suppressed), ID (immune desert), and A (angiogenic).

In some aspects the classification of the output layer is normalized to a probability distribution over predicted output classes, and the components will add up to 1, so that they can be interpreted as probabilities.

In some aspects, the multi-class classification of the output layer values into four phenotype classes (IA, ID, A, and IS) is supported by applying a logistic regression function. In some aspects, the multi-class classification of the output layer values into four phenotype classes (IA, ID, A, and IS) is supported by applying a logistic regression classifier, e.g., the Softmax function. Softmax assigns decimal probabilities to each class that adds up to 1.0. In some aspects, the use of a logistic regression classifier such as the Softmax function helps training converge more quickly. In some aspects, the logistic regression classifier comprising a Softmax function is implemented through a neural network layer just before the output layer. In some aspects, such neural network layer just before the output layer has the same number of nodes as the output layer.

In some aspects, various cut-offs are applied to the results of the logistic regression classifier (e.g., Softmax function) depending on the particular dataset used (see, e.g., cut-offs applied to select a particular population of subjects, e.g., those responding to a particular therapy). Thus, applying different sets of cut-offs can classify a cancer or a patient in not only one of the four TMEs disclosed above, IA (immune active), IS (immune suppressed), ID (immune desert), or A (angiogenic), but also classify a cancer or a patient in more than one TME disclosed above. Accordingly, in some aspects, a cancer or a patient can be classified as being biomarker-positive for IA, IS, ID, A, and any combination thereof. Conversely, in some aspects, a cancer or a patient can be classified as being biomarker-negative for IA, IS, ID, A, and any combination thereof.

In some aspects, the two neurons in the hidden layer of the MLP ANN disclosed herein correspond to Signature 1 and Signature 2 identified in the population-based classifier of the present disclosure, which can be used to generate the training dataset.

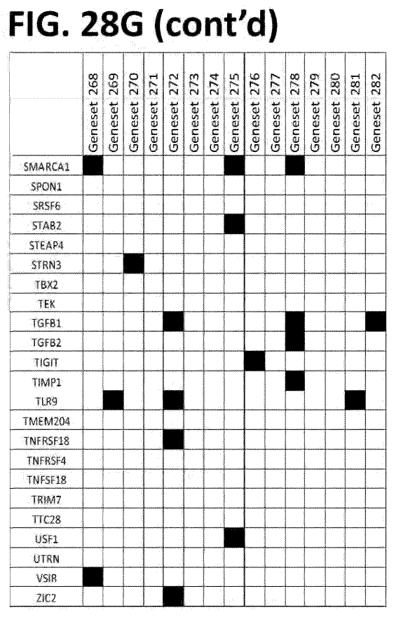

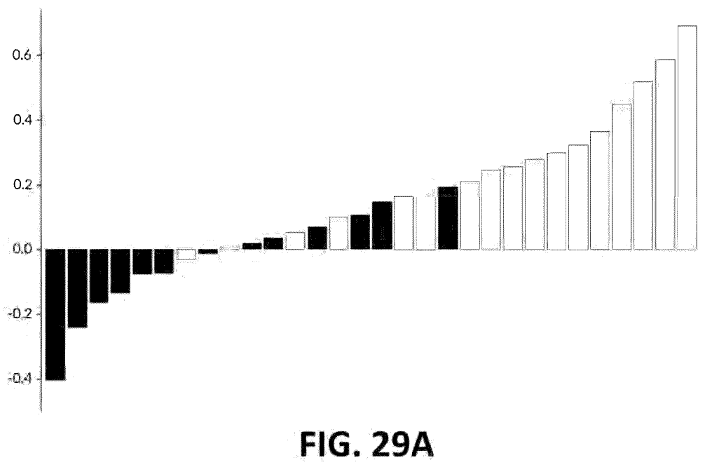

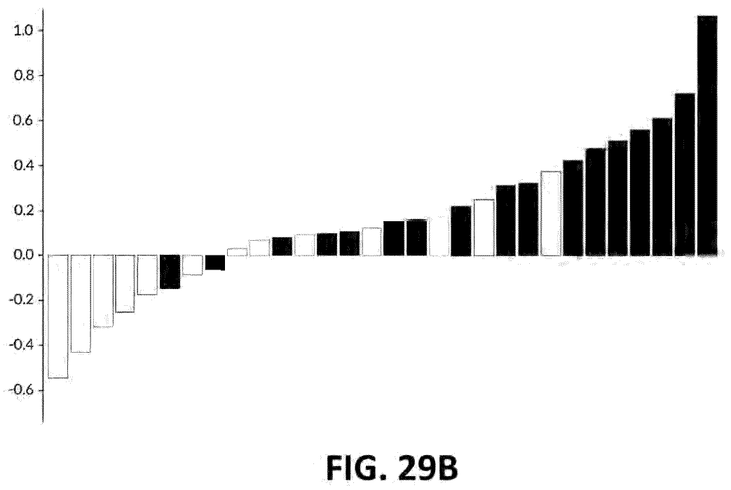

In some aspects, all, or a subset of genes of Signature 1, and all, or a subset of genes of Signature 2, have positive or negative gene weights in the ANN model for each hidden layer (FIG. 29).

In some aspects, a machine-learning method disclosed herein, e.g., an ANN disclosed herein, has been trained using a geneset provided in the table below.

TABLE 5
Genesets for use in machine-learning (e.g., ANN) training.

| | GENES |
|---|---|
| Training set 1 | ABCC9, ADAMTS4, AFAP1L2, AGR2, BACE1, BGN, BMP5, C11ORF9, CAPG, |
| (n = 124) | CAVIN2, CCL2, CCL3, CCL4, CD19, CD274, CD3E, CD4, CD79A, CD8B, |
| | COL4A2, COL8A1, COL8A2, CPXM2, CTLA4, CTSB, CXCL10, CXCL11, |
| | CXCL12, CXCL9, DUSP4, EBF1, ECM2, EDNRA, EIF5A, ELN, EPHA3, ETV5, |
| | FBLN5, FOLR2, GAD1, GNAS, GNB4, GUCY1A1, GZMB, HAVCR2, HEY2, |
| | HFE, HMOX1, HP, HSPB2, IDO1, IFNA2, IFNB1, IFNG, IGFBP3, IGLL5, IL1B, |
| | IQGAP3, ITGA9, ITPR1, JAM2, JAM3, KCNJ8, LAG3, LAMB2, LHFPL6, |
| | LTBP4, MEOX1, MEST, MGP, MMP12, MMP13, MST1, MT2A, MTA2, |
| | NAALAD2, NFATC1, NOV, OLFML2A, PCDH17, PDCD1, PDCD1LG2, |
| | PDE5A, PDGFRB, PEG3, PLA2G4A, PLAU, PLSCR2, PLXDC2, RAC2, REG4, |
| | RGS4, RGS5, RNF144A, RNH1, RRAS, RUNX1T1, SELP, SERPINE1, |
| | SERPINE2, SGIP1, SMARCA1, SPON1, SRSF6, STAB2, STEAP4, STRN3, |
| | TBX2, TEK, TGFB1, TGFB2, TIGIT, TIMP1, TLR9, TMEM204, TNFRSF18, |
| | TNFRSF4, TNFSF18, TRIM7, TTC28, USF1, UTRN, VSIR, ZIC2 |
| Training set 2 | ABCC9, ADAMTS4, AFAP1L2, AGR2, BACE1, BGN, BMP5, C11ORF9, CAPG, |
| (n = 119) | CAVIN2, CCL2, CCL3, CCL4, CD19, CD274, CD3E, CD4, CD79A, CD8B, |
| | COL8A1, COL8A2, CPXM2, CTLA4, CTSB, CXCL10, CXCL11, CXCL12, |
| | CXCL9, DUSP4, EBF1, ECM2, EDNRA, EIF5A, ELN, EPHA3, ETV5, FBLN5, |
| | GAD1, GNAS, GNB4, GUCY1A1, GZMB, HAVCR2, HEY2, HFE, HMOX1, HP, |
| | HSPB2, IDO1, IFNA2, IFNB1, IFNG, IGFBP3, IGLL5, IL1B, IQGAP3, ITGA9, |
| | JAM2, JAM3, KCNJ8, LAG3, LAMB2, LHFPL6, LTBP4, MEOX1, MEST, MGP, |
| | MMP12, MMP13, MST1, MT2A, MTA2, NAALAD2, NFATC1, NOV, |
| | OLFML2A, PCDH17, PDCD1, PDCD1LG2, PDE5A, PDGFRB, PEG3, PLAU, |
| | PLSCR2, PLXDC2, RAC2, REG4, RGS4, RGS5, RNF144A, RNH1, RRAS, |
| | RUNX1T1, SELP, SERPINE1, SERPINE2, SGIP1, SMARCA1, SPON1, SRSF6, |
| | STAB2, STEAP4, STRN3, TBX2, TEK, TGFB1, TGFB2, TIGIT, TIMP1, TLR9, |
| | TNFRSF18, TNFRSF4, TNFSF18, TRIM7, TTC28, USF1, UTRN, VSIR, ZIC2 |
| Training set 3 | ABCC9, ADAMTS4, AFAP1L2, AGR2, BACE1, BGN, BMP5, C11ORF9, CAPG, |
| (n = 114) | CAVIN2, CCL2, CCL3, CCL4, CD19, CD274, CD3E, CD4, CD79A, CD8B, |
| | COL8A2, CPXM2, CTLA4, CTSB, CXCL10, CXCL11, CXCL12, CXCL9, |
| | DUSP4, EBF1, ECM2, EDNRA, EIF5A, ELN, EPHA3, ETV5, FBLN5, GAD1, |
| | GNAS, GNB4, GUCY1A1, GZMB, HAVCR2, HEY2, HFE, HMOX1, HP, IDO1, |
| | IFNA2, IFNB1, IFNG, IGFBP3, IGLL5, IL1B, IQGAP3, ITGA9, JAM2, JAM3, |
| | KCNJ8, LAG3, LAMB2, LHFPL6, LTBP4, MEOX1, MEST, MGP, MMP12, |
| | MMP13, MST1, MT2A, MTA2, NAALAD2, NFATC1, NOV, PCDH17, PDCD1, |
| | PDCD1LG2, PDE5A, PDGFRB, PEG3, PLAU, PLSCR2, PLXDC2, RAC2, REG4, |
| | RGS4, RGS5, RNF144A, RNH1, RRAS, RUNX1T1, SELP, SERPINE2, SGIP1, |
| | SMARCA1, SPON1, SRSF6, STAB2, STEAP4, STRN3, TBX2, TEK, TGFB1, |
| | TGFB2, TIGIT, TIMP1, TLR9, TNFRSF18, TNFRSF4, TRIM7, TTC28, USF1, |
| | UTRN, VSIR, ZIC2 |
| Training set 4 | ABCC9, ADAMTS4, AFAP1L2, AGR2, BACE1, BGN, BMP5, C11ORF9, CAPG, |
| (n = 106) | CAVIN2, CCL2, CCL3, CCL4, CD19, CD274, CD3E, CD4, CD79A, CD8B, |
| | CPXM2, CTLA4, CTSB, CXCL10, CXCL11, CXCL12, CXCL9, DUSP4, EBF1, |
| | ECM2, EDNRA, EIF5A, ELN, EPHA3, ETV5, FBLN5, GAD1, GNAS, GNB4, |
| | GZMB, HAVCR2, HEY2, HFE, HMOX1, HP, IDO1, IFNA2, IFNB1, IFNG, |
| | IGFBP3, IGLL5, IL1B, IQGAP3, ITGA9, JAM2, JAM3, KCNJ8, LAG3, LAMB2, |
| | LTBP4, MEOX1, MEST, MGP, MMP12, MMP13, MST1, MT2A, MTA2, |
| | NFATC1, NOV, PCDH17, PDCD1, PDE5A, PDGFRB, PEG3, PLAU, PLSCR2, |
| | PLXDC2, RAC2, REG4, RGS4, RGS5, RNH1, RRAS, RUNX1T1, SELP, SGIP1, |
| | SMARCA1, SPON1, SRSF6, STAB2, STEAP4, STRN3, TBX2, TEK, TGFB1, |
| | TGFB2, TIGIT, TIMP1, TLR9, TNFRSF4, TRIM7, TTC28, USF1, UTRN, VSIR, |
| | ZIC2 |
| Training set 5 | ABCC9, AFAP1L2, BACE1, BGN, BMP5, COL4A2, COL8A1, COL8A2, |
| (n = 98) | CPXM2, CXCL12, EBF1, ECM2, EDNRA, ELN, EPHA3, FBLN5, GNAS, GNB4, |
| | GUCY1A3, HEY2, HSPB2, IL1B, ITGA9, ITPR1, JAM2, JAM3, KCNJ8, |
| | LAMB2, LHFP, LTBP4, MEOX1, MGP, MMP12, MMP13, NAALAD2, |
| | NFATC1, NOV, OLFML2A, PCDH17, PDE5A, PDGFRB, PEG3, PLSCR2, |
| | PLXDC2, RGS4, RGS5, RNF144A, RRAS, RUNX1T1, CAV2, SELP, SERPINE2, |
| | SGIP1, SMARCA1, SPON1, STAB2, STEAP4, TBX2, TEK, TGFB2, TMEM204, |
| | TTC28, UTRN, AGR2, C11orf9, DUSP4, EIF5A, ETV5, GAD1, IQGAP3, MST1, |
| | MT2A, MTA2, PLA2G4A, REG4, SRSF6, STRN3, TRIM7, USF1, ZIC2, |
| | C10orf54, CCL3, CCL4, CD19, CD274, CD3E, CD4, CD8B, CTLA4, CXCL10, |
| | IFNA2, IFNB1, IFNG, LAG3, PDCD1, PDCD1LG2, TGFB1, TIGIT |
| Training set 6 | ELN, EPHA3, FBLN5, GNAS, GNB4, GUCY1A3, HEY2, HSPB2, IL1B, ITGA9, |
| (n = 98) | ITPR1, JAM2, JAM3, KCNJ8, LAMB2, LHFP, LTBP4, MEOX1, MGP, MMP12, |
| | MMP13, NAALAD2, NFATC1, NOV, OLFML2A, PCDH17, PDE5A, PDGFRB, |
| | PEG3, PLSCR2, PLXDC2, RGS4, RGS5, RNF144A, RRAS, RUNX1T1, CAV2, |
| | SELP, SERPINE2, SGIP1, SMARCA1, SPON1, STAB2, STEAP4, TBX2, TEK, |
| | TGFB2, TMEM204, TTC28, UTRN, REG4, SRSF6, STRN3, TRIM7, USF1, |
| | ZIC2, C10orf54, CCL3, CCL4, CD19, CD274, CD3E, CD4, CD8B, CTLA4, |
| | CXCL10, IFNA2, IFNB1, IFNG, LAG3, PDCD1, PDCD1LG2, TGFB1, TIGIT, |
| | TNFRSF18, TNFRSF4, TNFSF18, TLR9, HAVCR2, CD79A, CXCL11, CXCL9, |
| | GZMB, IDO1, IGLL5, ADAMTS4, CAPG, CCL2, CTSB, FOLR2, HFE, HMOX1, |
| | HP, IGFBP3, MEST, PLAU, RAC2, RNH1 |
| Training set 7 | ITPR1, JAM2, JAM3, KCNJ8, LAMB2, LHFP, LTBP4, MEOX1, MGP, MMP12, |
| (n = 97) | MMP13, NAALAD2, NFATC1, NOV, OLFML2A, PCDH17, PDE5A, PDGFRB, |
| | PEG3, PLSCR2, PLXDC2, RGS4, RGS5, RNF144A, RRAS, RUNX1T1, CAV2, |
| | SELP, SERPINE2, SGIP1, SMARCA1, SPON1, STAB2, STEAP4, TBX2, TEK, |
| | TGFB2, TMEM204, TTC28, UTRN, AGR2, C11orf9, DUSP4, EIF5A, ETV5, |
| | GAD1, IQGAP3, MST1, MT2A, MTA2, PLA2G4A, REG4, SRSF6, STRN3, |
| | TRIM7, USF1, ZIC2, C10orf54, CCL3, CCL4, CD19, CD274, CD3E, CD4, CD8B, |
| | CTLA4, CXCL10, IFNA2, IFNB1, IFNG, LAG3, PDCD1, PDCD1LG2, TGFB1, |
| | TIGIT, TNFRSF18, TNFRSF4, TNFSF18, TLR9, HAVCR2, CD79A, CXCL11, |
| | CXCL9, GZMB, IDO1, IGLL5, ADAMTS4, CAPG, CCL2, CTSB, FOLR2, HFE, |
| | HMOX1, HP, IGFBP3, MEST, PLAU |
| Training set 8 | CD19, CD274, CD3E, CD4, EDNRA, EPHA3, FBLN5, FOLR2, GAD1, GNB4, |
| (n = 97) | GUCY1A3, GZMB, HAVCR2, HMOX1, HP, HSPB2, IDO1, IFNG, IGFBP3, |
| | IGLL5, IL1B, IQGAP3, ITGA9, ITPR1, JAM2, JAM3, KCNJ8, LAG3, LAMB2, |
| | LHFP, CD79A, COL4A2, COL8A2, CPXM2, CTSB, CXCL10, CXCL11, |
| | CXCL12, CXCL9, DUSP4, EBF1, LTBP4, MEOX1, AFAP1L2, SMARCA1, |
| | SPON1, STEAP4, STRN3, TBX2, TEK, TGFB2, TIGIT, TIMP1, TLR9, |
| | TMEM204, AGR2, BACE1, BGN, BMP5, C10orf54, CAPG, CAV2, CCL2, |
| | CCL3, CCL4, MEST, MGP, MMP13, MST1, MT2A, NFATC1, OLFML2A, |
| | PCDH17, PDCD1LG2, PDE5A, PDGFRB, PEG3, PLA2G4A, PLAU, PLSCR2, |
| | PLXDC2, RAC2, REG4, RGS4, RGS5, RRAS, RUNX1T1, SELP, SERPINE1, |
| | SGIP1, TNFRSF18, TNFRSF4, TNFSF18, TRIM7, TTC28, UTRN, ZIC2 |
| Training set 9 | MMP13, NAALAD2, NFATC1, NOV, OLFML2A, PCDH17, PDE5A, PDGFRB, |
| (n = 87) | PEG3, PLSCR2, PLXDC2, RGS4, RGS5, RNF144A, RRAS, RUNX1T1, CAV2, |
| | SELP, SERPINE2, SGIP1, SMARCA1, SPON1, STAB2, STEAP4, TBX2, TEK, |
| | TGFB2, TMEM204, TTC28, UTRN, REG4, SRSF6, STRN3, TRIM7, USF1, |
| | ZIC2, C10orf54, CCL3, CCL4, CD19, CD274, CD3E, CD4, CD8B, CTLA4, |
| | CXCL10, IFNA2, IFNB1, IFNG, LAG3, PDCD1, PDCD1LG2, TGFB1, TIGIT, |
| | TNFRSF18, TNFRSF4, TNFSF18, TLR9, HAVCR2, CD79A, CXCL11, CXCL9, |
| | GZMB, IDO1, IGLL5, ADAMTS4, CAPG, CCL2, CTSB, FOLR2, HFE, HMOX1, |
| | HP, IGFBP3, MEST, PLAU, RAC2, RNH1, SERPINE1, TIMP1, AGR2, C11orf9, |
| | DUSP4, EIF5A, ETV5, GAD1, IQGAP3 |
| Training set 10 | CPXM2, CTSB, CXCL10, CXCL11, CXCL12, CXCL9, DUSP4, EBF1, EDNRA, |
| (n = 86) | EPHA3, FBLN5, FOLR2, GAD1, GNB4, GUCY1A3, GZMB, HAVCR2, |
| | HMOX1, HP, HSPB2, IDO1, IFNG, IGFBP3, LTBP4, MEOX1, MEST, MGP, |
| | MMP13, AFAP1L2, OLFML2A, PCDH17, PDCD1LG2, PDE5A, SMARCA1, |
| | SPON1, STEAP4, STRN3, TBX2, TEK, TGFB2, TIGIT, AGR2, BACE1, BGN, |
| | BMP5, C10orf54, CAPG, CAV2, CCL2, CCL3, CCL4, CD19, PDGFRB, PEG3, |
| | PLA2G4A, PLAU, PLSCR2, PLXDC2, RAC2, REG4, RGS4, RGS5, RRAS, |
| | RUNX1T1, SELP, SERPINE1, SGIP1, CD274, CD3E, CD4, CD79A, COL4A2, |
| | COL8A2, MST1, MT2A, NFATC1, TIMP1, TLR9, TMEM204, TNFRSF18, |
| | TNFRSF4, TNFSF18, TRIM7, TTC28, UTRN, ZIC2 |
| Training set 11 | EPHA3, FBLN5, FOLR2, GAD1, GNB4, GUCY1A3, GZMB, HAVCR2, |
| (n = 79) | HMOX1, HP, HSPB2, IDO1, IFNG, IGFBP3, IGLL5, IL1B, IQGAP3, ITGA9, |
| | ITPR1, CD3E, CD4, CD79A, COL4A2, COL8A2, CPXM2, CTSB, CXCL10, |
| | CXCL11, CXCL12, CXCL9, DUSP4, EBF1, EDNRA, NFATC1, OLFML2A, |
| | PCDH17, PDCD1LG2, PDE5A, PDGFRB, PEG3, PLA2G4A, PLAU, PLSCR2, |
| | JAM2, JAM3, KCNJ8, LAG3, LAMB2, LHFP, LTBP4, MEOX1, MEST, MGP, |
| | MMP13, MST1, MT2A, AFAP1L2, AGR2, BACE1, BGN, BMP5, C10orf54, |
| | CAPG, CAV2, CCL2, CCL3, CCL4, CD19, CD274, PLXDC2, RAC2, REG4, |
| | RGS4, RGS5, RRAS, RUNX1T1, SELP, SERPINE1, SGIP1 |
| Training set 12 | LAG3, LAMB2, LHFP, PCDH17, PDCD1LG2, PDE5A, PDGFRB, PEG3, |
| (n = 68) | PLA2G4A, PLAU, PLSCR2, PLXDC2, RAC2, REG4, RGS4, CCL4, CXCL11, |
| | CXCL12, CXCL9, DUSP4, EBF1, EDNRA, EPHA3, FBLN5, FOLR2, GAD1, |
| | IGLL5, IL1B, IQGAP3, ITGA9, ITPR1, JAM2, JAM3, RGS5, RRAS, RUNX1T1, |
| | SELP, SERPINE1, SGIP1, SMARCA1, SPON1, STEAP4, STRN3, TBX2, TEK, |
| | TGFB2, TIGIT, TIMP1, TLR9, AFAP1L2, AGR2, BACE1, BGN, BMP5, |
| | C10orf54, CAPG, CAV2, CCL2, CCL3, KCNJ8, TMEM204, TNFRSF18, |
| | TNFRSF4, TNFSF18, TRIM7, TTC28, UTRN, ZIC2 |
| Training set 13 | FBLN5, FOLR2, GAD1, GNB4, GUCY1A3, GZMB, HAVCR2, HMOX1, HP, |
| (n = 68) | HSPB2, IDO1, IFNG, IGFBP3, LTBP4, MEOX1, MEST, MGP, CCL3, CCL4, |
| | CD19, CD274, CD3E, CD4, CD79A, COL4A2, COL8A2, CPXM2, CTSB, |
| | CXCL10, CXCL11, CXCL12, CXCL9, DUSP4, EBF1, EDNRA, EPHA3, |
| | OLFML2A, PCDH17, PDCD1LG2, PDE5A, PDGFRB, PEG3, PLA2G4A, |
| | MMP13, MST1, MT2A, NFATC1, AFAP1L2, AGR2, BACE1, BGN, BMP5, |
| | C10orf54, CAPG, CAV2, CCL2, PLAU, PLSCR2, PLXDC2, RAC2, REG4, |
| | RGS4, RGS5, RRAS, RUNX1T1, SELP, SERPINE1, SGIP1 |
| Training set 14 | GAD1, GNB4, GUCY1A3, GZMB, HAVCR2, HMOX1, HP, HSPB2, IDO1, |
| (n = 61) | IFNG, IGFBP3, IGLL5, IL1B, IQGAP3, ITGA9, ITPR1, CD19, CD274, CD3E, |
| | CD4, CD79A, COL4A2, COL8A2, CPXM2, CTSB, CXCL10, CXCL11, CXCL12, |
| | CXCL9, DUSP4, EBF1, EDNRA, EPHA3, FBLN5, JAM2, JAM3, KCNJ8, LAG3, |
| | AFAP1L2, AGR2, BACE1, BGN, BMP5, C10orf54, CAPG, CAV2, CCL2, CCL3, |
| | CCL4, FOLR2, LAMB2, LHFP, LTBP4, MEOX1, MEST, MGP, MMP13, MST1, |
| | MT2A, NFATC1, OLFML2A |
| Training set 15 | COL8A2, CPXM2, CTSB, GZMB, HAVCR2, HMOX1, HP, HSPB2, IDO1, IFNG, |
| (n = 51) | IGFBP3, IGLL5, IL1B, IQGAP3, ITGA9, ITPR1, JAM2, AFAP1L2, AGR2, |
| | CXCL10, CXCL11, CXCL12, CXCL9, DUSP4, EBF1, EDNRA, EPHA3, FBLN5, |
| | FOLR2, GAD1, GNB4, GUCY1A3, BACE1, BGN, BMP5, C10orf54, CAPG, |
| | CAV2, CCL2, CCL3, CCL4, CD19, CD274, CD3E, CD4, CD79A, COL4A2, |
| | JAM3, KCNJ8, LAG3, LAMB2, LHFP |
| Training set 16 | CTSB, CXCL10, CXCL11, HMOX1, HP, HSPB2, IDO1, AFAP1L2, AGR2, |
| (n = 41) | BACE1, BGN, BMP5, C10orf54, CAPG, CAV2, CCL2, CCL3, CCL4, CD19, |
| | CXCL12, CXCL9, DUSP4, EBF1, EDNRA, EPHA3, FBLN5, FOLR2, GAD1, |
| | GNB4, GUCY1A3, GZMB, HAVCR2, CD274, CD3E, CD4, CD79A, COL4A2, |
| | COL8A2, CPXM2, IFNG, IGFBP3 |
| Training set 17 | CD79A, COL4A2, CD19, CD274, CAV2, CCL2, CCL3, CCL4, CXCL11, |
| (n = 31) | CXCL12, CXCL9, DUSP4, EBF1, EDNRA, EPHA3, FBLN5, FOLR2, CD3E, |
| | CD4, CXCL10, COL8A2, CPXM2, CTSB, AFAP1L2, AGR2, BACE1, BGN, |
| | BMP5, C10orf54, CAPG, GAD1 |

The practical behavior of a machine learning model of the present disclosure is to represent high dimensional data in a compressed form. The compressed data can be represented visually in what is known as the latent space. A common example of this is a two dimensional graph (X & Y axes), where each patient is plotted as the value of some vector X and vector Y. Thus, the latent space is a projection of the signatures generated by the method of the present disclosure, e.g., whether is a projection of the Z-scores or the values of the hidden neurons. In some aspects, the latent space can be plotted in three-dimensions.

Disease score values of each patient can be plotted in the latent space (i.e., the probability result of the ANN model). Over time, patient data can be accumulated, or the results of a retrospective analysis of patient data with disease scores can be used as a reference plot, on which the subject patient's ANN probability result is plotted.

In some aspects, the latent space is a plot of the hidden neurons of the ANN model, and could include all 2-way combinations of those neurons. In some aspects, the ANN model predicts four phenotype classes based on the data compressed in the two hidden neurons, and plotting those neurons in the latent space also serves as a projection of the four output phenotype classes. In some aspects, the phenotype class assignments of each patient are visualized in the Neuron 1 versus Neuron 2 latent space.

The latent space projection may be enhanced by displaying the probability contours of the output (phenotype) assignments. In this way, the projection can show not only where subjects fall in the latent space, but also the confidence of each phenotype classification. In some aspects, clinical reporting can use the phenotype class as the biomarker logic that is, IA=positive, or IA+IS=positive-then report out to the clinician the probability of the phenotype assignment, which is already an output of the model. The latent space plot can also be used to visualize the distance of that patient from the decision boundary to assist clinical decision makers in evaluating edge cases and exceptions.

In some aspects, the boundaries between the TME phenotype classes are not on the cartesian axes (x=0, y=0), but elsewhere in the plot.

In some aspects, a second model can learn the biomarker boundary from the ANN model latent space. In some aspects, that second model can be a logistic regression model. In some aspects it could be any other kind of regression or machine learning algorithm. In some aspects, a logistic regression function may be applied to the latent space. In some aspects, combining phenotypes to define the biomarker positive class, i.e. IA+IS, the confidence of the individual phenotype assignments does not equal the confidence of the combined class assignment. A logistic regression function is used to learn what it means to be biomarker positive and directly reports statistics on being biomarker positive. A logistic regression function can be used to fine-tune the biomarker positive/negative decision boundary based on real patient outcome data. In some aspects, the accuracy of the ANN model can be improved by slicing the latent space according to a secondary model.

In some aspects, the probability function can be plotted in two dimensions, one axis representing the probability that the signal is dominated by the genes of Signature 1, and the other axis representing the probability that the that the signal is dominated by the genes of Signature 2. In some aspects, genes that play a role in angiogenesis and in immune functions contribute to each of the probability functions. Each quadrant of the latent space plot represents a stromal phenotype. In a further aspect, the threshold is applied by using a logistic regression. In some aspects, the logistic regression can be linear or polynomial. After a threshold is set, individual patient results can be analyzed according to the methods described herein.

### I.E. TME-Specific Methods of Treatment

The present disclosure provides methods for classifying/stratifying patients and/or cancer samples from those patients according to a tumor microenvironment (TME) determination resulting from applying a classifier derived from a combined biomarker (e.g., a set of gene expression data corresponding to a gene panel). In some aspects, the classifier is a non-population based classifier disclosed herein, e.g., an ANN model. In other aspects, the classifier is population-based classifier disclosed herein that, e.g., integrates several signature scores (e.g., Signature 1 and Signature 2 in an exemplary aspect). Based on the identification of the presence of a particular TME or a combination thereof (i.e., whether the patient is biomarker-positive and/or biomarker-negative for one or more stromal phenotypes disclosed herein), a preferred therapy (e.g., a TME-class therapy disclosed herein or a combination thereof) can be selected to treat the patient's cancer.

In one aspect, the present disclosure provides a method for treating a human subject afflicted with a cancer comprising administering “IA-class TME therapy” to the subject, wherein, prior to the administration, the subject is identified via a population-based classifier as exhibiting a combined biomarker comprising (a) a negative Signature 1 score; and (b) a positive Signature 2 score, wherein (i) the Signature 1 score is determined by measuring the expression levels of a gene panel selected from TABLE 3 in a first sample obtained from the subject; and, (ii) the Signature 2 score is determined by measuring the expression levels of a gene panel selected from TABLE 4 in a second sample obtained from the subject.

In one aspect, the present disclosure provides a method for treating a human subject afflicted with a cancer comprising administering “IA-class TME therapy” to the subject, wherein, prior to the administration, the subject is identified via a non-population-based classifier, e.g., an ANN classifier, disclosed herein as exhibiting an IA class TME, wherein the presence of an IA class TME is determined by applying the ANN classifier model to a set of data comprising expression levels of a gene panel selected from TABLE 1 and TABLE 2 (or a gene panels (Genesets) disclosed in FIGS. 28A-G) in a sample obtained from the subject.

The present disclosure also provides a method for treating a human subject afflicted with a cancer comprising
(A) identifying via a population-based classifier, prior to the administration, a subject exhibiting a combined biomarker comprising
(a) a negative Signature 1 score; and
(b) a positive Signature 2 score, wherein
(i) the Signature 1 score is determined by measuring the expression levels of a gene panel selected from TABLE 3 in a first sample obtained from the subject; and,
(ii) the Signature 2 score is determined by measuring the expression levels of a gene panel selected from TABLE 4 in a second sample obtained from the subject; and,
(B) administering to the subject an IA-class TME therapy.

Also provided is a method for identifying a human subject afflicted with a cancer suitable for treatment with an IA-class TME therapy, the method comprising
(i) determining a Signature 1 score by measuring the expression levels of a gene panel selected from TABLE 3 in a first sample obtained from the subject; and,
(ii) determining a Signature 2 score by measuring the expression levels of a gene panel selected from TABLE 4 in a second sample obtained from the subject,
wherein the presence of a combined biomarker comprising
(a) a negative Signature 1 score; and
(b) a positive Signature 2 score, identified via the population-based classifier prior to the administration,
indicates that a IA-class TME therapy can be administered to treat the cancer.

The present disclosure also provides a method for treating a human subject afflicted with a cancer comprising
(A) identifying via a non-population-based classifier (e.g., an ANN), prior to the administration, a subject exhibiting an IA-class TME as determined by measuring the expression levels of a gene panel selected from TABLE 1 and TABLE 2 (or any of the gene panels (Genesets) disclosed in FIGS. 28A-G) in a sample obtained from the subject; and,
(B) administering to the subject an IA-class TME therapy.

In some aspects, the IA-class TME therapy can be administered in combination with additional TME-class therapies disclosed herein if the subject is biomarker-positive for additional stromal phenotypes.

Also provided is a method for identifying a human subject afflicted with a cancer suitable for treatment with an IA-class TME therapy, the method comprising determining the presence of an IA class in the subject via a non-population classifier (e.g., an ANN) disclosed herein as determined by measuring the expression levels of a gene panel selected from TABLE 1 and TABLE 2 (or any of the gene panels (Genesets) disclosed in FIGS. 28A-G) in a sample obtained from the subject; wherein the presence of a combined IA class TME indicates that a IA-class TME therapy can be administered to treat the cancer.

In some aspects, the IA-class TME therapy comprises a checkpoint modulator therapy.

In some aspects, the checkpoint modulator therapy comprises administering an activator of a stimulatory immune checkpoint molecule. In some aspects, the activator of a stimulatory immune checkpoint molecule is, e.g., an antibody molecule against GITR (glucocorticoid-induced tumor necrosis factor receptor, TNFRSF18), OX-40 (TNFRSF4, ACT35, CD134, IMD16, TXGP1L, tumor necrosis factor receptor superfamily member 4, TNF receptor superfamily member 4), ICOS (Inducible T Cell Costimulator), 4-1BB (TNFRSF9, CD137, CDw137, ILA, tumor necrosis factor receptor superfamily member 9, TNF receptor superfamily member 9), or a combination thereof. In some aspects, the checkpoint modulator therapy comprises the administration of a RORγ (RORC, NR1F3, RORG, RZR-GAMMA, RZRG, TOR, RAR-related orphan receptor gamma, IMD42, RAR related orphan receptor C) agonist.

In some aspects, the checkpoint modulator therapy comprises the administration of an inhibitor of an inhibitory immune checkpoint molecule. In some aspects, the inhibitor of an inhibitory immune checkpoint molecule is, e.g., (1) an antibody against PD-1 (PDCD1, CD279, SLEB2, hPD-1, hPD-1, hSLE1, Programmed cell death 1), e.g., sintilimab, tislelizumab, pembrolizumab, or an antigen binding portion thereof, an antibody against PD-L1 (CD274, B7-H, B7H1, PDCD1L1, PDCD1LG1, PDL1, CD274 molecule, Programmed cell death ligand 1, hPD-L1), an antibody against PD-L2 (PDCD1LG2, B7DC, Btdc, CD273, PDCD1L2, PDL2, bA574F11.2, programmed cell death 1 ligand 2), an antibody against CTLA-4 (CTLA4, ALPS5, CD, CD152, CELIAC3, GRD4, GSE, IDDM12, cytotoxic T-lymphocyte associated protein 4), a bispecific antibody comprising at least a binding specificity for PD-L1, PD-L2, or CTLA-4, alone or a combination thereof, or (2) any of the antibodies in (1) in combination with an inhibitor of TIM-3 (T-cell immunoglobulin and mucin-domain containing-3), an inhibitor of LAG-3 (Lymphocyte-activation gene 3), an inhibitor of BTLA (B- and T-lymphocyte attenuator), an inhibitor of TIGIT (T cell immunoreceptor with Ig and ITIM domains), an inhibitor of VISTA (V-domain Ig suppressor of T cell activation), an inhibitor of TGF-β (transforming growth factor beta) or its receptors, a CD86 (Cluster of Differentiation 86) agonist, an inhibitor of LAIR1 (Leukocyte-associated immunoglobulin-like receptor 1), an inhibitor of CD160 (Cluster of Differentiation 160), an inhibitor of 2B4 (Natural Killer Cell Receptor 2B4; Cluster of Differentiation 244), an inhibitor of GITR, an inhibitor of OX40, an inhibitor of 4-1BB (CD137), an inhibitor of CD2 (Cluster of Differentiation 2), an inhibitor of CD27 (Cluster of Differentiation 27), an inhibitor of CDS (CDP-Diacylglycerol Synthase 1), an inhibitor of ICAM-1 (Intercellular Adhesion Molecule 1), an inhibitor of LFA-1 (Lymphocyte function-associated antigen 1; CD11a/CD18), an inhibitor of ICOS (Inducible T-cell COStimulator; CD278), an inhibitor of CD30 (Cluster of Differentiation 30), an inhibitor of CD40 (Cluster of Differentiation 40), an inhibitor of BAFFR (B-cell activating factor receptor), an inhibitor of HVEM (Herpesvirus entry mediator), an inhibitor of CD7 (Cluster of Differentiation 7), an inhibitor of LIGHT (tumor necrosis factor superfamily member 14; TNFSF14), an inhibitor of NKG2C (killer cell lectin like receptor C2; KLRC2, CD159c), an inhibitor of SLAMF7 (SLAM family member 7), an inhibitor of NKp80 (Activating Coreceptor NKp80; Lectin-Like Receptor F1; KLRF1; Killer Cell Lectin Like Receptor F1), or any combination thereof.

In some aspects, the checkpoint modulator therapy comprises the administration of a modulator of TIM-3, a modulator of LAG-3, a modulator of BTLA, a modulator of TIGIT, a modulator of VISTA, a modulator of TGF-β or its receptor, a modulator of CD86, a modulator of LAIR1, a modulator of CD160, a modulator of 2B4, a modulator of GITR, a modulator of OX40, a modulator of 4-1BB (CD137), a modulator of CD2, a modulator of CD27, a modulator of CDS, a modulator of ICAM-1, a modulator of LFA-1 (CD11a/CD18), a modulator of ICOS (CD278), a modulator of CD30, a modulator of CD40, a modulator of BAFFR, a modulator of HVEM, a modulator of CD7, a modulator of LIGHT, a modulator of NKG2C, a modulator of SLAMF7, a modulator of NKp80, or a combination thereof.

As used herein, the term “modulator,” refers to a molecule that interacts with a target either directly or indirectly, and imparts an effect on a biological or chemical process or mechanism. For example, a modulator can increase, facilitate, upregulate, activate, inhibit, decrease, block, prevent, delay, desensitize, deactivate, down regulate, or the like, a biological or chemical process or mechanism. Accordingly, a modulator can be an “agonist” or an “antagonist” of the target. The term “agonist” refers to a compound that increases at least some of the effect of the endogenous ligand of a protein, receptor, enzyme or the like. The term “antagonist” refers to a compound that inhibits at least some of the effect of the endogenous ligand of a protein, receptor, enzyme or the like.

Thus, in some aspects, the checkpoint modulator therapy comprises the administration of an agonist or an antagonist of TIM-3, an agonist or an antagonist of LAG-3, an agonist or an antagonist of BTLA, an agonist or an antagonist of TIGIT, an agonist or an antagonist of VISTA, an agonist or an antagonist of TGF-β or its receptor, an agonist or an antagonist of CD86, an agonist or an antagonist of LAIR1, an agonist or an antagonist of CD160, an agonist or an antagonist of 2B4, an agonist or an antagonist of GITR, an agonist or an antagonist of OX40, an agonist or an antagonist of 4-1BB (CD137), an agonist or an antagonist of CD2, an agonist or an antagonist of CD27, an agonist or an antagonist of CDS, an agonist or an antagonist of ICAM-1, an agonist or an antagonist of LFA-1 (CD11a/CD18), an agonist or an antagonist of ICOS (CD278), an agonist or an antagonist of CD30, an agonist or an antagonist of CD40, an agonist or an antagonist of BAFFR, an agonist or an antagonist of HVEM, an agonist or an antagonist of CD7, an agonist or an antagonist of LIGHT, an agonist or an antagonist of NKG2C, an agonist or an antagonist of SLAMF7, an agonist or an antagonist of NKp80, or any combination thereof.

In some aspects, the anti-PD-1 antibody comprises, e.g., nivolumab, pembrolizumab, cemiplimab, sintilimab, tislelizumab, or an antigen-binding portion thereof. In some aspects, the anti-PD-1 antibody cross-competes, e.g., with nivolumab, pembrolizumab, cemiplimab, sintilimab, or tislelizumab for binding to human PD-1. In some aspects, the anti-PD-1 antibody binds, e.g., to the same epitope as nivolumab, pembrolizumab, cemiplimab, sintilimab, or tislelizumab.

In some aspects, the anti-PD-L1 antibody comprises, e.g., avelumab, atezolizumab, durvalumab, or an antigen-binding portion thereof. In some aspects, the anti-PD-1 antibody cross-competes, e.g., with avelumab, atezolizumab, or durvalumab for binding to human PD-1. In some aspects, the anti-PD-1 antibody binds, e.g., to the same epitope as avelumab, atezolizumab, or durvalumab.

In some aspects, the checkpoint modulator therapy comprises the administration of (i) an anti-PD-1 antibody, e.g., an antibody selected from the group consisting of nivolumab, pembrolizumab, sintilimab, tislelizumab, and cemiplimab; (ii) an anti-PD-L1 antibody, e.g., an antibody selected from the group consisting of avelumab, atezolizumab, and durvalumab; or (iii) a combination thereof.

The present disclosure provides a method for treating a human subject afflicted with a cancer comprising administering an “IS-class TME therapy” to the subject, wherein, prior to the administration, the subject is identified via a population-based classifier as exhibiting a combined biomarker comprising (a) a positive Signature 1 score; and (b) a positive Signature 2 score, wherein (i) the Signature 1 score is determined by measuring the expression levels of a gene panel selected from TABLE 3 in a first sample obtained from the subject; and, (ii) the Signature 2 score is determined by measuring the expression levels of a gene panel selected from TABLE 4 in a second sample obtained from the subject.

In one aspect, the present disclosure provides a method for treating a human subject afflicted with a cancer comprising administering “IS-class TME therapy” to the subject, wherein, prior to the administration, the subject is identified via a non-population-based classifier, e.g., an ANN classifier, disclosed herein as exhibiting an IS class TME, wherein the presence of a IS class TME is determined by applying the ANN classifier model to a set of data comprising expression levels of a gene panel selected from TABLE 1 and TABLE 2 (or from any of the gene panels (Genesets) disclosed in FIGS. 28A-G), in a sample obtained from the subject.

The present disclosure also provides a method for treating a human subject afflicted with a cancer comprising
(A) identifying via a population-based classifier, prior to the administration, a subject exhibiting a combined biomarker comprising
(a) a positive Signature 1 score; and
(b) a positive Signature 2 score,
wherein
(i) the Signature 1 score is determined by measuring the expression levels of a gene panel selected from TABLE 3 in a first sample obtained from the subject; and,
(ii) the Signature 2 score is determined by measuring the expression levels of a gene panel selected from TABLE 4 in a second sample obtained from the subject; and,
(B) administering to the subject an IS-class TME therapy.

Also provided is a method for identifying a human subject afflicted with a cancer suitable for treatment with an IS-class TME therapy, the method comprising
(i) determining a Signature 1 score by measuring the expression levels of a gene panel selected from TABLE 3 in a first sample obtained from the subject; and,
(ii) determining a Signature 2 score by measuring the expression levels of a gene panel selected from TABLE 4 in a second sample obtained from the subject,
wherein the presence of a combined biomarker comprising
(a) a positive Signature 1 score; and
(b) a positive Signature 2 score, identified via a population-based classifier prior to the administration,
indicates that a IS-class TME therapy can be administered to treat the cancer.

The present disclosure also provides a method for treating a human subject afflicted with a cancer comprising
(A) identifying via a non-population-based classifier (e.g., an ANN), prior to the administration, a subject exhibiting an IS-class TME as determined by measuring the expression levels of a gene panel selected from TABLE 1 and TABLE 2 (or any of the gene panels (Genesets) disclosed in FIGS. 28A-G) in a sample obtained from the subject; and,
(B) administering to the subject an IS-class TME therapy.

In some aspects, the IS-class TME therapy can be administered in combination with additional TME-class therapies disclosed herein if the subject is biomarker-positive for additional stromal phenotypes.

Also provided is a method for identifying a human subject afflicted with a cancer suitable for treatment with an IS-class TME therapy, the method comprising determining the presence of an IS class in the subject via a non-population classifier (e.g., an ANN) disclosed herein as determined by measuring the expression levels of a gene panel selected from TABLE 1 and TABLE 2 (or any of the gene panels (Genesets) disclosed in FIGS. 28A-G) in a sample obtained from the subject; wherein the presence of a combined IS-class TME indicates that a IS-class TME therapy can be administered to treat the cancer.

In some aspects, the IS-class TME therapy comprises, e.g., the administration of (1) a checkpoint modulator therapy and an anti-immunosuppression therapy (e.g., a combination therapy comprising the administration of pembrolizumab and bavituximab) and/or (2) an antiangiogenic therapy. In some aspects, the checkpoint modulator therapy comprises, e.g., the administration of an inhibitor of an inhibitory immune checkpoint molecule. In some aspects, the inhibitor of an inhibitory immune checkpoint molecule is, e.g., an antibody against PD-1 (e.g., sintilimab, tislelizumab, pembrolizumab, or an antigen binding portion thereof), PD-L1, PD-L2, CTLA-4, or a combination thereof.

In some aspect, the anti-PD-1 antibody comprises, e.g., nivolumab, pembrolizumab, cemiplimab, spartalizumab (PDR001), sintilimab, tislelizumab, or geptanolimab (CBT-501), or an antigen-binding portion thereof. In some aspects, the anti-PD-1 antibody cross-competes, e.g., with nivolumab, pembrolizumab, cemiplimab, PDR001, sintilimab, tislelizumab, or CBT-501, for binding to human PD-1. In some aspects, the anti-PD-1 antibody binds, e.g., to the same epitope as nivolumab, pembrolizumab, cemiplimab, sintilimab, tislelizumab, PDR001, or CBT-501.

In some aspects, the anti-PD-L1 antibody comprises, e.g., avelumab, atezolizumab, durvalumab, or an antigen-binding portion thereof. In some aspects, the anti-PD-L1 antibody cross-competes, e.g., with avelumab, atezolizumab, or durvalumab for binding to human PD-L1. In some aspects, the anti-PD-L1 antibody binds, e.g., to the same epitope as avelumab, atezolizumab, or durvalumab.

In some aspects, the anti-CTLA-4 antibody comprises ipilimumab, or an antigen-binding portion thereof. In some aspects, the anti-CTLA-4 antibody cross-competes with ipilimumab for binding to human CTLA-4. In some aspects, the anti-CTLA-4 antibody binds to the same epitope as ipilimumab.

In some aspects, the check point modulator therapy comprises, e.g., the administration of (i) an anti-PD-1 antibody selected, e.g., from the group consisting of nivolumab, pembrolizumab, sintilimab, tislelizumab, and cemiplimab; (ii) an anti-PD-L1 antibody selected, e.g., from the group consisting of avelumab, atezolizumab, and durvalumab; (iii) an anti-CTLA-4 antibody, e.g., ipilimumab, or (iii) a combination thereof.

In some aspects, the antiangiogenic therapy comprises, e.g., the administration of an anti-VEGF (Vascular endothelial growth factor) antibody selected from the group consisting of varisacumab, bevacizumab, navicixizumab (an anti-DLL4/anti-VEGF bispecific antibody), and a combination thereof. In some aspects, the antiangiogenic therapy comprises, e.g., the administration of an anti-VEGFR antibody. In some aspects, the anti-VEGFR antibody is an anti-VEGFR2 Vascular endothelial growth factor receptor 2) antibody. In some aspects, the anti-VEGFR2 antibody comprises ramucirumab. In some aspects, the antiangiogenic therapy comprises, e.g., navicixizumab, ABL101 (NOV1501), or dilpacimab (ABT165).

In some aspects, the anti-immunosuppression therapy comprises, e.g., the administration of an anti-PS (phosphatidyl serine) antibody, anti-PS targeting antibody, antibody that binds β2-glycoprotein 1, inhibitor of PI3Kγ (phosphatidylinositol-4,5-bisphosphate 3-kinase catalytic subunit gamma isoform), adenosine pathway inhibitor, inhibitor of IDO, inhibitor of TIM, inhibitor of LAG3, inhibitor of TGF-β, CD47 inhibitor, or a combination thereof.

In some aspects, the anti-PS targeting antibody is, e.g., bavituximab or an antibody that binds A2-glycoprotein 1. In some aspects, the PI3Kγ inhibitor is, e.g., LY3023414 (samotolisib) or IPI-549 (eganelisib). In some aspects, the adenosine pathway inhibitor is, e.g., AB-928. In some aspects, the TGFβ inhibitor is, e.g., LY2157299 (galunisertib) or the TGFβR1 inhibitor LY3200882. In some aspects, the CD47 inhibitor is, e.g., magrolimab (5F9). In some aspects, the CD47 inhibitor targets SIRPα.

In some aspects, the anti-immunosuppression therapy comprises the administration of an inhibitor of TIM-3, an inhibitor of LAG-3, an inhibitor of BTLA, an inhibitor of TIGIT, an inhibitor of VISTA, an inhibitor of TGF-β or its receptor, an inhibitor of CD86, an inhibitor of LAIR1, an inhibitor of CD160, an inhibitor of 2B4, an inhibitor of GITR, an inhibitor of OX40, an inhibitor of 4-1BB (CD137), an inhibitor of CD2, an inhibitor of CD27, an inhibitor of CDS, an inhibitor of ICAM-1, an inhibitor of LFA-1 (CD11a/CD18), an inhibitor of ICOS (CD278), an inhibitor of CD30, an inhibitor of CD40, an inhibitor of BAFFR, an inhibitor of HVEM, an inhibitor of CD7, an inhibitor of LIGHT, an inhibitor of NKG2C, an inhibitor of SLAMF7, an inhibitor of NKp80, or a combination thereof.

In some aspects, the anti-immunosuppression therapy comprises the administration of a modulator of TIM-3, a modulator of LAG-3, a modulator of BTLA, a modulator of TIGIT, a modulator of VISTA, a modulator of TGF-β or its receptor, a modulator of CD86, a modulator of LAIR1, a modulator of CD160, a modulator of 2B4, a modulator of GITR, a modulator of OX40, a modulator of 4-1BB (CD137), a modulator of CD2, a modulator of CD27, a modulator of CDS, a modulator of ICAM-1, a modulator of LFA-1 (CD11a/CD18), a modulator of ICOS (CD278), a modulator of CD30, a modulator of CD40, a modulator of BAFFR, a modulator of HVEM, a modulator of CD7, a modulator of LIGHT, a modulator of NKG2C, a modulator of SLAMF7, a modulator of NKp80, or a combination thereof.

Thus, in some aspects, the anti-immunosuppression therapy comprises the administration of an agonist or an antagonist of TIM-3, an agonist or an antagonist of LAG-3, an agonist or an antagonist of BTLA, an agonist or an antagonist of TIGIT, an agonist or an antagonist of VISTA, an agonist or an antagonist of TGF-β or its receptor, an agonist or an antagonist of CD86, an agonist or an antagonist of LAIR1, an agonist or an antagonist of CD160, an agonist or an antagonist of 2B4, an agonist or an antagonist of GITR, an agonist or an antagonist of OX40, an agonist or an antagonist of 4-1BB (CD137), an agonist or an antagonist of CD2, an agonist or an antagonist of CD27, an agonist or an antagonist of CDS, an agonist or an antagonist of ICAM-1, an agonist or an antagonist of LFA-1 (CD11a/CD18), an agonist or an antagonist of ICOS (CD278), an agonist or an antagonist of CD30, an agonist or an antagonist of CD40, an agonist or an antagonist of BAFFR, an agonist or an antagonist of HVEM, an agonist or an antagonist of CD7, an agonist or an antagonist of LIGHT, an agonist or an antagonist of NKG2C, an agonist or an antagonist of SLAMF7, an agonist or an antagonist of NKp80, or any combination thereof.

The present disclosure also provides a method for treating a human subject afflicted with a cancer comprising administering an “ID-class TME therapy” to the subject, wherein, prior to the administration, the subject is identified via a population-based classifier as exhibiting a combined biomarker comprising (a) a negative Signature 1 score; and (b) a negative Signature 2 score, wherein (i) the Signature 1 score is determined by measuring the expression levels of a gene panel selected from TABLE 3 in a first sample obtained from the subject; and, (ii) the Signature 2 score is determined by measuring the expression levels of a gene panel selected from TABLE 4 in a second sample obtained from the subject.

In one aspect, the present disclosure provides a method for treating a human subject afflicted with a cancer comprising administering “ID-class TME therapy” to the subject, wherein, prior to the administration, the subject is identified via a non-population-based classifier, e.g., an ANN classifier, disclosed herein as exhibiting an ID class TME, wherein the presence of a ID class TME is determined by applying the ANN classifier model to a set of data comprising expression levels of a gene panel selected from TABLE 1 and TABLE 2 (or any of the gene panels (Genesets) disclosed in FIGS. 28A-G) in a sample obtained from the subject.

Also provided is a method for treating a human subject afflicted with a cancer comprising
(A) identifying via a population-based classifier, prior to the administration, a subject exhibiting a combined biomarker comprising
(a) a negative Signature 1 score; and
(b) a negative Signature 2 score, wherein
(i) the Signature 1 score is determined by measuring the expression levels of a gene panel selected from TABLE 3 in a first sample obtained from the subject; and,
(ii) the Signature 2 score is determined by measuring the expression levels of a gene panel selected from TABLE 4 in a second sample obtained from the subject; and,
(B) administering to the subject an ID-class TME therapy.

Also provided is method for identifying a human subject afflicted with a cancer suitable for treatment with an ID-class TME therapy, the method comprising
(i) determining a Signature 1 score by measuring the expression levels of a gene panel selected from TABLE 3 in a first sample obtained from the subject; and,
(ii) determining a Signature 2 score by measuring the expression levels of a gene panel selected from TABLE 4 in a second sample obtained from the subject, wherein the presence of a combined biomarker comprising
(a) a negative Signature 1 score; and
(b) a negative Signature 2 score, identified via a population-based classifier prior to the administration, indicates that an ID-class TME therapy can be administered to treat the cancer.

The present disclosure also provides a method for treating a human subject afflicted with a cancer comprising
(A) identifying via a non-population-based classifier (e.g., an ANN), prior to the administration, a subject exhibiting an ID-class TME as determined by measuring the expression levels of a gene panel selected from TABLE 1 and TABLE 2 (or any of the gene panels (Genesets) disclosed in FIGS. 28A-G) in a sample obtained from the subject; and,
(B) administering to the subject an ID-class TME therapy.

In some aspects, the ID-class TME therapy can be administered in combination with additional TME-class therapies disclosed herein if the subject is biomarker-positive for additional stromal phenotypes.

Also provided is a method for identifying a human subject afflicted with a cancer suitable for treatment with an ID-class TME therapy, the method comprising determining the presence of an ID-class in the subject via a non-population classifier (e.g., an ANN) disclosed herein as determined by measuring the expression levels of a gene panel selected from TABLE 1 and TABLE 2 (or any of the gene panels (Genesets) disclosed in FIGS. 28A-G) in a sample obtained from the subject; wherein the presence of a combined ID-class TME indicates that an ID-class TME therapy can be administered to treat the cancer.

In some aspects, the ID-class TME therapy comprises the administration of a checkpoint modulator therapy concurrently or after the administration of a therapy that initiates an immune response.

In some aspects, the therapy that initiates the immune response is a vaccine (e.g., a cancer vaccine), a CAR-T, or a neo-epitope vaccine.

In some aspects, the checkpoint modulator therapy is administered concurrently or after the administration of a therapy that initiates an immune response and comprises, e.g., the administration of an inhibitor of an inhibitory immune checkpoint molecule. In some aspects, the inhibitor of an inhibitory immune checkpoint molecule is, e.g., an antibody against PD-1 (e.g., sintilimab, tislelizumab, pembrolizumab, or an antigen binding portion thereof), PD-L1, PD-L2, CTLA-4, or a combination thereof.

In some aspects, the anti-PD-1 antibody comprises, e.g., nivolumab, pembrolizumab, cemiplimab, PDR001, or CBT-501, or an antigen-binding portion thereof. In some aspects, the anti-PD-1 antibody cross-competes, e.g., with nivolumab, pembrolizumab, cemiplimab, PDR001, sintilimab, tislelizumab, or CBT-501, for binding to human PD-1. In some aspects, the anti-PD-1 antibody binds, e.g., to the same epitope as nivolumab, pembrolizumab, cemiplimab, PDR001, sintilimab, tislelizumab, or CBT-501.

In some aspects, the anti-PD-L1 antibody comprises, e.g., avelumab, atezolizumab, durvalumab, or an antigen-binding portion thereof. In some aspects, the anti-PD-L1 antibody cross-competes, e.g., with avelumab, atezolizumab, or durvalumab for binding to human PD-L1. In some aspects, the anti-PD-L1 antibody binds, e.g., to the same epitope as avelumab, atezolizumab, or durvalumab.

In some aspects, the anti-CTLA-4 antibody comprises ipilimumab, or an antigen-binding portion thereof. In some aspects, the anti-CTLA-4 antibody cross-competes with ipilimumab for binding to human CTLA-4. In some aspects, the anti-CTLA-4 antibody binds to the same epitope as ipilimumab.

In some aspects, the check point modulator therapy administered concurrently or after the administration of a therapy that initiates an immune response comprises, e.g., the administration of (i) an anti-PD-1 antibody selected, e.g., from the group consisting of nivolumab, pembrolizumab, sintilimab, tislelizumab, and cemiplimab; (ii) an anti-PD-L1 antibody selected, e.g., from the group consisting of avelumab, atezolizumab, and durvalumab; (iii) an anti-CTLA-4 antibody, e.g., ipilimumab, or (iii) a combination thereof.

The present disclosure also provides a method for treating a human subject afflicted with a cancer comprising administering an “A-class TME therapy” to the subject, wherein, prior to the administration, the subject is identified via a population-based classifier as exhibiting a combined biomarker comprising (a) a positive Signature 1 score; and (b) a negative Signature 2 score, wherein (i) the Signature 1 score is determined by measuring the expression levels of a gene panel selected from TABLE 3 in a first sample obtained from the subject; and, (ii) the Signature 2 score is determined by measuring the expression levels of a gene panel selected from TABLE 4 in a second sample obtained from the subject.

In one aspect, the present disclosure provides a method for treating a human subject afflicted with a cancer comprising administering “A-class TME therapy” to the subject, wherein, prior to the administration, the subject is identified via a non-population-based classifier, e.g., an ANN classifier, disclosed herein as exhibiting an A class TME, wherein the presence of an A-class TME is determined by applying the ANN classifier model to a set of data comprising expression levels of a gene panel selected from TABLE 1 and TABLE 2 (or any of the gene panels (Genesets) disclosed in FIGS. 28A-G) in a sample obtained from the subject.

Also provided is a method for treating a human subject afflicted with a cancer comprising
(A) identifying via a population-based classifier, prior to the administration, a subject exhibiting a combined biomarker comprising
(a) a positive Signature 1 score; and
(b) a negative Signature 2 score, wherein
(i) the Signature 1 score is determined by measuring the expression levels of a gene panel selected from TABLE 3 in a first sample obtained from the subject; and,
(ii) the Signature 2 score is determined by measuring the expression levels of a gene panel selected from TABLE 4 in a second sample obtained from the subject; and,
(B) administering to the subject an A-class TME therapy.

The present disclosure also provides a method for identifying a human subject afflicted with a cancer suitable for treatment with an A-class TME therapy, the method comprising
(i) determining a Signature 1 score by measuring the expression levels of a gene panel selected from TABLE 3 in a first sample obtained from the subject; and,
(ii) determining a Signature 2 score by measuring the expression levels of a gene panel selected from TABLE 4 in a second sample obtained from the subject,
wherein the presence of a combined biomarker comprising
(a) a positive Signature 1 score; and
(b) a negative Signature 2 score, identified via a population-based classifier prior to the administration,
indicates that an A-class TME therapy can be administered to treat the cancer.

The present disclosure also provides a method for treating a human subject afflicted with a cancer comprising
(A) identifying via a non-population-based classifier (e.g., an ANN), prior to the administration, a subject exhibiting an A-class TME as determined by measuring the expression levels of a gene panel selected from TABLE 1 and TABLE 2 (or any of the gene panels (Genesets) disclosed in FIGS. 28A-G) in a sample obtained from the subject; and,
(B) administering to the subject an A-class TME therapy.

In some aspects, the A-class TME therapy can be administered in combination with additional TME-class therapies disclosed herein if the subject is biomarker-positive for additional stromal phenotypes.

Also provided is a method for identifying a human subject afflicted with a cancer suitable for treatment with an A-class TME therapy, the method comprising determining the presence of an A-class in the subject via a non-population classifier (e.g., an ANN) disclosed herein as determined by measuring the expression levels of a gene panel selected from TABLE 1 and TABLE 2 (or any of the gene panels (Genesets) disclosed in FIGS. 28A-G) in a sample obtained from the subject; wherein the presence of a combined A-class TME indicates that an A-class TME therapy can be administered to treat the cancer.

In some aspects, the A-class TME therapy comprises a VEGF-targeted therapy and other anti-angiogenics, Angiopoietin 1 and 2 (Ang1 and Ang2), DLL4 (Delta Like Canonical Notch Ligand 4), bispecifics of anti-VEGF and anti-DLL4, TKI (tyrosine kinase inhibitors) such as fruquintinib, anti-FGF (Fibroblast growth factor) antibodies and antibodies or small molecules that inhibit the FGF receptor family (FGFR1 and FGFR2); anti-PLGF (Placental growth factor) antibodies and small molecules and antibodies against PLGF receptors, anti-VEGFB (Vascular endothelial growth factor B) antibodies, anti-VEGFC (Vascular endothelial growth factor C) antibodies, anti-VEGFD (Vascular endothelial growth factor D); antibodies to VEGF/PLGF trap molecules such as aflibercept, or ziv-aflibercet; anti-DLL4 antibodies or anti-Notch therapies, such as inhibitors of gamma-secretase.

In some aspects, the anti-angiogenic therapy comprises that administration of antagonists to endoglin, e.g., carotuximab (TRC105).

As used herein the term “VEGF-targeted therapy” refers to targeting the ligands, i.e., VEGF A (vascular endothelial growth factor A), VEGF B (vascular endothelial growth factor B), VEGF C (vascular endothelial growth factor C), VEGF D (vascular endothelial growth factor D), or PLGF (placental growth factor); the receptors, e.g, VEGFR1 (vascular endothelial growth factor receptor 1), VEGFR2 (vascular endothelial growth factor receptor 2), or VEGFR3 (vascular endothelial growth factor receptor 3); or any combination thereof.

In some aspects, the VEGF-target therapy comprises the administration of an anti-VEGF antibody or an antigen-binding portion thereof. In some aspects, the anti-VEGF antibody comprises, e.g., varisacumab, bevacizumab, or an antigen-binding portion thereof. In some aspects, the anti-VEGF antibody cross-competes, e.g., with varisacumab or bevacizumab for binding to human VEGF A. In some aspects, the anti-VEGF antibody binds, e.g. to the same epitope as varisacumab or bevacizumab.

In some aspects, the VEGF-targeted therapy comprises the administration of an anti-VEGFR antibody. In some aspects, the anti-VEGFR antibody is an anti-VEGFR2 antibody. In some aspects, the anti-VEGFR2 antibody comprises ramucirumab or an antigen-binding portion thereof.

In some aspects, the A-class TME therapy comprises the administration of an angiopoietin/TIE2 (TEK receptor tyrosine kinase; CDC202B)-targeted therapy. In some aspects, the angiopoietin/TIE2-target therapy comprises the administration of endoglin and/or angiopoietin.

In some aspects, the A-class TME therapy comprises the administration of a DLL4-targeted therapy. In some aspects, the DLL4-targeted therapy comprises the administration of navicixizumab, ABL101 (NOV1501), or ABT165.

In all methods disclosed above, e.g., methods of treating a subject or selecting a subject for treatment with a specific therapy, wherein the specific therapy (e.g., a TME-class therapy disclosed herein or a combination thereof) is selected according to the classification of the cancer's TME (i.e., whether cancer is biomarker-positive and/or biomarker-negative for at least one of the TME classes, i.e., stromal phenotypes, disclosed herein) using a classifier disclosed herein (e.g., the population and/or non-population based classifiers of the present disclosure), the administration of the specific therapy (e.g., a TME-class therapy disclosed herein or a combination thereof) can effectively treat the cancer.

In some aspects, the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype), reduces the cancer burden. In some aspects, the administration of a specific therapy disclosed herein, e.g., a TME-class therapy disclosed herein or a combination thereof (e.g., when the subject is biomarker-positive for more than one stromal phenotype) to the subject reduces the cancer burden by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the cancer burden prior to the administration of the therapy (e.g., a TME-class therapy disclosed herein or a combination thereof).

In some aspects, the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype), results in progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some aspects, the subject exhibits stable disease after the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype). The term “stable disease” refers to a diagnosis for the presence of a cancer, however the cancer has been treated and remains in a stable condition, i.e. one that that is not progressive, as determined, e.g., by imaging data and/or best clinical judgment. The term “progressive disease” refers to a diagnosis for the presence of a highly active state of a cancer, i.e., one that has not been treated and is not stable or has been treated and has not responded to therapy, or has been treated and active disease remains, as determined by imaging data and/or best clinical judgment.

“Stable disease” can encompass a (temporary) tumor shrinkage/reduction in tumor volume during the course of the treatment compared to the initial tumor volume at the start of the treatment (i.e. prior to treatment). In this context, “tumor shrinkage” can refer to a reduced volume of the tumor upon treatment compared to the initial volume at the start of (i.e. prior to) the treatment. A tumor volume of, for example, less than 100% (e.g., of from about 99% to about 66% of the initial volume at the start of the treatment) can represent a “stable disease”.

“Stable disease” can alternatively encompass a (temporary) tumor growth/increase in tumor volume during the course of the treatment compared to the initial tumor volume at the start of the treatment (i.e. prior to treatment). In this context, “tumor growth” can refer to an increased volume of the tumor upon treatment inhibitor compared to the initial volume at the start of (i.e. prior to) the treatment. A tumor volume of, for example, more than 100% (e.g. of from about 101% to about 135% of the initial volume, preferably of from about 101% to about 110% of the initial volume at the start of the treatment) can represent a “stable disease”.

The term “stable disease” can include the following aspects. For example, the tumor volume does, for example, either not shrink after treatment (i.e. tumor growth is halted) or it does, for example, shrink at the start of the treatment but does not continue to shrink until the tumor has disappeared (i.e. tumor growth is first reverted but, before the tumor has, for example, less than 65% of the initial volume, the tumor grows again.

The term “response” when used in reference to the patients or the tumors to a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype), can be reflected in a “complete response” or “partial response” of the patients or the tumors.

The term “complete response” as used herein can refer to the disappearance of all signs of cancer in response to a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype).

The term “complete response” and the term “complete remission” can be used interchangeably herein. For example, a “complete response” can be reflected in the continued shrinkage of the tumor (as shown in the appended example) until the tumor has disappeared. A tumor volume of, for example, 0% compared to the initial tumor volume (100%) at the start of (i.e. prior to) the treatment can represent a “complete response”.

Treatment with a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype), can result in a “partial response” (or partial remission; e.g. a decrease in the size of a tumor, or in the extent of cancer in the body, in response to the treatment). A “partial response” can encompass a (temporary) tumor shrinkage/reduction in tumor volume during the course of the treatment compared to the initial tumor volume at the start of the treatment (i.e. prior to treatment).

Thus, in some aspects, the subject exhibits a partial response after the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype). In other aspects, the subject exhibits a complete response after the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype).

The term “response” can refer to a “tumor shrinkage.” Accordingly, the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype) to a subject in need thereof can result in a reduction in volume or shrinkage of the tumor.

In some aspects, following the administration of a specific therapy disclosed herein, e.g., an IA-class TME therapy, an IS-class TME therapy, an ID-class TME therapy, an A-class TME therapy, or a combination thereof, the tumor can be reduced in size by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to the tumor's volume prior to the treatment.

In some aspects, the volume of the tumor following the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype), is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the original volume of the tumor prior to the treatment.

In some aspects, the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype) can reduce the growth rate of the tumor by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to the growth rate of the tumor's prior to the treatment.

The term “response” can also refer to a reduction in the number of tumors, for example, when a cancer has metastasized.

In some aspects, the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype), improves progression-free survival probability of the subject by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 12%, at least about 130%, at least about 135%, at least about 140%, at least about 145%, or at least about 150%, compared to the progression-free survival probability of a subject not exhibiting the combined biomarker, or a subject not treated with a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype).

In some aspects, the administration of a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype) improves overall survival probability by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 125%, at least about 130%, at least about 140%, at least about 150%, at least about 160%, at least about 170%, at least about 175%, at least about 180%, at least about 190%, at least about 200%, at least about 210%, at least about 220%, at least about 225%, at least about 230%, at least about 240%, at least about 250%, at least about 260%, at least about 270%, at least about 275%, at least about 280%, at least about 290%, at least about 300%, at least about 310%, at least about 320%, at least about 325%, at least about 330%, at least about 340%, at least about 350%, at least about 360%, at least about 370%, at least about 375%, at least about 380%, at least about 390%, or at least about 400%, compared to the overall survival probability of a subject not exhibiting the combined biomarker or a subject not treated with a specific therapy disclosed herein, e.g., an IA-class TME therapy, an IS-class TME therapy, an ID-class TME therapy, an A-class TME therapy, or a combination thereof (e.g., when the subject is biomarker-positive for more than one stromal phenotype).

The present disclosure also provides a gene panel comprising at least a Signature 1 biomarker gene from TABLE 1 and a Signature 2 biomarker gene from TABLE 2, for use in determining the tumor microenvironment (TME), i.e., the stromal phenotype, of a tumor in a subject in need thereof via a population-based method disclosed herein, wherein the tumor microenvironment or a combination thereof (i.e., a determination of whether the subject is biomarker-positive or biomarker-negative for a TME disclosed herein or a combination thereof) is used for (i) identifying a subject suitable for an anticancer therapy; (ii) determining the prognosis of a subject undergoing anticancer therapy; (iii) initiating, suspending, or modifying the administration of an anticancer therapy; or, (iv) a combination thereof. In some aspects, the gene panel is used according to the methods disclosed here, e.g., to classify a tumor from a patient and to administer a specific therapy (e.g., a TME-class therapy disclosed herein or a combination thereof) based on that classification.

The present disclosure also provides a gene panel comprising at least a biomarker gene from TABLE 1 and a biomarker gene from TABLE 2, for use in determining the tumor microenvironment (TME), i.e., the stromal phenotype, of a tumor in a subject in need thereof via a non-population-based method disclosed herein, e.g., an ANN, wherein the presence or absence of a specific tumor microenvironment or a combination thereof (i.e., a determination of whether the subject is biomarker-positive or biomarker-negative for a TME disclosed herein or a combination thereof) is used for (i) identifying a subject suitable for an anticancer therapy; (ii) determining the prognosis of a subject undergoing anticancer therapy; (iii) initiating, suspending, or modifying the administration of an anticancer therapy; or, (iv) a combination thereof. In some aspects, the gene panel is used according to the methods disclosed here, e.g., to classify a tumor from a patient (e.g., to determine whether a tumor is biomarker-positive or biomarker-negative for a TME disclosed herein or a combination thereof) and to administer a specific therapy (e.g., a TME-class therapy disclosed herein or a combination thereof) based on that classification.

The present disclosure also provides a combined biomarker for identifying via a population-based classifier a human subject afflicted with a cancer suitable for treatment with an anticancer therapy, wherein the combined biomarker comprises a Signature 1 score and a Signature 2 score measured in a sample obtained from the subject wherein (i) the Signature 1 score is determined by measuring the expression levels of the genes in a gene panel of TABLE 3 in a first sample obtained from the subject; and, (ii) the Signature 2 score is determined by measuring the expression levels of the genes in a gene panel of TABLE 4 in a second sample obtained from the subject, and wherein (a) the therapy is an IA-Class TME therapy if the Signature 1 score is negative and the Signature 2 score is positive; (b) the therapy is an IS-Class TME therapy if the Signature 1 score is positive and the Signature 2 score is positive; (c) the therapy is an ID-Class TME therapy if the Signature 1 score is negative and the Signature 2 score is negative; or (d) the therapy is an A-Class TME therapy if the Signature 1 score is positive and the Signature 2 score is negative. In some aspects, e.g., when the subject is identified via a population-based classifier as biomarker-positive or biomarker-negative for more than one of stromal phenotypes disclosed herein, e.g., the subject is biomarker-positive for IA and IS, the subject can be administered a combination therapy corresponding to stromal phenotypes for which the subject is biomarker positive, e.g., a combination therapy comprising an IA-class TME therapy and an IS-class TME therapy.

The present disclosure also provides a combined biomarker for identifying via a non-population-based classifier (e.g., an ANN) a human subject afflicted with a cancer suitable for treatment with an anticancer therapy, wherein the cancer's TME (i.e., stromal phenotype) is determined by measuring the expression levels, e.g., mRNA expression levels, of the genes in a gene panel obtained from TABLE 1 and TABLE 2 (or any of the gene panels (Genesets) disclosed in FIGS. 28A-G), or any of the gene panels (Genesets) disclosed in FIGS. 28A-G, in a sample obtained from the subject, and wherein (a) the therapy is an IA-Class TME therapy if the TME assigned in IA class; (b) the therapy is an IS-Class TME therapy if the TME assigned in IS class (c) the therapy is an ID-Class TME therapy if the TME assigned on ID; or (d) the therapy is an A-Class TME therapy if the TME assigned is A class. In some aspects, e.g., when the subject is identified via a non-population-based classifier (e.g., an ANN) as biomarker-positive or biomarker-negative for more than one of stromal phenotypes disclosed herein, e.g., the subject is biomarker-positive for IA and IS, the subject can be administered a combination therapy corresponding to stromal phenotypes for which the subject is biomarker positive, e.g., a combination therapy comprising an IA-class TME therapy and a IS-class TME therapy.

The present disclosure also provides an anticancer therapy for treating a cancer in a human subject in need thereof, wherein the subject is identified via a population-based classifier as exhibiting (i.e., being biomarker-positive) or not exhibiting (i.e., being biomarker-negative) a combined biomarker comprising a Signature 1 score and a Signature 2 score, wherein (i) the Signature 1 score is determined by measuring the expression levels of the genes in a gene panel of TABLE 3 in a first sample obtained from the subject; and, (ii) the Signature 2 score is determined by measuring the expression levels of the genes in a gene panel of TABLE 4 in a second sample obtained from the subject, and wherein (a) the therapy is an IA-Class TME therapy if the Signature 1 score is negative and the Signature 2 score is positive; (b) the therapy is an IS-Class TME therapy if the Signature 1 score is positive and the Signature 2 score is positive; (c) the therapy is an ID-Class TME therapy if the Signature 1 score is negative and the Signature 2 score is negative; or (d) the therapy is an A-Class TME therapy if the Signature 1 score is positive and the Signature 2 score is negative.

The present disclosure also provides an anticancer therapy for treating a cancer in a human subject in need thereof, wherein the subject is identified via a non-population-based classifier (e.g., an ANN) as exhibiting or not exhibiting a specific class TME (i.e., whether the subject is biomarker-positive and/or biomarker-negative for one of more of the stromal phenotypes disclosed herein) determined by measuring the expression levels, e.g., mRNA expression levels, of the genes in a gene panel obtained from TABLE 1 and TABLE 2 (or any of the gene panels (Genesets) disclosed in FIGS. 28A-G), or any of the gene panels (Genesets) disclosed in FIGS. 28A-G, in a sample obtained from the subject, and wherein (a) the therapy is an IA-Class TME therapy if the TME assigned is IA class; (b) the therapy is an IS-Class TME therapy if the TME assigned is IS class (c) the therapy is an ID-Class TME therapy if the TME assigned is ID class; or (d) the therapy is an A-Class TME therapy if the TME assigned is A class. In some aspects, if the patient is biomarker-positive for more than one TME class, the patient can receive a therapy combining TME specific therapies corresponding to each of the TME class for which the patient is biomarker-positive.

In some aspects, the term “administering” can also comprise commencing a therapy, discontinuing or suspending a therapy, temporarily suspending a therapy, or modifying a therapy (e.g., increasing dosage or frequency of doses, or adding one of more therapeutic agents in a combination therapy).

In some aspects, samples can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the quantification of the expression level of a biomarker disclosed herein; comparisons between biomarker scores or protein expression levels; evaluation of the absence or presence of biomarkers; determination of biomarker levels with respect to a certain threshold; treatment decisions; or combinations thereof, can be performed by one or more healthcare providers, healthcare benefits providers, and/or clinical laboratories.

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a therapy disclosed herein to treat a cancer. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification of a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits provides can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

The assignment of a patient to a specific TME class or classes disclosed herein (resulting from the application of a population-based classifier and/or a non-population classifier disclosed herein) can be applied, in addition to the treatment of patients or to the selection of a patient for treatment, to other therapeutic or diagnostic methods. For example, to methods to devise new methods of treatment (e.g., by selecting patients as candidates for a certain therapy or for participation in a clinical trial), to methods to monitor the efficacy of therapeutic agents, or to methods to adjust a treatment (e.g., formulations, dosage regimens, or routes of administration).

The methods disclosed herein can also include additional steps such as prescribing, initiating, and/or altering prophylaxis and/or treatment, based at least in part on the determination of the presence or absence of a particular TME in a subject's cancer through the application of a population-based classifier and/or non-population-based classifier disclosed herein (i.e., whether the subject is biomarker-positive and/or biomarker-negative for one of more of the stromal phenotypes disclosed herein).

The present disclosure also provides a method of determining whether to treat with a specific TME-class therapy disclosed herein or a combination thereof a patient having a particular TME identified through the application of a population-based classifier and/or non-population-based classifier disclosed herein (i.e., whether the patient is biomarker-positive and/or biomarker-negative for one of more of the stromal phenotypes disclosed herein). Also provided are methods of selecting a patient diagnosed with a cancer as a candidate for treatment with a specific TME-class therapy disclosed herein or a combination thereof based on the presence and/or absence of a particular TME identified through the application of a population-based classifier and/or a non-population-based classifier disclosed herein (i.e., whether the patient is biomarker-positive and/or biomarker-negative for one of more of the stromal phenotypes disclosed herein).

In one aspect, the methods disclosed herein include making a diagnosis, which can be a differential diagnosis, based at least in part on the classification of the TME of a cancer in a subject (i.e., whether the subject is biomarker-positive and/or biomarker-negative for one of more of the stromal phenotypes disclosed herein), wherein the TME has been classified through the application of a population-based classifier and/or a non-population-based classifier disclosed herein. This diagnosis can be recorded in a patient medical record. For example, in various aspects, the classification of the cancer's TME (i.e., whether the subject is biomarker-positive and/or biomarker-negative for one of more of the stromal phenotypes disclosed herein), the diagnosis of the patients as treatable with a specific TME-class specific therapy disclosed herein or a combination thereof, or the selected treatment can be recorded in a medical record. The medical record can be in paper form and/or can be maintained in a computer-readable medium. The medical record can be maintained by a laboratory, physician's office, a hospital, a healthcare maintenance organization, an insurance company, and/or a personal medical record website.

In some aspects, a diagnosis, based on the application of a population and/or non-population-based classifier disclosed herein can be recorded on or in a medical alert article such as a card, a worn article, and/or a radio-frequency identification (RFID) tag. As used herein, the term “worn article” refers to any article that can be worn on a subject's body, including, but not limited to, a tag, bracelet, necklace, or armband.

In some aspects, the sample can be obtained by a healthcare professional treating or diagnosing the patient, for measurement of the biomarker levels in the sample according to the healthcare professional's instructions (e.g., using a particular assay as described herein). In some aspects, the clinical laboratory performing the assay can advise the healthcare provider as to whether the patient can benefit from treatment with a specific TME-class therapy disclosed herein or a combination thereof based on whether the patient's cancer is classified as belonging to a particular TME class (i.e., whether the subject is biomarker-positive and/or biomarker-negative for one of more of the stromal phenotypes disclosed herein). In some aspects, results of a TME classification (i.e., whether one or more stromal phenotypes disclosed herein are present or absent in the subject, i.e., whether the subject is biomarker-positive and/or biomarker-negative for one of more of the stromal phenotypes disclosed herein) conducted by applying a population-based classifier and/or a non-population-based classifier disclosed herein can be submitted to a healthcare benefits provider for determination of whether the patient's insurance will cover treatment with a specific TME-class therapy disclosed herein or a combination thereof. In some aspects, the clinical laboratory performing the assay can advise the healthcare provide as to whether the patient can benefit from treatment with a specific TME-class therapy disclosed herein or combination thereof based on the cancer's TME classification (i.e., whether the subject is biomarker-positive and/or biomarker-negative for one of more of the stromal phenotypes disclosed herein).

### I.F TME-Class Specific Therapies

The four stromal phenotypes or classes used to identify the dominant biology of the tumor microenvironment (TME), i.e., a specific type of stromal phenotype, can be used to predict which therapies are more effective to treat a specific class. See, e.g., FIG. 10.

### I.F.1 IA-Class TME Therapy

For the TME that is dominated by immune activity, such as the IA (Immune Active) phenotype, a patient with this biology (i.e., an IA biomarker-positive patient) is likely to be responsive to immune checkpoint inhibitors (CPIs) such as anti-PD-1 (e.g., sintilimab, tislelizumab, pembrolizumab, or an antigen binding portion thereof), anti-PD-L1, or anti-CTLA-4, or to RORγ agonist therapeutics.

Checkpoint inhibitors: In some aspects, the immune checkpoint inhibitors are blocking antibodies that bind to PD-1, e.g., nivolumab, cemiplimab (REGN2810), geptanolimab (CBT-501), pacmilimab (CX-072), dostarlimab (TSR-042), sintilimab, tislelizumab, and pembrolizumab; PD-L1, e.g., durvalumab (MEDI4736), avelumab, lodapolimab (LY-3300054), CX-188, and atezolizumab; or CTLA-4, e.g., ipilimumab and tremelimumab. In some aspect, combination of one or more of such antibodies can be used.

Tremelimumab, nivolumab, durvalumab and atezolizumab are described, for example, in U.S. Pat. Nos. 6,682,736, 8,008,449, 8,779,108 and 8,217,149, respectively. In some aspects, atezolizumab can be replaced by another immune checkpoint antibody, such as another blocking antibody that binds to CTLA-4, PD-1 (e.g., sintilimab, tislelizumab, pembrolizumab, or an antigen binding portion thereof), PD-L1, or a bispecific blocking antibody that binds to any checkpoint inhibitor. In selecting a different blocking antibody, those of ordinary skill in the art will know the suitable dose and administration schedule from the literature. Suitable examples of anti-CTLA-4 antibodies are those described in U.S. Pat. No. 6,207,156. Other suitable examples of anti-PD-L1 antibodies are those described in U.S. Pat. No. 8,168,179, which particularly concerns treating PD-L1 over-expressing cancers with human anti-PD-L1 antibodies, including chemotherapy combinations; U.S. Pat. No. 9,402,899, which particularly concerns treating tumors with antibodies to PD-L1, including chimeric, humanized and human antibodies; and U.S. Pat. No. 9,439,962, which particularly concerns treating cancers with anti-PD-L1 antibodies and chemotherapy.

Further suitable antibodies to PD-L1 are those in U.S. Pat. Nos. 7,943,743, 9,580,505 and 9,580,507, kits thereof (U.S. Pat. No. 9,580,507) and nucleic acids encoding the antibodies (U.S. Pat. No. 8,383,796). Such antibodies bind to PD-L1 and compete for binding with a reference antibody; are defined by VH and VL genes; or are defined by heavy and light chain CDR3 (U.S. Pat. No. 7,943,743), or heavy chain CDR3 (U.S. Pat. No. 8,383,796), of defined sequences or conservative modifications thereof; or have 90% or 95% sequence identity to reference antibodies. These anti-PD-L1 antibodies also include those with defined quantitative (including binding affinity) and qualitative properties, immunoconjugates and bispecific antibodies. Further included are methods of using such antibodies, and those with defined quantitative (including binding affinity) and qualitative properties, including antibodies in single chain format and those that are in the format of an isolated CDR, in enhancing an immune response (U.S. Pat. No. 9,102,725). Enhancing an immune response, as in U.S. Pat. No. 9,102,725, can be used to treat cancer or an infectious disease, such as a pathogenic infection by a virus, bacterium, fungus or parasite.

Further suitable antibodies to PD-L1 are those in U.S. Patent Application No. 2016/0009805, which concerns antibodies to particular epitopes on PD-L1, including antibodies of defined CDR sequences and competing antibodies; nucleic acids, vectors, host cells, immunoconjugates; detection, diagnostic, prognostic and biomarker methods; and treatment methods.

Specific treatments comprising ipilimumab are disclosed, e.g., in U.S. Pat. Nos. 7,605,238; 8,318,916; 8,784,815; and 8,017,114. Treatments comprising tremelimumab are disclosed, e.g., in U.S. Pat. Nos. 6,682,736, 7,109,003, 7,132,281, 7,411,057, 7,807,797, 7,824,679, 8,143,379, 8,491,895, and 8,883,984. Treatments with nivolumab are disclosed, e.g., in U.S. Pat. Nos. 8,008,449, 8,779,105, 9,387,247, 9,492,539, 9,492,540, 8,728,474, 9,067,999, 9,073,994, and 7,595,048. Treatments with pembrolizumab are disclosed, e.g., in U.S. Pat. Nos. 8,354,509, 8,900,587, and 8,952,136. Treatments with cemiplimab are disclosed, e.g., in US20150203579A1. Treatment with durvalumab are disclosed, e.g., in U.S. Pat. Nos. 8,779,108 and 9,493,565. Treatment with atezolizumab are disclosed, e.g., in U.S. Pat. No. 8,217,149. Treatments with CX-072 are disclosed, e.g., in Ser. No. 15/069,622. Treatments with LY300054 are disclosed, e.g., in U.S. Ser. No. 10/214,586B2. Treating of tumors with combination of antibodies to PD-1 and CTLA-4 is disclosed, e.g., in U.S. Pat. Nos. 9,084,776, 8,728,474, 9,067,999 and 9,073,994. Treating tumors with antibodies to PD-1 and CTLA-4, including sub-therapeutic doses and PD-L1 negative tumors is disclosed, e.g., in U.S. Pat. No. 9,358,289. Treating tumors with antibodies to PD-L1 and CTLA-4 is disclosed, e.g., in US9,393,301 and U.S. Pat. No. 9,402,899. All these patents and publication are incorporated herein by reference in their entireties.

Specific therapeutic agents and suitable cancer indication are identified in the table below.

TABLE 6

| Target | Generic Name | Other name | Target |
|---|---|---|---|
| PD-1 | Nivolumab | OPDIVO ™ | Melanoma |
| | | | Non-Small Cell Lung Cancer |
| | | | Renal Cell Carcinoma |
| | | | Classical Hodgkin Lymphoma |
| | | | Squamous Cell Carcinoma of the Head and |
| | | | Neck |
| | | | Bladder Cancer |
| | | | Small Cell Lung Cancer |
| | | | Brain Cancer (Malignant Glioma; AA and |
| | | | GBM) |
| | | | Hepatocellular Cancer |
| | | | Esophageal Cancer |
| | | | Gastric Cancer |
| | | | Mesothelioma |
| | | | Multiple Myeloma |
| | Pembrolizumab | KEYTRUDA ™ | Non-Small Cell Lung Cancer |
| | | | Classical Hodgkin Lymphoma) |
| | | | Squamous Cell Carcinoma of the Head and |
| | | | Neck |
| | | | Gastric Cancer |
| | | | Breast Cancer |
| | | | Bladder Cancer |
| | | | Solid Tumors |
| | | | Colorectal Cancer |
| | | | Renal Cell Carcinoma |
| | | | Multiple Myeloma |
| | | | Esophageal Cancer |
| | | | Hepatocellular Cancer |
| | Cemiplimab | REGN2810 | Non-Small Cell Lung Cancer |
| | Spartalizumab | PDR001 | Melanoma |
| | Geptanolimab | CBT-501 | Solid Tumors |
| | Sintilimab | TYVYT ™, | Hodgkin's lymphoma |
| | | IBI308 | |
| | Tislelizumab | BGB-A317 | Solid tumors |
| PD-L1 | Atezolizumab | TECENTRIQ ™ | Bladder Cancer |
| | | MPDL3280A | Non-Small Cell Lung Cancer |
| | | | Renal Cell Carcinoma |
| | | | Colorectal Cancer |
| | | | Prostate Cancer |
| | | | Melanoma |
| | | | Breast Cancer |
| | | | Ovarian Cancer |
| | | | Small Cell Lung Cancer |
| | Avelumab | BAVENCIO ™ | Metastatic Merkel Cell Carcinoma |
| | | | Non-Small Cell Lung Cancer |
| | | | Ovarian Cancer |
| | | | Gastric Cancer |
| | | | Bladder Cancer |
| | | | Renal Cell Carcinoma |
| | | | Diffuse Large B-Cell Lymphoma |
| | | | (DLBCL) - NHL |
| | | | Head & Neck Cancer |
| | Durvalumab | MEDI4736 | Non-Small Cell Lung Cancer |
| | | | Head & Neck Carcinoma |
| | | | Bladder Cancer |
| | | | Small Cell Lung Cancer |
| | Pacmilimab | CX-072 | Solid Tumors or Lymphomas |
| | | PROBODY ™ | |
| | Lodapolimab | LY-3300054 | Solid Tumors |
| CTLA-4 | Ipilimumab | YERVOY ™ | Unresectable or Metastatic Melanoma |
| | | MDX-010 | Adjuvant Melanoma |
| | Tremelimumab | AZD9150 | Melanoma |

RORγ agonist therapeutics: In some aspects, RORγ agonist therapeutics are small molecule agonists of RORγ (Retinoid-related orphan receptor gamma), which belongs to the nuclear hormone receptor family. RORγ plays a critical role in control apoptosis during thymopoiesis and T cell homeostasis. Small molecule agonists in clinical development include LYC-55716 (cintirorgon).

### Tislelizumab

Tislelizumab (BGB-A317) is a humanized monoclonal antibody directed against PD-1. It prevents PD-1 from binding to the ligands PD-L1 and PD-L2 (hence it is a checkpoint inhibitor). Tislelizumab can be used for the treatment of solid cancers, e.g., Hodgkin's lymphoma (alone or in combination with an adjuvant therapy such as platinum-containing chemotherapy), urothelial cancer, NSCLC, or hepatocellular carcinoma. In some aspects, a tislelizumab molecule to be administered to a subject, e.g., in accordance with methods described herein, comprises tislelizumab. Sequences relating to tislelizumab are provided in the table below. In some aspects of the present disclosure, tislelizumab or an antigen binding portion thereof can be administered in combination with bavituximab.

TABLE 7
Tislelizumab Sequences

| SEQ | | |
|---|---|---|
| ID | | |
| NO | Description | Sequence |
| 28 | VH CDR1 | GFSLTSYG |
| 29 | VH CDR2 | IYADGST |
| 30 | VH CDR3 | ARAYGNYWYIDV |
| 31 | VL CDR1 | ESVSND |
| n/a | VL CDR2 | YAF |
| 33 | VL CDR3 | HQAYSSPYT |
| 34 | VH | QVQLQESGPGLVKPSETLSLTCTVSG |
| | | FSLTSYGVHWIRQPPGKGLEWIGVIY |
| | | ADGSTNYNPSLK.SRVTISKDTSKNQ |
| | | VSLKLSSVTAADTAVYYCARAYGNYW |
| | | YIDVWGQGTTVTVSS |
| 35 | VL | DIVMTQSPDSLAVSLGERATINCKSS |
| | | ESVSNDVAWYQQKPGQPPKLLINYAF |
| | | HRFTGVPDRFSGSGYGTDFTLTISSL |
| | | QAEDVAVYYCHQAYSSPYTFGQGTKL |
| | | EIK |

### Sintilimab

Sintilimab (TYVYT®) is a fully human IgG4 monoclonal antibody directed against PD-1. It prevents PD-1 from binding to the ligands PD-L1 and PD-L2 (hence it is a checkpoint inhibitor). Sintilimab can be used for the treatment of solid cancers, e.g., Hodgkin's lymphoma, alone or in combination with an adjuvant therapy. In some aspects, a sintilimab molecule to be administered to a subject, e.g., in accordance with methods described herein, comprises sintilimab. Sequences relating to sintilimab are provided in the table below. In some aspects of the present disclosure, sintilimab or an antigen binding portion thereof can be administered in combination with bavituximab.

TABLE 8
Sintilimab Sequences

| SEQ | | |
|---|---|---|
| ID | | |
| NO | Description | Sequence |
| 36 | VH CDR1 | GGTFSSYA |
| 37 | VH CDR2 | IIPMFDTA |
| 38 | VH CDR3 | ARAEHSSTGTFDY |
| 39 | VL CDR1 | QGISSW |
| n/a | VL CDR2 | AAS |
| 41 | VL CDR3 | QQANHLPFT |
| 42 | VH | QVQLVQSGAEVKKPGSSVKVSCKASGG |
| | | TFSSYAISWVRQAPGQGLEWMGLIIPM |
| | | FDTAGYAQKFQ.GRVAITVDESTSTAY |
| | | MELSSLRSEDTAVYYCARAEHSSTGTF |
| | | DYWGQGTLVTVSS |
| 43 | VL | DIQMTQSPSSVSASVGDRVTITCRASQ |
| | | GISSWLAWYQQKPGKAPKLLISAASSL |
| | | QSGVP.SRFSGSGSGTDFTLTISSLQP |
| | | EDFATYYCQQANHLPFTFGGGTKVEIK |

### I.F.2 IS-Class TME Therapy

For the TME that is dominated by immune suppression, such a patient classified as the IS (Immune Suppressed) phenotype (i.e., an IS biomarker-positive patient) might be resistant to checkpoint inhibitors unless also given a drug to reverse immunosuppression such as anti-phosphatidylserine (anti-PS) and anti-phosphatidylserine-targeting. therapeutics, PI3Kγ inhibitors, adenosine pathway inhibitors, IDO, TIMs, LAG3, TGFβ, and CD47 inhibitors.

Bavituximab is a preferred anti-PS-targeting therapeutic. A patient with this biology also has underlying angiogesis and can also get benefit from anti-angiogenics, such as those used for the A stromal subtype.

Specific therapeutics for IS biomarker-positive patients are now discussed. Anti-PS and PS-targeting antibodies, include, but are not limited to bavituximab; PI3Kγ inhibitors such as LY3023414 (samotolisib), IPI-549; Adenosine Pathway inhibitors such as AB-928 (an oral antagonist of the adenosine 2a and 2b receptors); IDO inhibitors; anti-TIMs, both TIMs and TIM-3; anti-LAG3; TGFβ inhibitors, such as LY2157299 (galunisertib); CD47 inhibitors, such as Forty Seven's magrolimab (5F9).

Specific therapeutics for IS biomarker-positive patients also includes: Anti-TIGIT drugs, which are immunosuppressive through triggering of CD155 (Cluster of Differentiation 155) on dendritic cells (among other activities) and expression of subset of Tregs in tumors. A preferred anti-TIGIT antibody is AB-154. Anti-activin A therapeutics, because Activin A promotes differentiation of M2-like tumor macrophages and inhibits generation of NK cells. Anti-BMP therapeutics are useful, because bone morphogenic protein (BMP) also promotes differentiation of M2-like tumor macrophages and inhibits CTLs and DCs.

Further specific therapeutics for IS biomarker-positive patients also includes: TAM (Tyro3, Axl, and Mer receptors) inhibitors or TAM product inhibitors; anti-IL-10 (interleukin) or anti-IL-10R (interleukin 10 receptor), since IL-10 is immunosuppressive; anti-M-CSF, as macrophage-colony stimulating factor (M-CSF) antagonism has been shown to deplete TAMs; anti-CCL2 (C-C Motif Chemokine Ligand 2) or anti-CCL2R (C-C Motif Chemokine Ligand 2 receptor), the particular pathway targeted by those drugs recruits myeloid cells to tumors; MERTK (Tyrosine-protein kinase Mer) antagonists, as inhibition of this receptor tyrosine kinase triggers a pro-inflammatory TAM phenotype and increases tumor CD8+ cells.

Other therapeutics for IS biomarker-positive patients include: STING agonists, as cytosolic DNA sensing by Stimulator of Interferon Genes (STING) enhances DC-stimulation of anti-tumor CD8+ T cells, and agonists are part of STINGVAX®; antibodies to CCL3 (C-C motif chemokine 3), CCL4 (C-C motif chemokine 4), CCL5 (C-C motif chemokine 5) or their common receptor CCR5 (C-C motif chemokine receptor type 5), as these chemokines are products of myeloid-derived suppressor cells (MDSCs) and activate CCR5 on regulatory T cells (Tregs); inhibitors of arginase-1 because arginase-1 is produced by M2-like TAMs, decreases production of tumor infiltrating lymphocytes (TILs) and increases production of Tregs; antibodies to CCR4 (C-C motif chemokine receptor type 4) can be used to deplete Tregs; antibodies to CCL17 (C-C motif chemokine 17) or CCL22 (C-C motif chemokine 22) can inhibit CCR4 (C-C motif chemokine receptor type 4) activation on Tregs; antibodies to GITR (glucocorticoid-induced TNFR-related protein) can be used to deplete Tregs; inhibitors of DNA methyltransferases (DNMTs) or histone deacetylases (HDACs) that cause the reversal of epigenetic silencing of immune genes, such as entinostat.

In pre-clinical models, inhibitors of phosphodiesterase-5, sildenafil, and tadalafil significantly inhibited the MDSC functions, which can provide benefit to IS patients. All-trans retinoic acid (ATRA) used to differentiate MDSCs into mature dendritic cells (DCs) and macrophages may provide benefit to IS patients. VEGF and c-kit signaling is reported to be involved in the generation of MDSC. Sunitinib treatment of metastatic renal cell carcinoma patients was reported to decrease the number of circulating MDSC, which may provide benefit to IS patients.

Cancers that are the IS phenotype (i.e., IS biomarker-positive), that is, high for both Signatures 1 and 2 in a population-based classifier disclosed, or classified as a IS-class TME according to a non-population-based classifier disclosed herein, represent the target population for bavituximab treatment in combination with a checkpoint inhibitor such as an anti-PD-1 (e.g., sintilimab, tislelizumab, pembrolizumab, or an antigen binding portion thereof), anti-PD-L1 or anti-CTLA-4. This is because the present disclosure notes that immune responses that take place in the presence of angiogenesis show signs of immunosuppression, and bavituximab can restore immune activity to immunosuppressed cells. For single agent bavituximab to work, the ongoing immune response would have to be highly active to the extent that blocking immunosuppression would be sufficient to unleash the full potential of the patient's immune response. However, most late stage cancer patients are in need of keeping their immune response going, and are likely to need a combination with bavituximab and checkpoint inhibitors. Thus, the IS phenotype disclosed herein can be used to determine the cancer patients that are likely to respond to bavituximab and checkpoint inhibitors.

### Bavituximab

Bavituximab is a PS-targeting antibody. Bavituximab binds strongly to anionic phospholipids in the presence of serum. Bavituximab binding to PS is mediated by β2-glycoprotein 1 (β2GPI), a serum protein. β2GPI is also known as apolipoprotein H.

In some aspects, a bavituximab molecule to be administered to a subject, e.g., in accordance with methods described herein, comprises bavituximab. Sequences relating to bavituximab are provided in the table below.

TABLE 9
Bavituximab Sequences

| SEQ | | |
|---|---|---|
| ID NO | Description | Sequence |
| 1 | VH CDR1 | GYNMN |
| 2 | VH CDR2 | HIDPYYG |
| 3 | VH CDR3 | YCVKGGYY |
| 4 | VL CDR1 | RASQDIGSSLN |
| 5 | VL CDR2 | ATSSLDS |
| 6 | VL CDR3 | LQYVSSPPT |
| 22 | VH | EVQLQQSGPELEKPGASVKLSCKASGYSFT |
| | | GYNMNWVKQSHGKSLEWIGHIDPYYGDTSY |
| | | NQKFRGKATLTVDKSSSTAYMQLKSLTSED |
| | | SAVYYCVKGGYYGHWYFDVWGAGTTVTVSS |
| 23 | VL | DIQMTQSPSSLSASLGERVSLTCRASQDIG |
| | | SSLNWLQQGPDGTIKRLIYATSSLDSGVPK |
| | | RFSGSRSGSDYSLTISSLESEDFVDYYCLQ |
| | | YVSSPPTFGAGTKLELK |

In some aspects, the bavituximab molecule is administered in combination with an anti-PD-1 antibody (e.g., sintilimab, tislelizumab, pembrolizumab, or an antigen binding portion thereof). In some aspects, the bavituximab molecule is administered in combination with pembrolizumab. In some aspects, the bavituximab molecule is administered in combination with sintilimab. In some aspects, the bavituximab molecule is administered in combination with tislelizumab. In some aspects, the bavituximab molecule is administered to a subject having hepatocellular carcinoma, gastric cancer, NSCLC, ovarian cancer, breast cancer, head and neck cancer, or pancreatic cancer.

### I.F.3 ID-Class TME Therapy

For the TME with no immune activity, such as a patient classified as the ID (Immune Desert) phenotype (i.e., an ID biomarker-positive patient), a patient with this biology would not respond to a monotherapy of checkpoint inhibitors, anti-angiogenics or other TME targeted therapies, and so should not be treated with anti-PD-1s, anti-PD-L1s, anti-CTLA-4s, or RORγ agonists as monotherapies. A patient with this biology might be treated with therapies that induce immune activity allowing them to then get benefit from checkpoint inhibitors or other TME targeted therapies. Therapies that might induce immune activity for these patients include vaccines, CAR-Ts, neo-epitope vaccines, including personalized vaccines, and TLR-based therapies.

CAR-T therapy is a type of treatment in which a patient's T cells (a type of immune system cell) are changed in the laboratory so they will attack cancer cells. T cells are taken from a patient's blood. Then the gene for a special receptor that binds to a certain protein on the patient's cancer cells is added in the laboratory. The special receptor is called a chimeric antigen receptor (CAR). Large numbers of the CAR T-cells are grown in the laboratory and given to the patient by infusion. CAR T-cell therapy is being studied in the treatment of some types of cancer. Also called chimeric antigen receptor T-cell therapy. In some aspects, a CAR-T therapy comprises the administration of IMM-3, axicabtagene ciloleucel, AUTO, Immunotox, sparX/ARC-T therapies, or BCMA CAR-T.

Toll-like receptors (TLRs), mammalian homolog of drosophila Toll protein, are regarded as critical pattern recognition receptors (PRRs) of innate immunity. Some TLRs on cancer cells may favor cancer progress in an inflammation-dependent or -independent way. Inflammatory response stimulated by TLR signaling could promote oncogenesis by boosting tumor inflammatory microenvironment. In addition, elevated expression levels of certain types of cancer cell TLRs promotes tumorigenesis which is required for TLR adapter molecules, but independent of inflammation. Some TLR agonists have been found to induce strong antitumor activity by indirectly activating tolerant host immune system to destroy cancer cells. Therefore, specific agonists or antagonists of TLRs can be used to treat cancer. In some aspects, the TLR-based comprises the administration of poly(I:C). Multiple TLR agonists have been considered for clinical application. BCG (Bacillus Calmette-Guérin) can be used, e.g., for therapy of superficial bladder cancer or colorectal cancer. TLR3 (Toll-like receptor 3) ligand IPH-3102 (IPH-31XX) can be used to treat, e.g., breast cancer. TLR4 (Toll-like receptor 4) agonist monophosphoryl lipid A (MPL) can be used, e.g., for the treatment of colorectal cancer. In some aspects, MPL can be administered with CERVARIX™ vaccines as an adjuvant for the prophylaxis of HPV (human papilloma virus)-associated cervical cancer. In some aspects, flagellin-derived agonist CBLB502 (entolimod) can be used to treat advanced solid tumors.

In some aspect, the TLR-based therapy comprises the administration of BCG (Bacillus Calmette-Guerin), monophosphoryl lipid A (MPL), entolimod (CBLB502), imiquimod (ALDARA®), 852A (small molecule ssRNA), IMOxine (CpG-ODN), lefitolimod (MGN1703), dSLIM® (Double-Stem Loop ImmunoModulator), CpG oligodeoxynucleotides (CpG-ODN), PF3512676 (also known as CpG7909; alone or combined with chemotherapy), 1018 ISS (alone or in combination with chemotherapy or RITUXAN®), lefitolimod, SD-101, motolimod (VTX-2337), IMO-2055 (IMOxine; EMD 1201081), tilsotolimod (IMO-2125), DV281, CMP-101, or CPG7907.

Therapeutic cancer vaccines are based on specific stimulation of the immune system using tumor antigens to elicit an antitumor response. In some aspects, the cancer vaccine comprises, e.g., IGV-001 (IMVAX™), ilixadencel, IMM-2, TG4010 (MVA expressing MUC-1 and IL-2), TROVAX® (MVA expressing fetal oncogene 5T4 (MVA-5T4)), PROSTVAC® (or PSA-TRICOM®) (MVA expressing PSA), GVAX®, recMAGE-A3 (recombinant Melanoma-associated antigen 3) protein plus AS15 immunostimulant, rindopepimut with GM-CSF plus temozolomide, IMA901 (10 different synthetic tumor-associated peptides), recemotide (L-BLP25) (MUC-1-derived lipopeptide), a DC-based vaccine (expressing, e.g., a cytokine such as IL-12), a multiepitope vaccine composed of tyrosinase, gp100 and MART-1 peptides, a peptide vaccine (EGFRvIII, EphA2, Her2/neu peptide) (alone or in combination with bevacizumab), HSPPC-96 (personalized peptide-based vaccine) (alone or in combination with bevacizumab, INTUVAX® (allogenic cell-based therapy) (alone or in combination with sunitinib), PF-06755990 (vaccine) (alone or in combination with sunitinib and/or tremelimumab), NEOVAX® (neoantigen peptide) (alone or in combination with pembrolizumab and/or radiotherapy), the peptide vaccine used in clinical trial NCT02600949 (alone or in combination with pembrolizumab), DPX-Survivac (encapsulated peptide) (alone or in combination with pembrolizumab and/or chemotherapy, e.g., with cyclophosphamide), pTVG-HP (DNA vaccine encoding PAP antigen) (alone or in combination with nivolumab and/or CM-CSF), GVAX® (GM-CSF-secreting tumor cells) (alone or in combination with nivolumab and/or chemotherapy, e.g., with cyclophosphamide), PROSTVAC® (poxviral vector expressing PSA) (alone or in combination with nivolumab), PROSTVAC® (poxviral vector expressing PSA) (alone or in combination with ipilimumab), GVAX® (GM-CSF-secreting tumor cells) (alone or in combination with nivolumab and ipilimumab, and in combination with CRS-207 and cyclophosphamide), dendritic cell-based p53 vaccine (alone or in combination with nivolumab and ipilimumab), neoantigen DNA vaccine (in combination with durvalumab), or CDX-1401 vaccine (DEC-205/NY-ESO-1 fusion protein) (alone or in combination with atezolizumab and chemotherapy, e.g., guadecitabine).

### I.F.4 A-Class TME Therapy

For the TME that is dominated by angiogenic activity, such as a patient classified as the A (Angiogenic) phenotype (i.e., an A biomarker-positive patient), a patient with this biology might be responsive to VEGF-targeted therapies, DLL4-targeted therapies, Angiopoietin/TIE2-targeted therapies, anti-VEGF/anti-DLL4 bispecific antibodies, such as navicixizumab, and anti-VEGF or anti-VEGF receptor antibodies such as varisacumab, ramucirumab, bevacizumab, etc.

In some aspects, a dual-variable domain immunoglobulin molecule, drug, or therapy with anti-angiogenic effects, such as those that have anti-DLL4 and/or anti-VEGF activity, can be selected to treat a patient identified as being biomarker positive for an angiogenic signature, or identified as having the A stromal phenotype. In some aspects, the dual-variable domain immunoglobulin molecule, drug, or therapy is dilpacimab (ABT165). In some aspects, a dual-targeting protein, drug, or therapy with anti-angiogenic effects, such as those that have anti-DLL4 and/or anti-VEGF activity, can be selected to treat a patient identified as being biomarker positive for the angiogenic signature, or identified as having the A stromal phenotype. In some aspects, the dual-targeting protein, drug, or therapy is ABL001 (NOV1501, TR009), as taught by U.S. Publication No. 2016/0159929, which is herein incorporated by reference in its entirety.

### Navicixizumab

Navicixizumab, an anti-VEGF/anti-DLL4 bispecific antibody, is described in detail, for example, in U.S. Pat. Nos. 9,376,488, 9,574,009 and 9,879,084, each of which is incorporated herein by reference in its entirety.

TABLE 10
Navicixizumab Sequences

| SEQ | | |
|---|---|---|
| ID | | |
| NO | Description | Sequence |
| 13 | VEGF VH CDR1 | NYWMH |
| 14 | VEGF VH CDR2 | DINPSNGRTSYKEKFKR |
| 15 | VEGF VH CDR3 | HYDDKYYPLMDY |
| 16 | DLL4 VH CDR1 | TAYYIH |
| 17 | DLL4 VH CDR2 | YISNYNRATNYNQKFKG |
| 18 | DLL4 V4 CDR3 | RDYDYDVGMDY |
| 19 | VL CDR1 | RASESVDNYGISFMK |
| 20 | VL CDR2 | AASNQGS |
| 21 | VL CDR3 | QQSKEVPWTFGG |
| 24 | VH | QVQLVQSGAEVKKPGASVKISCKASGYS |
| | | FTAYYIHWVKQAPGQGLEWIGYISNYNR |
| | | ATNYNQKFKGRVTFTTDTSTSTAYMELR |
| | | SLRSDDTAVYYCARDYDYDVGMDYWGQG |
| | | TLVTVSS |
| 25 | VL | DIVMTQSPDSLAVSLGERATISCRASES |
| | | VDNYGISFMKWFQQKPGQPPKLLIYAAS |
| | | NQGSGVPDRFSGSGSGTDFTLTISSLQA |
| | | EDVAVYYCQQSKEVPWTFGGGTKVEIK |

### Varisacumab

Varisacumab, an anti-VEGFA monoclonal antibody, is described in detail, for example, in U.S. Pat. Nos. 8,394,943, 9,421,256, and 8,034,905, each of which is incorporated herein by reference in its entirety.

TABLE 11
Varisacumab Sequences

| SEQ ID | | |
|---|---|---|
| NO | Description | Sequence |
| 7 | VH CDR1 | SYAIS |
| 8 | VH CDR2 | GFDPEDGETIYAQKFQG |
| 9 | VH CDR3 | GRSMVRGVIIPFNGMDV |
| 10 | VL CDR1 | RASQSISSYLN |
| 11 | VL CDR2 | AASSLQS |
| 12 | VL CDR3 | QQSYSTPLT |
| 26 | VH | QVQLVQSGAEVKKPGASVKVSCKASG |
| | | GTFSSYAISWVRQAPGQGLEWMGGFD |
| | | PEDGETIYAQKFQGRVTMTEDTSTDT |
| | | AYMELSSLRSEDTAVYYCATGRSMVR |
| | | GVIIPFNGMDVWGQGTTVTVSS |
| 27 | VL | DIRMTQSPSSLSASVGDRVTITCRAS |
| | | QSISSYLNWYQQKPGKAPKLLIYAAS |
| | | SLQSGVPSRFSGSGSGTDFTLTISSL |
| | | QPEDFATYYCQQSYSTPLTFGGGTKV |
| | | EIK |

In some aspects, the varisacumab molecule is administered in combination with a second antibody, e.g., an anti-PD-1 antibody (e.g., sintilimab, tislelizumab, pembrolizumab, or an antigen binding portion thereof). In some aspects, the varisacumab molecule is administered in combination with a chemotherapeutic, e.g., taxane, e.g, paclitaxel or docetaxel.

In some aspects, tyrosine kinase inhibitors (TKIs) are used in anti-angiogenic therapies. Example TKIs include cabozantinib, vandetanib, tivozanib, axitinib, lenvatinib, sorafenib, regorafenib, sunitinib, fruquitinib, and pazopanib. In some aspects, c-MET inhibitors can be used.

Specific therapeutic agents that can be administered as part of the TME-Class specific therapies disclosed herein as include in TABLE 12.

TABLE 12
Therapeutic agents for administration as part of TME-Class specific therapies

| TME-Class | Therapy | | |
|---|---|---|---|
| Therapy | family | Therapeutic agent type | Specific examples |
| IA | CPM | Anti-GITR | TRX518, INCAGN01876, BMS-986156 |
| IA | CPM | Anti-OX40 | Oxelumab |
| IA | CPM | Anti-ICOS (CD278) | vopratelimab. XmAb23104 (anti-PD-1/anti-ICOS) |
| IA | CPM | Anti-4-1BB (CD137) | urelumab, utomilumab, INBRX-105 (anti-PD- |
| | | | L1/anti-4-1BB), MCL A-145 (anti-PD-L1/anti-4- |
| | | | 1BB) |
| IA | CPM | RORγ agonist | LYC-55716 (cintirorgon) |
| IA, IS, ID | CPI | Anti-PD-1 | nivolumab, pembrolizumab, cemiplimab, PDR001, |
| | | | CBT-501, CX-188, TSR-042, XmAb20717 (anti PD- |
| | | | 1/anti-CTLA-4), cetrelimab (JNJ-63723283), |
| | | | Gilvetmab (for canine veterinarian use), sintilimab |
| | | | (IBI308), tilselizumab, pidilizumab, prolgolimab |
| | | | (BCD 100), camrelizumab (SHR-1210), XmAb23104 |
| | | | (anti-PD-i/anti-ICOS), AK104 (anti-PD-1/anti- |
| | | | CTLA-4), MGD019 (anti-PD-i/anti-CTLA-4), |
| | | | XmAb20717 (anti-PD-1/anti-CTLA-4), MEDI5752 |
| | | | (anti-PD-i/anti-CTLA-4), MGD013 (anti-PD-1/anti- |
| | | | LAG3), RO7121661 (RG7769) (anti-PD-1/anti- |
| | | | TIM3), IBI318 (anti-PD-1/undisclosed TAA) |
| IA, IS, ID | CPI | Anti-PD-L1 | atezolizumab, avelumab, durvalumab, CX-072, |
| | | | LY3300054, INBRX-105 (anti-PD-L1/anti-4-1BB), |
| | | | MCL A-145 (anti-PD-L1/anti-4-1BB), KN046 (anti- |
| | | | PD-L1/anti-CTLA4), FS118 (anti-PD-L1/anti- |
| | | | LAG3), LY3415244 (anti-PD-L1/anti-TIM3), |
| | | | YW243.55.570, MDPL3280A |
| IA, IS, ID | CPI | Anti-PD-L2 | AMP-224 |
| IA, IS, ID | CPI | Anti-CTLA-4 | ipilimumab, XmAb20717 (anti PD-1/anti-CTLA-4), |
| | | | tremelimumab, AK104 (anti-PD-1/anti-CTLA-4), |
| | | | MGD019 (anti-PD-1/anti-CTLA-4), XmAb20717 |
| | | | (anti-PD-1/anti-CTLA-4), MEDI5752 (anti-PD- |
| | | | 1/anti-CTLA-4), KN046 (anti-PD-L1/anti-CTLA4), |
| IA, IS | CPI, | TIM-3 inhibitor | RO7121661 (RG7769) (anti-PD-1/anti-TIM3), |
| | AIT | | LY3415244 (anti-PD-L1/anti-TIM3) |
| IA, IS | CPI, | LAG-3 inhibitor | relatlimab, MGD013 (anti-PD-1/anti-LAG3), FS118 |
| | AIT | | (anti-PD-L1/anti-LAG3), BMS-986016 |
| IA, IS | CPI, | BTLA inhibitor | |
| | AIT | | |
| IA, IS | CPI, | TIGIT inhibitor | Etigilimab (OMP 313M32) |
| | AIT | | |
| IA, IS | CPI, | VISTA inhibitor | |
| | AIT | | |
| IA, IS | CPI, | TGF-β inhibitor | LY2157299 (galunisertib) |
| | AIT | | |
| IA, IS | CPI, | TGF-β R1 inhibitor | LY3200882 |
| | AIT | | |
| IA, IS | CPI, | CD86 agonist | |
| | AIT | | |
| IA, IS | CPI, | LAIR1 inhibitor | |
| | AIT | | |
| IA, IS | CPI, | CD160 inhibitor | |
| | AIT | | |
| IA, IS | CPI, | 2B4 inhibitor | |
| | AIT | | |
| IA, IS | CPI, | GITR inhibitor | |
| | AIT | | |
| IA, IS | CPI, | OX40 inhibitor | |
| | AIT | | |
| IA, IS | CPI, | 4-1BB (CD137) inhibitor | |
| | AIT | | |
| IA, IS | CPI, | CD2 inhibitor | |
| | AIT | | |
| IA, IS | CPI, | CD27 inhibitor | |
| | AIT | | |
| IA, IS | CPI, | CDS inhibitor | |
| | AIT | | |
| IA, IS | CPI, | ICAM-1 inhibitor | |
| | AIT | | |
| IA, IS | CPI, | LFA-1 (CD11a/CD18) | |
| | AIT | inhibitor | |
| IA, IS | CPI, | ICOS (CD278) inhibitor | |
| | AIT | | |
| IA, IS | CPI, | CD30 inhibitor | |
| | AIT | | |
| IA, IS | CPI, | CD40 inhibitor | |
| | AIT | | |
| IA, IS | CPI, | BAFFR inhibitor | |
| | AIT | | |
| IA, IS | CPI, | HVEM inhibitor | |
| | AIT | | |
| IA, IS | CPI, | CD7 inhibitor | |
| | AIT | | |
| IA, IS | CPI, | LIGHT inhibitor | |
| | AIT | | |
| IA, IS | CPI, | NKG2C inhibitor | |
| | AIT | | |
| IA, IS | CPI, | SLAMF7 inhibitor | |
| | AIT | | |
| IA, IS | CPI, | NKp80 inhibitor | |
| | AIT | | |
| IS, A | AAT | Anti-VEGF | varisacumab, bevacizumab, navicixizumab (OMP- |
| | | | 305B83) (anti-DLL4/anti-VEGF), ABL101 |
| | | | (NOV1501)(anti-DLL4/anti-VEGF), ranibizumab, |
| | | | faricimab (anti-Ang2/anti-VEGFA), vanucizumab |
| | | | (anti-Ang2/Anti-VEGF), BI83 6880 (anti-Ang2/anti- |
| | | | VEGFA), ABT165 (anti-DLL4/anti-VEGF), |
| IS | AAT | Anti-VEGFR1 | icrucumab (IMC-18F1) |
| IS, A | AAT | Anti-VEGFR2 | ramucirumab, alacizumab, 33C3 |
| IS | AIT | Anti-PS targeting | Bavituximab |
| IS | AIT | Anti-32-g1ycoprotein 1 | Bavituximab |
| IS, A, ID | AIT | PI3K inhibitor | LY3023414 (samotolisib), IPI-549, BKM120, |
| | | | BYL719 |
| IS | AIT | Adenosine pathway inhibitor | AB-928 |
| IS | AIT | IDO inhibitor | epacadostat (INCB24360), navoximod (GDC-0919), |
| | | | BMS-986205 |
| IS | AIT | CD47 inhibitor | magrolimab (5F9), TG-1801 (NI-1701) (anti- |
| | | | CD47/anti-CD19) |
| ID | IRIT | Cancer vaccines | IGV-001 (Imvax), ilixadence, IMM-2, TG4010 |
| | | | (MVA expressing MUC-1 and IL-2), TroVax (MVA |
| | | | expressing fetal oncogene 5T4 (MVA-5T4)), |
| | | | PROSTVAC (or PSA-TRICOM) (MVA expressing |
| | | | PSA), GVAX, recMAGE-A3 protein + AS15 |
| | | | immunostimulant, Rindopepimut with GM-CSF plus |
| | | | temozolomide, IMA901 (10 different synthetic |
| | | | tumor-associated peptides), Tecemotide (L-BLP25) |
| | | | (MUC-1-derived lipopeptide), a DC-based vaccine |
| | | | (expressing, e.g., a cytokine such as IL-12), a |
| | | | multiepitope vaccine composed of tyrosinase, gp100 |
| | | | and MART-1 peptides, a peptide vaccine (EGFRvIII, |
| | | | EphA2, Her2/neu peptide) (alone or in combination |
| | | | with bevacizumab), HSPPC-96 (personalized |
| | | | peptide-based vaccine) (alone or in combination with |
| | | | bevacizumab, Intuvax (allogenic cell-based therapy) |
| | | | (alone or in combination with Sunitinib), PF- |
| | | | 06755990 (vaccine) (alone or in combination with |
| | | | sunitinib and/or tremelimumab), NeoVax (neoantigen |
| | | | peptide) (alone or in combination with |
| | | | pembrolizumab and/or radiotherapy), the peptide |
| | | | vaccine used in clinical trial NCT02600949 (alone or |
| | | | in combination with pembrolizumab), DPX-Survivac |
| | | | (encapsulated peptide) (alone or in combination with |
| | | | pembrolizumab and/or chemotherapy, e.g., with |
| | | | cyclophosphamide), pTVG-HP (DNA vaccine |
| | | | encoding PAP antigen) (alone or in combination with |
| | | | nivolumab and/or CM-CSF), GVAX (GM-CSF- |
| | | | secreting tumor cells) (alone or in combination with |
| | | | nivolumab and/or chemotherapy, e.g., with |
| | | | cyclophosphamide), PROSTVAC (poxviral vector |
| | | | expressing PSA) (alone or in combination with |
| | | | nivolumab), PROSTVAC (poxviral vector expressing |
| | | | PSA) (alone or in combination with ipilimumab), |
| | | | GVAX (GM-CSF-secreting tumor cells) (alone or in |
| | | | combination with nivolumab and ipilimumab, and in |
| | | | combination with CRS-207 and cyclophosphamide), |
| | | | Dendritic cell-based p53 vaccine (alone or in |
| | | | combination with nivolumab and ipilimumab), |
| | | | Neoantigen DNA vaccine (in combination with |
| | | | durvalumab), or CDX-1401 vaccine (DEC-205/NY- |
| | | | ESO-1 fusion protein) (alone or in combination with |
| | | | atezolizumab and chemotherapy, e.g., guadecitabine) |
| ID | IRIT | CAR-T therapies | IMM-3, axicabtagene ciloleucel, AUTO, |
| | | | Immunotox, sparX/ARC-T therapies, BCMA CAR-T |
| ID | IRIT | TLR-based therapies | poly(I:C), BCG (Bacillus Calmette Guerin), IPH |
| | | | 31XX, monophosphoryl lipid A (MPL), CBLB502 |
| | | | (entolimod), CBLB502, imiquimod (ALDARA), |
| | | | 852A (ssRNA), IMOxine (CpG-ODN), MGN1703 |
| | | | (dSLIM, CpG-ODN), PF3512676, 1018 ISS, |
| | | | lefitolimod, SD-101, VTX-2337, EMD 1201081, |
| | | | IMO-2125, DV281, CMP-101, or CPG7907 |
| A, IS | VTT/A | Angiopoietin 1 (Ang1) | |
| | | inhibitor | |
| A, IS | VTT/A | Angiopoietin 2 (Ang2) | vanucizumab (anti-Ang2/Anti-VEGF), faricimab |
| | | inhibitor | (anti-Ang2/anti-VEGFA), nesvacumab, BI836880 |
| | | | (anti-Ang2/anti-VEGFA) |
| A, IS | VTT/A | DLL4 inhibitor | |
| A, IS | VTT/A | TKI inhibitor | cabozantinib, vandetanib, tivozanib, axitinib, |
| | | | lenvatinib, sorafenib, regorafenib, sunitinib, |
| | | | fruquitinib, pazopanib, apatinib |
| A, IS, ID | VTT/A | c-MET inhibitor | |
| A, IS, ID | VTT/A | Anti-FGF | |
| A, IS, ID | VTT/A | anti-FGFR1 | BFKB8488A (RG7992) (anti-FGFR1/anti-KLB) |
| A, IS, ID | VTT/A | Anti-FGFR2 | bemarituzumab (FPA144), aprutumab (BAY |
| | | | 1179470) |
| A, IS, ID | VTT/A | FGFR1 inhibitor | |
| A, IS, ID | VTT/A | FGFR2 inhibitor | |
| A, IS | VTT/A | Anti-PLGF | |
| A, IS | VTT/A | PLGF inhibitor | |
| A, IS | VTT/A | Anti-VEGFB | |
| A, IS | VTT/A | Anti-VEGFC | |
| A, IS | VTT/A | Anti-VEGFD | |
| A, IS | VTT/A | Anti-VEGF/PLGF trap | ziv-aflibercept |
| A, IS | VTT/A | Anti-DLL4/anti-VEGF | navicixizumab (anti-DLL4/anti-VEGF), ABL101 |
| | | | (NOV1501) (anti-DLL4/anti-VEGF), ABT165 (anti- |
| | | | DLL4/anti-VEGF) |
| A, IS, ID | VTT/A | Anti-Notch | Brontictuzumab, tarextumab |
| A, IS | ATTT | Endoglin | |
| A, IS | ATTT | Angiopoietin | |
| A, IS | ATTT | Antagonist to endoglin | TRC105 |
| A, IS | VTT/A | Anti-DLL4 | navicixizumab (anti-DLL4/anti-VEGF), ABL101 |
| | | | (NOV1501) (anti-DLL4/anti-VEGF), ABT165 (anti- |
| | | | DLL4/anti-VEGF), demcizumab |
| IA, IS, ID, | Chemo | Taxanes | Paclitaxel, docetaxel |
| A | | | |
| IA, IS, ID, | Chemo | Vinca alkaloyds | Vinblastine, vincristine |
| A | | | |
| IA, IS, ID, | Chemo | Anthracyclines | Daunorubicin, doxorubicin, aclacinomycin, |
| A | | | dihydroxy anthracin dione, mitoxantrone, |
| IA, IS, ID, | Chemo | Topoisomerase inhibitor | camptothecin, topotecan, irinotecan, 20-S |
| A | | | camptothecin, 9-nitro-camptothecin, 9-amino- |
| | | | camptothecin, G1147211 |
| IA, IS, ID, | Chemo | Antimetabolites | methotrexate, 6-mercaptopurine, 6-thioguanine, |
| A | | | cytarabine, 5-fluorouracil decarbazine |
| IA, IS, ID, | Chemo | Alkylating agents | mechlorethamine, thioepa chlorambucil, CC-1065, |
| A | | | melphalan, carmustine (BSNU), lomustine (CCNU), |
| | | | cyclophosphamide, busulfan, dibromomannitol, |
| | | | streptozotocin, mitomycin C, cysplatin, cis- |
| | | | dichlorodiamine platinum (II) (DDP) cisplatin |
| IA, IS, ID, | Chemo | Other | etoposide, hydroxyurea, cytochalasin B, gramicidin |
| A | | | D, emetine, mitomycin, tenoposide, colchicine, |
| | | | mithramycin, actinomycin D, 1-dehydrotestosterone, |
| | | | glucocorticoids, maytansinoid (e.g., maytansinol or |
| | | | CC-1065) |
| ID | Chemo | Antibody-Drug Conjugates | DS-8201a, glembatumumab vedotin, ABBV-085, |
| | | (ADC) | IMMU-130, SGN-15, brentuximab vedotin, SYD985, |
| | | | BA3011, inotuzumab ozogamicin. |

CPM: Check Point Modulator;
CPI: Check Point Inhibitor;
AAT: Anti-Angiogenic Therapy;
AIT: Anti-Immunosuppression Therapy;
IRIT: Immune Response Initiation Therapy;
VTT/A: VEGF-targeted therapy/Other Antiogenics;
ATTT: Angiopoietin/TIE2-Targeted Therapy;
Chemo: Chemotherapy

### I.F.5 Adjuvant Therapies

The methods to select patients for treatment with a certain therapy and well as the methods of treatment disclosed herein can also comprise (i) the administration of additional therapies, for example, chemotherapy, hormonal therapy, or radiotherapy, (ii) surgery, or (iii) combinations thereof. In some aspects, there additional (adjuvant) therapies can be administered simultaneously or sequentially (before or after) the administration of the TME-specific therapies disclosed above or a combination thereof.

When one or more adjuvant therapies are used in combination with a TME-specific therapy as described herein or a combination thereof, there is no requirement for the combined results to be additive of the effects observed when each treatment is conducted separately. Although at least additive effects are generally desirable, any increased therapeutic effect or benefit (e.g., reduced side-effects) above one of the single therapies would be of value. Also, there is no particular requirement for the combined treatment to exhibit synergistic effects, although this is possible and advantageous.

“Neoadjuvant therapy” may be given as a first step to shrink a tumor before the main treatment, which is usually surgery, is given. Examples of neoadjuvant therapy include chemotherapy, radiation therapy, and hormone therapy. It is a type of induction therapy.

In a particular aspect, A-class TME therapy can be administered in combination with chemotherapeutics, e.g., taxanes such as paclitaxel or docetaxel. In some aspects, A-class TME therapy can comprise chemotherapy (e.g., taxanes such as paclitaxel or docetaxel) combined with VEGF-targeted therapies and/or DLL-4-targeted therapies.

Chemotherapy can be administered as standard of care for IA-class TME therapy, IS-class TME therapy, ID-class TME therapy, or a combination thereof. Thus, if a patient or a patient's cancer is assigned to a particular TME class or a combination thereof (i.e., the patient is biomarker-positive for one of more TME classes and/or biomarker-negative for one or more TME classes), the specific therapy for that TME class or combination thereof (i.e., IA-class TME therapy, IS-class TME therapy, ID-class TME therapy, A-class therapy or a combination thereof) can be added to the standard of care chemotherapy.

Promising anti-tumor effects have been reported from clinical trials using bavituximab in combination with paclitaxel in patients with HER2 negative metastatic breast cancer (Chalasani et al., Cancer Med. 2015 July; 4(7):1051-9); paclitaxel-carboplatin in advanced non-small cell lung cancer, NSCLC (Digumarti et al., Lung Cancer. 2014 November; 86(2):231-6); sorafenib in hepatocellular carcinoma (Cheng et al., Ann Surg Oncol. 2016 December; 23(Suppl. 5):583-5912016); and with docetaxel in previously treated, advanced non-squamous NSCLC (Gerber et al., Clin Lung Cancer. 2016 May; 17(3):169-762016), all of which agents are chemotherapeutics.

### I.F.5.a Chemotherapy

TME-specific therapies as described herein may be administered in combination with one or more adjuvant chemotherapeutic agents or drugs.

The term “chemotherapy” refers to various treatment modalities affecting cell proliferation and/or survival. The treatment may include administration of alkylating agents, antimetabolites, anthracyclines, plant alkaloids, topoisomerase inhibitors, and other antitumor agents, including monoclonal antibodies and kinase inhibitors. The term “neoadjuvant chemotherapy” relates to a preoperative therapy regimen consisting of a panel of hormonal, chemotherapeutic and/or antibody agents, which is aimed to shrink the primary tumor, thereby rendering local therapy (surgery or radiotherapy) less destructive or more effective, enabling breast conserving surgery and evaluation of responsiveness of tumor sensitivity towards specific agents in vivo.

Chemotherapeutic drugs can kill proliferating tumor cells, enhancing the necrotic areas created by the overall treatment. The drugs can thus enhance the action of the primary therapeutic agents of the present disclosure.

Chemotherapeutic agents used in cancer treatment can be divided into several groups, depending on their mechanism of action. Some chemotherapeutic agents directly damage DNA and RNA. By disrupting replication of the DNA such chemotherapeutics either completely halt replication, or result in the production of nonsense DNA or RNA. This category includes, for example, cisplatin (PLATINOL®), daunorubicin (CERUBIDINE®), doxorubicin (ADRIAMYCIN®), and etoposide (VEPESID®). Another group of cancer chemotherapeutic agents interferes with the formation of nucleotides or deoxyribonucleotides, so that RNA synthesis and cell replication is blocked. Examples of drugs in this class include methotrexate (ABITREXATE®), mercaptopurine (PURINETHOL®), fluorouracil (ADRUCIL®), and hydroxyurea (HYDREA®). A third class of chemotherapeutic agents affects the synthesis or breakdown of mitotic spindles, and, as a result, interrupts cell division. Examples of drugs in this class include vinblastine (VELBAN®), vincristine (ONCOVIN®) and taxenes, such as, paclitaxel (TAXOL®), and docetaxel (TAXOTERE®).

In some aspects, the methods disclosed herein include treatment with a taxane derivative, e.g., paclitaxel or docetaxel. In some aspects, the method disclosed herein includes treatment with an anthracycline derivative, such as, for example, doxorubicin, daunorubicin, and aclacinomycin. In some aspects, the method disclosed herein include treatment with a topoisomerase inhibitor, such as, for example, camptothecin, topotecan, irinotecan, 20-S camptothecin, 9-nitro-camptothecin, 9-amino-camptothecin, or water soluble camptothecin analog G1147211. Treatment with any combination of these and other chemotherapeutic drugs is specifically contemplated.

Patients can receive chemotherapy immediately following surgical removal of a tumor. This approach is commonly referred to as adjuvant chemotherapy. However, chemotherapy can be administered also before surgery, as so-called neoadjuvant chemotherapy.

### I.F.5.a Radiotherapy

TME-specific therapies as described herein may be administered in combination with radiotherapy.

The terms “radiation therapy” and “radiotherapy” refers to the treatment of cancer with ionizing radiation, which comprises particles having sufficient kinetic energy to emit electrons from atoms or molecules and thereby generate ions. The term includes treatments with direct ionizing radiation, such as those produced by alpha particles (helium nuclei), beta particles (electrons), and atomic particles such as protons, and indirect ionizing radiation, such as photons (including gamma and x-rays). Examples of ionizing radiation used in radiation therapy include high energy X-rays, 7-irradiation, electron beams, UV irradiation, microwaves, and photon beams. The direct delivery of radioisotopes to tumor cells is also contemplated.

Most patients receive radiotherapy immediately following surgical removal of a tumor. This approach is commonly referred to as adjuvant radiotherapy. However, radiotherapy can be administered also before surgery, as so-called neoadjuvant radiotherapy.

### II. Cancer Indications

The methods and compositions disclosed herein can be used for the treatment of cancer. A “cancer” refers to a broad group of various proliferative diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. As used herein, the term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth. In some aspects, the cancer is a tumor. “Tumor,” as used herein, refers to all neoplastic cell growth and proliferation, whether malignant or benign, and all pre-cancerous and cancerous cells and tissues.

In some aspects, the methods and compositions disclosed herein are used to reduce or decrease a size of a tumor or inhibit a tumor growth in a subject in need thereof. In some aspects, the tumor is a carcinoma (i.e., a cancer of epithelial origin). In some aspects, the tumor is, e.g., selected from the group consisting of gastric cancer, gastroesophageal junction cancer (GEJ), esophageal cancer, colorectal cancer, liver cancer (hepatocellular carcinoma, HCC), ovarian cancer, breast cancer, NSCLC (non-small cell lung cancer), bladder cancer, lung cancer, pancreatic cancer, head and neck cancer, lymphoma, uterine cancer, renal or kidney cancer, biliary cancer, prostate cancer, testicular cancer, urethral cancer, penile cancer, thoracic cancer, rectal cancer, brain cancer (glioma and glioblastoma), cervical cancer, parotid cancer, larynx cancer, thyroid cancer, adenocarcinomas, neuroblastomas, melanoma, and Merkel cell carcinoma.

In some aspects, the cancer is relapsed. The term “relapsed” refers to a situation where a subject, that has had a remission of cancer after a therapy, has a return of cancer cells. In some aspects, the cancer is refractory. As used herein, the term “refractory” or “resistant” refers to a circumstance where a subject, even after intensive treatment, has residual cancer cells in the body. In some aspects, the cancer is refractory following at least one prior therapy comprising administration of at least one anticancer agent. In some aspects, the cancer is metastatic.

A “cancer” or “cancer tissue” can include a tumor at various stages. In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In other aspects, the cancer or tumor is stage II or stage III, such that, e.g., the cancer or tumor is larger than in stage 0 or stage I, and it has grown into neighboring tissues but it has not metastasized, except potentially to the lymph nodes. In other aspects, the cancer or tumor is stage IV, such that, e.g., the cancer or tumor has metastasized. Stage IV can also be referred to as advanced or metastatic cancer.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, the tumor is a solid tumor. A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated according to the methods disclosed herein include, e.g., leukemia, Hodgkin's disease, non-Hodgkin's lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

### III. Kits and Articles of Manufacture

The present disclosure also provides a kit comprising (i) a plurality of oligonucleotide probes capable of specifically detecting an RNA encoding a gene biomarker from TABLE 1, and (ii) a plurality of oligonucleotide probes capable of specifically detecting an RNA encoding a gene biomarker from TABLE 2. Also provided is an article of manufacture comprising (i) a plurality of oligonucleotide probes capable of specifically detecting an RNA encoding a gene biomarker from TABLE 1, and (ii) a plurality of oligonucleotide probes capable of specifically detecting an RNA encoding a gene biomarker from TABLE 2, wherein the article of manufacture comprises a microarray.

Such kits and articles of manufacture can comprise containers, each with one or more of the various reagents (e.g., in concentrated form) utilized in the method, including, for example, one or more oligonucleotides (e.g., oligonucleotide capable of hybridizing to an mRNA corresponding to a biomarker gene disclosed herein), or antibodies (i.e., antibodies capable of detecting the protein expression product of a biomarker gene disclosed herein).

One or more oligonucleotides or antibodies, e.g., capture antibodies, can be provided already attached to a solid support. One or more oligonucleotides or antibodies can be provided already conjugated to a detectable label.

The kit can also provide reagents, buffers, and/or instrumentation to support the practice of the methods provided herein.

In some aspects, a kit comprises one or more nucleic acid probes (e.g., oligonucleotides comprising naturally occurring and/or chemically modified nucleotide units) capable of hybridizing a subsequence of the gene sequence of a biomarker gene disclosed herein, e.g., under high stringency conditions. In some aspects, one or more nucleic acid probes (e.g., oligonucleotides comprising naturally occurring and/or chemically modified nucleotide units) capable of hybridizing a subsequence of the gene sequence of a biomarker gene disclosed herein, e.g., under high stringency conditions are attached to a microarray, e.g., a microarray chip. In some aspects, the microarray is, e.g., an Affymetrix, Agilent, Applied Microarrays, Arrayjet, or Illumina microarray. In some aspects, the array is a DNA microarray. In some aspects, the microarray is a cDNA microarray, an RNA microarray, an oligonucleotide microarray, a protein microarray, a peptide microarray, a tissue microarray, or a phenotype microarray.

A kit provided according to this disclosure can also comprise brochures or instructions describing the methods disclosed herein or their practical application to classify a patient's cancer sample. Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some aspects, the kit is an HTG Molecular Edge-Seq sequencing kit. In other aspects, the kit is an Illumina sequencing kit, e.g., for the NovaSEq, NextSeq, of HiSeq 2500 platforms.

### IV. Companion Diagnostic System

The methods disclosed herein can be provided as a companion diagnostic, for example available via a web server, to inform the clinician or patient about potential treatment choices. The methods disclosed herein can comprise collecting or otherwise obtaining a biological sample and performing an analytical method (e.g., apply a population-based classifier such as a Signature 1- and Signature 2-based classifier disclosed herein; or a non-population-based classifier such as a classification model based on an ANN disclosed herein) to classify a sample from a patient's tumor, alone or in combination with other biomarkers, into a TME class, and based on the TME class assignment (e.g., presence or absence of a specific stromal phenotype, i.e., whether the subject is biomarker-positive and/or biomarker-negative for a stromal phenotype or a combination thereof) provide a suitable treatment (e.g., a TME class-specific therapy disclosed herein or a combination thereof) for administration to the patient.

At least some aspects of the methods described herein, due to the complexity of the calculations involved, e.g., calculation of Signature scores, preprocessing of input data to apply a ANN model, preprocessing of input data to train an ANN, post-processing the output of an ANN, training an ANN, or any combination thereof, can be implemented with the use of a computer. In some aspects, the computer system comprises hardware elements that are electrically coupled via bus, including a processor, input device, output device, storage device, computer-readable storage media reader, communications system, processing acceleration (e.g., DSP or special-purpose processors), and memory. The computer-readable storage media reader can be further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource.

A single architecture might be utilized to implement one or more servers that can be further configured in accordance with currently desirable protocols, protocol variations, extensions, etc. However, it will be apparent to those skilled in the art that aspects may well be utilized in accordance with more specific application requirements. Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system further comprises one or more devices for providing input data to the one or more processors. The system further comprises a memory for storing a dataset of ranked data elements. In another aspect, the device for providing input data comprises a detector for detecting the characteristic of the data element, e.g., such as a fluorescent plate reader, mass spectrometer, or gene chip reader.

The system additionally may comprise a database management system. User requests or queries can be formatted in an appropriate language understood by the database management system that processes the query to extract the relevant information from the database of training sets. The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests. The system can be in communication with an input device for providing data regarding data elements to the system (e.g., expression values). In one aspect, the input device can include a gene expression profiling system including, e.g., a mass spectrometer, gene chip or array reader, and the like.

Some aspects described herein can be implemented so as to include a computer program product. A computer program product may include a computer readable medium having computer readable program code embodied in the medium for causing an application program to execute on a computer with a database. As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements.

Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents. In one aspect, a computer program product is provided to implement the treatment, diagnostic, prognostic, or monitoring methods disclosed herein, for example, to determine whether to administer an certain therapy based on the classification of a tumor sample or tumor microenvironment sample from a patient according to the classifiers disclosed herein, e.g., a population-based classifier (e.g. based on a Signature 1 and a Signature 2 as disclosed herein) or a non-population-based classifier (e.g., a classification model based on an ANN as disclosed herein).

The computer program product includes a computer readable medium embodying program code executable by a processor of a computing device or system, the program code comprising:
(a) code that retrieves data attributed to a biological sample from a subject, wherein the data comprises expression level data (or data otherwise derived from expression level values) corresponding to biomarkers genes in the biological sample (e.g., a panel a genes from TABLE 1 to derive a Signature 1 and a panel of gene from TABLE 2 to derive a Signature 2, or a panel of genes from TABLE 1 and TABLE 2, or from any of the genesets disclosed in FIGS. 28A-G, or from TABLE 5 that has been used to train an ANN). These values can also be combined with values corresponding, for example, the patient's current therapeutic regimen or lack thereof; and,
(b) code that executes a classification method that indicates, e.g., whether to administer a therapeutic agent to a patient in need thereof based on the TME classification of the patient's cancer, e.g., a population-based classifier (e.g. based on a Signature 1 and a Signature 2 as disclosed herein) or a non-population-based classifier (e.g., a classification model based on an ANN as disclosed herein).

While various aspects have been described as methods or apparatuses, it should be understood that aspects can be implemented through code coupled with a computer, e.g., code resident on a computer or accessible by the computer. For example, software and databases could be utilized to implement many of the methods discussed above. Thus, in addition to aspects accomplished by hardware, it is also noted that these aspects can be accomplished through the use of an article of manufacture comprised of a computer usable medium having a computer readable program code embodied therein, which causes the enablement of the functions disclosed in this description. Therefore, it is desired that aspects also be considered protected by this patent in their program code means as well.

Furthermore, some aspects can be code stored in a computer-readable memory of virtually any kind including, without limitation, RAM, ROM, magnetic media, optical media, or magneto-optical media. Even more generally, some aspects could be implemented in software, or in hardware, or any combination thereof including, but not limited to, software running on a general purpose processor, microcode, PLAs, or ASICs.

It is also envisioned that some aspects could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### V. Additional Techniques and Tests

Factors known in the art for diagnosing and/or suggesting, selecting, designating, recommending or otherwise determining a course of treatment for a patient or class of patients suspected of having cancer can be employed, e.g., in combination with measurements of the target sequence expression, or with the methods disclosed herein. Accordingly, the methods disclosed herein can include additional techniques such as cytology, histology, ultrasound analysis, MRI results, CT scan results, and measurements of PSA levels.

Certified tests for classifying disease status and/or designating treatment modalities can also be used in diagnosing, predicting, and/or monitoring the status or outcome of a cancer in a subject. A certified test can comprise a means for characterizing the expression levels of one or more of the target sequences of interest, and a certification from a government regulatory agency endorsing use of the test for classifying the disease status of a biological sample.

In some aspects, the certified test can comprise reagents for amplification reactions used to detect and/or quantitate expression of the target sequences to be characterized in the test. An array of probe nucleic acids can be used, with or without prior target amplification, for use in measuring target sequence expression.

The test can be submitted to an agency having authority to certify the test for use in distinguishing disease status and/or outcome. Results of detection of expression levels of the target sequences used in the test and correlation with disease status and/or outcome can be submitted to the agency. A certification authorizing the diagnostic and/or prognostic use of the test can be obtained.

Also provided are portfolios of expression levels comprising a plurality of normalized expression levels of any of the genesets disclosed herein. In some aspects, the genes in the geneset are selected from TABLE 1. In some aspects, the genes in the geneset are selected from TABLE 2. In some aspects, the genes in the geneset are selected from TABLE 1 and TABLE 2 (or any of the gene panels (Genesets) disclosed in FIGS. 28A-G). In some aspects, the geneset is selected from the genesets disclosed in TABLE 3 or TABLE 4, or any of the genesets disclosed in FIG. 28A, 28B, 28C, 28D, 28E, 28F, or 28G. Such portfolios can be provided by performing the methods described herein to obtain expression levels from an individual patient or from a group of patients. The expression levels can be normalized by any method known in the art; exemplary normalization methods that can be used in various aspects include Robust Multichip Average (RMA), probe logarithmic intensity error estimation (PLIER), non-linear fit (NLFIT) quantile-based and nonlinear normalization, and combinations thereof. Background correction can also be performed on the expression data; exemplary techniques useful for background correction include mode of intensities, normalized using median polish probe modeling and sketch-normalization.

In some aspects, portfolios are established such that the combination of genes in the portfolio exhibit improved sensitivity and specificity relative to known methods. In considering a group of genes for inclusion in a portfolio, a small standard deviation in expression measurements correlates with greater specificity. Other measurements of variation such as correlation coefficients can also be used in this capacity. The disclosure also encompasses the above methods where the expression level determines the status or outcome of a cancer in the subject with at least about 45% specificity, at least about 50% specificity, at least about 55%, at least about 60% specificity, at least about 65% specificity, at least about 70% specificity, at least about 75% specificity, at least about 80% specificity, at least about 85% specificity, at least about 90% specificity, or at least about 95% specificity.

In some aspects, the accuracy of the methods disclosed herein for diagnosing, monitoring, and/or predicting a status or outcome of a cancer is at least about 45%, at least about 50%, at least about 55%, at least about 60%, at least about 65%, at least about 70%, at least about 75%, at least about 80%, at least about 85%, at least about 90%, or at least about 95%.

The accuracy of a classifier or biomarker can be determined by the 95% confidence interval (CI). Generally, a classifier or biomarker is considered to have good accuracy if the 95% CI does not overlap 1. In some aspects, the 95% CI of a classifier or biomarker is at least about 1.08, at least about 1.10, at least about 1.12, at least about 1.14, at least about 1.15, at least about 1.16, at least about 1.17, at least about 1.18, at least about 1.19, at least about 1.20, at least about 1.21, at least about 1.22, at least about 1.23, at least about 1.24, at least about 1.25, at least about 1.26, at least about 1.27, at least about 1.28, at least about 1.29, at least about 1.30, at least about 1.31, at least about 1.32, at least about 1.33, at least about 1.34, or at least about 1.35 or more. The 95% CI of a classifier or biomarker may be at least about 1.14, at least about 1.15, at least about 1.16, at least about 1.20, at least about 1.21, at least about 1.26, or at least about 1.28. The 95% CI of a classifier or biomarker may be less than about 1.75, less than about 1.74, less than about 1.73, less than about 1.72, less than about 1.71, less than about 1.70, less than about 1.69, less than about 1.68, less than about 1.67, less than about 1.66, less than about 1.65, less than about 1.64, less than about 1.63, less than about 1.62, less than about 1.61, less than about 1.60, less than about 1.59, less than about 1.58, less than about 1.57, less than about 1.56, less than about 1.55, less than about 1.54, less than about 1.53, less than about 1.52, less than about 1.51, less than about 1.50 or less. The 95% CI of a classifier or biomarker may be less than about 1.61, less than about 1.60, less than about 1.59, less than about 1.58, less than about 1.56, 1.55, or 1.53. The 95% CI of a classifier or biomarker may be between about 1.10 to 1.70, between about 1.12 to about 1.68, between about 1.14 to about 1.62, between about 1.15 to about 1.61, between about 1.15 to about 1.59, between about 1.16 to about 1.160, between about 1.19 to about 1.55, between about 1.20 to about 1.54, between about 1.21 to about 1.53, between about 1.26 to about 1.63, between about 1.27 to about 1.61, or between about 1.28 to about 1.60.

In some aspects, the accuracy of a biomarker or classifier is dependent on the difference in range of the 95% CI (e.g., difference in the high value and low value of the 95% CI interval). Generally, biomarkers or classifiers with large differences in the range of the 95% CI interval have greater variability and are considered less accurate than biomarkers or classifiers with small differences in the range of the 95% CI intervals. In some aspects, a biomarker or classifier is considered more accurate if the difference in the range of the 95% CI is less than about 0.60, less than about 0.55, less than about 0.50, less than about 0.49, less than about 0.48, less than about 0.47, less than about 0.46, less than about 0.45, less than about 0.44, less than about 0.43, less than about 0.42, less than about 0.41, less than about 0.40, less than about 0.39, less than about 0.38, less than about 0.37, less than about 0.36, less than about 0.35, less than about 0.34, less than about 0.33, less than about 0.32, less than about 0.31, less than about 0.30, less than about 0.29, less than about 0.28, less than about 0.27, less than about 0.26, less than about 0.25 or less. The difference in the range of the 95% CI of a biomarker or classifier may be less than about 0.48, less than about 0.45, less than about 0.44, less than about 0.42, less than about 0.40, less than about 0.37, less than about 0.35, less than about 0.33, or less than about 0.32. In some aspects, the difference in the range of the 95% CI for a biomarker or classifier is between about 0.25 to about 0.50, between about 0.27 to about 0.47, or between about 0.30 to about 0.45.

In some aspects, the sensitivity of the methods disclosed herein is at least about 45%. In some aspects, the sensitivity is at least about 50%. In some aspects, the sensitivity is at least about 55%. In some aspects, the sensitivity is at least about 60%. In some aspects, the sensitivity is at least about 65%. In some aspects, the sensitivity is at least about 70%. In some aspects, the sensitivity is at least about 75%. In some aspects, the sensitivity is at least about 80%. In some aspects, the sensitivity is at least about 85%. In some aspects, the sensitivity is at least about 90%. In some aspects, the sensitivity is at least about 95%.

In some aspects, the classifiers or biomarkers disclosed herein are clinically significant. In some aspects, the clinical significance of the classifiers or biomarkers is determined by the AUC value. In order to be clinically significant, the AUC value is at least about 0.5, at least about 0.55, at least about 0.6, at least about 0.65, at least about 0.7, at least about 0.75, at least about 0.8, at least about 0.85, at least about 0.9, or at least about 0.95. The clinical significance of the classifiers or biomarkers can be determined by the percent accuracy. For example, a classifier or biomarker is determined to be clinically significant if the accuracy of the classifier or biomarker is at least about 50%, at least about 55%, at least about 60%, at least about 65%, at least about 70%, at least about 72%, at least about 75%, at least about 77%, at least about 80%, at least about 82%, at least about 84%, at least about 86%, at least about 88%, at least about 90%, at least about 92%, at least about 94%, at least about 96%, or at least about 98%.

In other aspects, the clinical significance of the classifiers or biomarkers is determined by the median fold difference (MDF) value. In order to be clinically significant, the MDF value is at least about 0.8, at least about 0.9, at least about 1.0, at least about 1.1, at least about 1.2, at least about 1.3, at least about 1.4, at least about 1.5, at least about 1.6, at least about 1.7, at least about 1.9, or at least about 2.0. In some aspects, the MDF value is greater than or equal to 1.1. In other aspects, the MDF value is greater than or equal to 1.2. Alternatively, or additionally, the clinical significance of the classifiers or biomarkers is determined by the t-test P-value. In some aspects, in order to be clinically significant, the t-test P-value is less than about 0.070, less than about 0.065, less than about 0.060, less than about 0.055, less than about 0.050, less than about 0.045, less than about 0.040, less than about 0.035, less than about 0.030, less than about 0.025, less than about 0.020, less than about 0.015, less than about 0.010, less than about 0.005, less than about 0.004, or less than about 0.003. The t-test P-value can be less than about 0.050. Alternatively, the t-test P-value is less than about 0.010.

In some aspects, the clinical significance of the classifiers or biomarkers is determined by the clinical outcome. For example, different clinical outcomes can have different minimum or maximum thresholds for AUC values, MDF values, t-test P-values, and accuracy values that would determine whether the classifier or biomarker is clinically significant. In another example, a classifier or biomarker is considered clinically significant if the P-value of the t-test was less than about 0.08, less than about 0.07, less than about 0.06, less than about 0.05, less than about 0.04, less than about 0.03, less than about 0.02, less than about 0.01, less than about 0.005, less than about 0.004, less than about 0.003, less than about 0.002, or less than about 0.001.

In some aspects, the performance of the classifier or biomarker is based on the odds ratio. A classifier or biomarker may be considered to have good performance if the odds ratio is at least about 1.30, at least about 1.31, at least about 1.32, at least about 1.33, at least about 1.34, at least about 1.35, at least about 1.36, at least about 1.37, at least about 1.38, at least about 1.39, at least about 1.40, at least about 1.41, at least about 1.42, at least about 1.43, at least about 1.44, at least about 1.45, at least about 1.46, at least about 1.47, at least about 1.48, at least about 1.49, at least about 1.50, at least about 1.52, at least about 1.55, at least about 1.57, at least about 1.60, at least about 1.62, at least about 1.65, at least about 1.67, at least about 1.70 or more. In some aspects, the odds ratio of a classifier or biomarker is at least about 1.33.

The clinical significance of the classifiers and/or biomarkers may be based on Univariable Analysis Odds Ratio P-value (uvaORPval). The Univariable Analysis Odds Ratio P-value (uvaORPval) of the classifier and/or biomarker may be between about 0 and about 0.4. The Univariable Analysis Odds Ratio P-value (uvaORPval) of the classifier and/or biomarker may be between about 0 and about 0.3. The Univariable Analysis Odds Ratio P-value (uvaORPval)) of the classifier and/or biomarker may be between about 0 and about 0.2. The Univariable Analysis Odds Ratio P-value (uvaORPval)) of the classifier and/or biomarker may be less than or equal to 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11.

The Univariable Analysis Odds Ratio P-value (uvaORPval) of the classifier and/or biomarker may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Univariable Analysis Odds Ratio P-value (uvaORPval) of the classifier and/or biomarker may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of the classifiers and/or biomarkers may be based on multivariable analysis Odds Ratio P-value (mvaORPval). The multivariable analysis Odds Ratio P-value (mvaORPval)) of the classifier and/or biomarker may be between about 0 and about 1. The multivariable analysis Odds Ratio P-value (mvaORPval)) of the classifier and/or biomarker may be between about 0 and about 0.9. The multivariable analysis Odds Ratio P-value (mvaORPval)) of the classifier and/or biomarker may be between about 0 and about 0.8. The multivariable analysis Odds Ratio P-value (mvaORPval) of the classifier and/or biomarker may be less than or equal to about 0.90, less than or equal to about 0.88, less than or equal to about 0.86, less than or equal to about 0.84, less than or equal to about 0.82, or less than or equal to about 0.80. The multivariable analysis Odds Ratio P-value (mvaORPval)) of the classifier and/or biomarker may be less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The multivariable analysis Odds Ratio P-value (mvaORPval) of the classifier and/or biomarker may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The multivariable analysis Odds Ratio P-value (mvaORPval)) of the classifier and/or biomarker may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The multivariable analysis Odds Ratio P-value (mvaORPval)) of the classifier and/or biomarker may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of the classifiers and/or biomarkers may be based on the Kaplan Meier P-value (KM P-value). The Kaplan Meier P-value (KM P-value) of the classifier and/or biomarker may be between about 0 and about 0.8. The Kaplan Meier P-value (KM P-value) of the classifier and/or biomarker may be between about 0 and about 0.7. The Kaplan Meier P-value (KM P-value) of the classifier and/or biomarker may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The Kaplan Meier P-value (KM P-value) of the classifier and/or biomarker may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Kaplan Meier P-value (KM P-value) of the classifier and/or biomarker may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Kaplan Meier P-value (KM P-value) of the classifier and/or biomarker may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of the classifiers and/or biomarkers may be based on the survival AUC value (survAUC). The survival AUC value (survAUC) of the classifier and/or biomarker may be between about 0-1. The survival AUC value (survAUC) of the classifier and/or biomarker may be between about 0 to about 0.9. The survival AUC value (survAUC) of the classifier and/or biomarker may be less than or equal to about 1, less than or equal to about 0.98, less than or equal to about 0.96, less than or equal to about 0.94, less than or equal to about 0.92, less than or equal to about 0.90, less than or equal to about 0.88, less than or equal to about 0.86, less than or equal to about 0.84, less than or equal to about 0.82, or less than or equal to about 0.80. The survival AUC value (survAUC) of the classifier and/or biomarker may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The survival AUC value (survAUC) of the classifier and/or biomarker may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The survival AUC value (survAUC) of the classifier and/or biomarker may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The survival AUC value (survAUC) of the classifier and/or biomarker may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001

The clinical significance of the classifiers and/or biomarkers may be based on the Univariable Analysis Hazard Ratio P-value (uvaHRPval). The Univariable Analysis Hazard Ratio P-value (uvaHRPval) of the classifier and/or biomarker may be between about 0 to about 0.4. The Univariable Analysis Hazard Ratio P-value (uvaHRPval) of the classifier and/or biomarker may be between about 0 to about 0.3. The Univariable Analysis Hazard Ratio P-value (uvaHRPval) of the classifier and/or biomarker may be less than or equal to about 0.40, less than or equal to about 0.38, less than or equal to about 0.36, less than or equal to about 0.34, or less than or equal to about 0.32. The Univariable Analysis Hazard Ratio P-value (uvaHRPval) of the classifier and/or biomarker may be less than or equal to about 0.30, less than or equal to about 0.29, less than or equal to about 0.28, less than or equal to about 0.27, less than or equal to about 0.26, less than or equal to about 0.25, less than or equal to about 0.24, less than or equal to about 0.23, less than or equal to about 0.22, less than or equal to about 0.21, or less than or equal to about 0.20. The Univariable Analysis Hazard Ratio P-value (uvaHRPval) of the classifier and/or biomarker may be less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Univariable Analysis Hazard Ratio P-value (uvaHRPval) of the classifier and/or biomarker may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Univariable Analysis Hazard Ratio P-value (uvaHRPval) of the classifier and/or biomarker may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of the classifiers and/or biomarkers may be based on the Multivariable Analysis Hazard Ratio P-value (mvaHRPval)mva HRPval. The Multivariable Analysis Hazard Ratio P-value (mvaHRPval)mva HRPval of the classifier and/or biomarker may be between about 0 to about 1. The Multivariable Analysis Hazard Ratio P-value (mvaHRPval)mva HRPval of the classifier and/or biomarker may be between about 0 to about y 0.9. The Multivariable Analysis Hazard Ratio P-value (mvaHRPval)mva HRPval of the classifier and/or biomarker may be less than or equal to about 1, less than or equal to about 0.98, less than or equal to about 0.96, less than or equal to about 0.94, less than or equal to about 0.92, less than or equal to about 0.90, less than or equal to about 0.88, less than or equal to about 0.86, less than or equal to about 0.84, less than or equal to about 0.82, or less than or equal to about 0.80. The Multivariable Analysis Hazard Ratio P-value (mvaHRPval)mva HRPval of the classifier and/or biomarker may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The Multivariable Analysis Hazard Ratio P-value (mvaHRPval)mva HRPval of the classifier and/or biomarker may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Multivariable Analysis Hazard Ratio P-value (mvaHRPval)mva HRPval of the classifier and/or biomarker may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Multivariable Analysis Hazard Ratio P-value (mvaHRPval)mva HRPval of the classifier and/or biomarker may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of the classifiers and/or biomarkers may be based on the Multivariable Analysis Hazard Ratio P-value (mvaHRPval). The Multivariable Analysis Hazard Ratio P-value (mvaHRPval) of the classifier and/or biomarker may be between about 0 to about 0.60. Significance of the classifier and/or biomarker may be based on the Multivariable Analysis Hazard Ratio P-value (mvaHRPval). The Multivariable Analysis Hazard Ratio P-value (mvaHRPval) of the classifier and/or biomarker may be between about 0 to about 0.50. Significance of the classifier and/or biomarker may be based on the Multivariable Analysis Hazard Ratio P-value (mvaHRPval). The Multivariable Analysis Hazard Ratio P-value (mvaHRPval) of the classifier and/or biomarker may be less than or equal to about 0.50, less than or equal to about 0.47, less than or equal to about 0.45, less than or equal to about 0.43, less than or equal to about 0.40, less than or equal to about 0.38, less than or equal to about 0.35, less than or equal to about 0.33, less than or equal to about 0.30, less than or equal to about 0.28, less than or equal to about 0.25, less than or equal to about 0.22, less than or equal to about 0.20, less than or equal to about 0.18, less than or equal to about 0.16, less than or equal to about 0.15, less than or equal to about 0.14, less than or equal to about 0.13, less than or equal to about 0.12, less than or equal to about 0.11, or less than or equal to about 0.10. The Multivariable Analysis Hazard Ratio P-value (mvaHRPval) of the classifier and/or biomarker may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Multivariable Analysis Hazard Ratio P-value (mvaHRPval) of the classifier and/or biomarker may be less than or equal to about 0.01, less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The classifiers and/or biomarkers disclosed herein may outperform current classifiers or clinical variables in providing clinically relevant analysis of a sample from a subject. In some aspects, the classifiers or biomarkers may more accurately predict a clinical outcome or status as compared to current classifiers or clinical variables. For example, a classifier or biomarker may more accurately predict metastatic disease. Alternatively, a classifier or biomarker may more accurately predict no evidence of disease. In some aspects, the classifier or biomarker may more accurately predict death from a disease. The performance of a classifier or biomarker disclosed herein may be based on the AUC value, odds ratio, 95% CI, difference in range of the 95% CI, p-value or any combination thereof.

The performance of the classifiers and/or biomarkers disclosed herein may be determined by AUC values and an improvement in performance may be determined by the difference in the AUC value of the classifier or biomarker disclosed herein and the AUC value of current classifiers or clinical variables. In some aspects, a classifier and/or biomarker disclosed herein outperforms current classifiers or clinical variables when the AUC value of the classifier and/or or biomarker disclosed herein is greater than the AUC value of the current classifiers or clinical variables by at least about 0.05, by at least about 0.06, by at least about 0.07, by at least about 0.08, by at least about 0.09, by at least about 0.10, by at least about 0.11, by at least about 0.12, by at least about 0.13, by at least about 0.14, by at least about 0.15, by at least about 0.16, by at least about 0.17, by at least about 0.18, by at least about 0.19, by at least about 0.20, by at least about 0.022, by at least about 0.25, by at least about 0.27, by at least about 0.30, by at least about 0.32, by at least about 0.35, by at least about 0.37, by at least about 0.40, by at least about 0.42, by at least about 0.45, by at least about 0.47, or by at least about 0.50 or more. In some aspects, the AUC value of the classifier and/or or biomarker disclosed herein is greater than the AUC value of the current classifiers or clinical variables by at least about 0.10. In some aspects, the AUC value of the classifier and/or or biomarker disclosed herein is greater than the AUC value of the current classifiers or clinical variables by at least about 0.13. In some aspects, the AUC value of the classifier and/or or biomarker disclosed herein is greater than the AUC value of the current classifiers or clinical variables by at least about 0.18.

The performance of the classifiers and/or biomarkers disclosed herein may be determined by the odds ratios and an improvement in performance may be determined by comparing the odds ratio of the classifier or biomarker disclosed herein and the odds ratio of current classifiers or clinical variables. Comparison of the performance of two or more classifiers, biomarkers, and/or clinical variables can generally be based on the comparison of the absolute value of (1-odds ratio) of a first classifier, biomarker or clinical variable to the absolute value of (1-odds ratio) of a second classifier, biomarker or clinical variable. Generally, the classifier, biomarker or clinical variable with the greater absolute value of (1-odds ratio) can be considered to have better performance as compared to the classifier, biomarker or clinical variable with a smaller absolute value of (1-odds ratio).

In some aspects, the performance of a classifier, biomarker or clinical variable is based on the comparison of the odds ratio and the 95% confidence interval (CI). For example, a first classifier, biomarker or clinical variable may have a greater absolute value of (1-odds ratio) than a second classifier, biomarker or clinical variable, however, the 95% CI of the first classifier, biomarker or clinical variable may overlap 1 (e.g., poor accuracy), whereas the 95% CI of the second classifier, biomarker or clinical variable does not overlap 1. In this instance, the second classifier, biomarker or clinical variable is considered to outperform the first classifier, biomarker or clinical variable because the accuracy of the first classifier, biomarker or clinical variable is less than the accuracy of the second classifier, biomarker or clinical variable. In another example, a first classifier, biomarker or clinical variable may outperform a second classifier, biomarker or clinical variable based on a comparison of the odds ratio; however, the difference in the 95% CI of the first classifier, biomarker or clinical variable is at least about 2 times greater than the 95% CI of the second classifier, biomarker or clinical variable. In this instance, the second classifier, biomarker or clinical variable is considered to outperform the first classifier.

In some aspects, a classifier or biomarker disclosed herein more accurate than a current classifier or clinical variable. The classifier or biomarker disclosed herein is more accurate than a current classifier or clinical variable if the range of 95% CI of the classifier or biomarker disclosed herein does not span or overlap 1 and the range of the 95% CI of the current classifier or clinical variable spans or overlaps 1.

In some aspects, a classifier or biomarker disclosed herein more accurate than a current classifier or clinical variable. The classifier or biomarker disclosed herein is more accurate than a current classifier or clinical variable when difference in range of the 95% CI of the classifier or biomarker disclosed herein is about 0.70, about 0.60, about 0.50, about 0.40, about 0.30, about 0.20, about 0.15, about 0.14, about 0.13, about 0.12, about 0.10, about 0.09, about 0.08, about 0.07, about 0.06, about 0.05, about 0.04, about 0.03, or about 0.02 times less than the difference in range of the 95% CI of the current classifier or clinical variable. The classifier or biomarker disclosed herein is more accurate than a current classifier or clinical variable when difference in range of the 95% CI of the classifier or biomarker disclosed herein between about 0.20 to about 0.04 times less than the difference in range of the 95% CI of the current classifier or clinical variable.

### VI. Embodiments

The present disclosure provides population methods for determining the tumor microenvironment (TME) of a cancer in a subject in need thereof. In some aspects, the population method comprises determining a combined biomarker comprising (a) a Signature 1 score; and, (b) a Signature 2 score, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIGS. 28A-28G) in a second sample obtained from the subject.

Also provided is a method for treating a human subject afflicted with a cancer comprising administering IA-class TME therapy to the subject, wherein, prior to the administration, the subject's tumor is identified as having a particular TME. This TME can be, e.g., defined as combined biomarker comprising (a) a negative Signature 1 score; and (b) a positive Signature 2 score, wherein (i) the Signature 1 score is determined by measuring the expression levels of a gene panel selected from TABLE 3 (or from FIG. 28A-28G) in a first sample obtained from the subject; and, (ii) the Signature 2 score is determined by measuring the expression levels of a gene panel selected from TABLE 4 (or from FIGS. 28A-28G) in a second sample obtained from the subject.

The present disclosure also provides a method for treating a human subject afflicted with a cancer comprising (A) identifying, prior to the administration, a subject exhibiting a combined biomarker comprising (a) a negative Signature 1 score; and (b) a positive Signature 2 score, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIG. 28A-28G) in a second sample obtained from the subject; and, (B) administering to the subject an IA-class TME therapy.

Also provided is a method for identifying a human subject afflicted with a cancer suitable for treatment with an IA-class TME therapy, the method comprising (i) determining a Signature 1 score by measuring the expression levels of a gene panel selected from TABLE 3 (or from FIGS. 28A-28G) in a first sample obtained from the subject; and, (ii) determining a Signature 2 score by measuring the expression levels of a gene panel selected from TABLE 4 (or from FIGS. 28A-28G) in a second sample obtained from the subject, wherein the presence of a combined biomarker comprising (a) a negative Signature 1 score; and (b) a positive Signature 2 score, prior to the administration, indicates that a IA-class TME therapy can be administered to treat the cancer.

In some aspects, the IA-class TME therapy comprises a checkpoint modulator therapy. In some aspects, the checkpoint modulator therapy comprises administering an activator of a stimulatory immune checkpoint molecule. In some aspects, the activator of a stimulatory immune checkpoint molecule is an antibody molecule against GITR, OX-40, ICOS, 4-1BB, or a combination thereof. In some aspects, the checkpoint modulator therapy comprises the administration of a RORγ agonist.

In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, alone or a combination thereof, or in combination with an inhibitor of TIM-3, an inhibitor of LAG-3, an inhibitor of BTLA, an inhibitor of TIGIT, an inhibitor of VISTA, an inhibitor of TGF-β or its receptors, an inhibitor of LAIR1, an inhibitor of CD160, an inhibitor of 2B4, an inhibitor of GITR, an inhibitor of OX40, an inhibitor of 4-1BB (CD137), an inhibitor of CD2, an inhibitor of CD27, an inhibitor of CDS, an inhibitor of ICAM-1, an inhibitor of LFA-1 (CD11a/CD18), an inhibitor of ICOS (CD278), an inhibitor of CD30, an inhibitor of CD40, an inhibitor of BAFFR, an inhibitor of HVEM, an inhibitor of CD7, an inhibitor of LIGHT, an inhibitor of NKG2C, an inhibitor of SLAMF7, an inhibitor of NKp80, a CD86 agonist, or a combination thereof.

In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, alone or a combination thereof, or in combination with a modulator (e.g., an agonist or an antagonist) of TIM-3, a modulator (e.g., an agonist or an antagonist) of LAG-3, a modulator (e.g., an agonist or an antagonist) of BTLA, a modulator (e.g., an agonist or an antagonist) of TIGIT, a modulator (e.g., an agonist or an antagonist) of VISTA, a modulator (e.g., an agonist or an antagonist) of TGF-β or its receptors, a modulator (e.g., an agonist or an antagonist) of LAIR1, a modulator (e.g., an agonist or an antagonist) of CD160, a modulator (e.g., an agonist or an antagonist) of 2B4, a modulator (e.g., an agonist or an antagonist) of GITR, a modulator (e.g., an agonist or an antagonist) of OX40, a modulator (e.g., an agonist or an antagonist) of 4-1BB (CD137), a modulator (e.g., an agonist or an antagonist) of CD2, a modulator (e.g., an agonist or an antagonist) of CD27, a modulator (e.g., an agonist or an antagonist) of CDS, a modulator (e.g., an agonist or an antagonist) of ICAM-1, a modulator (e.g., an agonist or an antagonist) of LFA-1 (CD11a/CD18), a modulator (e.g., an agonist or an antagonist) of ICOS (CD278), a modulator (e.g., an agonist or an antagonist) of CD30, a modulator (e.g., an agonist or an antagonist) of CD40, a modulator (e.g., an agonist or an antagonist) of BAFFR, a modulator (e.g., an agonist or an antagonist) of HVEM, a modulator (e.g., an agonist or an antagonist) of CD7, a modulator (e.g., an agonist or an antagonist) of LIGHT, a modulator (e.g., an agonist or an antagonist) of NKG2C, a modulator (e.g., an agonist or an antagonist) of SLAMF7, a modulator (e.g., an agonist or an antagonist) of NKp80, a modulator (e.g., an agonist or an antagonist) of CD86, or any combination thereof.

In some aspects, the anti-PD-1 antibody comprises nivolumab, pembrolizumab, cemiplimab, PDR001, CBT-501, CX-188, TSR-042, sintilimab, tislelizumab, or an antigen-binding portion thereof. In some aspects, the anti-PD-1 antibody cross-competes with nivolumab, pembrolizumab, cemiplimab, PDR001, CBT-501, CX-188, sintilimab, tislelizumab, or TSR-042 for binding to human PD-1. In some aspects, the anti-PD-1 antibody binds to the same epitope as nivolumab, pembrolizumab, cemiplimab, PDR001, CBT-501, CX-188, sintilimab, tislelizumab, or TSR-042. In some aspects, the anti-PD-L1 antibody comprises avelumab, atezolizumab, durvalumab, CX-072, LY3300054, or an antigen-binding portion thereof. In some aspects, the anti-PD-1 antibody cross-competes with avelumab, atezolizumab, or durvalumab for binding to human PD-1. In some aspects, the anti-PD-1 antibody binds to the same epitope as avelumab, atezolizumab, CX-072, LY3300054, sintilimab, tislelizumab, or durvalumab.

In some aspects, the check point modulator therapy comprises the administration of (i) an anti-PD-1 antibody selected from the group consisting of nivolumab, pembrolizumab, cemiplimab, PDR001, CBT-501, CX-188, sintilimab, tislelizumab, or TSR-042; (ii) an anti-PD-L1 antibody selected from the group consisting of avelumab, atezolizumab, CX-072, LY3300054, and durvalumab; or (iii) a combination thereof.

The present disclosure also provides a method for treating a human subject afflicted with a cancer comprising administering an IS-class TME therapy to the subject, wherein, prior to the administration, the subject is identified as exhibiting a combined biomarker comprising (a) a positive Signature 1 score; and (b) a positive Signature 2 score, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIGS. 28A-28G) in a second sample obtained from the subject.

Also provided is a method for treating a human subject afflicted with a cancer comprising (A) identifying, prior to the administration, a subject exhibiting a combined biomarker comprising (a) a positive Signature 1 score; and (b) a positive Signature 2 score, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIGS. 28A-28G) in a second sample obtained from the subject; and, (B) administering to the subject an IS-class TME therapy.

Also provided is a method for identifying a human subject afflicted with a cancer suitable for treatment with an IS-class TME therapy, the method comprising (i) determining a Signature 1 score by measuring the expression levels of a gene panel selected from TABLE 3 (or from FIGS. 28A-28G) in a first sample obtained from the subject; and, (ii) determining a Signature 2 score by measuring the expression levels of a gene panel selected from TABLE 4 (or from FIGS. 28A-28G) in a second sample obtained from the subject, wherein the presence of a combined biomarker comprising (a) a positive Signature 1 score; and (b) a positive Signature 2 score, prior to the administration, indicates that a IS-class TME therapy can be administered to treat the cancer.

In some aspects, the IS-class TME therapy comprises the administration of (1) a checkpoint modulator therapy and an anti-immunosuppression therapy, and/or (2) an antiangiogenic therapy. In some aspects, the checkpoint modulator therapy comprises the administration of an inhibitor of an inhibitory immune checkpoint molecule. In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, or a combination thereof. In some aspect, the anti-PD-1 antibody comprises nivolumab, pembrolizumab, cemiplimab, PDR001, CBT-501, CX-188, TSR-042, sintilimab, tislelizumab, or an antigen-binding portion thereof. In some aspects, the anti-PD-1 antibody cross-competes with nivolumab, pembrolizumab, cemiplimab, PDR001, CBT-501, CX-188, sintilimab, tislelizumab, or TSR-042, for binding to human PD-1. In some aspects, the anti-PD-1 antibody binds to the same epitope as nivolumab, pembrolizumab, cemiplimab, PDR001, CBT-501, CX-188, sintilimab, tislelizumab, or TSR-042. In some aspects, the anti-PD-L1 antibody comprises avelumab, atezolizumab, CX-072, LY3300054, durvalumab, or an antigen-binding portion thereof. In some aspects, the anti-PD-L1 antibody cross-competes with avelumab, atezolizumab, CX-072, LY3300054, or durvalumab for binding to human PD-1.

In some aspects, the anti-PD-L1 antibody binds to the same epitope as avelumab, atezolizumab, CX-072, LY3300054, or durvalumab. In some aspects, the anti-CTLA-4 antibody comprises ipilimumab or the bispecific antibody XmAb20717 (anti PD-1/anti-CTLA-4), or an antigen-binding portion thereof. In some aspects, the anti-CTLA-4 antibody cross-competes with ipilimumab or the bispecific antibody XmAb20717 (anti PD-1/anti-CTLA-4) for binding to human CTLA-4. In some aspects, the anti-CTLA-4 antibody binds to the same CTLA-4 epitope as ipilimumab or the bispecific antibody XmAb20717 (anti PD-1/anti-CTLA-4). In some aspects, the check point modulator therapy comprises the administration of (i) an anti-PD-1 antibody selected from the group consisting of nivolumab, pembrolizumab, cemiplimab, PDR001, CBT-501, CX-188, and TSR-042; (ii) an anti-PD-L1 antibody selected from the group consisting of avelumab, atezolizumab, CX-072, LY3300054, and durvalumab; (iii) an anti-CTLA-4 antibody, which is ipilimumab or the bispecific antibody XmAb20717 (anti PD-1/anti-CTLA-4), or (iv) a combination thereof.

In some aspects, the antiangiogenic therapy comprises the administration of an anti-VEGF antibody selected from the group consisting of varisacumab, bevacizumab, navicixizumab (anti-DLL4/anti-VEGF bispecific), and a combination thereof. In some aspects, the antiangiogenic therapy comprises the administration of an anti-VEGFR antibody. In some aspects, the anti-VEGFR antibody is an anti-VEGFR2 antibody. In some aspects, the anti-VEGFR2 antibody comprises ramucirumab.

In some aspects, the antiangiogenic therapy comprises the administration of navicixizumab, ABL101 (NOV1501), or ABT165. In some aspects, the anti-immunosuppression therapy comprises the administration of an anti-PS antibody, anti-PS targeting antibody, antibody that binds β2-glycoprotein 1, inhibitor of PI3Kγ, adenosine pathway inhibitor, inhibitor of IDO, inhibitor of TIM, inhibitor of LAG3, inhibitor of TGF-β, CD47 inhibitor, or a combination thereof. In some aspects, the anti-PS targeting antibody is bavituximab, or an antibody that binds β2-glycoprotein 1. In some aspects, the PI3Kγ inhibitor is LY3023414 (samotolisib) or IPI-549.

In some aspects, the adenosine pathway inhibitor is AB-928. In some aspects, the TGFβ inhibitor is LY2157299 (galunisertib) or the TGFβR1 inhibitor is LY3200882. In some aspects, the CD47 inhibitor is magrolimab (5F9). In some aspects, the CD47 inhibitor targets SIRPα. In some aspects, the anti-immunosuppression therapy comprises the administration of an inhibitor of TIM-3, an inhibitor of LAG-3, an inhibitor of BTLA, an inhibitor of TIGIT, an inhibitor of VISTA, an inhibitor of TGF-β or its receptors, an inhibitor of LAIR1, an inhibitor of CD160, an inhibitor of 2B4, an inhibitor of GITR, an inhibitor of OX40, an inhibitor of 4-1BB (CD137), an inhibitor of CD2, an inhibitor of CD27, an inhibitor of CDS, an inhibitor of ICAM-1, an inhibitor of LFA-1 (CD11a/CD18), an inhibitor of ICOS (CD278), an inhibitor of CD30, an inhibitor of CD40, an inhibitor of BAFFR, an inhibitor of HVEM, an inhibitor of CD7, an inhibitor of LIGHT, an inhibitor of NKG2C, an inhibitor of SLAMF7, an inhibitor of NKp80, an agonist to CD86, or a combination thereof.

In some aspects, the anti-immunosuppression therapy comprises the administration of a modulator (e.g., an agonist or an antagonist) of TIM-3, a modulator (e.g., an agonist or an antagonist) of LAG-3, a modulator (e.g., an agonist or an antagonist) of BTLA, a modulator (e.g., an agonist or an antagonist) of TIGIT, a modulator (e.g., an agonist or an antagonist) of VISTA, a modulator (e.g., an agonist or an antagonist) of TGF-β or its receptors, a modulator (e.g., an agonist or an antagonist) of LAIR1, a modulator (e.g., an agonist or an antagonist) of CD160, a modulator (e.g., an agonist or an antagonist) of 2B4, a modulator (e.g., an agonist or an antagonist) of GITR, a modulator (e.g., an agonist or an antagonist) of OX40, a modulator (e.g., an agonist or an antagonist) of 4-1BB (CD137), a modulator (e.g., an agonist or an antagonist) of CD2, a modulator (e.g., an agonist or an antagonist) of CD27, a modulator (e.g., an agonist or an antagonist) of CDS, a modulator (e.g., an agonist or an antagonist) of ICAM-1, a modulator (e.g., an agonist or an antagonist) of LFA-1 (CD11a/CD18), a modulator (e.g., an agonist or an antagonist) of ICOS (CD278), a modulator (e.g., an agonist or an antagonist) of CD30, a modulator (e.g., an agonist or an antagonist) of CD40, a modulator (e.g., an agonist or an antagonist) of BAFFR, a modulator (e.g., an agonist or an antagonist) of HVEM, a modulator (e.g., an agonist or an antagonist) of CD7, a modulator (e.g., an agonist or an antagonist) of LIGHT, a modulator (e.g., an agonist or an antagonist) of NKG2C, a modulator (e.g., an agonist or an antagonist) of SLAMF7, a modulator (e.g., an agonist or an antagonist) of NKp80, a modulator (e.g., an agonist or an antagonist) of CD86, or any combination thereof.

The present disclosure also provides a method for treating a human subject afflicted with a cancer comprising administering an ID-class TME therapy to the subject, wherein, prior to the administration, the subject is identified as exhibiting a combined biomarker comprising (a) a negative Signature 1 score; and (b) a negative Signature 2 score, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIGS. 28A-28G) in a second sample obtained from the subject.

Also provided is a method for treating a human subject afflicted with a cancer comprising (A) identifying, prior to the administration, a subject exhibiting a combined biomarker comprising (a) a negative Signature 1 score; and (b) a negative Signature 2 score, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIGS. 28A-28G) in a second sample obtained from the subject; and, (B) administering to the subject an ID-class TME therapy.

Also provided is a method for identifying a human subject afflicted with a cancer suitable for treatment with an ID-class TME therapy, the method comprising (i) determining a Signature 1 score by measuring the expression levels of a gene panel selected from TABLE 3 (or from FIGS. 28A-28G) in a first sample obtained from the subject; and, (ii) determining a Signature 2 score by measuring the expression levels of a gene panel selected from TABLE 4 (or from FIGS. 28A-28G) in a second sample obtained from the subject, wherein the presence of a combined biomarker comprising (a) a negative Signature 1 score; and (b) a negative Signature 2 score, prior to the administration, indicates that a ID-class TME therapy can be administered to treat the cancer.

In some aspects, the ID-class TME therapy comprises the administration of a checkpoint modulator therapy concurrently or after the administration of a therapy that initiates an immune response. In some aspects, the therapy that initiates an immune response is a vaccine, a CAR-T, or a neo-epitope vaccine. In some aspects, the checkpoint modulator therapy comprises the administration of an inhibitor of an inhibitory immune checkpoint molecule. In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, or a combination thereof.

In some aspects, the anti-PD-1 antibody comprises nivolumab, pembrolizumab, cemiplimab, PDR001, CBT-501, CX-188, sintilimab, tislelizumab, or TSR-042, or an antigen-binding portion thereof. In some aspects, the anti-PD-1 antibody cross-competes with nivolumab, pembrolizumab, cemiplimab, PDR001, CBT-501, CX-188, sintilimab, tislelizumab, or TSR-042, for binding to human PD-1. In some aspects, the anti-PD-1 antibody binds to the same epitope as nivolumab, pembrolizumab, cemiplimab, PDR001, CBT-501, CX-188, sintilimab, tislelizumab, or TSR-042. In some aspects, the anti-PD-L1 antibody comprises avelumab, atezolizumab, CX-072, LY3300054, durvalumab, or an antigen-binding portion thereof. In some aspects, the anti-PD-L1 antibody cross-competes with avelumab, atezolizumab, CX-072, LY3300054, or durvalumab for binding to human PD-L1. In some aspects, the anti-PD-L1 antibody binds to the same epitope as avelumab, atezolizumab, CX-072, LY3300054, or durvalumab.

In some aspects, the anti-CTLA-4 antibody comprises ipilimumab or the bispecific antibody XmAb20717 (anti PD-1/anti-CTLA-4), or an antigen-binding portion thereof. In some aspects, the anti-CTLA-4 antibody cross-competes with ipilimumab or the bispecific antibody XmAb20717 (anti PD-1/anti-CTLA-4) for binding to human CTLA-4. In some aspects, the anti-CTLA-4 antibody binds to the same CTLA-4 epitope as ipilimumab or the bispecific antibody XmAb20717 (anti PD-1/anti-CTLA-4). In some aspects, the check point modulator therapy comprises the administration of (i) an anti-PD-1 antibody selected from the group consisting of nivolumab, pembrolizumab, cemiplimab PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iv) an anti-CTLA-4 antibody, which is ipilimumab or the bispecific antibody XmAb20717 (anti PD-1/anti-CTLA-4), or (iii) a combination thereof.

The present disclosure provides a method for treating a human subject afflicted with a cancer comprising administering an A-class TME therapy to the subject, wherein, prior to the administration, the subject is identified as exhibiting a combined biomarker comprising (a) a positive Signature 1 score; and (b) a negative Signature 2 score, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIGS. 28A-28G) in a second sample obtained from the subject.

Also provided is a method for treating a human subject afflicted with a cancer comprising (A) identifying, prior to the administration, a subject exhibiting a combined biomarker comprising (a) a positive Signature 1 score; and (b) a negative Signature 2 score, prior to the administration, wherein (i) the Signature 1 score is determined by measuring the expression levels of a gene panel selected from TABLE 3 (or from FIGS. 28A-28G) in a first sample obtained from the subject; and, (ii) the Signature 2 score is determined by measuring the expression levels of a gene panel selected from TABLE 4 (or from FIG. 28A-28G) in a second sample obtained from the subject; and, (B) administering to the subject an A-class TME therapy.

Also provided is a method for identifying a human subject afflicted with a cancer suitable for treatment with an A-class TME therapy, the method comprising (i) determining a Signature 1 score by measuring the expression levels of a gene panel selected from TABLE 3 (or from FIGS. 28A-28G) in a first sample obtained from the subject; and, (ii) determining a Signature 2 score by measuring the expression levels of a gene panel selected from TABLE 4 (or from FIGS. 28A-28G) in a second sample obtained from the subject, wherein the presence of a combined biomarker comprising (a) a positive Signature 1 score; and (b) a negative Signature 2 score, prior to the administration, indicates that a A-class TME therapy can be administered to treat the cancer.

In some aspects, the A-class TME therapy comprises a VEGF-targeted therapy and other anti-angiogenics, an inhibitor of angiopoietin 1 (Ang1), an inhibitor of angiopoietin 2 (Ang2), an inhibitor of DLL4, a bispecific of anti-VEGF and anti-DLL4, a TKI inhibitor, an anti-FGF antibody, an anti-FGFR1 antibody, an anti-FGFR2 antibody, a small molecule that inhibits FGFR1, a small molecule that inhibits FGFR2, an anti-PLGF antibody, a small molecule against a PLGF receptor, an antibody against a PLGF receptor, an anti-VEGFB antibody, an anti-VEGFC antibody, an anti-VEGFD antibody, an antibody to a VEGF/PLGF trap molecule such as aflibercept, or ziv-aflibercet, an anti-DLL4 antibody, or an anti-Notch therapy such as an inhibitor of gamma-secretase.

In some aspects, the TKI inhibitor is selected from the group consisting of cabozantinib, vandetanib, tivozanib, axitinib, lenvatinib, sorafenib, regorafenib, sunitinib, fruquitinib, pazopanib, and any combination thereof. In some aspects, TKI inhibitor is fruquintinib. In some aspects, the VEGF-targeted therapy comprises the administration of an anti-VEGF antibody or an antigen-binding portion thereof.

In some aspects, the anti-VEGF antibody comprises varisacumab, bevacizumab, or an antigen-binding portion thereof. In some aspects, the anti-VEGF antibody cross-competes with varisacumab, or bevacizumab for binding to human VEGF A. In some aspects, the anti-VEGF antibody binds to the same epitope as varisacumab, or bevacizumab. In some aspects, the VEGF-targeted therapy comprises the administration of an anti-VEGFR antibody. In some aspects, the anti-VEGFR antibody is an anti-VEGFR2 antibody. In some aspects, the anti-VEGFR2 antibody comprises ramucirumab or an antigen-binding portion thereof.

In some aspects, the bispecific anti-VEGF/anti-DLL4 antibody comprises navicixizumab or an antigen-binding portion thereof. In some aspects, the bispecific anti-VEGF/anti-DLL4 antibody cross-competes with navicixizumab for binding to human VEGF and/or DLL4. In some aspects, the bispecific anti-VEGF/anti-DLL4 antibody binds to the same VEGF and/or DLL4 epitopes as navicixizumab.

In some aspects, the A-class TME therapy comprises the administration of an angiopoietin/TIE2-targeted therapy. In some aspects, the angiopoietin/TIE2-target therapy comprises the administration of endoglin and/or angiopoietin. In some aspects, the A-class TME therapy comprises the administration of a DLL4-targeted therapy. In some aspects, the DLL4-targeted therapy comprises the administration of navicixizumab, ABL101 (NOV1501), or ABT165. In some aspects of the methods disclosed herein, the method further comprises (a) administering chemotherapy; (b) performing surgery; (c) administering radiation therapy; or, (d) any combination thereof.

In some aspects, the gene panel selected from TABLE 4 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes selected from TABLE 2, or 1 to 124 genes selected from FIGS. 28A-28G. In some aspects, the gene panel is a gene panel selected from TABLE 4 or FIGS. 28A-28G. In some aspects, the gene panel selected from TABLE 3 comprises 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 genes selected from TABLE 1 or 1 to 124 genes selected from FIGS. 28A-28G. In some aspects, the gene panel is a gene panel selected from TABLE 3 or FIGS. 28A-28G.

In some aspects, the first sample and the second sample are the same sample. In some aspects, the first sample and the second sample are different samples. In some aspects, the first sample and/or the second sample comprises intratumoral tissue. In some aspects, the expression levels are expressed protein levels. In some aspects, the expression levels are transcribed RNA expression levels. In some aspects, the RNA expression levels are determined using sequencing or any technology that measures RNA. In some aspects, the sequencing is Next Generation Sequencing (NGS). In some aspects, the NGS is selected from the group consisting of RNA-Seq, EdgeSeq, PCR, Nanostring, or combinations thereof. In some aspects, the RNA expression levels are determined using fluorescence. In some aspects, the RNA expression levels are determined using an Affymetrix microarray or an Agilent microarray. In some aspects, RNA expression levels are subject to quantile normalization. In some aspects, the quantile normalization comprises binning input RNA level values into quantiles. In some aspects, the input RNA levels are binned into 100 quantiles. In some aspects, the quantile normalization comprises quantile transforming the RNA expression levels to a normal output distribution function.

In some aspects, the calculation of a signature score comprises (i) measuring the expression level for each gene in the gene panel in a test sample from the subject; (ii) for each gene, subtracting the mean expression value obtained from the expression levels of that gene in a reference sample from the expression level of step (i); (iii) for each gene, dividing the value obtained in step (ii) by the standard deviation per gene obtained from the expression levels of the reference sample; and, (iv) adding all the values obtained in step (iii) and dividing the resulting number by the square root of the number of genes in the gene panel; wherein, if the value obtained in (iv) is above zero, the signature score is a positive signature score, and wherein if the value obtained in (iv) is below zero, the signature score is a negative signature score.

In some aspects, the reference sample comprises a collection of reference expression levels. In some aspects, the reference expression values are standardized reference values. In some aspects, the reference expression values are obtained from a sample population. In some aspects, the reference expression levels are derived from a publicly available database or a combination of databases that are normalized to one another. In some aspects, the reference sample comprises a tissue sample obtained from a different population. In some aspects, the reference sample comprises a sample taken at a different time point. In some aspects, the different time point is an earlier time point.

In some aspects, the cancer is a tumor. In some aspects, the tumor is a carcinoma. In some aspects, the tumor is selected from the group consisting gastric cancer, colorectal cancer, liver cancer (hepatocellular carcinoma, HCC), ovarian cancer, breast cancer, NSCLC, bladder cancer, lung cancer, pancreatic cancer, head and neck cancer, lymphoma, uterine cancer, renal or kidney cancer, biliary cancer, prostate cancer, testicular cancer, urethral cancer, penile cancer, thoracic cancer, rectal cancer, brain cancer (glioma and glioblastoma), cervicalparotid cancer, esophageal cancer, gastroesophageal cancer, larynx cancer, thyroid cancer, adenocarcinomas, neuroblastomas, melanoma, and Merkel Cell carcinoma. In some aspects, the cancer is relapsed. In some aspects, the cancer is refractory. In some aspects, the cancer is refractory following at least one prior therapy comprising administration of at least one anticancer agent. In some aspects, the cancer is metastatic.

In some aspects, the administering effectively treats the cancer. In some aspects, the administering reduces the cancer burden. In some aspects, cancer burden is reduced by at least about 10%, at least about 20%, at least about 30%, at least about 40%, or about 50% compared to the cancer burden prior to the administration. In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some aspects, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration. In some aspects, the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

In some aspects, the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

In some aspects, the administering improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject not exhibiting the combined biomarker.

In some aspects, the administering improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not exhibiting the combined biomarker.

The present disclosure also provides a kit comprising (i) a plurality of oligonucleotide probes capable of specifically detecting an RNA encoding a gene biomarker from TABLE 1 (or from FIGS. 28A-28G), and (ii) a plurality of oligonucleotide probes capable of specifically detecting an RNA encoding a gene biomarker from TABLE 2 (or from FIGS. 28A-28G). Also provides in an article of manufacture comprising (i) a plurality of oligonucleotide probes capable of specifically detecting an RNA encoding a gene biomarker from TABLE 1 (or from FIGS. 28A-28G), and (ii) a plurality of oligonucleotide probes capable of specifically detecting an RNA encoding a gene biomarker from TABLE 2 (or from FIGS. 28A-28G), wherein the article of manufacture comprises a microarray.

Also provided is a gene panel comprising at least a biomarker gene from TABLE 1 (or from FIGS. 28A-28G) and a biomarker gene from TABLE 2 (or from FIGS. 28A-28G), for use in determining the tumor microenvironment of a tumor in a subject in need thereof, wherein the tumor microenvironment is used for (i) identifying a subject suitable for an anticancer therapy; (ii) determining the prognosis of a subject undergoing anticancer therapy; (iii) initiating, suspending, or modifying the administration of an anticancer therapy; or, (iv) a combination thereof.

The present disclosure provides a combined biomarker for identifying a human subject afflicted with a cancer suitable for treatment with an anticancer therapy, wherein the combined biomarker comprises a Signature 1 score and a Signature 2 score measured in a sample obtained from the subject wherein (i) the Signature 1 score is determined by measuring the expression levels of the genes in a gene panel of TABLE 3 (or FIGS. 28A-28G) in a first sample obtained from the subject; and, (ii) the Signature 2 score is determined by measuring the expression levels of the genes in a gene panel of TABLE 4 (or FIGS. 28A-28G) in a second sample obtained from the subject; and wherein (a) the therapy is an IA Class TME therapy if the Signature 1 score is negative and the Signature 2 score is positive; (b) the therapy is an IS Class TME therapy if the Signature 1 score is positive and the Signature 2 score is positive; (c) the therapy is an ID Class TME therapy if the Signature 1 score is negative and the Signature 2 score is negative; or, (d) the therapy is an A Class TME therapy if the Signature 1 score is positive and the Signature 2 score is negative.

Also provided is an anticancer therapy for treating a cancer in a human subject in need thereof, wherein the subject is identified as exhibiting a combined biomarker comprising a Signature 1 score and a Signature 2 score, wherein (i) the Signature 1 score is determined by measuring the expression levels of the genes in a gene panel of TABLE 3 (or FIGS. 28A-28G) in a first sample obtained from the subject; and, (ii) the Signature 2 score is determined by measuring the expression levels of the genes in a gene panel of TABLE 4 (or FIGS. 28A-28G) in a second sample obtained from the subject, and wherein (a) the therapy is an IA-Class TME therapy if the Signature 1 score is negative and the Signature 2 score is positive; (b) the therapy is an IS-Class TME therapy if the Signature 1 score is positive and the Signature 2 score is positive; (c) the therapy is an ID-Class TME therapy if the Signature 1 score is negative and the Signature 2 score is negative; or, (d) the therapy is an A-Class TME therapy if the Signature 1 score is positive and the Signature 2 score is negative.

E1. A method for determining the tumor microenvironment (TME) of a cancer in a subject in need thereof, comprising determining a combined biomarker comprising
(a) a Signature 1 score; and,
(b) a Signature 2 score,
wherein
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject.

E2. A method for treating a human subject afflicted with a cancer comprising administering IA-class TME therapy to the subject, wherein, prior to the administration, the subject is identified as exhibiting a combined biomarker comprising
(a) a negative Signature 1 score; and
(b) a positive Signature 2 score,
wherein
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject.

E3. A method for treating a human subject afflicted with a cancer comprising (A) identifying, prior to the administration, a subject exhibiting a combined biomarker comprising
(a) a negative Signature 1 score; and
(b) a positive Signature 2 score,
wherein
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject;
and,
(B) administering to the subject an IA-class TME therapy.

E4. A method for identifying a human subject afflicted with a cancer suitable for treatment with an IA-class TME therapy, the method comprising
(i) determining a Signature 1 score by measuring the expression levels of a gene panel selected from TABLE 3 (or FIGS. 28A-28G) in a first sample obtained from the subject; and,
(ii) determining a Signature 2 score by measuring the expression levels of a gene panel selected from TABLE 4 (or FIGS. 28A-28G) in a second sample obtained from the subject,
wherein the presence of a combined biomarker comprising
(a) a negative Signature 1 score; and
(b) a positive Signature 2 score, prior to the administration,
indicates that a IA-class TME therapy can be administered to treat the cancer.

E5. The method of any one of embodiments E2 to E4, wherein the IA-class TME therapy comprises a checkpoint modulator therapy.

E6. The method of any one of embodiments E2 to E5, wherein the checkpoint modulator therapy comprises administering an activator of a stimulatory immune checkpoint molecule.

E7. The method of embodiment E6, wherein the activator of a stimulatory immune checkpoint molecule is an antibody molecule against GITR, OX-40, ICOS, 4-1BB, or a combination thereof.

E8. The method of embodiment E5, wherein the checkpoint modulator therapy comprises the administration of a RORγ agonist.

E9. The method of embodiment E5, wherein the checkpoint modulator therapy comprises the administration of an inhibitor of an inhibitory immune checkpoint molecule.

E10. The method of embodiment E9, wherein the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, alone or a combination thereof, or in combination with an inhibitor of TIM-3, an inhibitor of LAG-3, an inhibitor of BTLA, an inhibitor of TIGIT, an inhibitor of VISTA, an inhibitor of TGF-β or its receptors, an inhibitor of LAIR1, an inhibitor of CD160, an inhibitor of 2B4, an inhibitor of GITR, an inhibitor of OX40, an inhibitor of 4-1BB (CD137), an inhibitor of CD2, an inhibitor of CD27, an inhibitor of CDS, an inhibitor of ICAM-1, an inhibitor of LFA-1 (CD11a/CD18), an inhibitor of ICOS (CD278), an inhibitor of CD30, an inhibitor of CD40, an inhibitor of BAFFR, an inhibitor of HVEM, an inhibitor of CD7, an inhibitor of LIGHT, an inhibitor of NKG2C, an inhibitor of SLAMF7, an inhibitor of NKp80, or a CD86 agonist.

E11. The method of embodiment E10, wherein the anti-PD-1 antibody comprises nivolumab, pembrolizumab, cemiplimab, PDR001, CBT-501, CX-188, TSR-042, sintilimab, tislelizumab, or an antigen-binding portion thereof.

E12. The method of embodiment E10, wherein the anti-PD-1 antibody cross-competes with nivolumab, pembrolizumab, cemiplimab, PDR001, CBT-501, CX-188, sintilimab, tislelizumab, or TSR-042 for binding to human PD-1.

E13. The method of embodiment E10, wherein the anti-PD-1 antibody binds to the same epitope as nivolumab, pembrolizumab, cemiplimab, PDR001, CBT-501, CX-188, sintilimab, tislelizumab, or TSR-042.

E14. The method of embodiment E10, wherein the anti-PD-L1 antibody comprises avelumab, atezolizumab, durvalumab, CX-072, LY3300054, or an antigen-binding portion thereof.

E15. The method of embodiment E10, wherein the anti-PD-1 antibody cross-competes with avelumab, atezolizumab, or durvalumab for binding to human PD-1.

E16. The method of embodiment E10, wherein the anti-PD-1 antibody binds to the same epitope as avelumab, atezolizumab, CX-072, LY3300054, or durvalumab.

E17. The method of embodiment E5, where the check point modulator therapy comprises the administration of (i) an anti-PD-1 antibody selected from the group consisting of nivolumab, pembrolizumab, cemiplimab, PDR001, CBT-501, CX-188, sintilimab, tislelizumab, or TSR-042; (ii) an anti-PD-L1 antibody selected from the group consisting of avelumab, atezolizumab, CX-072, LY3300054, and durvalumab; or (iii) a combination thereof.

E18. A method for treating a human subject afflicted with a cancer comprising administering an IS-class TME therapy to the subject, wherein, prior to the administration, the subject is identified as exhibiting a combined biomarker comprising
(a) a positive Signature 1 score; and
(b) a positive Signature 2 score,
wherein
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject.

E19. A method for treating a human subject afflicted with a cancer comprising
(A) identifying, prior to the administration, a subject exhibiting a combined biomarker comprising
(a) a positive Signature 1 score; and
(b) a positive Signature 2 score,
wherein
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject;
and,
(B) administering to the subject an IS-class TME therapy.

E20. A method for identifying a human subject afflicted with a cancer suitable for treatment with an IS-class TME therapy, the method comprising
(i) determining a Signature 1 score by measuring the expression levels of a gene panel selected from TABLE 3 (or FIGS. 28A-28G) in a first sample obtained from the subject; and,
(ii) determining a Signature 2 score by measuring the expression levels of a gene panel selected from TABLE 4 (or FIGS. 28A-28G) in a second sample obtained from the subject,
wherein the presence of a combined biomarker comprising
(a) a positive Signature 1 score; and
(b) a positive Signature 2 score,
prior to the administration,
indicates that a IS-class TME therapy can be administered to treat the cancer.

E21. The method of embodiments E18 to E20, wherein the IS-class TME therapy comprises the administration of (1) a checkpoint modulator therapy and an anti-immunosuppression therapy, and/or (2) an antiangiogenic therapy.

E22. The method of embodiment E21, wherein the checkpoint modulator therapy comprises the administration of an inhibitor of an inhibitory immune checkpoint molecule.

E23. The method of embodiment E22, wherein the inhibitor of an inhibitory immune checkpoint molecule is an antibody against PD-1, PD-L1, PD-L2, CTLA-4, or a combination thereof.

E24. The method of embodiment E23, wherein the anti-PD-1 antibody comprises nivolumab, pembrolizumab, cemiplimab, PDR001, CBT-501, CX-188, TSR-042, sintilimab, tislelizumab, or an antigen-binding portion thereof.

E25. The method of embodiment E23, wherein the anti-PD-1 antibody cross-competes with nivolumab, pembrolizumab, cemiplimab, PDR001, CBT-501, CX-188, sintilimab, tislelizumab, or TSR-042, for binding to human PD-1.

E26. The method of embodiment E23, wherein the anti-PD-1 antibody binds to the same epitope as nivolumab, pembrolizumab, cemiplimab, PDR001, CBT-501, CX-188, sintilimab, tislelizumab, or TSR-042.

E27. The method of embodiment E23, wherein the anti-PD-L1 antibody comprises avelumab, atezolizumab, CX-072, LY3300054, durvalumab, or an antigen-binding portion thereof.

E28. The method of embodiment E23, wherein the anti-PD-L1 antibody cross-competes with avelumab, atezolizumab, CX-072, LY3300054, or durvalumab for binding to human PD-1.

E29. The method of embodiment E23, wherein the anti-PD-L1 antibody binds to the same epitope as avelumab, atezolizumab, CX-072, LY3300054, or durvalumab.

E30. The method of embodiment E23, wherein the anti-CTLA-4 antibody comprises ipilimumab or the bispecific antibody XmAb20717 (anti PD-1/anti-CTLA-4), or an antigen-binding portion thereof.

E31. The method of embodiment E23, wherein the anti-CTLA-4 antibody cross-competes with ipilimumab or the bispecific antibody XmAb20717 (anti PD-1/anti-CTLA-4) for binding to human CTLA-4.

E32. The method of embodiment E23, wherein the anti-CTLA-4 antibody binds to the same CTLA-4 epitope as ipilimumab or the bispecific antibody XmAb20717 (anti PD-1/anti-CTLA-4).

E33. The method of embodiment E21, where the check point modulator therapy comprises the administration of (i) an anti-PD-1 antibody selected from the group consisting of nivolumab, pembrolizumab, cemiplimab, PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iii) an anti-CTLA-4 antibody, which is ipilimumab or the bispecific antibody XmAb20717 (anti PD-1/anti-CTLA-4), or (iv) a combination thereof.

E34. The method of embodiments E21 to E33, wherein the antiangiogenic therapy comprises the administration of an anti-VEGF antibody selected from the group consisting of varisacumab, bevacizumab, navicixizumab (anti-DLL4/anti-VEGF bispecific), and a combination thereof.

E35. The method of embodiments E21 to E34, wherein the antiangiogenic therapy comprises the administration of an anti-VEGFR antibody.

E36. The method of embodiment E35, wherein the anti-VEGFR antibody is an anti-VEGFR2 antibody.

E37. The method of embodiment E36, wherein the anti-VEGFR2 antibody comprises ramucirumab.

E38. The method of embodiments E21 to E37, wherein the antiangiogenic therapy comprises the administration of navicixizumab, ABL101 (NOV1501), or ABT165.

E39. The method of embodiments E21 to E38, wherein the anti-immunosuppression therapy comprises the administration of an anti-PS antibody, anti-PS targeting antibody, antibody that binds A2-glycoprotein 1, inhibitor of PI3Kγ, adenosine pathway inhibitor, inhibitor of IDO, inhibitor of TIM, inhibitor of LAG3, inhibitor of TGF-β, CD47 inhibitor, or a combination thereof.

E40. The method of embodiment E39, wherein the anti-PS targeting antibody is bavituximab, or an antibody that binds A2-glycoprotein 1.

E41. The method of embodiment E39, wherein the PI3Kγ inhibitor is LY3023414 (samotolisib) or IPI-549.

E42. The method of embodiment E39, wherein the adenosine pathway inhibitor is AB-928.

E43. The method of embodiment E39, wherein the TGFβ inhibitor is LY2157299 (galunisertib) or the TGFβR1 inhibitor is LY3200882.

E44. The method of embodiment E39, wherein the CD47 inhibitor is magrolimab (5F9).

E45. The method of embodiment E39, wherein the CD47 inhibitor targets SIRPα.

E46. The methods of embodiments E21 to E45 wherein the anti-immunosuppression therapy comprises the administration of an inhibitor of TIM-3, an inhibitor of LAG-3, an inhibitor of BTLA, an inhibitor of TIGIT, an inhibitor of VISTA, an inhibitor of TGF-β or its receptors, an inhibitor of LAIR1, an inhibitor of CD160, an inhibitor of 2B4, an inhibitor of GITR, an inhibitor of OX40, an inhibitor of 4-1BB (CD137), an inhibitor of CD2, an inhibitor of CD27, an inhibitor of CDS, an inhibitor of ICAM-1, an inhibitor of LFA-1 (CD11a/CD18), an inhibitor of ICOS (CD278), an inhibitor of CD30, an inhibitor of CD40, an inhibitor of BAFFR, an inhibitor of HVEM, an inhibitor of CD7, an inhibitor of LIGHT, an inhibitor of NKG2C, an inhibitor of SLAMF7, an inhibitor of NKp80, an agonist to CD86, or a combination thereof.

E47. A method for treating a human subject afflicted with a cancer comprising administering an ID-class TME therapy to the subject, wherein, prior to the administration, the subject is identified as exhibiting a combined biomarker comprising
(a) a negative Signature 1 score; and
(b) a negative Signature 2 score,
wherein
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject.

E48. A method for treating a human subject afflicted with a cancer comprising
(A) identifying, prior to the administration, a subject exhibiting a combined biomarker comprising
(a) a negative Signature 1 score; and
(b) a negative Signature 2 score, wherein
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject;
and,
(B) administering to the subject an ID-class TME therapy.

E49. A method for identifying a human subject afflicted with a cancer suitable for treatment with an ID-class TME therapy, the method comprising
(i) determining a Signature 1 score by measuring the expression levels of a gene panel selected from TABLE 3 (or FIGS. 28A-28G) in a first sample obtained from the subject; and,
(ii) determining a Signature 2 score by measuring the expression levels of a gene panel selected from TABLE 4 (or FIGS. 28A-28G) in a second sample obtained from the subject,
wherein the presence of a combined biomarker comprising
(a) a negative Signature 1 score; and
(b) a negative Signature 2 score, prior to the administration, indicates that a ID-class TME therapy can be administered to treat the cancer.

E50. The method of any one of embodiments E47 to E49, wherein the ID-class TME therapy comprises the administration of a checkpoint modulator therapy concurrently or after the administration of a therapy that initiates an immune response.

E51. The method of embodiment E50, wherein the therapy that initiates an immune response is a vaccine, a CAR-T, or a neo-epitope vaccine.

E52. The method of embodiment E50, wherein the checkpoint modulator therapy comprises the administration of an inhibitor of an inhibitory immune checkpoint molecule.

E53. The method of embodiment E52, wherein the inhibitor of an inhibitory immune checkpoint molecule is an antibody against PD-1, PD-L1, PD-L2, CTLA-4, or a combination thereof.

E54. The method of embodiment E53, wherein the anti-PD-1 antibody comprises nivolumab, pembrolizumab, cemiplimab, PDR001, CBT-501, CX-188, sintilimab, tislelizumab or TSR-042, or an antigen-binding portion thereof.

E55. The method of embodiment E53, wherein the anti-PD-1 antibody cross-competes with nivolumab, pembrolizumab, cemiplimab, PDR001, CBT-501, CX-188, sintilimab, tislelizumab, or TSR-042, for binding to human PD-1.

E56. The method of embodiment E53, wherein the anti-PD-1 antibody binds to the same epitope as nivolumab, pembrolizumab, cemiplimab, PDR001, CBT-501, CX-188, sintilimab, tislelizumab, or TSR-042.

E57. The method of embodiment E53, wherein the anti-PD-L1 antibody comprises avelumab, atezolizumab, CX-072, LY3300054, durvalumab, or an antigen-binding portion thereof.

E58. The method of embodiment E53, wherein the anti-PD-L1 antibody cross-competes with avelumab, atezolizumab, CX-072, LY3300054, or durvalumab for binding to human PD-L1.

E59. The method of embodiment E53, wherein the anti-PD-L1 antibody binds to the same epitope as avelumab, atezolizumab, CX-072, LY3300054, or durvalumab.

E60. The method of embodiment E53, wherein the anti-CTLA-4 antibody comprises ipilimumab or the bispecific antibody XmAb20717 (anti PD-1/anti-CTLA-4), or an antigen-binding portion thereof.

E61. The method of embodiment E53, wherein the anti-CTLA-4 antibody cross-competes with ipilimumab or the bispecific antibody XmAb20717 (anti PD-1/anti-CTLA-4) for binding to human CTLA-4.

E62. The method of embodiment E53, wherein the anti-CTLA-4 antibody binds to the same CTLA-4 epitope as ipilimumab or the bispecific antibody XmAb20717 (anti PD-1/anti-CTLA-4).

E63. The method of embodiment E50, where the check point modulator therapy comprises the administration of (i) an anti-PD-1 antibody selected from the group consisting of nivolumab, pembrolizumab, cemiplimab PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iv) an anti-CTLA-4 antibody, which is ipilimumab or the bispecific antibody XmAb20717 (anti PD-1/anti-CTLA-4), or (iii) a combination thereof.

E64. A method for treating a human subject afflicted with a cancer comprising administering an A-class TME therapy to the subject, wherein, prior to the administration, the subject is identified as exhibiting a combined biomarker comprising
(a) a positive Signature 1 score; and
(b) a negative Signature 2 score, wherein,
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject.

E65. A method for treating a human subject afflicted with a cancer comprising
(A) identifying, prior to the administration, a subject exhibiting a combined biomarker comprising
(a) a positive Signature 1 score; and,
(b) a negative Signature 2 score, prior to the administration,
wherein,
(i) the Signature 1 score is determined by measuring the expression levels of a gene panel selected from TABLE 3 (or FIGS. 28A-28G) in a first sample obtained from the subject; and,
(ii) the Signature 2 score is determined by measuring the expression levels of a gene panel selected from TABLE 4 (or FIGS. 28A-28G) in a second sample obtained from the subject;
and,
(B) administering to the subject an A-class TME therapy.

E66. A method for identifying a human subject afflicted with a cancer suitable for treatment with an A-class TME therapy, the method comprising
(i) determining a Signature 1 score by measuring the expression levels of a gene panel selected from TABLE 3 (or FIGS. 28A-28G) in a first sample obtained from the subject; and,
(ii) determining a Signature 2 score by measuring the expression levels of a gene panel selected from TABLE 4 (or FIGS. 28A-28G) in a second sample obtained from the subject,
wherein the presence of a combined biomarker comprising
(a) a positive Signature 1 score; and
(b) a negative Signature 2 score, prior to the administration,
indicates that a A-class TME therapy can be administered to treat the cancer.

E67. The method of embodiments E64 to E66, wherein the A-class TME therapy comprises a VEGF-targeted therapy and other anti-angiogenics, an inhibitor of angiopoietin 1 (Ang1), an inhibitor of angiopoietin 2 (Ang2), an inhibitor of DLL4, a bispecific of anti-VEGF and anti-DLL4, a TKI inhibitor, an anti-FGF antibody, an anti-FGFR1 antibody, an anti-FGFR2 antibody, a small molecule that inhibits FGFR1, a small molecule that inhibits FGFR2, an anti-PLGF antibody, a small molecule against a PLGF receptor, an antibody against a PLGF receptor, an anti-VEGFB antibody, an anti-VEGFC antibody, an anti-VEGFD antibody, an antibody to a VEGF/PLGF trap molecule such as aflibercept, or ziv-aflibercet, an anti-DLL4 antibody, or an anti-Notch therapy such as an inhibitor of gamma-secretase.

E68. The method of embodiment E67, wherein the TKI inhibitor is selected from the group consisting of cabozantinib, vandetanib, tivozanib, axitinib, lenvatinib, sorafenib, regorafenib, sunitinib, fruquitinib, pazopanib, and any combination thereof.

E69. The method of embodiment E68, wherein the TKI inhibitor is fruquintinib.

E70. The method of embodiment E67, wherein the VEGF-targeted therapy comprises the administration of an anti-VEGF antibody or an antigen-binding portion thereof.

E71. The method of embodiment E70, wherein the anti-VEGF antibody comprises varisacumab, bevacizumab, or an antigen-binding portion thereof.

E72. The method of embodiment E70, wherein the anti-VEGF antibody cross-competes with varisacumab, or bevacizumab for binding to human VEGF A.

E73. The method of embodiment E70, wherein the anti-VEGF antibody binds to the same epitope as varisacumab, or bevacizumab.

E74. The method of embodiment E67, wherein the VEGF-targeted therapy comprises the administration of an anti-VEGFR antibody.

E75. The method of embodiment E74, wherein the anti-VEGFR antibody is an anti-VEGFR2 antibody.

E76. The method of embodiment E75, wherein the anti-VEGFR2 antibody comprises ramucirumab or an antigen-binding portion thereof.

E77. The method of any one of embodiments E64 to E76, wherein the A-class TME therapy comprises the administration of an angiopoietin/TIE2-targeted therapy.

E78. The method of embodiment E77, wherein the angiopoietin/TIE2-target therapy comprises the administration of endoglin and/or angiopoietin.

E79. The method of any one of embodiments E64 to E78, wherein the A-class TME therapy comprises the administration of a DLL4-targeted therapy.

E80. The method of embodiment E79, wherein the DLL4-targeted therapy comprises the administration of navicixizumab, ABL101 (NOV1501), or ABT165.

E81. The method of any one of embodiments E1 to E80, comprising
(a) administering chemotherapy;
(b) performing surgery;
(c) administering radiation therapy; or,
(d) any combination thereof.

E82. The method of any one of embodiments E1 to E81, wherein the gene panel selected from TABLE 4 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes selected from TABLE 2, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 genes selected from FIGS. 28A-28G.

E83. The method of any one of embodiments E1 to E82, wherein the gene panel is a gene panel selected from TABLE 4, or from FIGS. 28A-28G.

E84. The method of any one embodiments ES1 to E83, wherein the gene panel selected from TABLE 3 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 genes selected from TABLE 1, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 genes selected from FIG. 28A-28G.

E85. The method of any one of embodiments E1 to E84, wherein the gene panel is a gene panel selected from TABLE 3, or from FIGS. 28A-28G.

E86. The method of any one of embodiments E1 to E85, wherein the first sample and the second sample are the same sample.

E87. The method of any one of embodiments E1 to E85, wherein the first sample and the second sample are different samples.

E88. The method of any one of embodiments E1 to E87, wherein the first sample and/or the second sample comprises intratumoral tissue.

E89. The method of any one of embodiments E1 to E88, wherein the expression levels are expressed protein levels.

E90. The method of any one of embodiments E1 to E88, wherein the expression levels are transcribed RNA expression levels.

E91. The method of any one of embodiments E1 to E90, wherein the RNA expression levels are determined using sequencing or any technology that measures RNA.

E92. The method of embodiment E91, wherein the sequencing is Next Generation Sequencing (NGS).

E93. The method of embodiment E92, wherein the NGS is selected from the group consisting of RNA-Seq, EdgeSeq, PCR, Nanostring, WES, or combinations thereof.

E94. The method of embodiment E90, wherein the RNA expression levels are determined using fluorescence.

E95. The method of embodiment E90, wherein the RNA expression levels are determined using an Affymetrix microarray or an Agilent microarray.

E96. The method of embodiments E90 to E95, wherein RNA expression levels are subject to quantile normalization.

E97. The method of embodiment E96, wherein the quantile normalization comprises binning input RNA level values into quantiles.

E98. The method of embodiment E97, wherein the input RNA levels are binned into 100 quantiles.

E99. The method of embodiments E96 to E98, wherein the quantile normalization comprises quantile transforming the RNA expression levels to a normal output distribution function.

E100. The method of any one of embodiments E1 to E99, wherein the calculation of a signature score comprises
(i) measuring the expression level for each gene in the gene panel in a test sample from the subject;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and,
(iv) adding all the values obtained in step (iii) and dividing the resulting number by the square root of the number of genes in the gene panel;
wherein if the value obtained in (iv) is above zero, the signature score is a positive signature score, and wherein if the value obtained in (iv) is below zero, the signature score is a negative signature score.

E101. The method of embodiment E100, wherein the reference sample comprises a collection of reference expression levels.

E102. The method of embodiment E101, wherein the reference expression values are standardized reference values.

E103. The method of embodiment E101, wherein the reference expression values are obtained from a sample population.

E104. The method of embodiment E101, wherein the reference expression levels are derived from a publicly available database or a combination of databases that are normalized to one another.

E105. The method of embodiment E100, wherein the reference sample comprises a tissue sample obtained from a different population.

E106. The method of any one of embodiments E100 to E105, wherein the reference sample comprises a sample taken at a different time point.

E107. The method of embodiment E106, wherein the different time point is an earlier time point.

E108. The method of any one of embodiments E1 to E107, wherein the cancer is a tumor.

E109. The method of embodiment E108, wherein the tumor is a carcinoma.

E110. The method of embodiment E108, wherein the tumor is selected from the group consisting gastric cancer, colorectal cancer, liver cancer (hepatocellular carcinoma, HCC), ovarian cancer, breast cancer, NSCLC, bladder cancer, lung cancer, pancreatic cancer, head and neck cancer, lymphoma, uterine cancer, renal or kidney cancer, biliary cancer, prostate cancer, testicular cancer, urethral cancer, penile cancer, thoracic cancer, rectal cancer, brain cancer (glioma and glioblastoma), cervicalparotid cancer, esophageal cancer, gastroesophageal cancer, larynx cancer, thyroid cancer, adenocarcinomas, neuroblastomas, melanoma, and Merkel Cell carcinoma.

E111. The method of any one of embodiments E1 to E110, wherein the cancer is relapsed.

E112. The method of any one of embodiments E1 to E110, wherein the cancer is refractory.

E113. The method of embodiment E112, wherein the cancer is refractory following at least one prior therapy comprising administration of at least one anticancer agent.

E114. The method of any one of embodiments E1 to E113, wherein the cancer is metastatic.

E115. The method of any one of embodiments E2 to E114, wherein the administering effectively treats the cancer.

E116. The method of any one of embodiments E2 to E115, wherein the administering reduces the cancer burden.

E117. The method of embodiment E116, wherein cancer burden is reduced by at least about 10%, at least about 20%, at least about 30%, at least about 40%, or about 50% compared to the cancer burden prior to the administration.

E118. The method of any one of embodiments E2 to E117, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

E119. The method of any one of embodiments E2 to E118, wherein the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

E120. The method of any one of embodiments E2 to E119, wherein the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

E121. The method of any one of embodiments E2 to E120, wherein the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

E122. The method of any one of embodiments E2 to E121, wherein the administering improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject not exhibiting the combined biomarker.

E123. The method of any one of embodiments E2 to E122, wherein the administering improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not exhibiting the combined biomarker.

E124. A kit comprising (i) a plurality of oligonucleotide probes capable of specifically detecting an RNA encoding a gene biomarker from TABLE 1, and (ii) a plurality of oligonucleotide probes capable of specifically detecting an RNA encoding a gene biomarker from TABLE 2.

E125. An article of manufacture comprising (i) a plurality of oligonucleotide probes capable of specifically detecting an RNA encoding a gene biomarker from TABLE 1 (or FIGS. 28A-28G), and (ii) a plurality of oligonucleotide probes capable of specifically detecting an RNA encoding a gene biomarker from TABLE 2 (or FIGS. 28A-28G), wherein the article of manufacture comprises a microarray.

E126. A gene panel comprising at least a biomarker gene from TABLE 1 (or FIG. 28A-28G) and a biomarker gene from TABLE 2 (or FIGS. 28A-28G), for use in determining the tumor microenvironment of a tumor in a subject in need thereof, wherein the tumor microenvironment is used for
(i) identifying a subject suitable for an anticancer therapy;
(ii) determining the prognosis of a subject undergoing anticancer therapy;
(iii) initiating, suspending, or modifying the administration of an anticancer therapy; or,
(iv) a combination thereof.

E127. A combined biomarker for identifying a human subject afflicted with a cancer suitable for treatment with an anticancer therapy, wherein the combined biomarker comprises a Signature 1 score and a Signature 2 score measured in a sample obtained from the subject wherein
(i) the Signature 1 score is determined by measuring the expression levels of the genes in a gene panel of TABLE 3 (or FIGS. 28A-28G) in a first sample obtained from the subject; and,
(ii) the Signature 2 score is determined by measuring the expression levels of the genes in a gene panel of TABLE 4 (or FIGS. 28A-28G) in a second sample obtained from the subject,
and wherein
the therapy is an IA Class TME therapy if the Signature 1 score is negative and the Signature 2 score is positive;
the therapy is an IS Class TME therapy if the Signature 1 score is positive and the Signature 2 score is positive;
the therapy is an ID Class TME therapy if the Signature 1 score is negative and the Signature 2 score is negative;
the therapy is an A Class TME therapy if the Signature 1 score is positive and the Signature 2 score is negative.

E128. An anticancer therapy for treating a cancer in a human subject in need thereof, wherein the subject is identified as exhibiting a combined biomarker comprising a Signature 1 score and a Signature 2 score, wherein
(i) the Signature 1 score is determined by measuring the expression levels of the genes in a gene panel of TABLE 3 (or FIGS. 28A-28G) in a first sample obtained from the subject; and,
(ii) the Signature 2 score is determined by measuring the expression levels of the genes in a gene panel of TABLE 4 (or FIGS. 28A-28G) in a second sample obtained from the subject,
and wherein
the therapy is an IA-Class TME therapy if the Signature 1 score is negative and the Signature 2 score is positive;
the therapy is an IS-Class TME therapy if the Signature 1 score is positive and the Signature 2 score is positive;
the therapy is an ID-Class TME therapy if the Signature 1 score is negative and the Signature 2 score is negative;
the therapy is an A-Class TME therapy if the Signature 1 score is positive and the Signature 2 score is negative.

### EXAMPLES

### Example 1

### Tumor Microenvironment (TME) Classification: Population-Based Classifier

The present disclosure describes the methodology to create a population-based Z-score classifier (a population-based classifier) that is able to stratify (or classify) tumor samples into four classes based on gene expression. As used herein, the four classes can also be referred to as tumor microenvironments (TME), stromal types, stromal subtypes, or phenotypes, or variations thereof. Also herein is described the analytical pipelines used to generate expression values from raw microarray (RNA) and RNA-sequencing data.

For data preprocessing, various technologies exist for measuring gene expression where each platform technology requires specific preprocessing of the raw data. The population-based classifier supports Affymetrix DNA microarray, high throughput next generation RNA sequencing, and in some aspects, can be extended to other technologies.

For microarray data, the Affymetrix chip procedure measures the intensity pixel values per cell (each containing a unique probe) which are stored in a CEL file. CEL files were processed using the Affy R package. The expresso function was applied using the following parameters: RMA (Robust Multichip Average) background correction method, quantile normalization, no probe-specific correction, and medianpolish summarization (J. W. Tukey, Exploratory Data Analysis, Addison-Wesley, 1977). The expression values returned by the expresso function were log 2-transformed. Finally, expressions were quantile transformed to normal output distribution, binning input values into 100 quantiles (FIG. 1).

Illumina RNA-Seq sequencing reads were processed by cleaning up reads, aligning them to a reference genome and quantifying gene expression. The analysis steps thus included three key steps: trimming (BBDuk), mapping (STAR), and expression quantification (featureCounts). Reference human genome was Ensembl, version 92, extended with references for common spike-in standards (ERCC and SIRV). As an additional quality control step, a subsample of a million reads (Seqtk tool) was mapped to rRNA and globin sequences of the selected species to determine the overall proportion of these kinds of reads in the sample. Results were reported in the summary table of the multiqc report.

Raw and normalized (TPM, FPKM) expression values were generated using the cloud-based Genialis Expressions software, and reported together with all the technical details needed to reproduce them. Prior to stratifying the samples with the Z-score-based model, TPM normalized expressions were quantile transformed to normal output distribution, binning input values into 100 quantiles (FIG. 1).

For other platform technologies, for example EdgeSeq (HTG Molecular Diagnostics, Inc.), quantile normalization should be used for cross-platform analysis, binning input values into 100 quantiles and applying a normal output distribution function. The accuracy of any method increases with the population distribution reaching normal distribution.

Classification of samples. The population-based classifier (or population-based method) of the present disclosure assumed a zero-centered normal distribution (μ=0) of gene expression levels.

Across the whole patient population, the mean and standard deviation per gene were calculated from the expression levels of that gene. For an individual patient, per each of the genes, the patient's standardized expression level was taken, the population mean was subtracted, then divided by the standard deviation. This was the Z-score. In some aspects, there was no correction for degrees of freedom.

For an individual patient, all the Z-scores within a Signature were added and then divided by the square root of the number of genes. The result was the Activation Score, zₛ, according to Equation 1:

$${z}_{s} = {\sum }_{g ∈ G} {z}_{s , g} / \sqrt{\vert G \vert} , \qquad ( Equation 1 )$$

where z refers to Z-score, s to sample (patient), g to gene, and G to the Signature geneset. |G| indicates the size of geneset G. When the Activation Score was greater than zero, i.e., zₛ>=0, then that Signature was said to be positive, and so zₛ<0 meant negative for Signature. z_{s,g} is a vector that describes the magnitude and direction away from the mean of population, and is unitless; the Activation Score zₛ is also unitless.

Prognostications or predictions were made by correlating the Activation Score with TABLE 13. Put another way, based on the sign of patient Z-scores, and thresholds used (positive or negative zₛ), the patients were classified into one of the four stromal subtypes, by applying the rules in TABLE 13 (patient classification rules based on the sign of the summed Signature 1 and Signature 2 Z-scores). See also FIG. 10.

TABLE 13
Prognostic or Predictive Biologies of Four Classes of Stromal Subtypes
based on Activation Scores for Signature 1 and Signature 2 genes.

| Signature 1 | Signature 2 | Class of Stromal Subtype |
|---|---|---|
| − | + | IA (Immune Active) |
| + | + | IS (Immune Suppressed) |
| − | − | ID (Immune Desert) |
| + | − | A (Angiogenic) |

The first biological signature, Signature 1, was determined by the Activation Score, zₛ of one or more (e.g., at least 1, 2, 3, 4, 5, 10, 15, 20, 25, 30, 40, 50, 60, 61, 62, or 63) of the genes in TABLE 1.

In some aspects, the genes, which can also be called biomarkers in the present disclosure, included one or more of the following: ABCC9, AFAP1L2, BACE1, BGN, BMP5, COL4A2, COL8A1, COL8A2, CPXM2, CXCL12, EBF1, ECM2, EDNRA, ELN, EPHA3, FBLN5, GNAS, GNB4, GUCY1A3, HEY2, HSPB2, IL1B, ITGA9, ITPR1, JAM2, JAM3, KCNJ8, LAMB2, LHFP, LTBP4, MEOX1, MGP, MMP12, MMP13, NAALAD2, NFATC1, NOV, OLFML2A, PCDH17, PDE5A, PDGFRB, PEG3, PLSCR2, PLXDC2, RGS4, RGS5, RNF144A, RRAS, RUNX1T1, CAV2, SELP, SERPINE2, SGIP1, SMARCA1, SPON1, STAB2, STEAP4, TBX2, TEK, TGFB2, TMEM204, TTC28, and UTRN.

The second biological signature, Signature 2, was determined by the sum of Activation Score, zₛ, of one or more (e.g., at least 1, 2, 3, 4, 5, 10, 15, 20, 25, 30, 40, 50, 60, or 61) of the genes in TABLE 2.

In some aspects, the genes included one or more of the following: AGR2, C11orf9, DUSP4, EIF5A, ETV5, GAD1, IQGAP3, MST1, MT2A, MTA2, PLA2G4A, REG4, SRSF6, STRN3, TRIM7, USF1, ZIC2, C10orf54, CCL3, CCL4, CD19, CD274, CD3E, CD4, CD8B, CTLA4, CXCL10, IFNA2, IFNB1, IFNG, LAG3, PDCD1, PDCD1LG2, TGFB1, TIGIT, TNFRSF18, TNFRSF4, TNFSF18, TLR9, HAVCR2, CD79A, CXCL11, CXCL9, GZMB, IDO1, IGLL5, ADAMTS4, CAPG, CCL2, CTSB, FOLR2, HFE, HMOX1, HP, IGFBP3, MEST, PLAU, RAC2, RNH1, SERPINE1, and TIMP1.

### Example 2

### Application of Classifiers to Public Datasets

The classifiers described in Example 1 were used to analyze three publicly available datasets according to the population-based method, or classifier, as described herein. Datasets were normalized as described herein (FIG. 1). In FIG. 1, the top row of histograms shows the distribution of log 2 expressions of the Signature 1 and 2 genes, and shows that the datasets have different ranges and distributions. The RNA expression levels in the ACRG and Singapore were analyzed by micro-array (Affymetrix), whereas the RNA expression levels in the TCGA data are derived from RNA sequencing.

In the middle row of plots of FIG. 1, the population medians and Z-scores were computed. The distributions were all centered around 0 as expected, but that the overall shape of the distributions are different due to platform differences (micro-array and RNA-Seq). The bottom row of panels of FIG. 1 shows the expression (Z-score) values after quantile normalization. As a result of the normalization, it was possible to classify relative to the median across all three datasets.

The population-based method of the present disclosure was used to classify 298 patients of the Asian Cancer Research Group (ACRG) dataset into four stromal subtypes. The ACRG is a not-for-profit pharmaceutical industry consortium that provides curated and comprehensive genomic datasets of patients affected with the most commonly diagnosed cancers in Asia (liver, gastric, and lung). The RNA expression data in the ACRG dataset was provided as Affymetrix microarray data. There are 300 patients in the gastric cancer dataset, for which two patients' outcome data (overall survival) are not available. Thus, some tables in the present disclosure refer to 298 patients, while other tables or figures can refer to 300 patients. The patients received chemotherapy only, and overall survival rates were curated by the consortium.

Gastric cancer data from The Cancer Genome Atlas (TCGA) Program (available at www.cancer.gov/about-nci/organization/ccg/research/structural-genomics/tcga) was used for the population-based method of the present disclosure was used to classify 388 patients into four stromal subtypes. The RNA expression data in TCGA was provided as RNA-Seq, and outcome data was provided as overall survival for 388 patients, however not all co-variate data was available, so certain tables and figures herein refer to smaller subsets of patients.

The Singapore gastric cancer dataset, or Singapore cohort, as used by the inventors, comes from the Gastric Cancer Project '08, as found at www.ncbi.nlm.nih.gov/geo/query/acc.cgi?acc=GSE15459. Two hundred primary gastric tumors were profiled on Affymetrix GeneChip Human Genome U133 Plus 2.0 Array, of which 192 were used (Liu, et al., (2013) Gastroenterology). Outcome data was reported as overall survival in Lei Z, Tan I B, Das K, Deng N et al. Identification of molecular subtypes of gastric cancer with different responses to PI3-kinase inhibitors and 5-fluorouracil. Gastroenterology 2013 September; 145(3):554-65.

The population-based method, with the threshold set to the mean, or zero, was used to classify each of the three datasets. TABLE 14 shows the distribution of the four stromal subtypes of the patients in each of the three cohorts after classification.

TABLE 14
Prevalence of the four classes of stromal subtypes of the present
disclosure in three publicly available gastric cancer datasets (ACRG,
TCGA, and Singapore).

| Stromal | | | |
|---|---|---|---|
| Subtype | ACRG | TCGA | Singapore |
| A | 15.2% | 19.5% | 24.4% |
| IA | 26.5% | 20.7% | 27.5% |
| ID | 34.8% | 32.6% | 23.1% |
| IS | 23.5% | 27.2% | 25.1% |

Tumor subtypes, as defined by ACRG, were compared to the four stromal subtypes. ACRG tumor subtypes in the dataset are not strongly associated with stromal subtypes of the present disclosure. The ACRG data was described as having 4 tumor subtypes: MSI—Microsatellite Instable; MSS—Microsatellite Stable/EMT—Epithelial-mesenchymal transition (occurs during wound healing and the initiation of metastasis in cancer); TP53-, the normal phenotype for (tumor) protein p53; and TP53+ is abnormal phenotype for (tumor) protein p53 (TABLE 15).

TABLE 15
Tumor subtypes in the ACRG Dataset, n = 300, were not strongly
associated with the four stromal subtypes.

| Stromal | MSI | MSS/EMT | MSS/TP53− | MSS TP53+ |
|---|---|---|---|---|
| Subtype | N = 68 | N = 46 | N = 107 | N = 79 |
| IA | 43 (63%) | 0 (0%) | 20 (19%) | 22 (28%) |
| ID | 12 (18%) | 0 (0%) | 54 (50%) | 33 (42%) |
| A | 0 (0%) | 26 (57%) | 13 (12%) | 7 (9%) |
| IS | 13 (19%) | 20 (43%) | 20 (19%) | 17 (22%) |

TCGA described four gastric cancer subtypes. TCGA gastric cancer subtypes C1, C2, C3, and C4 (n=232) were compared to stromal subtypes, as classified according to the present disclosure, and the analysis reveals no strong association between the gastric cancer subtypes and the stromal subtypes (TABLE 16).

TABLE 16
TCGA gastric cancer subtypes C1, C2, C3, and C4 (n = 232) compared
to stromal subtypes reveals no strong association.

| Stromal | | | | |
|---|---|---|---|---|
| Subtype | Cl | C2 | C3 | C4 |
| N = 32 | N = 47 | N = 53 | N = 89 | N = 43 |
| IA | 0 (0%) | 23 (43%) | 22 (25%) | 14 (33%) |
| ID | 0 (0%) | 2 (4%) | 38 (43%) | 0 (0%) |
| A | 23 (49%) | 4 (8%) | 16 (18%) | 8 (19%) |
| IS | 24 (51%) | 24 (45%) | 13 (15%) | 13 (30%) |

The Singapore gastric cancer dataset were reported with four distinct cancer subtypes: Mesenchymal, Metabolic, Proliferative, and Unstable. TABLE 17 shows the lack of correlation between the stromal subtypes of the 192 patients classified with the population-based method (threshold at the mean, or zero) of the present disclosure.

TABLE 17
The Singapore dataset gastric cancer subtypes (Mesenchymal,
Metabolic, Proliferative, and Unstable) did not associate strongly
with the stromal subtypes.

| Stromal | Mesenchymal | Metabolic | Proliferative | Unstable |
|---|---|---|---|---|
| Subtype | N = 51 | N = 40 | N = 70 | N = 31 |
| IA | 0 (0%) | 6 (15%) | 30 (43%) | 4 (6%) |
| ID | 0 (0%) | 25 (63%) | 22 (31%) | 7 (23%) |
| A | 32 (63%) | 3 (8%) | 6 (9%) | 6 (19%) |
| IS | 19 (37%) | 6 (15%) | 12 (17%) | 14 (45%) |

For all patients of the three datasets for whom the co-variate of age was reported, the relationship of age to the four stromal subtypes of the classified patients was explored (TABLE 18). There was no obvious association between age and the four stromal subtypes, when the patients of all three datasets were classified with the population-based method (threshold at the mean, or zero) of the present disclosure.

TABLE 18
The co-variate of age did not associate with the stromal subtypes of the present
disclosure in the three publicly available gastric cancer datasets. Only 252 of the 388 subjects
reported age for TCGA data cohort, while age was reported for all 300 ACRG and 192 Singapore
patients.

| | IA | ID | IS | A | Overall |
|---|---|---|---|---|---|
| ACRG | N = 85 | N = 99 | N = 70 | N = 46 | N = 300 |
| Age 20-29 | 0 (0%) | 1 (1%) | 0 (0%) | 1 (2.2%) | 2 (0.6%) |
| 30-39 | 4 (4.7%) | 5 (5.1%) | 2 (2.9%) | 1 (2.2%) | 12 (4%) |
| 40-49 | 4 (4.7%) | 4 (4.0%) | 5 (7.1%) | 11 (23.9%) | 24 (8%) |
| 50-59 | 20 (23.5%) | 14 (14%) | 20 (28.6%) | 14 (30.4%) | 68 (22.6%) |
| 60-69 | 27 (31.8%) | 46 (46.5%) | 26 (37.1%) | 11 (23.9%) | 110 (36.6%) |
| 70-79 | 25 (29.4%) | 28 (28.3%) | 15 (21.4%) | 6 (13.0%) | 74 (24.6%) |
| 80-89 | 5 (5.9%) | 1 (1%) | 2 (2.9%) | 2 (4.3%) | 10 (3.3%) |
| | IA | ID | IS | A | Overall |
| TCGA | N = 63 | N = 59 | N = 78 | N = 52 | N = 252 |
| Age 20-29 | 0 (0%) | 0 (0%) | 0 (0%) | 0 (0%) | 0 (0%) |
| 30-39 | 0 (0%) | 1 (1.7%) | 0 (0%) | 1 (1.9%) | 2 (0.7%) |
| 40-49 | 1 (1.6%) | 3 (5.1%) | 8 (10.3%) | 4 (7.7%) | 16 (6.3%) |
| 50-59 | 14 (22.2%) | 14 (23.7%) | 19 (24.4%) | 15 (28.8%) | 62 (24.6%) |
| 60-69 | 17 (27.0%) | 20 (33.9%) | 22 (28.2%) | 14 (26.9%) | 73 (29.0%) |
| 70-79 | 24 (38.1%) | 17 (28.8%) | 21 (26.9%) | 17 (32.7%) | 79 (31.3%) |
| 80-89 | 7 (11.1%) | 4 (6.8%) | 8 (10.3%) | 1 (1.9%) | 20 (7.9%) |
| | IA | ID | IS | A | Overall |
| Singapore | N = 40 | N = 54 | N = 51 | N = 47 | N = 192 |
| Age 20-29 | 0 (0%) | 1 (1.9%) | 1 (2%) | 2 (4.3%) | 4 (2.1%) |
| 30-39 | 0 (0%) | 2 (3.7%) | 4 (7.8%) | 1 (2.1%) | 7 (3.6%) |
| 40-49 | 5 (12.5%) | 3 (5.6%) | 6 (11.8%) | 4 (8.5%) | 18 (9.4%) |
| 50-59 | 4 (10.0%) | 9 (16.7%) | 7 (13.7%) | 10 (21.3%) | 30 (15.6%) |
| 60-69 | 10 (25.0%) | 21 (38.9%) | 17 (33.3%) | 18 (38.3%) | 66 (34.4%) |
| 70-79 | 17 (42.5%) | 11 (20.4%) | 14 (27.5)% | 8 (17.0%) | 50 (26.0%) |
| 80-89 | 4 (10.0%) | 7 (13.0%) | 2 (3.9%) | 4 (8.5%) | 17 (8.9%) |

For all patients of the three datasets for whom the co-variate of gender was reported, the relationship of gender to the four stromal subtypes of the classified patients was explored (TABLE 19). There was no obvious association between gender and the four stromal subtypes, when the patients of all three datasets were classified with the population-based method (threshold at the mean, or zero) of the present disclosure.

TABLE 19
The co-variate of gender did not associate with the stromal subtypes of the present
disclosure in the three publicly available gastric cancer datasets. Only 254 of the 388 subjects
report gender for TCGA data cohort.

| | IA | ID | IS | A | Overall |
|---|---|---|---|---|---|
| ACRG | N = 85 | N = 99 | N = 70 | N = 46 | N = 300 |
| Male | 57 (67%) | 75 (75.7%) | 42 (60%) | 25 (54.3%) | 199 (66.3%) |
| Female | 28 (33%) | 24 (24.3%) | 28 (40%) | 21 (45.7%) | 101 (33.7%) |
| | IA | ID | IS | A | Overall |
| TCGA | N = 65 | N = 59 | N = 78 | N = 52 | N = 254 |
| Male | 36 (55.4%) | 41 (69.5%) | 50 (64.1%) | 21 (59.6%) | 158 (66.3%) |
| Female | 29 (44.6%) | 18 (30.5%) | 28 (35.9%) | 31 (40.4%) | 96 (33.7%) |
| | IA | ID | IS | A | Overall |
| Singapore | N = 40 | N = 54 | N = 51 | N = 47 | N = 192 |
| Male | 25 (62.5%) | 40 (74.1%) | 33 (64.7%) | 27 (57.4%) | 125 (65.1%) |
| Female | 15 (37.5%) | 14 (25.9%) | 18 (35.3%) | 20 (42.6%) | 67 (34.9%) |

For all patients of the three datasets for whom the co-variate of cancer stage was reported, the relationship of cancer stage to the four stromal subtypes of the classified patients was explored (TABLE 20). There was no obvious association between cancer stage and the four stromal subtypes, when the patients of all three datasets were classified with the population-based method (threshold at the mean, or zero) disclosed herein.

TABLE 20
The co-variate of cancer stage did not correlate with the stromal subtypes of the
present disclosure in the three publicly available gastric cancer datasets. 298 of 300 subjects
reported stage of disease in the ACRG; 375 of the 388 TCGA data subjects reported stage;192
Singapore subjects reported stage.

| | IA | ID | IS | A | Overall |
|---|---|---|---|---|---|
| ACRG | N = 85 | N = 98 | N = 69 | N = 46 | N = 298 |
| Stage 1 | 6 (7.1%) | 8 (8.2%) | 9 (13%) | 7 (15.2%) | 30 (10.1%) |
| 2 | 26 (30.6%) | 29 (29.6%) | 25 (36.2%) | 16 (34.8%) | 96 (32.2%) |
| 3 | 29 (34.1%) | 33 (33.7%) | 20 (29%) | 13 (28.3%) | 95 (31.9%) |
| 4 | 24 (28.2%) | 28 (28.65%) | 15 (21.7%) | 10 (21.7%) | 77 (25.6%) |
| | IA | ID | IS | A | Overall |
| TCGA | N = 86 | N = 117 | N = 100 | N = 72 | N = 375 |
| Stage 1 | 13 (15.1%) | 24 (20.5%) | 7 (7.0%) | 7 (9.7%) | 51 (13.6%) |
| 2 | 29 (33.7%) | 34 (29.1%) | 32 (32.0%) | 26 (36.1%) | 121 (32.3%) |
| 3 | 34 (39.5%) | 49 (41.9%) | 52 (52.0%) | 30 (41.7%) | 165 (44.0%) |
| 4 | 10 (11.6%) | 10 (8.5%) | 9 (9.0%) | 9 (12.5%) | 38 (10.1%) |
| | IA | ID | IS | A | Overall |
| Singapore | N = 40 | N = 54 | N = 51 | N = 47 | N = 192 |
| Stage 1 | 8 (20.0%) | 12 (22.2%) | 1 (2.0%) | 10 (21.3%) | 31 (16.1%) |
| 2 | 3 (7.5%) | 10 (18.5%) | 9 (17.6%) | 7 (14.9%) | 29 (15.1%) |
| 3 | 19 (47.5%) | 17 (31.5%) | 16 (31.4%) | 20 (42.6%) | 72 (37.5%) |
| 4 | 10 (25.0%) | 15 (27.8%) | 25 (49.0%) | 10 (21.3%) | 60 (31.3%) |

For all patients of the ACRG for whom the co-variate of Lauren Tumor Classification was reported, the relationship of Lauren Tumor Classification to the four stromal subtypes of the classified patients was explored (TABLE 21). Lauren Tumor Classification of gastric tumors is known in the art; there are three types: diffuse, intestinal, and mixed. There was no obvious association between Lauren Tumor Classification and the four stromal subtypes, when the ACRG patients were classified with the population-based method (threshold at the mean, or zero) of the present disclosure.

TABLE 21
Comparison of stromal subtypes of the present disclosure
(population-based) to the Lauren Tumor Classification of the
ACRG gastric cancer dataset, n = 300.

| | Diffuse | Intestinal | Mixed |
|---|---|---|---|
| Stromal Subtype | N = 142 | N = 150 | N = 8 |
| IA | 32 (23%) | 50 (33%) | 3 (38%) |
| ID | 29 (20%) | 68 (45%) | 2 (25%) |
| A | 34 (24%) | 11 (7%) | 1 (13%) |
| IS | 47 (33%) | 21 (14%) | 2 (25%) |

Survival curves, known in the art as Kaplan-Meier curves, were generated based on the three datasets individually and combined, according to the population-based method of the present disclosure (threshold set to the mean, or zero, unless indicated otherwise).

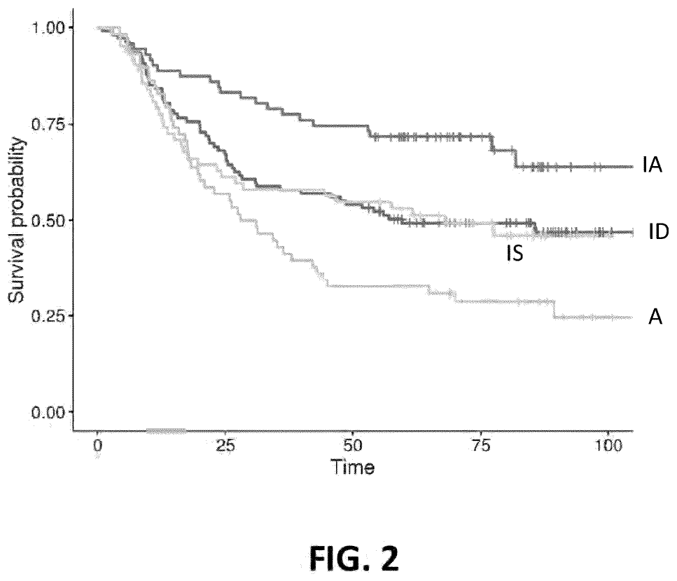

FIG. 2, a Kaplan-Meier Plot depicts survival curves, plotted as Survival Probability on the y-axis versus Time (in months) on the x-axis, for the classified ACRG cohort. Survival outcomes were statistically different between the stromal subtypes ID and IA, as well as ID and A, but not between ID and IS; see also TABLE 22. The most favorable stromal subtype for survivability was IA, or Immune Active, consistent with the observation that gastric cancer patients with immune-inflamed tumors have the best prognosis. The A and IS groups represent the worst survival risk.

In the IA patients, immune cells were mounting a response to the neoantigen load of the cancer. The IS, or Immune Suppressed, patients were not mounting an immune response to the cancer. ID, or Immune Desert, patients were not having a lot of transcription of stromal genes tabulated in TABLE 1 and TABLE 2 of the present disclosure. The patients appeared to not be mounting an immune response, but nor were they having angiogenic proliferation. A, or Angiogenic patients, likely had a rapidly proliferating tumor vasculature.

TABLE 22
Data corresponding to the Survival Risk Curve of FIG. 2 of the ACRG
dataset, classified using the population-based method, (threshold set to
the mean, or zero).

| Risk curve comparison from Kaplan | | | Log rank |
|---|---|---|---|
| Meier-Plot of ACRG data | HR | 95% CI | P-value |
| ID versus IA | 0.519 | 0.316-0.851 | 0.023 |
| ID versus A | 1.611 | 1.078-2.41 | 0.026 |
| ID versus IS | 1.059 | 0.685-1.637 | 0.8110 |

TABLE 22 revealed that survival outcomes were statistically different between ID and IA, as well as ID and A, but not between ID and IS. In this survival analysis, the hazard ratio (HR) was the ratio of the hazard rates corresponding to the conditions described by two levels of an explanatory variable. In this example, the HR of 0.519 between ID and IA showed increased risk of death for the ID stromal subtype.

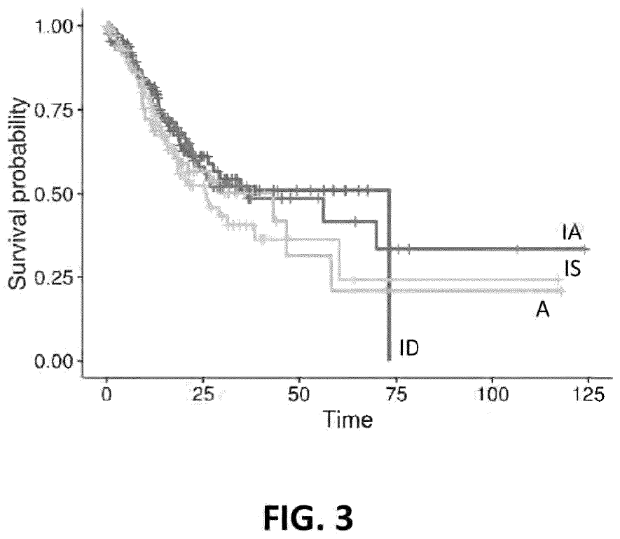

FIG. 3, a Kaplan-Meier Plot depicts survival curves, plotted as Survival Probability on the y-axis versus Time (in months) on the x-axis, for the classified TCGA cohort. In the TCGA dataset, the survival outcomes were not as statistically different between several stromal subtypes as seen in the ACRG dataset (TABLE 23; also compare to FIG. 2 and TABLE 22). However, when all three datasets were combined (the Singapore dataset is described below), the survival outcomes of the four classes of stromal subtypes, the data became statistically significant (see TABLE 25).

TABLE 23
Data corresponding to the Survival Risk Curve of FIG. 3 of the TCGA
dataset, classified using the population-based method.

| Risk curve comparison from | | | |
|---|---|---|---|
| Kaplan Meier-Plot of TCGA | | | Log rank |
| data | HR | 95% CI | P-value |
| ID versus IA | 1.068 | 0.683-1.668 | 0.811 |
| ID versus A | 1.296 | 1.296-2.068 | 0.539 |
| ID versus IS | 1.400 | 0.922-2.124 | 0.539 |

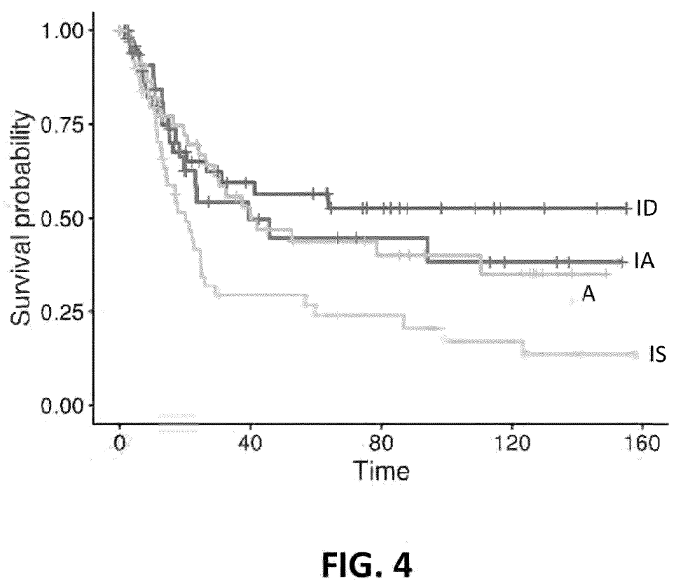

FIG. 4, a Kaplan-Meier Plot depicts survival curves, plotted as Survival Probability on the y-axis versus Time (in months) on the x-axis, for the classified Singapore cohort. In the Singapore dataset, the survival outcomes were not as statistically different between several stromal subtypes as seen in the ACRG dataset (TABLE 24; also compare to FIG. 2 and TABLE 22). However, the data became statistically significant when all three datasets were combined (see TABLE 25).

TABLE 24
Data corresponding to the Survival Risk Curve of FIG. 3 of
the Singapore dataset, classified using the population-based method.

| Risk curve comparison from | | | |
|---|---|---|---|
| Kaplan Meier-Plot of Singapore | | | Log rank |
| data | HR | 95% CI | P-value |
| ID versus IA | 0.869 | 0.547-1.383 | 0.0588 |
| ID versus A | 1.264 | 0.796-2.007 | 0.4772 |
| ID versus IS | 1.416 | 0.944-2.122 | 0.2970 |

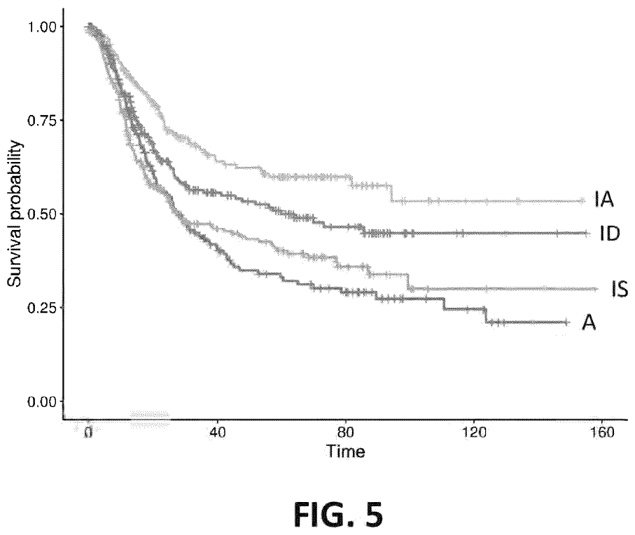

The Kaplan-Meier plot for the three combined datasets, classified with a threshold of zero, or the mean, can be seen in FIG. 5. Survival Probability was plotted on the y-axis versus Time (in months) on the x-axis. The statistics are reported in TABLE 25. The number of patients in each class when all the ACRG, TCGA, and Singapore datasets were combined was as follows: Class ID, n=286, or 32.5%; Class IA, n=199, or 22.6%; Class A, n=182, or 20.7%; Class IS, n=213, or 24.2%. Survival outcomes were statistically different between the stromal subtypes ID and IA, but not between ID and A, or ID and IS; see also TABLE 25. These data suggest that the different stromal biologies described by these subtypes differentially correlate to cancer outcomes.

TABLE 25
Data corresponding to the Combined Survival Risk
Curve of FIG. 5, classified using the population-based method.

| Risk curve comparison from | | | |
|---|---|---|---|
| Kaplan Meier-Plot of | | | |
| Combined ACRG, TCGA and | | | Log rank |
| Singapore data | HR | 95% CI | P-value |
| ID compared to IA | 0.731 | 0.544-0.982 | 0.0614 |
| ID compared to IS | 1.391 | 1.079-1.794 | 0.0246 |
| ID compared to A | 1.287 | 0.985-1.681 | 0.0678 |

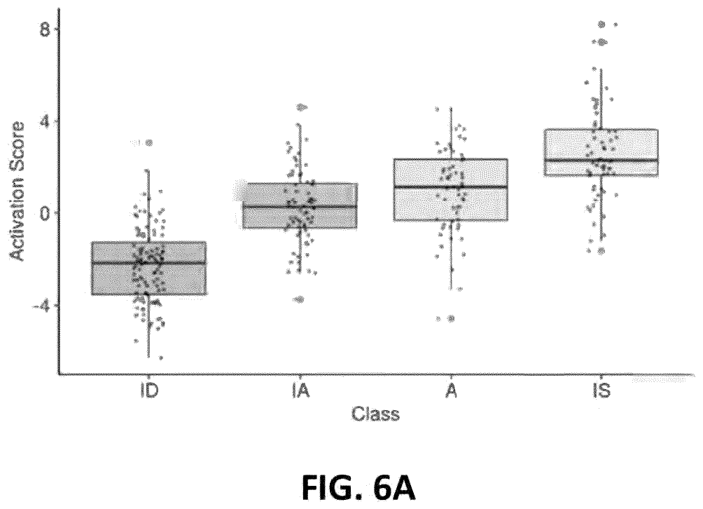

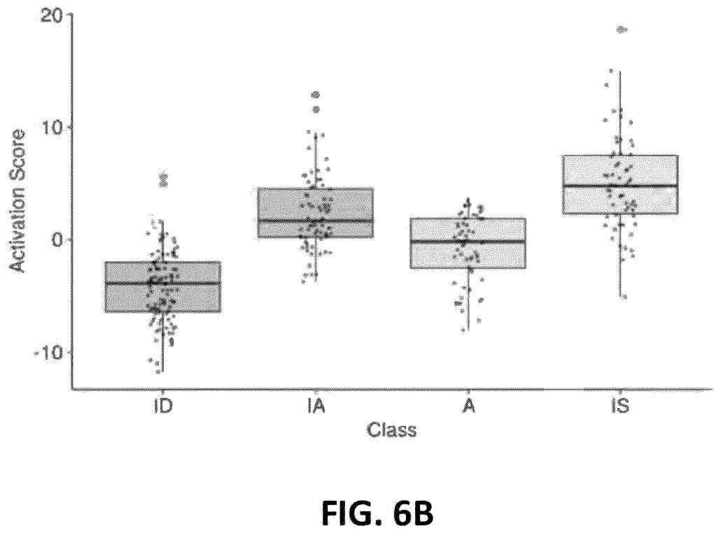

Gene ontology analyses were conducted. FIG. 6A shows box plots of the median and range of values the expression levels from the Treg signature (Angelova et al. (2015) Genome Biol. 16:64), as a function of the four stromal subtypes in the ACRG data. FIG. 6B shows a box plot of the median and range of values the expression levels of an inflammatory response signature (as defined by GO (Gene Ontology, GO_REF:0000022), as a function of the four stromal subtypes in the ACRG data.

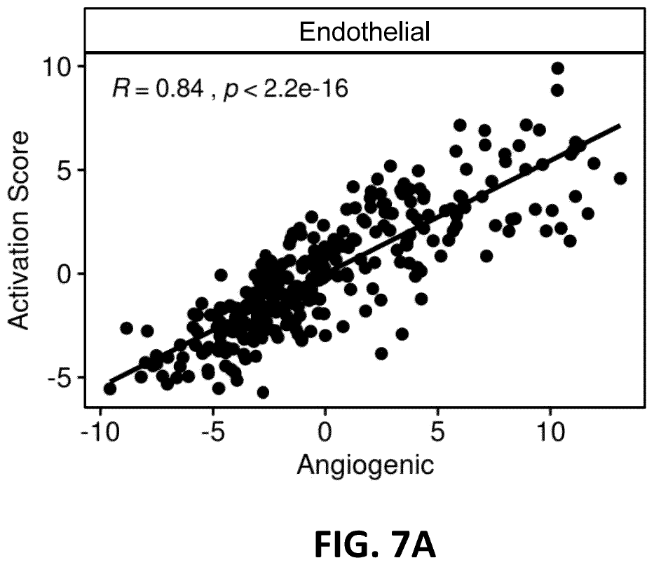

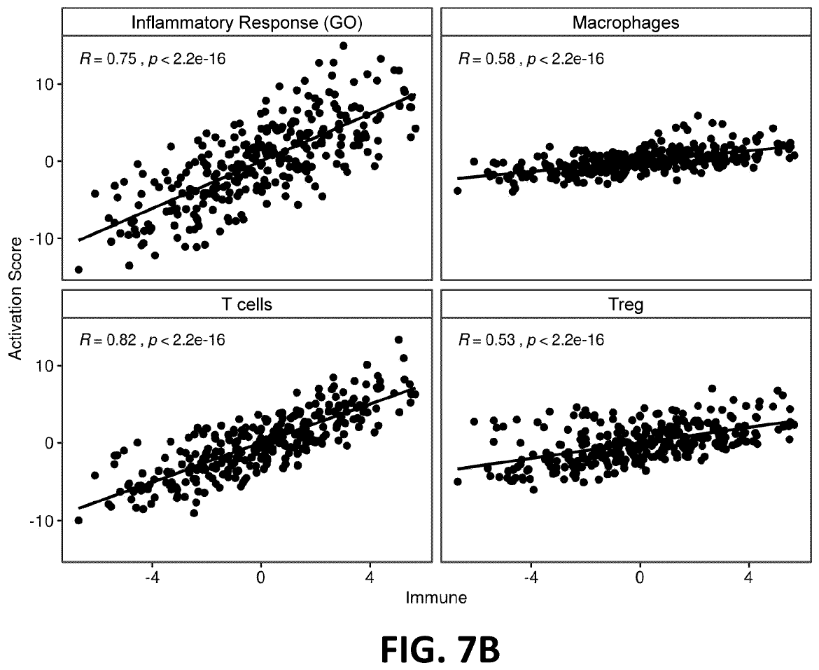

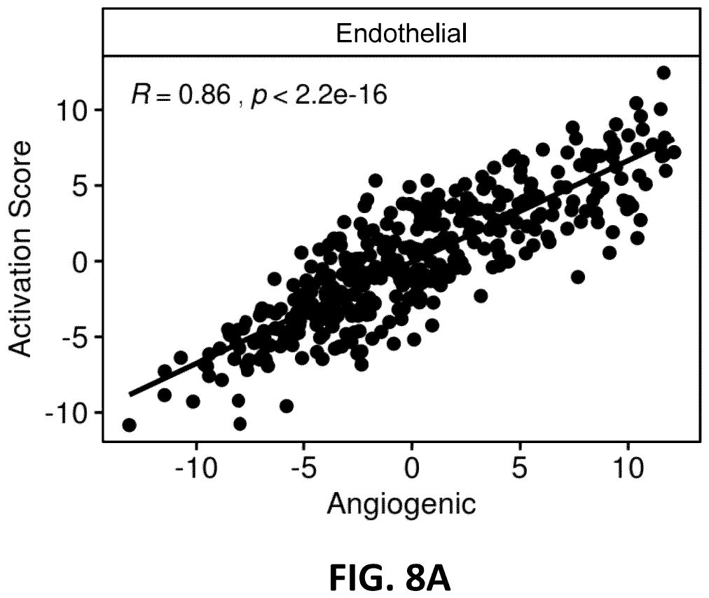

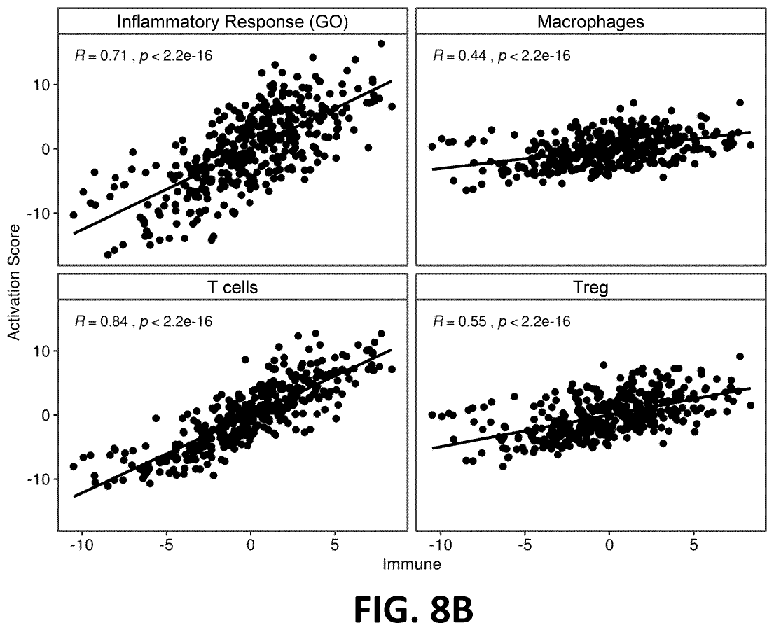

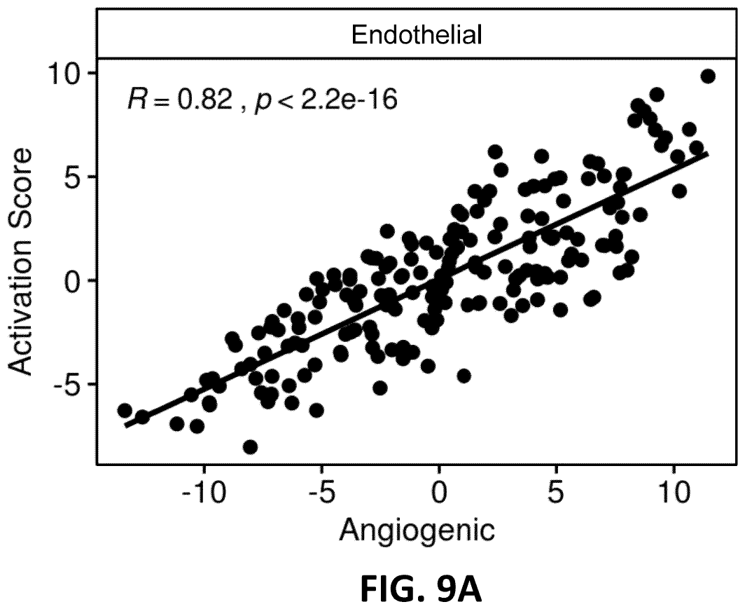

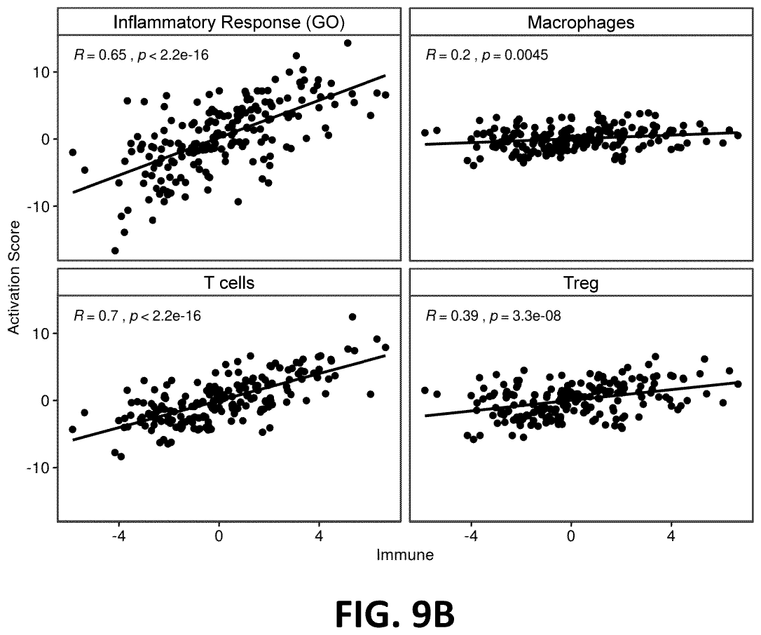

Further gene ontology analyses of the two Signatures, Signature 1 and Signature 2, were conducted. For the ACRG cohort, Signature 1 pathway activation scores for each patient were plotted on the x-axis and endothelial cell signature activation was plotted on the y-axis. Trend line represents a linear regression. The endothelial cell signature was obtained from Bhasin, et al., BMC Genomics 11:342, 2010. The positive slope indicated a positive correlation between the Signature 1 genes of patients in the ACRG cohort and endothelial cell signatures (FIG. 7A). Signature 2 pathway activation scores for each patient were plotted on the x-axis and pathway activation scores for the indicated pathways were plotted on the y-axis. Trend lines represent a linear regression. The positive slope indicated a positive correlation between the Signature 2 pathway activation scores of patients in the ACRG cohort and the pathways indicated in the titles of the plots. It can be seen from the slopes of the trend lines that genes involved in macrophage pathways were least correlated with Signature 2 genes, while genes involved in inflammatory response pathways (as defined by GO (Gene Ontology, GO_REF:0000022)), and in Tregs and T cell pathways (Angelova, et al.) were positively correlated (FIG. 7B). Similar analyses were conducted with the TCGA dataset (FIG. 8A and FIG. 8B) and the Singapore dataset (FIG. 9A and FIG. 9B).

Various thresholds were employed to stratify, or classify, the patients of the ACRG dataset (TABLE 26). It can be seen that applying a threshold of + or −0.4 (for example) on each individual Z-score (unitless) in will result in changes in the zₛ, or Activation Score, for the patient, and hence in the numbers of patients assigned to each of the four stromal subtypes. In some aspects, different thresholds, and different thresholds for each of the Signatures 1 and 2, are appropriate for the methods of the present disclosure.

TABLE 26
Varying the threshold of Signature 1 (“1”) and Signature 2
(“2”) during classification of the ACRG cohort.

| | threshold = 0 | 2 >= +0.4 | 1 >= −0.4 | 2 >= 0.4, 1 >= 0.4 | 1 >= −0.4 |
|---|---|---|---|---|---|
| IA | 24.8% | 21.1% | 29.2% | 22.8% | 22.5% |
| ID | 30.2% | 33.9% | 25.8% | 37.2% | 28.5% |
| A | 18.8% | 21.8% | 15.8% | 18.5% | 20.5% |
| IS | 26.2% | 23.2% | 29.2% | 21.5% | 28.5% |

### Example 3

### Pre-Treatment Gastric Tumor Microenvironment RNA Signature Correlates with Clinical Responses to Checkpoint Inhibitor Therapy

Summary: A retrospective data analysis indicated that gastric cancer tumor microenvironment phenotypes correlated to clinical responses when patients were treated with targeted therapy, such as a checkpoint inhibitor. The analysis included 45 gastric cancer tumor samples. Data indicated that the immune active (IA) phenotype was uniquely responsive to the checkpoint inhibitor relative to the immune suppressed (IS), immune desert (ID), and angiogenic (A) phenotypes.

Background information, methods and results: A retrospective classification of 45 patients with gastric cancer who received pembrolizumab, were classified according to the population-based method of the present disclosure. RNA expression levels were measured by paired-end RNA-Seq and normalized prior to classification. The data are reported according to the RECIST Criteria, e.g. Complete Responders (CR), Partial Responders (PR)) and SD/PD (Stable Disease/Progressive Disease (See TABLE 27). Overall Response Rate (ORR) is defined here as the number of CR+PR patients divided by the total number of patients. The ORR for all patients was 27% (12/45). Class response rate is defined here as the number of CR+PR patients in that stromal subtype class divided by the number of patients in that class. When the patients were retrospectively analyzed and placed in the Class IA, the response rate was 80%; and in Class IS, the response rate was 18%. The patients who retrospectively fell in the Class ID had a response rate of 12%, and Class A patients had a 0% response rate.

TABLE 27
Pre-treatment Classification of Patients who received
Pembrolizumab for Gastric Cancer (Mean threshold), n = 45.

| | ORR (CR + PR) | | | | |
|---|---|---|---|---|---|
| | or class response rate | CR | PR | SD | PD |
| All (ORR) | 12/45 (27%) | 3/45 | 9/45 | 15/45 | 18/45 |
| IA | 8/10 (80%) | 2/10 | 6/10 | 0/10 | 2/10 |
| ID | 2/16 (12%) | 0/16 | 2/16 | 7/16 | 7/16 |
| IS | 2/11 (18%) | 1/11 | 1/11 | 3/11 | 6/11 |
| A | 0/8 (0%) | 0/8 | 0/8 | 5/8 | 3/8 |

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from gastric cancer; and,
(c) the TME-class specific therapy comprises the administration of pembrolizumab.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from gastric cancer; and,
(c) the TME-class specific therapy comprises the administration of pembrolizumab.

### Example 4

### Pre-Treatment Gastric Tumor Microenvironment RNA Signature Correlated with Clinical Responses to Anti-Angiogenic Therapy

Summary: A retrospective data analysis indicated that gastric cancer stromal phenotypes correlated to clinical responses when patients were treated with targeted therapy, such as an angiogenic inhibitor. The analysis included 49 gastric cancer tumor samples. Data indicated that the angiogenic (A) and immune suppressed (IS) phenotypes were uniquely responsive to anti-angiogenic therapy relative to the immune active (IA) and immune desert (ID) phenotypes.

Background information, methods and results: The drug combination consisting of ramucirumab, a VEGF inhibitor, and paclitaxel is a commonly used regimen for second line treatment in PDL-1 negative gastric cancer patients. To test if stromal phenotypes correlated with clinical outcomes when patients were treated with ramucirumab and paclitaxel, the RNA gene signatures were analyzed in pre-treatment archival tissues from 49 gastric cancer patients and were classified according to the population-based method of the present disclosure. The correlation between each stromal phenotype was tested against clinical outcome data. With stratification of patients into one of four phenotypes, the effect size and clinical significance alters compared to historical data (Wilke et al 2014). The data are reported according to the RECIST criteria, e.g. Complete Responders (CR), Partial Responders (PR)) and SD/PD (Stable Disease/Progressive Disease (See TABLE 28). RNA expression levels were measured by paired-end RNA-Seq and normalized prior to classification. Overall Response Rate (ORR) is defined here as the Number of CR+PR patients divided by the total number of patients. For the 49 patients in the present Example, the ORR for all patients was 39% (19/49). Class response rate is defined here as the number of CR+PR patients in that stromal subtype class divided by the number of patients in that class. When the patients were retrospectively analyzed and placed in the Class IS, the class response rate was 56%; and in Class A, the class response rate was 37%. The patients who retrospectively fell in the Class IA had a class response rate of 33%, and Class ID patients had a 25% class response rate. Overall, in this relatively small patient sample set, the A and IS tumor microenvironment phenotypes correlated specifically with improved clinical outcomes with anti-angiogenic therapy.

TABLE 28
Pre-treatment Classification of Patients who received Ramucirumab
and Paclitaxel for Gastric Cancer (Mean threshold), n = 49.

| | ORR (CR + PR) | | | | |
|---|---|---|---|---|---|
| | or class response rate | CR | PR | SD | PD |
| All (ORR) | 19/49 (39%) | 0/49 | 19/49 | 25/49 | 5/49 |
| IA | 3/9 (33%) | 0/9 | 3/9 | 4/9 | 2/9 |
| ID | 4/16 (25%) | 0/16 | 4/16 | 11/16 | 1/16 |
| IS | 9/16 (56%) | 0/16 | 9/16 | 6/16 | 1/16 |
| A | 3/8 (37%) | 0/8 | 3/8 | 4/8 | 1/8 |

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from gastric cancer; and,
(c) the TME-class specific therapy comprises the administration of a VEGF inhibitor, e.g., ramucirumab, and paclitaxel.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein

(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from gastric cancer; and,
(c) the TME-class specific therapy comprises the administration of a VEGF inhibitor, e.g., ramucirumab, and paclitaxel.

### Example 5

### Pre-Treatment Gastric Tumor Microenvironment RNA Signature Correlates with Clinical Responses to Chemotherapy

Summary: A retrospective data analysis indicates that gastric cancer tumor microenvironment phenotypes correlate to clinical responses when patients are treated with chemotherapy. The analysis includes 50 gastric cancer tumor samples. Data indicate that the angiogenic (A) and immune suppressed (IS) phenotypes are less responsive to chemotherapy relative to the immune active (IA) and immune desert (ID) phenotypes.

Background information, methods and results: FOLFOX is a commonly used chemotherapy combination regimen consisting of fluorouracil, leucovorin and oxaliplatin. The overall response rate (ORR) with FOLFOX was reported as 34.8% in untreated advanced gastric cancer patients (Al-Batran et al. J Clin Oncol. 2008 Mar. 20; 26(9):1435-42). Median time to progression (PFS) and overall survival (OS) was 5.8 months and 10.7 months, respectively. To test if stromal phenotypes correlate with clinical outcomes when patients are treated with chemotherapy, RNA expression is analyzed in pre-treatment archival tissues from 50 gastric cancer patients (44 primary tumor samples, 6 metastatic tumor samples). The correlation between each stromal phenotype is tested against clinical outcome data. In A and IS patients, the use of FOLFOX confers less benefit in comparison to patients classified to IA and ID phenotypes: in IA and ID patients the median PFS and OS extends to approximately 7.8 months and 14.7 months, respectively. Overall, in this relatively small patient sample set, the A and IS tumor microenvironment phenotypes correlate specifically with improved clinical outcomes which could suggest that phenotypes are predictive for chemotherapy benefit.

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from gastric cancer; and,
(c) the TME-class specific therapy comprises the administration of chemotherapy, e.g., FOLFOX.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from gastric cancer; and,
(c) the TME-class specific therapy comprises the administration of chemotherapy, e.g., FOLFOX.

### Example 6

### Colorectal Cancer Tumor Microenvironment RNA Signature Correlates to Clinical Responses to Anti-Angiogenic Therapy

Summary: A retrospective data analysis indicates that colorectal cancer tumor microenvironment phenotypes correlate to clinical responses when patients are treated with targeted therapies, including angiogenesis inhibitors. The analysis includes analysis of 642 colorectal cancer tumor samples. Data indicate that the angiogenic (A) and immune suppressed (IS) phenotypes are uniquely responsive to anti-angiogenic therapy relative to the immune active (IA) and immune desert (ID) phenotypes.

Background information, methods and results: Bevacizumab in combination with chemotherapy increases PFS and OS in patient with advanced colorectal cancer (Snyder et al. Rev Recent Clin Trials. 2018; 13(2):139-149). The overall response rate (RR) in previously untreated metastatic colorectal cancer patients with was reported as 80% in left-sided tumors and 83% in right-sided tumors. Median time to progression (PFS) and overall survival (OS) in both left- and right-sided tumors was 13 months and 37 months, respectively. To test if tumor microenvironment phenotypes correlate with clinical outcomes when patients are treated with an angiogenesis inhibitor, tumor RNA gene signatures are being analyzed from archival tissues collected from 642 gastric cancer patients (321 left-sided, 321 right-sided). The correlation between each tumor phenotype was tested against clinical outcome data. With stratification of tumors into one of four phenotypes, the effect size and significance altered in comparison to historical data. In A and IS patients the use of bevacizumab confers modest gains in comparison to patients classified to IA and ID phenotypes: in A and IS patients median PFS and OS is predicted to shift to 15 months and 39 months, respectively. Progression-free survival and OS data in IA and ID patients are consistent with historical values. Overall, the A and IS tumor microenvironment phenotypes correlate specifically with improved clinical outcomes with angiogenesis inhibitors and have a predictive effect with respect to PFS.

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from colorectal cancer; and,
(c) the TME-class specific therapy comprises the administration of bevacizumab in combination with chemotherapy.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from colorectal cancer; and,
(c) the TME-class specific therapy comprises the administration of bevacizumab in combination with chemotherapy.

### Example 7

### Bavituximab Phase II Clinical Trial

This example concerns the use of bavituximab to enhance the activity of immunotherapy agents in humans, and particularly concerns treating cancer patients with bavituximab in combination with an anti-PD-1 or an anti-PD-L1 antibody, with a characterization of the patient's stromal subtypes according to the present disclosure.

An open-label, Phase II trial of bavituximab with pembrolizumab in patients that either (i) had relapsed after achieving a confirmed disease control (CR, PR, or SD) after treatment with any checkpoint inhibitor; or (ii) were naïve to an anti-PD-1 or an anti-PD-L1 therapy in advanced gastric or gastroesophageal cancer. The trial was conducted at approximately 19 centers world-wide, including in the U.S and Asia. The goals of the trial were (i) to see whether the combination was safe and provided a clinically meaningful improvement for the combination treatment compared to historical results with anti-PD-1 or an anti-PD-L1 monotherapy, and (ii) to see if there was a biomarker subgroup in which response to the combination therapy was meaningful over other biomarker subgroups in a RUO (research use only) scenario.

The test product, dose, and mode of administration were as follows: bavituximab was supplied as a sterile, preservative-free solution with 10 mM acetate at pH 5.0, and water for injection. Bavituximab was administered as an intravenous (IV) infusion at least 3 mg/kg body weight, weekly, according to the clinical protocol. A flat dose of 200 mg of pembrolizumab was administered Q3W.

Formalin-fixed tissue from a recent biopsy was used for generating RNA sequences according to the protocol established by a whole RNA sequencing technology company.

Patients whose stromal subtypes were IA or IS (as analyzed by the population-based method) or were biomarker positive (as analyzed by the ANN method) received benefit from the combination treatment of bavituximab and pembrolizumab (a representative checkpoint inhibitor).

TABLE 29 tabulates the results of the application of the ANN method with appropriate thresholds, cutoffs, or parameters, to the data of the 38 patients for whom was RNA sequencing data was available, as well as the ORR, DCR, and Best Objective Responses (CR, PR, SD, and PD).

TABLE 29
Biomarker positivity and negativity for the 38 patients with gastric/
gastroesophageal cancer treated with bavituximab and pembrolizumab
combination therapy for which biomarker data was available. Biomarker
positivity (i.e., presence of the biomarker) or negativity (i.e., absence of
the biomarker) was determined using the ANN method.

| | Biomarker status | |
|---|---|---|
| Clinical Benefit (%) | Positive (n = 22) | Negative (n = 16) |
| ORR¹ | 27% | 0% |
| DCR (Disease Control Rate) | 45% | 13% |
| CR¹ | 9% | 0% |
| PR¹ | 18% | 0% |
| SD | 18% | 13% |
| PD | 55% | 88% |

¹Confirmed Responses & unconfirmed where next scan is pending

Disease Control Rate (DCR) was defined as the percentage of patients with advanced or metastatic cancer who have achieved complete response (CR), partial response (PR) or stable disease (SD) to a therapeutic intervention in clinical trials of anticancer agents. PD is progressive disease.

A retrospective analysis of the 38 patients in the ONCG100 trial for whom there is biomarker data (i.e., RNA expression data as classified by the ANN method) was combined with NLR (neutrophil-leukocyte ratio) data. Performance data is given in TABLE 30.

TABLE 30
Performance values for the 22 patients
with biomarker data and NLR < 4.

| Biomarkers, | | ROC | | | | |
|---|---|---|---|---|---|---|
| threshold | ACC | AUC | Sensitivity | Specificity | PPV | NPV |
| IA + IS and | 0.64 | 0.75 | 1.00 | 0.50 | 0.43 | 1.00 |
| NLR < 4 | (14/22) | | (6/6) | (8/16) | (6/14) | (8/8) |

Accuracy (ACC): Number of correct predictions/Total number of predictions
ROC AUC: Receiver Operating Characteristics Area Under the Curve; degree to which model is capable of distinguishing between classes
Sensitivity: True biomarker responders/Total actual responders
Specificity: True biomarker non-responders/Total actual non-responders
Positive predictive value (PPV): True biomarker responders/Total predicted biomarker responders
Negative predictive value (NPV): True biomarker non-responders/Total predicted biomarker non-responders

In a group with 80 gastric/gastroesophageal cancer patients undergoing combination therapy with bavituximab and pembrolizumab, the biomarker positivity rate is approximately 30%.

TABLE 31 shows population-based Z-score stromal phenotype classifications and Best Objective Response for the 23 patients with biomarker data.

TABLE 31
Population-based Z-score classifications and best objective
response for the 23 patients with biomarker data.

| TME | N | # CR | # PR | # SD | # PD |
|---|---|---|---|---|---|
| IA | 8/23 | 1 | 1 | 1 | 5 |
| IS | 8/23 | 0 | 1 | 2 | 5 |
| A | 1/23 | 0 | 0 | 0 | 1 |
| ID | 6/23 | 0 | 1 | 2 | 3 |

TABLE 32 shows the interim results of the trial for all 44 patients. Objective responses were observed in 9 patients for an overall response rate (ORR) of 20% for all enrolled patients. Not all patients have confirmed responses.

TABLE 32
Bavituximab and pembrolizumab combination therapy in
gastric/gastroesophageal cancer study (unconfirmed results; N = 44,
MSS, PD-L1 positive and negative patients).

| | All Patients (N = 44) |
|---|---|
| ORR [CR + PR] | 9/44 (20%) |
| DCR [CR + PR + SD] | 17/44 (39%) |
| CR | 2/44 (5%) |
| PR | 7/44 (16%) |
| SD | 8/44 (18%) |
| PD | 27/44 (61%) |

In addition, other non-RNA signature based biomarkers were used to assess the baseline immune status of patients. These included microsatellite instability (MSI-H), mismatch repair deficiency (e.g., determined by IHC), EBV (Epstein-Barr virus) or HPV (human papilloma virus) positivity (either presence or absence), baseline β2GP1 (β2-glycoprotein 1) expression levels, IFNγ expression levels, and PD-1 or PD-L1 expression levels, using the Combined Positive Score (CPS). CPS is the number of PD-L1 staining cells (e.g., tumor cells, lymphocytes, macrophages) divided by the total number of viable tumor cells, multiplied by 100.

It is known in the art that patients who are MSI-H (i.e., high in microsatellite instability), and/or have positive EBV signals, and/or are high in PD-L1 expression levels have better responses to anti-PD-1 or anti-PD-LI monotherapy. In this clinical trial, it was expected that MSS (microsatellite stable, the opposite of MSI-H), EBV-negative, or PD-L1-low patients would benefit from bavituximab, making the patients better able to respond to the pembrolizumab. In the patient subset analyses for MSS (microsatellite stability), for the 28 MSS patients, the ORR was 21.0 (n=6); 16 patients had unknown MSS status. Twenty percent (20%) of patients with a CPS <1 responded to treatment; both patients who were complete responders (CR) had CPS scores less than 1.

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from advanced gastric or gastroesophageal cancer; and,
(c) the TME-class specific therapy comprises the administration of bavituximab and an anti-PD-1 immunotherapy antibody (e.g., nivolumab, pembrolizumab, cemiplimab, PDR001, CBT-501, CX-188, sintilimab, tislelizumab, or TSR-042) or an anti-PD-L1 immunotherapy antibody.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from advanced gastric or gastroesophageal cancer; and,
(c) the TME-class specific therapy comprises the administration of bavituximab and an anti-PD-1 immunotherapy antibody (e.g., nivolumab, pembrolizumab, cemiplimab, PDR001, CBT-501, CX-188, sintilimab, tislelizumab, or TSR-042) or an anti-PD-L1 immunotherapy antibody.

### Example 8

### Bavituximab Phase III Clinical Trial

The present example describes a Phase III, pivotal trial for bavituximab and an anti-PD-1 immunotherapy antibody in gastric cancer, using the methods of the present disclosure as a patient selection tool, i.e., an IUO (Investigator Use Only).

The trial is conducted similarly to the clinical trial described in the previous example, but in 30 trial centers and with 300 patients with advanced adenocarcinoma gastric or gastroesophageal cancer. Patients with gastric cancer have biopsies taken, and RNA expression levels for the Signature 1 and Signature 2 genes are measured and compared to a population-based reference, using the appropriate thresholds. Patients who are IS respond best to bavituximab and a checkpoint inhibitor, and there is clinically meaningful improvement from the combination treatment as defined by the statistical section of protocol. Patients who are IA will also respond, but ID and A patients will be ineligible for the trial.

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from gastric cancer; and,
(c) the TME-class specific therapy comprises the administration of bavituximab and an anti-PD-1 immunotherapy antibody (e.g., nivolumab, pembrolizumab, cemiplimab, PDR001, CBT-501, CX-188, sintilimab, tislelizumab, or TSR-042).

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from gastric cancer; and,
(c) the TME-class specific therapy comprises the administration of bavituximab and an anti-PD-1 immunotherapy antibody (e.g., nivolumab, pembrolizumab, cemiplimab, PDR001, CBT-501, CX-188, sintilimab, tislelizumab, or TSR-042).

### Example 9

### Anti-VEGF Therapy Phase II Trial

The present example concerns the use of anti-angiogenic antibodies (e.g., monoclonal antibodies specific to VEGF or anti-DLL4 monoclonal antibodies) and/or bispecifics antibodies (e.g., the anti-VEGF/anti-DLL4 bispecific navicixizumab) with one component associated with VEGF to enhance the activity as a single agent or in combination with standard of care such as chemotherapy, based on a patient's stromal subtypes according to the present disclosure.

The present example describes an open-label, Phase I/II trial of anti-VEGF therapy alone or in combination with standard of care in patients with the following diseases; advanced platinum-resistant ovarian cancer that have failed all lines of approved treatments for advanced disease (e.g. 4th line), refractory adenocarcinoma of the colon or rectum after least two prior regimens of standard chemotherapies (e.g., 3rd line), or post-operative advanced adenocarcinoma gastric or gastroesophageal cancer (e.g., 1st line). The trial is conducted at approximately 10 centers world-wide, including the U.S, EU, and Asia. The goals of the trial are to see if the monotherapy anti-VEGF treatment or combination treatment is safe and a clinically meaningful improvement compared to historical results. Including potential predictive outcome in a biomarker positive subgroup (A and IS) to the VEGF treatment or combination treatment with VEGF is clinically meaningful in a RUO (research use only) scenario.

The test product, dose, and mode of administration are as follows: Will be administered as an intravenous (IV) infusion according to the clinical protocol.

Formalin-fixed tissue from a recent biopsy is used for generating RNA sequences according to the protocol established by an RNA sequencing technology company, such as HTG Molecular Diagnostics (Tucson, Arizona, USA) or Almac (Craigavon, Northern Ireland, UK). The patient whose stromal subtype is A or IS will receive benefit from the anti-VEGF treatment or anti-VEGF combination treatment.

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from advanced platinum-resistant ovarian cancer that have failed all lines of approved treatments for advanced disease (e.g. 4th line), refractory adenocarcinoma of the colon or rectum after least two prior regimens of standard chemotherapies (e.g., 3rd line), or post-operative advanced adenocarcinoma gastric or gastroesophageal cancer (e.g., 1st line); and,
(c) the TME-class specific therapy comprises the administration of anti-angiogenic antibodies (e.g., monoclonal antibodies specific to VEGF or anti-DLL4 monoclonal antibodies) and/or bispecifics antibodies (e.g., the anti-VEGF/anti-DLL4 bispecific navicixizumab) with one component associated with VEGF to enhance the activity as a single agent or in combination with standard of care such as chemotherapy in patients with the cancers of (b).

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from advanced platinum-resistant ovarian cancer that have failed all lines of approved treatments for advanced disease (e.g. 4th line), refractory adenocarcinoma of the colon or rectum after least two prior regimens of standard chemotherapies (e.g., 3rd line), or post-operative advanced adenocarcinoma gastric or gastroesophageal cancer (e.g., 1st line); and,
(c) the TME-class specific therapy comprises the administration of anti-angiogenic antibodies (e.g., monoclonal antibodies specific to VEGF or anti-DLL4 monoclonal antibodies) and/or bispecifics antibodies (e.g., the anti-VEGF/anti-DLL4 bispecific navicixizumab) with one component associated with VEGF to enhance the activity as a single agent or in combination with standard of care such as chemotherapy in patients with the cancers of (b).

### Example 10

### Anti-VEGF Therapy Phase III Trial

The present example describes a Phase III, pivotal trial for one of the indications of the previous example with anti-VEGF therapy (e.g., with monoclonal antibodies specific to VEGF or anti-DLL4 monoclonal antibodies, and/or with bispecifics antibodies, e.g., the anti-VEGF/anti-DLL4 bispecific navicixizumab) alone or in combination with standard of care in patients with the following diseases: advanced platinum-resistant ovarian cancer that have failed all lines of approved treatments for advanced disease (e.g., 4th line), refractory adenocarcinoma of the colon or rectum after least two prior regimens of standard chemotherapies (e.g., 3rd line), or post-operative advanced adenocarcinoma gastric or gastroesophageal cancer (e.g., 1st line), using the methods of the present disclosure as a stratification tool, i.e., an IUO (Investigator Use Only).

Patients with above cancers have biopsies taken, and RNA expression levels for the Signature 1 and Signature 2 genes are measured and analyzed with the ANN model (trained on a population-based reference) and compared to a population-based reference, using the appropriate thresholds. Patients who are A or IS, i.e., patients who are biomarker-positive, respond best to anti-VEGF treatment or combination anti-VEGF therapy, and there is clinically meaningful improvement from the combination treatment compared to the predefined statistical plan in the protocol. Patients who are ID or IA will be ineligible for the study.

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from advanced platinum-resistant ovarian cancer that have failed all lines of approved treatments for advanced disease (e.g., 4th line), refractory adenocarcinoma of the colon or rectum after least two prior regimens of standard chemotherapies (e.g., 3rd line), or post-operative advanced adenocarcinoma gastric or gastroesophageal cancer (e.g., 1st line); and,
(c) the TME-class specific therapy comprises the administration of anti-VEGF therapy (e.g., with monoclonal antibodies specific to VEGF or anti-DLL4 monoclonal antibodies, and/or with bispecifics antibodies, e.g., the anti-VEGF/anti-DLL4 bispecific navicixizumab) alone or in combination with standard of care in patients with the cancers of (b).

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from advanced platinum-resistant ovarian cancer that have failed all lines of approved treatments for advanced disease (e.g., 4th line), refractory adenocarcinoma of the colon or rectum after least two prior regimens of standard chemotherapies (e.g., 3rd line), or post-operative advanced adenocarcinoma gastric or gastroesophageal cancer (e.g., 1st line); and,
(c) the TME-class specific therapy comprises the administration of anti-VEGF therapy (e.g., with monoclonal antibodies specific to VEGF or anti-DLL4 monoclonal antibodies, and/or with bispecifics antibodies, e.g., the anti-VEGF/anti-DLL4 bispecific navicixizumab) alone or in combination with standard of care in patients with the cancers of (b).

### Example 11

### Non-Population Machine-Learning Classifiers

The mechanics of three types of non-population-based classifiers based on machine-learning are provided. Non-population classifiers according to the present disclosure encompass, e.g., logistic regression, random forest, and artificial neural networks (e.g., a multilayer perceptron presented below). The fitted models (the classifiers), the mapping functions, and parameters are provided.

### Logistic Regression

Logistic Regression models the probability of a certain event, for example, a patient expressing a certain phenotype. This can be extended to model several classes of events, for example, four distinct manifestations of the phenotype.

The Logistic Regression predicted the probability of target class (e.g., TME class) using the following logistic function:

$$σ ( t ) = \frac{1}{1 - {e}^{- t}}$$

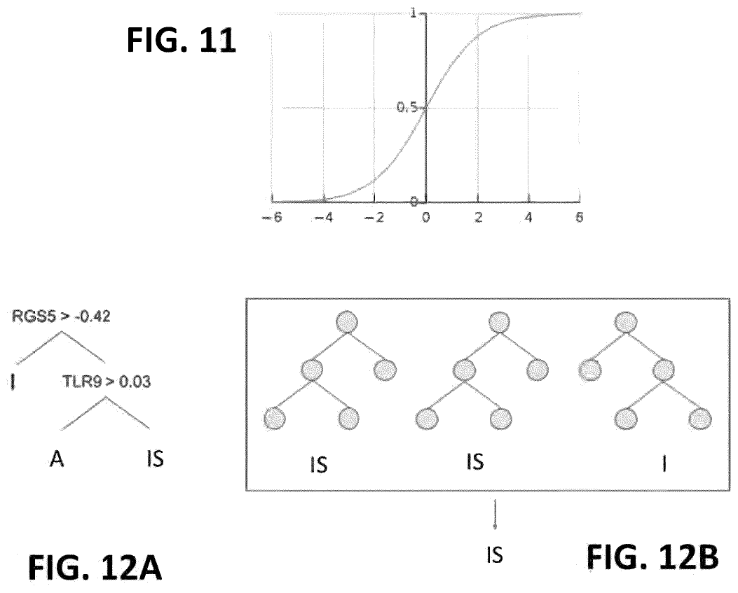

The logistic function (FIG. 11) can be interpreted as taking log-odds and output probability. When generalized to multiple features, we can express t as follows:

$$t = {β}_{0} + {β}_{1} {x}_{1} + {β}_{2} {x}_{2} + … + {β}_{m} {x}_{m}$$

And the general logistic function p can be written as:

$$p ( x ) = \frac{1}{1 + {e}^{- ( {β}_{0} + {β}_{1} {x}_{1} + {β}_{2} {x}_{2} + … + {β}_{m} {x}_{m} )}}$$

Model fitting: The model learns the parameters β for which the predictor (the logistic function) yields minimal error for the training dataset X (e.g., a set of rRNA expression levels corresponding to a gene panel disclosed herein together with assigned TME classifications resulting, e.g., from the application of a population-based classifier disclosed herein). The fitted model is represented as a set of parameters β and the logistic function. Intuitively, logistic regression searches for the model that makes the fewest assumptions in its parameters. Logistic regression also benefits from regularization, without which it is likely to overfit. Logistic regression can be generalized to multiple outcomes (for example when the target variable has multiple, say four, distinct values). Multinomial logistic regression is a classification method that generalizes logistic regression to multiclass problems.

Here, a set of parameters β (e.g., mRNA expression levels in a gene panel) was learned for each class (e.g., TME classes). Upon prediction, each class (e.g., a TME class) was assigned a probability, and the sample (e.g., a set of mRNA expression levels in a gene panel) was classified to the TME class with the highest probability. The parameters of the final Logistic Regression model that were fitted on the ACRG dataset are defined in the following table.

TABLE 33
Parameters of the Final Logistic Regression Model.
Logistic Regression Parameters

| | |
|---|---|
| C | 0.85 |
| max_iter | 10000 |
| Penalty | l2 |
| Solver | Saga |
| multi_class | multinomial |

| | TME Class | TME Class | TME Class | TME Class |
|---|---|---|---|---|
| | A | IA | ID | IS |
| Intercept bias | −1.072810 | 0.042660 | 11.441252 | −10.411102 |

Exemplary biomarkers in Logistic Regression Model

| Beta | TME Class | TME Class | TME Class | TME Class |
|---|---|---|---|---|
| coefficients* | A | IA | ID | IS |
| AFAP1L2 | −0.025072 | 0.182194 | −0.390321 | 0.233199 |
| AGR2 | −0.148136 | 0.448640 | −0.369014 | 0.068510 |
| BACE1 | 0.200963 | −0.146490 | −0.319934 | 0.265461 |
| BGN | 0.168208 | −0.087346 | −0.202203 | 0.121340 |
| BMP5 | 0.126860 | −0.106605 | 0.079634 | −0.099889 |

*Exemplary genes from a 98 gene set

### Random Forest

Random Forest (Breiman L, 2001) is an ensemble method that trains hundreds to thousands of decision trees. Individual trees are simple predictors (flow-chart-like structures) where each internal node denotes a test on a feature (gene), each branch represents the outcome of a test (expression being higher or lower than a given threshold), and each leaf holds a class label (a phenotype). The Random Forest model grows in number of trees without suffering from overfitting. This observation of a more complex classifier (a larger forest with more trees) getting more accurate contrasts with other techniques where growth in complexity almost always results in overfitting. This makes Random Forest a versatile classifier that can be applied to small datasets as well as large ones. See FIGS. 12A and 12B.

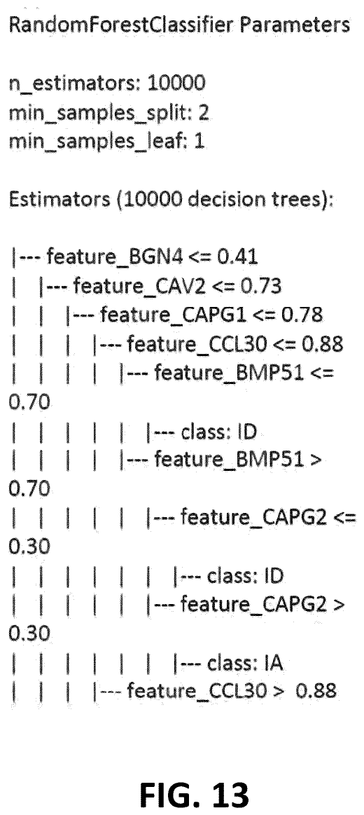

Model fitting: Individual trees were fitted by first drawing a random sample with replacement from the training set, and then fitting classification trees on the random draws of samples. The model was represented by a set of trees, each with a set of learned rules and decision thresholds on features. The parameters of the final Random Forest model fitted on the ACRG dataset are defined in FIG. 13.

### Artificial Neural Network

A multilayer perceptron (MLP) is a class of feed-forward artificial neural network. An MLP consists of at least three layers of nodes: an input layer, a hidden layer, and an output layer. Except for the input nodes, each node is a neuron that uses a nonlinear activation function. MLP utilizes a supervised learning technique called backpropagation for training. An MLP can distinguish data that is not linearly separable.

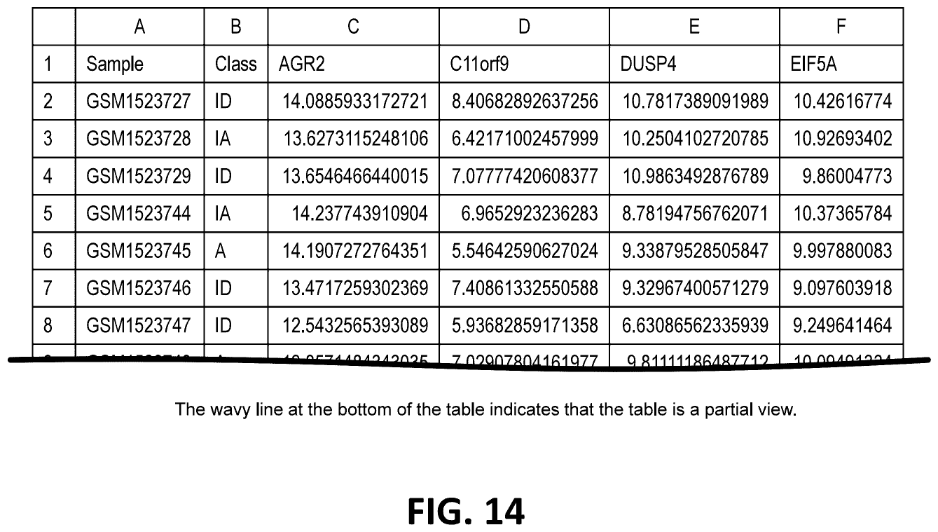

Training set: The ACRG gene expression dataset was used as training set. The ACRG training set comprised 235 samples out of 298 available, as 63 samples were identified as lying close to the decision boundary of the class labels; these samples affected the robustness of the model, and therefore were not included in the training set. Also included were 98 continuous variables (a 98 gene panel which comprises a subset of the genes presented in the Signature 1 and Signature 2 tables, i.e., TABLE 1 and TABLE 2), and corresponded to four target classes (A, IA, IS, and ID tumor microenvironments). Other training sets can be used, e.g., those disclosed in TABLE 5. As shown in FIG. 14, each sample included values (e.g., mRNA levels) for each gene in the gene panel and its classification into a specific Class (assigned, e.g., using the population method based on two Signatures disclosed herein).

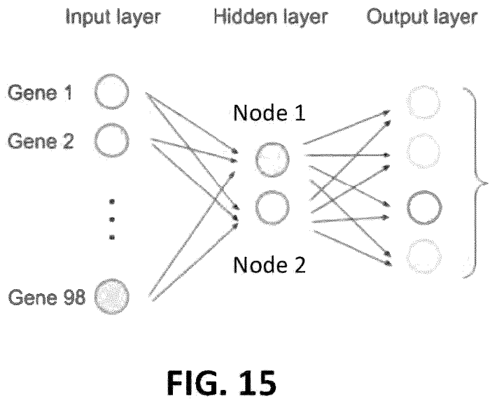

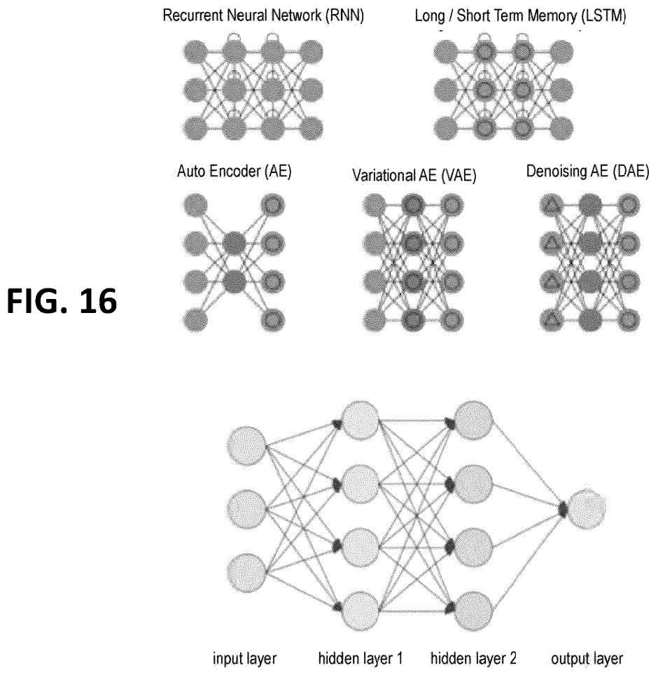

Neural Layer Architecture: The ANN used was a multi-layer perceptron (MLP) comprising an input layer, and output layer, and one hidden layer, as shown in a simplified form in FIG. 15. Each neuron in the input layer was connected to the two neurons in the hidden layer, and each of the neurons in the hidden layer was connected to each of the neurons in the output layer. Other architectures could be used to practice the present invention, for example any of the architectures shown in FIG. 16.

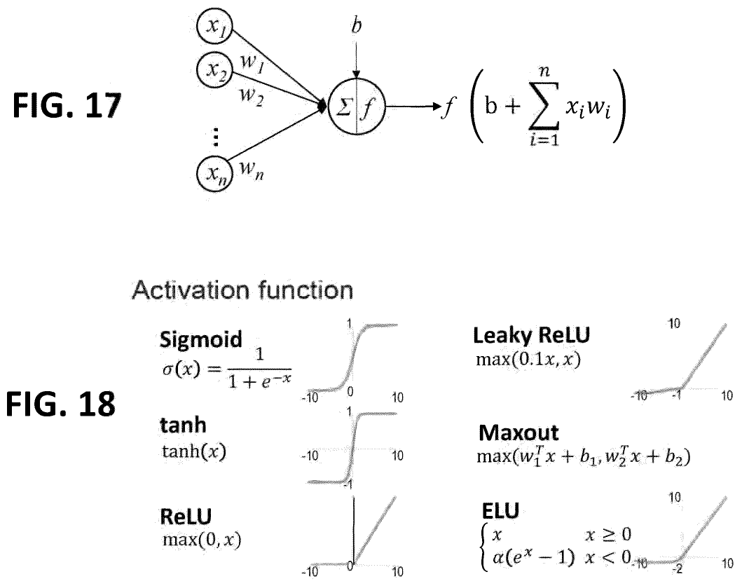

Training: A goal of the training process was to identify weights wi for each input and bias b in the hidden layer such that the neural network minimized the prediction error on the training set. See FIG. 17. As show in FIG. 17 each gene in the gene panel (x₁ . . . xₙ) was used as input for each neuron in the hidden layer and a bias b value for the hidden layer was identified through the training process. The output from each neuron was a function of each gene expression level (xᵢ), weight (wᵢ) and bias (b) as shown in FIG. 17.

A number of activation functions can be applied in the hidden layer, as illustrated in FIG. 18. A hyperbolic tangent activation function (tanh) that ranged from −1 to 1 was used to generate an ANN classifier as described herein

$$y ( {v}_{i} ) = tanh ( {v}_{i} )$$

wherein yᵢ was the output of the i th node (neuron) and vᵢ was the weighted sum of the input connections.

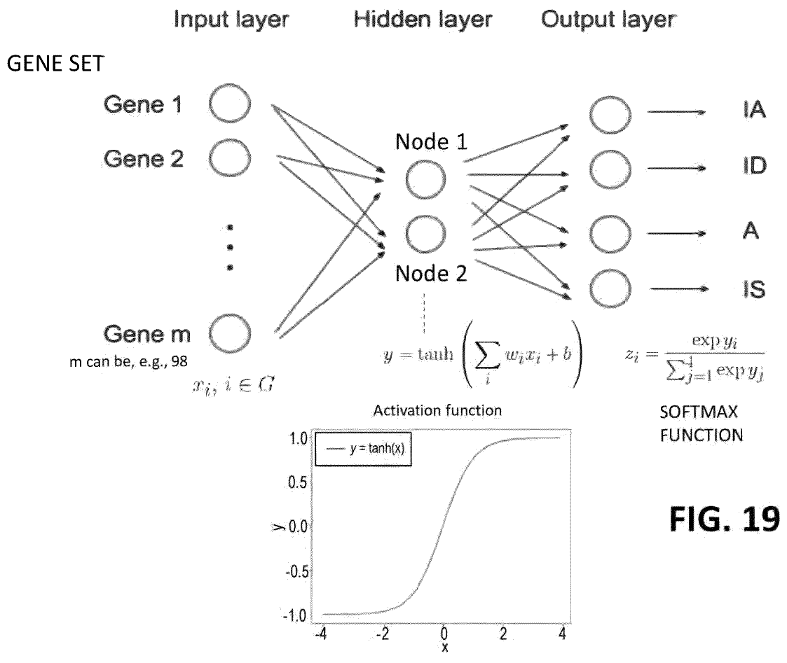

As described above, the artificial neural network classifier comprised gene expression values in the input layer (corresponding to a 98 gene panel), two neurons in the hidden layer that encoded the relation between the two stromal signatures, and four outputs which predicted the probability of four stromal phenotypes. See FIG. 19. Multi-class classification of the output layer values into four phenotype classes (IA, ID, A, and IS) was supported by applying a logistic regression classifier comprising the Softmax function. Softmax assigned decimal probabilities to each class that had to add up to 1.0. This additional constraint helped training converge more quickly. Softmax was implemented through a neural network layer just before the output layer and had the same number of nodes as the output layer.

As an additional refinement, various cut-offs were applied to the results of the Softmax function depending on the particular dataset used (see, e.g., cut-offs applied to pembrolizumab neural net output discussed in the following example.

Inspection of the artificial neural network classifier revealed that the training algorithm has indeed learned the weights (listed in TABLE 34) that represented the sign-based rule of the Signature 1 and Signature 2 signatures which was introduced in Population Model Based on the Z-Score Algorithm (i.e., the population-based classifier of the present disclosure, which was used to generate the training dataset).

The rule was inferred from the training data automatically. The algorithm was not given any assumptions about the Signatures 1 and 2 except for the hidden layer to include two neurons. For each hidden neuron, the genes from Signature 1 and Signature 2 contributed to at least some extent, either by a positive or negative gene weight, however one hidden neuron was more dominated by one signature, and vice versa (FIG. 29A and FIG. 29B).

TABLE 34
Artificial neural network weights on the output layer.

| | Output A | Output IA | Output ID | Output IS |
|---|---|---|---|---|
| Hidden Neuron 1 | 1.83 | −1.96 | 1.95 | −1.82 |
| Hidden Neuron 2 | −1.82 | 1.90 | 1.77 | −1.85 |

A list of parameters of the final Artificial Neural Network model fitted on the ACRG dataset is shown in TABLE 35.

TABLE 35
Parameters of the Final Artificial Neural Network Model.
MLP
Classifier Parameters

| hidden_layer | 2 | | | | | | |
|---|---|---|---|---|---|---|---|
| sizes | | | | | | | |
| Alpha | 2 | | | | | | |
| Solver | Lbfgs | | | | | | |
| Activation | Tanh | | | | | | |
| learning_rate | Constant | | | | | | |
| | | | | Output | Output | Output | Output |
| | Hidden | Hidden | | TME | TME | TME | TME |
| | Neuron 1 | Neuron 2 | | Class A | Class IA | Class ID | Class IS |
| Intercept bias | 5.750706 | 6.132147 | | −0.707687 | −0.641524 | −0.375602 | −0.413502 |
| | Hidden | Hidden | | | | | |
| Coefficients* | Neuron 1 | Neuron 2 | | Output A | Output IA | Output ID | Output IS |
| AFAPIL2 | −0.151264 | −0.117321 | Hidden | 1.83 | −1.96 | 1.95 | −1.82 |
| | | | Neuron 1 | | | | |
| AGR2 | −0.437438 | 0.049720 | Hidden | −1.82 | 1.90 | 1.77 | −1.85 |
| | | | Neuron 2 | | | | |
| BACE1 | −0.115562 | −0.271820 | | | | | |
| BGN | 0.029208 | −0.112965 | | | | | |

*Exemplary genes from a 98 gene set

### Example 12

### Application of ANN Method to Pembrolizumab monotherapy

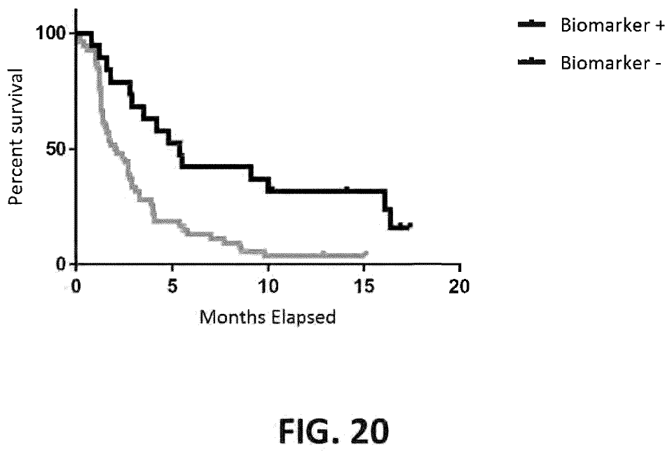

FIG. 20 shows that after treatment of gastric cancer with pembrolizumab monotherapy only the TME IS and IA class patients showed complete response, and that the number of complete responses in TME class IA was much higher than in IS. Furthermore, the number of partial responders was also much higher in the IA class.

FIG. 21 shows that the ANN classifier could be trained with a dataset comprising gene expression data including from patients having a particular cancer (gastric cancer) and being treated with a particular therapy (pembrolizumab). The output of the classifier is sorted into the TME classes A, IS, ID, IA, but complete responders (CR) and partial responders (PR) cluster at a neuron one output value close to 1. Accordingly, a new threshold could be implemented in the Softmax function that could effectively identify patients within the IS and IA TME classes that would have a higher likelihood of being complete or partial responders to pembrolizumab monotherapy. If the selection included both IS and IA class patients (Option A; dark area) a number of non-responders would be included in the selection. However, if the selection included only IA class patients (Option B; dark area) the entire population would presumably be composed of only complete responders and partial responders.

Option 1, i.e., optimizing the threshold but taking both IS and IA groups, modestly reduced the optimization of biomarker positive Overall Response Rate (ORR) from 80% to 70% ORR (10/14). This option minimized biomarker negatives and maximized the capture of total responders from 8/12 to 10/12.

Option 2, i.e., optimizing the threshold but taking only the IA subgroup, improved the optimization of biomarker-positive ORR from 80% to 100% ORR (8/8). However, there was no change to minimizing biomarker negatives or to maximizing the capture of total responders.

In order to find the boundary of the response, an additional optimization of the probability score was carried out. Compared to a probability score of 0.50, this resulted in a maximization of responders in the biomarker-positive (IA) group, which allowed for more accurate prediction of the patients who responded to pembrolizumab, while it also minimized the number of biomarker-negative patients in the responder group.

At 0.5 probability score, the performance is 80% PPV (positive predictive value) and 94% specificity. At 0.87 probability score, the performance rises to 100% PPV and 100% specificity without compromising sensitivity and NPV (negative predictive value). Sensitivity refers to the number of true biomarker responders divided by number of actual responders; specificity refers to the number of true biomarker non-responders divided by number of actual non-responders; PPV refers to the number of true biomarker responders divided by number of total predicted biomarker responders (how well the biomarker-positive rating performs); and NPV—refers to the number of true biomarker non-responders divided by number of total predicted biomarker non-responders (how well the biomarker-negative rating performs).

TABLE 36 shows that after second-line treatment of 73 patients with gastric cancer, (77% Pembrolizumab, 23% Nivolizumab) the specificity of the ANN biomarker (IA) was 83%.

TABLE 36
ANN Probability Score Optimization Compared to Industry
Gold Standard Biomarker for PD-1, alongside MSI-High Status
of 73 patients (77% Pembrolizumab, 23% Nivolizumab).

| | | ROC | | | | |
|---|---|---|---|---|---|---|
| Biomarker, threshold | ACC | AUC | Sensitivity | Specificity | PPV | NPV |
| Immune Active (IA) via | 0.79 | 0.72 | 0.62 | 0.83 | 0.44 | 0.91 |
| ANN | (58/73) | | (8/13) | (50/60) | (8/18) | (50/55) |
| PD-L1, CPS > 1 | 0.75 | 0.79 | 0.85 | 0.73 | 0.41 | 0.96 |
| (Industry Gold Standard) | (55/73) | | (11/13) | (44/60) | (11/27) | (44/46) |
| MSI-H | 0.85 | 0.67 | 0.38 | 0.95 | 0.62 | 0.88 |
| | (62/73) | | (5/13) | (57/60) | (5/8) | (57/65) |

Accuracy (ACC): Number of correct predictions/Total number of predictions.
ROC AUC: Receiver Operating Characteristics Area Under The Curve; degree to which model is capable of distinguishing between classes.
Sensitivity: True biomarker responders/Total actual responders.
Specificity: True biomarker non-responders/Total actual non-responders.
Positive predictive value (PPV): True biomarker responders/Total predicted biomarker responders.
Negative predictive value (NPV): True biomarker non-responders/Total predicted biomarker non-responders

TABLE 37
Comparison of biomarkers in second-line gastric cancer,
treated with pembrolizumab or nivolizumab (n = 73).

| | n | | PD- | PD- | | PD- | | |
|---|---|---|---|---|---|---|---|---|
| ORR | % | NLR ≤ 4 | L1 < 1 | L1 ≥ 1 | PDL1 < 10 | L1 ≥ 10 | MSI | MSS |
| All | 13/73 | 11/56 | 0/29 | 12/40 | 4/52 | 8/17 | 5/8 | 8/65 |
| patients | 18% | 20% | 0% | 30% | 8% | 47% | 63% | 12% |
| IA/IS | 11/32 | 9/27 | 0/11 | 11/20 | 4/22 | 7/9 | 4/4 | 7/28 |
| N = 32 | 34% | 33% | 0% | 55% | 18% | 78% | 100% | 25% |
| ID/A | 2/41 | 2/29 | 0/18 | 1/20 | 0/30 | 1/8 | 1/4 | 1/37 |
| N = 41 | 5% | 7% | 0% | 5% | 0% | 13% | 25% | 3% |
| IA | 8/18 | 6/14 | 0/3 | 8/14 | 3/10 | 5/7 | 3/3 | 5/15 |
| N = 18 | 44% | 43% | 0% | 57% | 30% | 71% | 100% | 33% |
| ID | 2/20 | 2/14 | 0/5 | 1/12 | 0/12 | 1/5 | 1/3 | 1/17 |
| N = 20 | 10% | 14% | 0% | 8% | 0% | 20% | 33% | 6% |
| A | 0/21 | 0/15 | 0/13 | 0/8 | 0/18 | 0/3 | 0/1 | 0/20 |
| N = 21 | 0% | 0% | 0% | 0% | 0% | 0% | 0% | 0% |
| IS | 3/14 | 3/13 | 0/8 | 3/6 | 1/12 | 2/2 | 1/1 | 2/13 |
| N = 14 | 21% | 23% | 0% | 50% | 8% | 100% | 100% | 15% |
| ≥60% | 9/19 | 7/16 | 0/4 | 9/15 | 4/12 | 5/7 | 3/3 | 6/16 |
| probability | 47% | 44% | 0% | 60% | 33% | 71% | 100% | 38% |
| for IS/IA | | | | | | | | |
| <60% | 4/54 | 4/41 | 0/25 | 3/25 | 0/38 | 3/11, | 2/5 | 2/49 |
| probability | 7% | 10% | 0% | 12% | 0% | 27% | 40% | 4% |
| for IS/IA | | | | | | | | |
| MSS | 8/65 | 7/50 | 0/27 | 7/34 | 4/49 | 3/12 | n/a | n/a |
| | 12% | 14% | 0% | 21% | 8% | 25% | | |

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from a gastric cancer; and,
(c) the TME-class specific therapy comprises the administration of pembrolizumab monotherapy.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from a gastric cancer; and,
(c) the TME-class specific therapy comprises the administration of pembrolizumab monotherapy.

### Example 13

### Application of ANN Method to Ramucirumab and Paclitaxel

ANN model performance on the Ramucirumab plus Paclitaxel data of Example 4. Ramucirumab targets angiogenesis, thus responders in the A and IS TMEs were expected. Accordingly, the results were combined into the A/IS (both angiogenic-responsive TMEs) to compare sensitivity and specificity versus the IA/ID TMEs.

TABLE 38
Use of ANN model to classify responders to angiogenesis therapy.

| | Z- Score | | | | | | | |
|---|---|---|---|---|---|---|---|---|
| | | | | Sig. | Sig. | Sig. | | |
| | | | | 2 ≥ 0.4, | 2 ≥ 0.4, | 2 ≥ −0.4, | | ANN |
| | Threshold = | Sig. | Sig. | Sig. | Sig. | Sig. | Sig. | 98 |
| | 0 | 2 ≥ 0.4 | 2 ≥ −0.4 | 1 ≥ 0.4 | 1 ≥ −0.4 | 1 ≥ 0.4 | 1 ≥ −0.4 | genes |
| Sensitivity | | | | | | | | |
| A + IS Responders/ | 58% | 58% | 58% | 58% | 63% | 58% | 63% | 63% |
| All Responders | | | | | | | | |
| Specificity | | | | | | | | |
| IA + ID Non-Responders/ | 63% | 63% | 63% | 63% | 53% | 63% | 53% | 60% |
| All Responders | | | | | | | | |
| Model Rank | 4 | 4 | 4 | 4 | 2 | 4 | 2 | 1 |

This methodology could be similarly applied to other types of cancers and to other therapies, for example, to select which individuals would be candidates for treatment with such specific therapy.

Without any patient selection, the overall ratio of responders to non-responders (19/48) was 39.6% (TABLE 39). Using the ANN method, and in order to find the boundary of the response, additional optimizations were carried out. This resulted in a maximization of responders in the biomarker-positive group, which allowed for more accurate prediction of the patients who responded to the combination therapy of ramucirumab and paclitaxel, while it also minimized the number of biomarker-negative patients in the responder group. After optimization, 73.7% of the responders were biomarker-positive compared to the no-selection percentage of 39.6%. The biomarker-positive patients had about 2.5 times the response rate: 73.7%, compared to the biomarker-negative rate of 27.7%. The median survival of the biomarker-positive group was 19 months versus 16.5 months for the biomarker-negative group.

TABLE 39

| | Responders (PR) | (SD/PD) |
|---|---|---|
| TME phenotype by biomarker +/− | N = 19 | (N = 29) |
| Biomarker positive N = 30 | 14 (73.7%) | 16 (55.5) |
| Biomarker negative N = 18 | 5 (27.8%) | 13 (44.8) |

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and,
(b) the TME-class specific therapy comprises the administration of ramucirumab and paclitaxel.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and,
(b) the TME-class specific therapy comprises the administration of ramucirumab and paclitaxel.

### Example 14

### Navicixizumab Phase 1A Trial

A retrospective data analysis of a Phase 1A dose-escalation trial for patients with solid tumors. Patients must have had a histologically confirmed malignancy that was metastatic or unresectable for which there was no remaining standard curative therapy and no therapy with a demonstrated survival benefit or they must have been ineligible to receive such therapy. Patients with the above cancers had biopsies collected. Exploratory predictive biomarkers such as DLL4 and VEGF were measured in FFPE tumor specimens, either archived or fresh core needle biopsied at study entry (2 FFPE cores were preferred whenever possible) by immunohistochemistry. RNA expression levels for the Signature 1 and Signature 2 genes were retrospectively measured from archived FFPE tumor specimens, and both the population-based method (Z-score) and the non-population-based ANN algorithm were applied using the appropriate thresholds for the tumor types, as each tumor type has a specific threshold Patients without an outcome label were excluded, leaving 39 in the total biomarker subset of the Phase 1A trial data. In this all-comers dose escalation trial, 38% achieved SD (Stable Disease) or better (RECIST 1.1 criteria). In the biomarker-positive subset, 48% achieved SD or better.

Notably, in the gynecological cancers (n=18), all of the patients with SD or better fell into the biomarker positive group, with benefit to 58% of the biomarker-positive (n=12) and 0% of biomarker-negative (n=6). Model performance is tabulated in TABLE 40; and abbreviations and definitions are as follows; ACC is accuracy; AUC ROC is area under the receiving operator characteristic curve; Sensitivity is the number of true biomarker responders divided by number of actual responders; Specificity is the number of true biomarker non-responders divided by number of actual non-responders; PPV is positive predictive value, i.e., number of true biomarker responders divided by number of total predicted biomarker responders; NPV is negative predictive value, i.e., number of true biomarker non-responders divided by number of total predicted biomarker non-responders.

TABLE 40
Z-score and ANN Model Performance in all Patients (n = 39) and in
Gynecological Cancers (n = 18).

| BASELINE | ACC | AUC ROC | Sensitivity | Specificity | PPV | NPV |
|---|---|---|---|---|---|---|
| Random | 0.53 | 0.50 | 0.38 | 0.62 | 0.38 | 0.62 |
| All subjects, | | | | | | |
| N = 39 | | | | | | |
| Positive class; | | | | | | |
| Normalization: quantile- | | | | | | |
| transformed TPM | | | | | | |
| Z-score | 0.59 | 0.62 | 0.73 | 0.50 | 0.48 | 0.75 |
| # of Patients | | | 11/15 | 12/24 | 11/23 | 12/16 |
| ANN | 0.56 | 0.56 | 0.53 | 0.58 | 0.44 | 0.67 |
| # of Patients | | | 8/15 | 14/24 | 8/18 | 14/21 |
| Gyne subjects only, | | | | | | |
| N = 18 | | | | | | |
| Positive class; | | | | | | |
| Normalization: quantile- | | | | | | |
| transformed TPM | | | | | | |
| Z-score | 0.72 | 0.77 | 1.00 | 0.55 | 0.58 | 1.00 |
| # of Patients | | | 7/7 | 6/11 | 7/12 | 6/6 |
| ANN | 0.61 | 0.63 | 0.71 | 0.55 | 0.50 | 0.75 |
| # of Patients | | | 5/7 | 6/11 | 5/10 | 6/8 |

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed). A (angiogenic), IA (immune active). ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a tumor from a gynecological cancer; and,
(c) the TME-class specific therapy comprises the administration of navicixizumab.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a tumor from a gynecological cancer; and,
(c) the TME-class specific therapy comprises the administration of navicixizumab.

### Example 15

### Navicixizumab Phase 1B Trial

The present example of the application of the ANN method in a retrospective analysis describes a Phase 1B dose escalation and expansion study of navicixizumab plus paclitaxel. The trial enrolled 44 platinum resistant ovarian cancer (PROC) patients who had failed >2 prior therapies and/or received prior bevacizumab. As of the last interim data analysis at the end of Q1 2019, the unconfirmed response rate was 43%, and the confirmed response rate was 36%.

Response data for 44 patients in the intent-to-treat population with PROC, uterine, or fallopian tube cancer in the trial with confirmed responses or progressive disease (RECIST criteria) were obtained. See TABLE 41.

TABLE 41
Navi 1B Reproductive Cancer Intent-to-Treat Population
Response Rate and Disease Control Rate
Best Objective Response Intent-to-Treat Population (N = 44)

| | |
|---|---|
| ORR | 43.2% |
| DCR | 77.3% |
| CR | 2.3% |
| PR | 40.9% |
| SD | 34.1% |
| PD | 15.9% |
| Not evaluable | 6.8% |

RNA expression levels for the Signature 1 and Signature 2 genes were measured from the patient biopsies. Biopsies were collected at the time of enrollment or archived biopsies were used. Population-based (Z-score) and the non-population-based ANN algorithms were applied using the appropriate thresholds for reproductive cancers. Patients without an outcome label were excluded, leaving 23 in the total biomarker subset of the 1B dataset.

Those patients who were positive after the application of the ANN model were considered biomarker positive. The ORR and DCR for patients with a known biomarker status are given in TABLE 42 and TABLE 43.

TABLE 42
Navi 1B Trial: Biomarker Status for the 23 patients who had
RNA Expression Data and Confirmed Response Data for Ovarian,
Uterine, and Fallopian Cancers.

| Best Objective Response for | Biomarker Status | |
|---|---|---|
| Confirmed and PD | Positive (N = 10) | Negative (N = 13) |
| ORR | 70.0% | 30.8% |
| DCR | 100.0% | 69.2% |
| CR | 0.0% | 7.7% |
| PR | 70.0% | 23.1% |
| SD | 30.0% | 38.5% |
| PD | 0.0% | 30.8% |
| PFS (months) | 9.2 | 3.5 |

TABLE 43
Population-Based Z-score Classifications and Best
Objective Response for the 23 Patients with Biomarker Data and
Confirmed Responses in the Navi 1B Trial Reproductive Cancer Cohort.

| TME | N | # CR | # PR | # SD | # PD |
|---|---|---|---|---|---|
| IA | 6/23 | 1 | 2 | 2 | 1 |
| IS | 9/23 | 0 | 5 | 3 | 1 |
| A | 2/23 | 0 | 2 | 0 | 0 |
| ID | 6/23 | 0 | 1 | 3 | 2 |

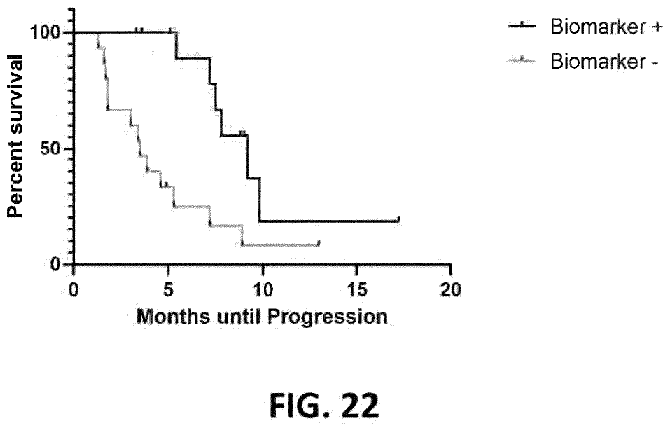

Confirmed response meant that the response was confirmed with a second imaging scan, taken after the first imaging scan, according to the protocol. By definition, progressive disease (PD) is not a confirmed response; PD patients were included in the denominators for calculating ORR and DCR. The progression-free survival (PFS) benefit for biomarker positive patients was 9.2 months compared to 3.5 months for the biomarker negative patients (p=0.0037). The Kaplan-Meier survival curve is provided in FIG. 22.

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a reproductive tumor selected from the group consisting of ovarian, uterine, and fallopian cancers; and,
(c) the TME-class specific therapy comprises the administration of navicixizumab and paclitaxel.

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a reproductive tumor selected from the group consisting of ovarian, uterine, and fallopian cancers; and,
(c) the TME-class specific therapy comprises the administration of navicixizumab and paclitaxel.

### Example 16

### Tumor Agnostic Models

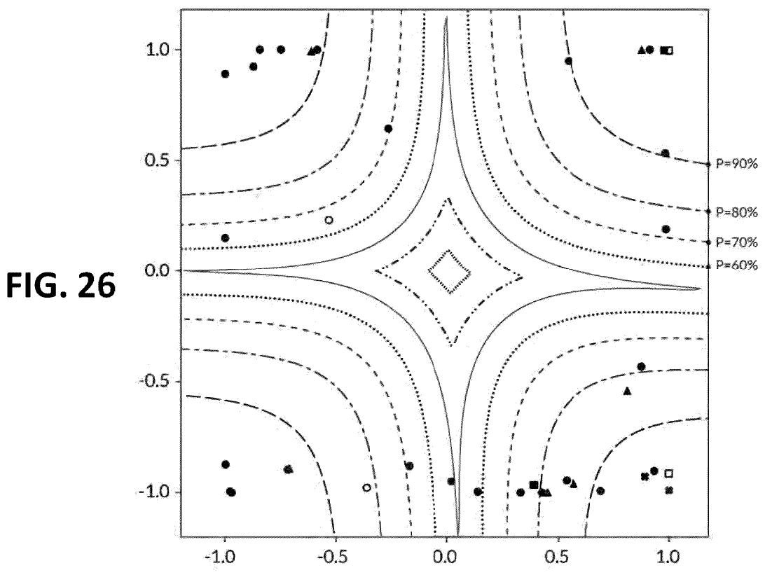

FIG. 26 shows the results of the application of an ANN model to 1200 patient samples sequences using RNA exome sequencing technology to 400 patients' samples, each of three different tumor types—colorectal, gastric, and ovarian. The consistency of the results across the probable stromal phenotypes revealed that the ANN model of the present disclosure is agnostic to tumor type.

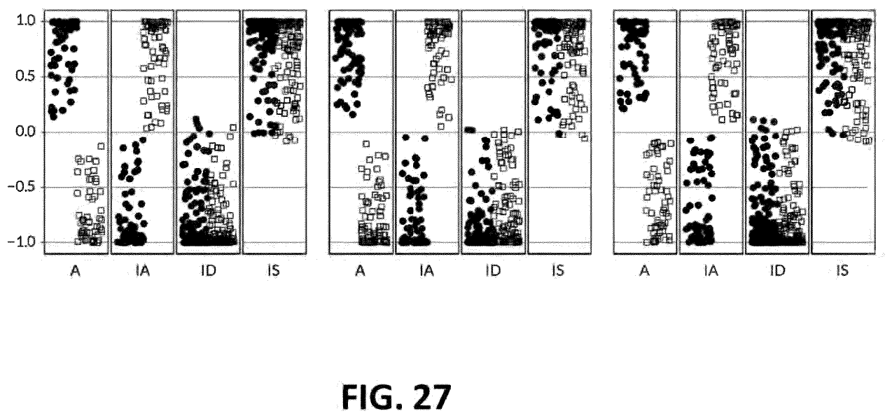

The Z-score population-based method and an ANN model were used on patient data (n=704) to retrospectively classify the stromal phenotypes of tumors from at least 17 different origins in the body (TABLE 44). No outcome data was associated with the classification, but the distribution of the four phenotypes was similar to the distribution of the four phenotypes classified in an analysis of 1,099 samples, representing samples of ovarian (n=392), colorectal (n=370), and gastric cancers (n=337), sequenced by RNA exome techniques as seen in FIG. 27.

TABLE 44
Stromal phenotypes of 704 patients from at least 17 different origins.

| Biomarker Call | N/Total Patient Samples | Percentage |
|---|---|---|
| IA (Z-score) | 102/704 | 14.5% |
| IA (ANN) | 120/704 | 17.1% |
| IS_(Z-score) | 246/704 | 34.9% |
| IS_(ANN) | 234/704 | 33.2% |
| A (Z-score) | 108/704 | 15.3% |
| A_(ANN) | 104/704 | 14.7% |
| ID (Z-score) | 247/704 | 35.1% |
| ID (ANN) | 245/704 | 34.8% |

### Example 17

### Latent Space

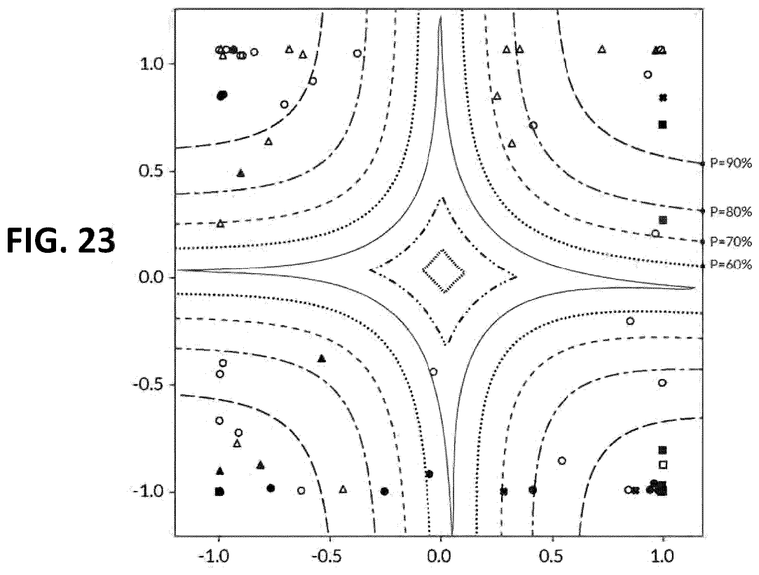

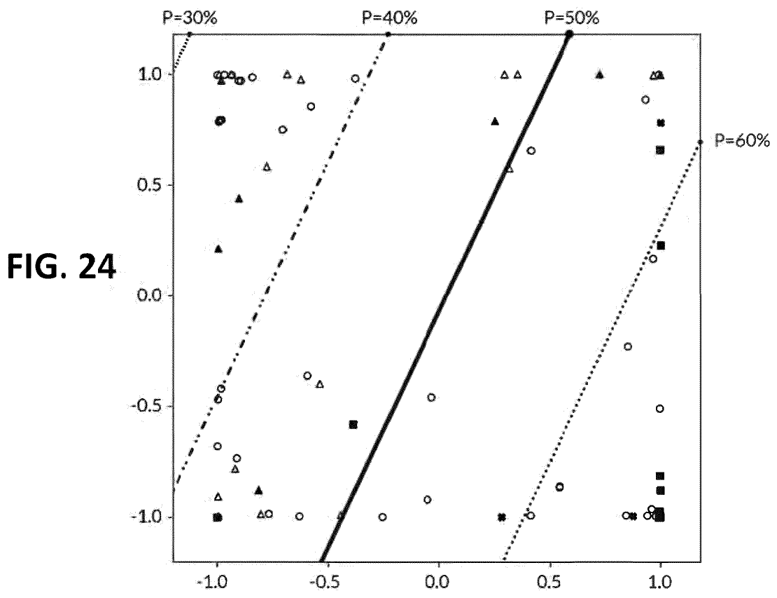

Projections of the probability function that resulted from the application of the ANN model to the data of Examples 7 and 12 were plotted in a latent space, represented by disease scores glyphs (Complete Response, CR; Partial Response, PR; Stable Disease, SD; Progressive Disease, PD). FIG. 23 shows a latent space visualization that provided a probability of the subtype call and could be used to inform physicians of biomarker confidence to help with treatment decisions. FIG. 24 shows a latent space visualization of a secondary logistic regression model that was trained on the latent space in order to learn the biomarker positive versus biomarker negative decision boundary based on patient outcome labels.

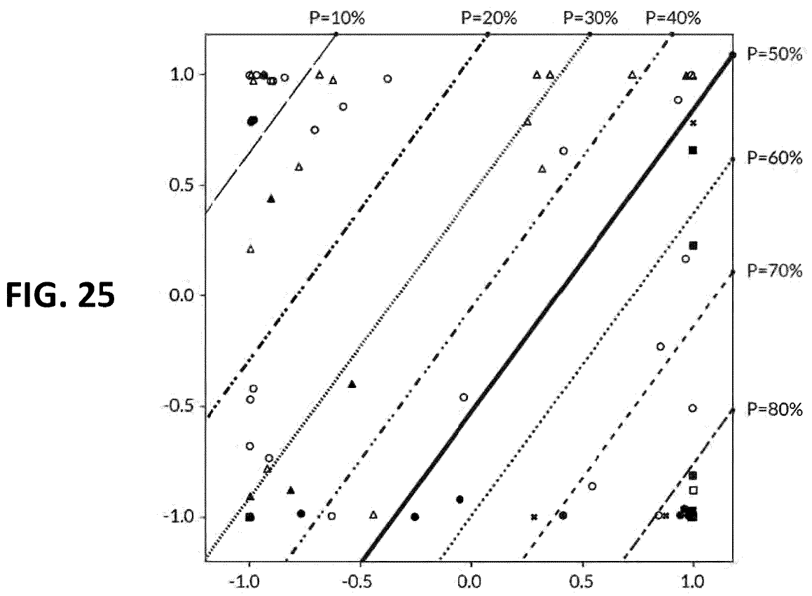

FIG. 25 shows a latent space visualization (logistic regression) trained with the patient data of Example 12, in which the subjects had Progression-Free Survival (PFS) of greater than 3 months. The disease scores of all the patients were used as glyphs to mark the probability score. In FIG. 26 a secondary logistic regression model was trained on the latent space in order to learn the biomarker positive versus biomarker negative decision boundary based on patient outcome labels for the ONCG100 data of Example 7, and the disease scores of the patients for whom there was biomarker data were plotted.

The curved contours in the figures occurred due to interaction terms between features in the model. In the latent space plots, the features were a Signature 1 score (e.g., a signature in which gene activation was correlated with endothelial cell signature activation) and a Signature 2 score (e.g., a signature in which activation was correlated with inflammatory and immune cell signature activation). In this context, the term interaction refers to a situation in which the effect of one feature on the prediction depends on the value of the other feature, i.e., when effects of the two features are not additive. For example, adding or subtracting features in the model implies no interaction; however, multiplying, dividing, or pairing features in the model implies interaction.

In the plots which predicted binary patient response, contours were parallel because the underlying logistic regression did not model interaction between features. The absence of interaction terms is one of the fundamental properties of logistic regression, which makes it less prone to overfitting and leads to good performance on small datasets. Thus, if there are no interaction terms in the model, the contours are always parallel.

On the other hand, the plots that predicted the phenotype (four classes, corresponding to four TME) had curved contours. Although the underlying model (a neuron) for each single phenotype class was equivalent to logistic regression, renormalization of the four phenotype class probabilities took place for the four logistic regressions, so the sum of the four phenotype class probabilities were equal to one. This was accomplished using the Softmax function, which is where interaction between the Signature 1 score and the Signature 2 score occurred. Consequently, this model produced curved contours.

### Example 18

### Application of ANN Method to Checkpoint Inhibitor Monotherapy in Cancer

In a clinical trial of any solid tumor with an anti-PD-1 or PD-1 therapy, such as tislelizumab, sintilimab, pembrolizumab, or nivolumab, a patient is selected for treatment based on RNA expression analysis of their TME. The patient has a solid tumor biopsy, which is processed, e.g., as a formalin-fixed paraffin-embedded block, and a recent slide cut from the block is transferred to a service provider for RNA expression determination via sequencing, e.g., using RNA-Seq, RNA exome, or microarray sequencing. RNA expression data are normalized and analyzed according to the algorithm of the instant invention.

Eligibility for treatment in the trial is based on greater than 60% probability of being biomarker positive (or IA+IS probability >60%), or on the basis of a logistical regression algorithm, trained, e.g., on the data of Example 7, and applied to the latent space based on progression-free survival rate greater than, e.g., 5 months (PFS>5) such that the patients in the PFS>5 subset are eligible for treatment.

The clinician is given one or more of the following outputs from this clinical trial assay that is used pursuant to an investigational device exemption (IDE): a binary yes/no answer, the probability for each TME class, the patient's probability plotted on the latent space plot with the probability contours and the historical outcome data, or the patient's probability plotted latent space plot overlaid with logistic regression of the probability based on PFS>5.

This clinical trial enrolls patients that were either naïve to checkpoint inhibitors or were ineligible to existing checkpoint inhibitors based on prior biomarker analyses (e.g. PD-L1 CPS>1). In this trial, greater than 20% of patients respond to treatment based on a PR or CR assessment (RECIST criteria).

The present disclosure provides a method for determining the tumor microenvironment (TME) of a cancer in a subject in need thereof (and, optionally, selecting the subject for a TME-class specific therapy) comprising applying a machine-learning classifier (e.g., an ANN disclosed herein) to a plurality of RNA expression levels obtained from a gene panel from a tumor tissue sample from the subject, wherein the machine-learning classifier identifies the subject as exhibiting or not exhibiting a TME selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G;
(b) the tumor is a solid tumor; and,
(c) the TME-class specific therapy comprises the administration of an anti-PD-1 or PD-1 therapy, such as tislelizumab, sintilimab, pembrolizumab, or nivolumab

The present disclosure also provides a method for treating a human subject afflicted with a cancer comprising administering a TME-class specific therapy to the subject, wherein, prior to the administration, the subject is identified as exhibiting or not exhibiting a TME determined by applying a machine-learning classifier (e.g., an ANN disclosed herein) to a plurality of RNA expression levels obtained from a gene panel from a tumor tissue sample obtained from the subject, wherein the TME is selected from the group consisting of IS (immune suppressed), A (angiogenic), IA (immune active), ID (immune desert), and combinations thereof, wherein
(a) the gene panel is (i) a geneset comprising the genes of Table 1 and 2, or a combination thereof or (ii) a geneset selected from the group consisting of genesets 23, 30, 46, 51, 60, 82, 108, 116, 139, 158, 73, 91, 121, 166, 169, 179, 185, 232, 200, 216, 241, 250, 263, and 278 of FIGS. 28A-28G; and
(b) the tumor is a solid tumor; and,
(c) the TME-class specific therapy comprises the administration of an anti-PD-1 or PD-1 therapy, such as tislelizumab, sintilimab, pembrolizumab, or nivolumab.

It is to be appreciated that the Detailed Description section, and not the Summary and Abstract sections, is intended to be used to interpret the embodiments. The Summary and Abstract sections can set forth one or more but not all exemplary embodiments of the present invention as contemplated by the inventor(s), and thus, are not intended to limit the present invention and the appended embodiments in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following embodiments and their equivalents.

The contents of all cited references (including literature references, patents, patent applications, and websites) that may be cited throughout this disclosure are hereby expressly incorporated by reference in their entirety for any purpose, as are the references cited therein.

What is claimed is:

1. A method for treating cancer in a human subject in need thereof, wherein the subject is afflicted with a tumor associated with an ovarian cancer, peritoneal cancer, fallopian cancer, uterine cancer, vaginal cancer, vulvar cancer, or cervical cancer and wherein the subject exhibits an angiogenic Tumor Microenvironment (TME), the method comprising:
(a) receiving a TME classification result of the subject indicating that the subject has been identified as exhibiting an angiogenic TME;
wherein the subject has been identified as exhibiting an angiogenic TME by applying, on a computer, an Artificial Neural Network (ANN) classifier to a plurality of RNA expression levels from a tumor tissue sample obtained from the subject;
wherein the RNA expression levels are obtained from a first signature gene panel comprising at least 30 genes selected from the group consisting of ABCC9, AFAP1L2, BACE1, BGN, BMP5, CAVIN2, COL4A2, COL8A1, COL8A2, CPXM2, CXCL12, EBF1, ECM2, EDNRA, ELN, EPHA3, FBLN5, GNAS, GNB4, GUCY1A1, HEY2, HSPB2, IL1B, ITGA9, ITPR1, JAM2, JAM3, KCNJ8, LAMB2, LHFPL6, LTBP4, MEOX1, MGP, MMP12, MMP13, NAALAD2, NFATC1, NOV, OLFML2A, PCDH17, PDE5A, PDGFRB, PEG3, PLSCR2, PLXDC2, RGS4, RGS5, RNF144A, RRAS, RUNX1T1, SELP, SERPINE2, SGIP1, SMARCA1, SPON1, STAB2, STEAP4, TBX2, TEK, TGFB2, TMEM204, TTC28, and UTRN, and from a second signature gene panel comprising at least 30 genes selected from the group consisting of ADAMTS4, AGR2, C10orf54, C11orf9, CAPG, CCL2, CCL3, CCL4, CD19, CD274, CD3E, CD4, CD79A, CD8B, CTLA4, CTSB, CXCL10, CXCL11, CXCL9, DUSP4, EIF5A, ETV5, FOLR2, GAD1, GZMB, HAVCR2, HFE, HMOX1, HP, IDO1, IFNA2, IFNB1, IFNG, IGFBP3, IGLL5, IQGAP3, LAG3, MEST, MST1, MT2A, MTA2, PDCD1, PDCD1LG2, PLA2G4A, PLAU, RAC2, REG4, RNH1, SERPINE1, SRSF6, STRN3, TGFB1, TIGIT, TIMP1, TLR9, TNFRSF18, TNFRSF4, TNFSF18, TRIM7, USF1, and ZIC2;
wherein applying the ANN classifier comprises determining a Signature 1 score based on the RNA expression levels obtained from the first signature gene panel and a Signature 2 score based on the RNA expression levels obtained from the second signature gene panel, wherein the subject is identified as exhibiting an angiogenic TME if the Signature 1 score is positive and the Signature 2 score is negative as compared to a population-based reference;
wherein the ANN classifier comprises an input layer, a hidden layer, and an output layer;
wherein the input layer comprises one or more nodes (neurons);
wherein each node (neuron) in the input layer corresponds to a gene in the gene panel; and
wherein the ANN classifier is trained with a training set comprising RNA expression levels for each gene in the gene panel in a plurality of samples obtained from a plurality of subjects, wherein each sample is assigned a TME classification; and
(b) administering an anti-VEGF/anti-DLL4 bispecific antibody that specifically binds to VEGF and DLL4 to the subject.

2. The method of claim 1, wherein the tumor tissue sample comprises intratumoral tissue.

3. The method of claim 1, wherein the RNA expression levels are transcribed RNA expression levels.

4. The method of claim 3, wherein the RNA expression levels are determined using Next Generation Sequencing (NGS).

5. The method of claim 4, wherein the RNA expression levels are subject to quantile normalization comprising transforming the RNA expression levels to a normal output distribution function.

6. The method of claim 1, wherein the TME classification assigned to each sample in the training set is determined by a population-based classifier.

7. The method of claim 1, wherein the hidden layer comprises 2 nodes (neurons).

8. The method of claim 7, wherein a hyperbolic tangent sigmoid activation function is applied to the hidden layer.

9. The method of claim 8, the method further comprising applying a logistic regression classifier comprising a Softmax function to the output layer of the ANN classifier, wherein the Softmax function is implemented through an additional neural network layer interposed between the hidden layer and the output layer.

10. The method of claim 9, wherein the Softmax function outputs an angiogenic TME class probability.

11. The method of claim 10, wherein the probability is overlaid on a latent space plot of the activation scores of the nodes of the ANN classifier.

12. The method of claim 11, wherein the logistic regression classifier is trained on the latent space.

13. The method of claim 9, wherein the logistic regression classifier is optimized for PFS (Progression-Free Survival).

14. The method of claim 9, wherein the logistic regression classifier is optimized for BOR (Best Objective Response), ORR (Overall Response Rate), MSS/MSI-high (Microsatellite Stable/Microsatellite Instability-high) status, PD-1/PD-L1 status, PFS (Progression-Free Survival), NLR (Neutrophil Leukocyte Ratio), Tumor Mutation Burden (TMB), or any combination thereof.

15. The method of claim 1, wherein the calculation of a Signature 1 score comprises:
(i) measuring the expression levels for each gene in the first signature gene panel;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and
(iv) adding all the values obtained in step (iii) and dividing the resulting number by the square root of the number of genes in the gene panel; wherein if the value obtained in (iv) is above zero, the signature score is a positive signature score, and wherein if the value obtained in (iv) is below zero, the signature score is a negative signature score.

16. The method of claim 1, wherein the calculation of a Signature 2 score comprises:
(i) measuring the expression level for each gene in the second signature gene panel;
(ii) for each gene, subtracting the mean expression value obtained from the expression levels of that gene in a reference sample from the expression level of step (i);
(iii) for each gene, dividing the value obtained in step (ii) by the standard deviation per gene obtained from the expression levels of the reference sample; and
adding all the values obtained in step (iii) and dividing the resulting number by the square root of the number of genes in the gene panel; wherein if the value obtained in (iv) is above zero, the signature score is a positive signature score, and wherein if the value obtained in (iv) is below zero, the signature score is a negative signature score.

17. The method of claim 1, wherein the anti-VEGF/anti-DLL4 bispecific antibody is navicixizumab.

18. The method of claim 1, the method further comprising (a) administering chemotherapy to the subject; (b) performing surgery on the subject; (c) administering radiation therapy to the subject; or (d) any combination thereof.

19. The method of claim 18, wherein the chemotherapy comprises paclitaxel or irinotecan.

20. The method of claim 1, wherein the tumor is relapsed.

21. The method of claim 1, wherein the tumor is refractory.

22. The method of claim 1, wherein the tumor is metastatic.

23. A method for treating cancer in a human subject in need thereof, wherein the subject is afflicted with a tumor associated with an ovarian cancer, peritoneal cancer, fallopian cancer, uterine cancer, vaginal cancer, vulvar cancer, or cervical cancer, the method comprising:
(a) on a computer, applying an ANN classifier to a plurality of RNA expression levels obtained from a tumor tissue sample obtained from the subject, wherein the RNA expression levels are obtained from a first gene panel comprising at least 30 genes selected from the group consisting of ABCC9, AFAP1L2, BACE1, BGN, BMP5, CAVIN2, COL4A2, COL8A1, COL8A2, CPXM2, CXCL12, EBF1, ECM2, EDNRA, ELN, EPHA3, FBLN5, GNAS, GNB4, GUCY1A1, HEY2, HSPB2, IL1B, ITGA9, ITPR1, JAM2, JAM3, KCNJ8, LAMB2, LHFPL6, LTBP4, MEOX1, MGP, MMP12, MMP13, NAALAD2, NFATC1, NOV, OLFML2A, PCDH17, PDE5A, PDGFRB, PEG3, PLSCR2, PLXDC2, RGS4, RGS5, RNF144A, RRAS, RUNX1T1, SELP, SERPINE2, SGIP1, SMARCA1, SPON1, STAB2, STEAP4, TBX2, TEK, TGFB2, TMEM204, TTC28 and from a second gene panel comprising at least 30 genes selected from the group consisting of ADAMTS4, AGR2, C10orf54, C11orf9, CAPG, CCL2, CCL3, CCL4, CD19, CD274, CD3E, CD4, CD79A, CD8B, CTLA4, CTSB, CXCL10, CXCL11, CXCL9, DUSP4, EIF5A, ETV5, FOLR2, GAD1, GZMB, HAVCR2, HFE, HMOX1, HP, IDO1, IFNA2, IFNB1, IFNG, IGFBP3, IGLL5, IQGAP3, LAG3, MEST, MST1, MT2A, MTA2, PDCD1, PDCD1LG2, PLA2G4A, PLAU, RAC2, REG4, RNH1, SERPINE1, SRSF6, STRN3, TGFB1, TIGIT, TIMP1, TLR9, TNFRSF18, TNFRSF4, TNFSF18, TRIM7, USF1, and ZIC2; wherein applying the ANN classifier comprises determining a Signature 1 score based on the RNA expression levels obtained from the first signature gene panel and a Signature 2 score based on the RNA expression levels obtained from the second signature gene panel, wherein the subject is identified as exhibiting an angiogenic TME if the Signature 1 score is positive and the Signature 2 score is negative as compared to a population-based reference; wherein the ANN classifier comprises an input layer, a hidden layer, and an output layer, wherein the input layer comprises one or more nodes (neurons), wherein each node (neuron) in the input layer corresponds to a gene in the gene panel; and wherein the ANN classifier is trained with a training set comprising RNA expression levels for each gene in the gene panel in a plurality of samples obtained from a plurality of subjects, wherein each sample is assigned a TME classification;
(b) classifying the TME as angiogenic based on the output of the ANN classifier; and
(c) administering an anti-VEGF/anti-DLL4 bispecific antibody that specifically binds to VEGF and DLL4 to the subject.

24. The method of claim 3, wherein the RNA expression levels are determined using RNA-Seq, EdgeSeq, PCR, Nanostring, whole exome sequencing (WES), or combinations thereof.

25. The method of claim 23, wherein the tumor is selected from the group consisting of tumors associated with ovarian cancer, peritoneal cancer, and fallopian cancer.

26. The method of claim 23, wherein the anti-VEGF/anti-DLL4 bispecific antibody is navicixizumab.
